(19) United States
(12) Patent Application Publication
(10) Pub. No.: US 2026/0183297 A1
(43) Pub. Date: Jul. 2, 2026

(54) SELECTION OF PATIENTS FOR THE TREATMENT OF FADS1-MEDIATED DISEASES OR DISORDERS USING FADS-1 INHIBITORS

(71) Applicant: AMGEN INC., Thousand Oaks, CA (US)

(72) Inventors: Murielle VENIANT-ELLISON, Thousand Oaks, CA (US); Ana E. MINATTI, Santa Monica, CA (US); Clarence H. HALE, Eugene, OR (US); Mei-Hsiu Michelle CHEN, Agoura Hills, CA (US); Marina STOLINA, Thousand Oaks, CA (US); Hubert ASTNER, Thousand Oaks, CA (US)

(73) Assignee: AMGEN INC., Thousand Oaks, CA (US)

(21) Appl. No.: 19/130,712

(22) PCT Filed: Nov. 21, 2023
(86) PCT No.: PCT/US2023/080701

§ 371 Date: May 16, 2025

Related U.S. Application Data
(60) Provisional application No. 63/384,862, filed on Nov. 23, 2022.
(60) Provisional application No. 63/592,804, filed on Oct. 24, 2023.

(51) Int. Cl. A61K 31/519; A61P 3/04; A61P 3/06; A61P 3/10; C07B 59/00; C07C 57/03; C12Q 1/6809; G01N 33/50; G01N 33/58

(52) U.S. Cl. CPC A61K 31/519; A61P 3/04; A61P 3/06; A61P 3/10; C07B 59/001; C07C 57/03; C12Q 1/6809; G01N 33/5044; G01N 33/58; C07B 2200/05

(57) ABSTRACT

The present disclosure provides techniques for accessing FADS1 activity in a patient. Also provided are techniques for determining the appropriateness of treatment of a patient with a FADS1 modulating (e.g., inhibiting) compound. This determination may be made by analyzing one or more biological indicators of FADS1-mediated disease or disorder in the subject. The one or more biological indicators may include one or more of a ratio of polyunsaturated fatty acids (“PUFAs”) in the subject, a relative abundance of one or more cell types, a relative abundance of one or more differentially expressed genes (“DEGs”) (or gene signatures, such as RNA, for such DEGs), and/or a relative abundance of one or more metabolites. Also disclosed are methods of using FADS1 inhibitors in methods of treating metabolic disorders and obesity.

### CROSS-REFERENCE TO RELATED APPLICATIONS

This application claims the benefit of priority to U.S. Provisional Patent Application No. 63/384,862, filed Nov. 23, 2022, and U.S. Provisional Patent Application No. 63/592,804, filed Oct. 24, 2023. Each of the foregoing applications is incorporated herein by reference in its entirety for all purposes.

### FIELD

The present disclosure provides techniques for using one or more biomarkers to determine fatty acid desaturase 1 (“FADS1”) activity levels in a subject. Also disclosed are methods of using FADS1 inhibitors in treating FADS1-mediated metabolic disorders and obesity in subjects in need of treatment. In several embodiments, subjects in need of treatment are those having one or more biomarkers of a FADS1-mediated disease or of a FADS1-mediated disorder. In several embodiments, subjects in need of treatment are those who have increased FADS1 activity relative to a healthy population.

### BACKGROUND

Polyunsaturated fatty acids (“PUFAs”) exert important physiological functions in the human body. PUFAs serve as sources of energy and structural components of cell membranes. PUFAs also regulate genes and are biosynthetic precursors of other physiologically relevant biomolecules, such as eicosanoids and endocannabinoids. Eicosanoids are signaling molecules that have multiple functions and regulate, among other things, the human inflammatory response. Endocannabinoids (e.g., N-arachidonoyl ethanolamine (anandamide) and 2-arachidonoyl glycerol (2-AG)) are endogenous ligands for the cannabinoid receptors which have been established to have a role in food intake and energy balance.

The pertinent part of the metabolic pathway of a certain PUFA, linoleic acid (“LA”), which leads to, among other things, the formation of anti- and pro-inflammatory eicosanoids and endocannabinoids, is shown in the scheme above. The desaturase enzymes, which catalyze certain steps in the conversion of LA to dihomo-gamma-linolenic acid (“DGLA”) and arachidonic acid (“AA”), are delta-6-desaturase (“D6D;” encoded by the gene Fatty Acid Desaturase 2 (“FADS2”)) and delta-5-desaturase (“D5D;” encoded by the gene Fatty Acid Desaturase 1 (“FADS1”)). Selectively inhibiting D5D activity reduces the amount of AA generated, while increasing the amount of DGLA. Such a pharmacological intervention reduces downstream generation of, for example, pro-inflammatory eicosanoids and endocannabinoids and leads to build-up of anti-inflammatory eicosanoids, both of which may overall ameliorate inflammation-related conditions and may improve energy balance. This is especially relevant in subjects with high intake of LA, for example, humans exposed to Western-style diets.

The FADS1-3 locus has been associated with many metabolic traits in human genome-wide association studies including fasting glucose, plasma lipids, and body weight. In addition to human genetic evidence supporting a role of FADS1/D5D in metabolic disorders, FADS1 knock out (“KO”) mice also show a phenotype with protection from diet-induced obesity including low body fat content, improved glycemic control, and decreased circulating lipid levels. In addition, the FADS1 KO mice are resistant to the development of arterial atheromatous plaque.

Desaturase enzyme activity has been linked to a variety of diseases, in particular metabolic and cardiovascular diseases, such as obesity, diabetes, nonalcoholic steatohepatitis (“NASH”), dyslipidemia, and coronary artery disease. Therefore, the pharmacological inhibition of D5D is a target of interest for treating metabolic, cardiovascular and other diseases.

### SUMMARY

One aspect of the disclosure provides a method of treating a FADS1-mediated disease or disorder in a subject in need of treatment, the method comprising: receiving information that compares a level of a biological indicator of FADS1-mediated disease from the subject to a reference level of the biological indicator; wherein the biological indicator is a ratio AA to DGLA; and administering to the subject a therapeutically effective amount of a FADS1 inhibitor compound if the level of biological indicator in the subject is greater than the reference level of biological indicator. Another aspect of the disclosure provides a method of detecting a FADS1-mediated disease or disorder in a subject, the method comprising: receiving information that compares a level of a biological indicator of FADS1-mediated disease from the subject to a reference level of the biological indicator; wherein the biological indicator is a ratio AA to DGLA. Another aspect of the disclosure provides a FADS1 inhibitor compound for use in a method of treating a subject in need of treatment who has a FADS1-mediated disease or disorder, the method comprising: receiving information that compares a level of a biological indicator of FADS1-mediated disease from the subject to a reference level of the biological indicator; wherein the biological indicator is a ratio AA to DGLA; and administering to the subject a therapeutically effective amount of the FADS1 inhibitor compound if the level of biological indicator in the subject is greater than the reference level of biological indicator.

Another aspect of the disclosure provides a method of treating a FADS1-mediated disease or disorder in a subject in need of treatment, the method comprising administering to the subject a therapeutically effective amount of a FADS1 inhibitor compound if the subject has an AA to DGLA ratio that is equal to or at least about 5:1. Another aspect of the disclosure provides a method of treating a FADS1-mediated disease or disorder in a subject in need of treatment, the method comprising administering to the subject a therapeutically effective amount of a FADS1 inhibitor compound if the subject has an AA to DGLA ratio that is equal to or at least about 6:1. Another aspect of the disclosure provides a method of treating a FADS1-mediated disease or disorder in a subject in need of treatment, the method comprising administering to the subject a therapeutically effective amount of a FADS1 inhibitor compound if the subject has an AA to DGLA ratio that is equal to or at least about 7:1. Another aspect of the disclosure provides a method of treating a FADS1-mediated disease or disorder in a subject in need of treatment, the method comprising administering to the subject a therapeutically effective amount of a FADS1 inhibitor compound if the subject has an AA to DGLA ratio that is equal to or at least about 15:2. Another aspect of the disclosure provides a method of treating a FADS1-mediated disease or disorder in a subject in need of treatment, the method comprising administering to the subject a therapeutically effective amount of a FADS1 inhibitor compound if the subject has an AA to DGLA ratio that is equal to or at least about 8:1. Another aspect of the disclosure provides a method of treating a FADS1-mediated disease or disorder in a subject in need of treatment, the method comprising administering to the subject a therapeutically effective amount of a FADS1 inhibitor compound if the subject has an AA to DGLA ratio that is equal to or at least about 17:2. Another aspect of the disclosure provides a method of treating a FADS1-mediated disease or disorder in a subject in need of treatment, the method comprising administering to the subject a therapeutically effective amount of a FADS1 inhibitor compound if the subject has an AA to DGLA ratio that is equal to or at least about 9:1.

Another aspect of the disclosure provides a method of treating a FADS1-mediated disease or disorder in a subject in need of treatment, the method comprising: receiving information that compares a level of a biological indicator of FADS1-mediated disease from the subject to a reference level of the biological indicator; wherein the biological indicator is a measured level of a PUFA; and administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator. Another aspect of the disclosure provides a method of treating a FADS1-mediated disease or disorder in a subject in need of treatment, the method comprising administering to the subject a therapeutically effective amount of a FADS1 inhibitor compound if the subject has a level of a biological indicator of FADS1-mediated disease that is greater than a reference level of the biological indicator; wherein the biological indicator is a measured level of a PUFA. Another aspect of the disclosure provides a method of detecting a FADS1-mediated disease or disorder in a subject, the method comprising: receiving information that compares a level of a biological indicator of FADS1-mediated disease from the subject to a reference level of the biological indicator; wherein the biological indicator is a measured level of a PUFA. Another aspect of the disclosure provides a FADS1 inhibitor compound for use in a method of treating a subject in need of treatment who has a FADS1-mediated disease or disorder, the method comprising: receiving information that compares a level of a biological indicator of FADS1-mediated disease from the subject to a reference level of the biological indicator; wherein the biological indicator is a measured level of a PUFA; and administering to the subject a therapeutically effective amount of the FADS1 inhibitor compound if the subject is determined to be one who would benefit from treatment with the FADS1 inhibitor compound as determined by the subject's level of the biological indicator compared to the reference level for the biological indicator. In several embodiments, the PUFA is linoleic acid, arachidonic acid, gamma-linoleic acid, adrenic acid, dihomo-gamma-linolenic acid, docosapenatenoic acid n-6, alpha-linolenic acid, eicosatetraenoic acid, eicosapentaenoic acid, docosahexaenoic acid, or a combination of the foregoing.

Another aspect of the disclosure provides a method of treating a FADS1-mediated disease or disorder in a subject in need of treatment, the method comprising: receiving information that compares a level of a biological indicator of FADS1-mediated disease from the subject to a reference level of the biological indicator; wherein the biological indicator is a measured level of a metabolite; and administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator. Another aspect of the disclosure provides a method of treating a FADS1-mediated disease or disorder in a subject in need of treatment, the method comprising administering to the subject a therapeutically effective amount of a FADS1 inhibitor compound if the subject has a level of a biological indicator of FADS1-mediated disease that is greater than a reference level of the biological indicator; wherein the biological indicator is a measured level of a metabolite. Another aspect of the disclosure provides a method of detecting a FADS1-mediated disease or disorder in a subject, the method comprising: receiving information that compares a level of a biological indicator of FADS1-mediated disease from the subject to a reference level of the biological indicator; wherein the biological indicator is a measured level of a metabolite. Another aspect of the disclosure provides a FADS1 inhibitor compound for use in a method of treating a subject in need of treatment who has a FADS1-mediated disease or disorder, the method comprising: receiving information that compares a level of a biological indicator of FADS1-mediated disease from the subject to a reference level of the biological indicator; wherein the biological indicator is a measured level of a metabolite; and administering to the subject a therapeutically effective amount of the FADS1 inhibitor compound if the subject is determined to be one who would benefit from treatment with the FADS1 inhibitor compound as determined by the subject's level of the biological indicator compared to the reference level for the biological indicator. In several embodiments, the metabolite is cholesterol, free cholesterol, total cholesterol, cholesterylester C20:4, malate, alpha-ketoglutarate, mannose, glucose, erythro-dihydrosphingosine (d18:0), 5-O-methylsphingosine (d18:1), erythro-sphingosine (d18:1), 3-O-methylsphingosine (d18:1), threo-sphingosine (d18:1), 1-hydroxy-2-amino-(cis,trans)-3,5-octadecadiene, 4-hydroxysphinganine (t18:0, phytosphingosine), sphingomyelin (d18:1,C23:0), sphingomyelin (d18:1,C24:0), ceramide (d18:1, C24:0), thromboxane B2, delta-12-prostaglandin D2, prostaglandin E2, prostaglandin D2, 12-hydroxyheptadecatrienoic acid (C17:[5,8,10]3), 14,15-dihydroxyeicosatrienoic acid (C20:cis[5,8,11]3), 11-hydroxyeicosatetraenoic acid (C20:cis[5,8,12,14]4), 13-hydroxyoctadecadienoic acid (13-HODE) (C18:cis[9]trans[11]2), arachidonic acid (C20:cis[5,8,11,14]4), docosahexaenoic acid (C22:cis[4,7,10,13,16,19]6), dihomo-gamma-linolenic acid (C20:cis[8,11,14]3), gamma-linolenic acid (C18:cis[6,9,12]3), docosapentaenoic acid (C22:cis[7,10,13,16,19]5), eicosapentaenoic acid (C20:cis[5,8,11,14,17]5), docosatetraenoic acid (C22:cis[7,10,13,16]4), stearic acid (C18:0), tryptophan, kynurenic acid, xanthurenic acid, histidine, leucine, isoleucine, valine, 3-hydroxyisobutyrate, glutamate, threonine, cysteine, sarcosine, plasma triglycerides, taurochenodeoxycholic acid, taurocholic acid, lysophosphatidylcholine (C18:0), lysophosphatidylcholine (C20:4), lysophosphatidylcholine (C17:0), lysophosphatidylethanolamine (C22:5), phosphatidylcholine (C18:0,C22:6), phosphatidylcholine (C18:0,C20:3), phosphatidylcholine (C18:1,C18:2), phosphatidylcholine (C16:1,C18:2), phosphatidylcholine (C18:0,C18:2), phosphatidylcholine (C16:0,C20:5), phosphatidylcholine (C16:0,C16:0), glycerol-3 phosphate, choline plasmalogen (C18,C20:4), myo-inositol, myo-inositolphospholipids, glycerol phosphate, phosphate lipid fraction, or a combination of the foregoing.

Another aspect of the disclosure provides a method of treating a FADS1-mediated disease or disorder in a subject in need of treatment, the method comprising: receiving information that compares a level of a biological indicator of FADS1-mediated disease from the subject to a reference level of the biological indicator; wherein the biological indicator is a measured level of a differentially expressed gene (“DEG”); and administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator. Another aspect of the disclosure provides a method of treating a FADS1-mediated disease or disorder in a subject in need of treatment, the method comprising administering to the subject a therapeutically effective amount of a FADS1 inhibitor compound if the subject has a level of a biological indicator of FADS1-mediated disease that is greater than a reference level of the biological indicator; wherein the biological indicator is a measured level of a DEG. Another aspect of the disclosure provides a method of detecting a FADS1-mediated disease or disorder in a subject, the method comprising: receiving information that compares a level of a biological indicator of FADS1-mediated disease from the subject to a reference level of the biological indicator; wherein the biological indicator is a measured level of a DEG. Another aspect of the disclosure provides a FADS1 inhibitor compound for use in a method of treating a subject in need of treatment who has a FADS1-mediated disease or disorder, the method comprising: receiving information that compares a level of a biological indicator of FADS1-mediated disease from the subject to a reference level of the biological indicator; wherein the biological indicator is a measured level of a DEG; and administering to the subject a therapeutically effective amount of the FADS1 inhibitor compound if the subject is determined to be one who would benefit from treatment with the FADS1 inhibitor compound as determined by the subject's level of the biological indicator compared to the reference level for the biological indicator. In several embodiments, the DEG is Serpinb1a, Gna14, Serpina3m, Hsd11b1, Cyp2c29, Akr1d1, Aldh1a1, Mmp19, Gyp27a1, Tymp, Elov12, Chkb, H2afj, Tnfaip811, Tmem86a, Sel113, Agap2, 4833411C07Rik, Elov4, Fat3, Eef1a2, Atp7a, Rgs2, Cenpe, Mfap2, Mlki67, Ctsh, Tnmb2, Ube, H6pd, Eepd1, Acss2, Aacs, Gm36827, Man2a2, Nudt18, Plagl1, TM4sf19, Atp6v0d2, Gm20056, Trem2, Il1rn, Mmp12, Cdk18, Efr3b, Tag1n2, Lurap1, Cp, I17rb, B230303O12Rik, Cfd, Sult1e1, Tdo2, Cyp2b9, Hao2, Cyp2b13, Cyp2a22, Acnat2, Ildr2, Rpl10a-ps1, Tm6sf2, Fitm1, Lpar1, C6, Cmah, Lbp, Arsg, Glra3, Lad1, A730063M14Rik, Ly6f, Foxi1, Crygc, Detb28, Wfdc9, Ph1da2, Aqp6, Gm16411, Adam7, Ppp2r5b, Slc6a7, Gpr50, Ahnak2, S100a6, Mmp14, Htr2b, Hpgds, Gm18537, Pclo, Adrb3, Gm38394, AC154232.2, Cadps, Adgrb2, Gm45470, Sdr9c7, Dsg1c, Slc17a1, Ces1c, Gss, 1810008I18Rik, Tlcd1, Snrk, Akr1c20, Gm19950, Ttr, Cbfa2t3, Acat2, Pmvk, Abcd3, Acacb, Arhgap27, Rnase9, Wfdc8, Ighv9-2, DerI3, Acap1, Ccl19, Tcf7, Xkrx, Trim46, Zfp369, Zfp871, Pcdhb21, Gm14288, Uprt, Atm, Dchs2, Cpcd1, Gm38357, Cck, Ckap2, Gm4419, 1600015I10Rik, Sez6I2, Prnd, Gn16702, S100a8, Pcdh12, Malat1, Kcnqlot1, ArI4c, Gm42549, Gm37310, Gm37776, Atp2a1, Ckm, Tnnt3, Adipoq, Fabp4, Lep, Retn, Hoxc8, Hoxc9, Cebpa, Dgat1, Dgat2, Elov13, Fas, Scd1, Srebf1, Hilpda, Lipe, Mg11, Plin1, Plin4, Pnpla2, Pnpla3, Ldah, Cs, Gckr, Me1, Pck1, Pdk4, Irs1, Acaa1a, Acads, Acox1, Cpt1a, Cpt1b, Hadhb, Ehhadh, Ppara, Ppargc1a, Cyp4a12a, Cyp4a12b, Cyp2e1, Adgre1, Agtr1a, Cc12, Cd14, Cd68, Il1b, Tnf, or a combination of the foregoing.

Another aspect of the disclosure provides a method of treating a FADS1-mediated disease or disorder in a subject in need of treatment, the method comprising: receiving information that compares a level of a biological indicator of FADS1-mediated disease from the subject to a reference level of the biological indicator; wherein the biological indicator is a measured level of a cell type; and administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator. Another aspect of the disclosure provides a method of treating a FADS1-mediated disease or disorder in a subject in need of treatment, the method comprising administering to the subject a therapeutically effective amount of a FADS1 inhibitor compound if the subject has a level of a biological indicator of FADS1-mediated disease that is greater than a reference level of the biological indicator; wherein the biological indicator is a measured level of a cell type. Another aspect of the disclosure provides a method of detecting a FADS1-mediated disease or disorder in a subject, the method comprising: receiving information that compares a level of a biological indicator of FADS1-mediated disease from the subject to a reference level of the biological indicator; wherein the biological indicator is a measured level of a cell type. Another aspect of the disclosure provides a FADS1 inhibitor compound for use in a method of treating a subject in need of treatment who has a FADS1-mediated disease or disorder, the method comprising: receiving information that compares a level of a biological indicator of FADS1-mediated disease from the subject to a reference level of the biological indicator; wherein the biological indicator is a measured level of a cell type; and administering to the subject a therapeutically effective amount of the FADS1 inhibitor compound if the subject is determined to be one who would benefit from treatment with the FADS1 inhibitor compound as determined by the subject's level of the biological indicator compared to the reference level for the biological indicator. In several embodiments, the cell type is an adipocyte (“Adipo”), a B cell (“Bcell”), an endothelial cell (“Endo”), a hepatocyte (“Hep”), a kupffer cell (“Kupff”), a myeloid cell (“Myel”), a natural killer cell (“NK”), a T cell (“Tcell”), or a combination of the foregoing.

As disclosed elsewhere herein, several embodiments pertain to treating a FADS1 mediated disease in a subject in need of treatment. Several embodiments disclosed herein pertain to the selection of patients for treatment using FADS1 inhibition (e.g., through administration of a FADS1 inhibitor). In several embodiments, in this selection step, subjects who are more likely to have a favorable to response to treatment are chosen for treatment. In several embodiments, patients with a higher likelihood of successful treatment are then treated using, for example, a FADS1 modulating compound (e.g., a FADS1 inhibitor compound). In several embodiments, the selection is based on patient data collected prior to administration of a FADS1 inhibitor. In several embodiments, the patient data is related to the measurement of one or more biological indicators for FADS1 activity (e.g., increased FADS1 activity). In several embodiments, the selection is based on patient data collected before, concurrent with, and/or after administration of a FADS1 inhibitor. In several embodiments, by inhibiting the FADS1 enzyme using one or more methods disclosed herein, FADS1 mediated diseases or disorders are treated.

Several embodiments disclosed herein provide a method of identifying a subject having increased FADS1 activity. In several embodiments, the method comprises measuring one or more biological indicators of FADS1-mediated disease or disorder in the subject. In several embodiments, the one or more biological indicators include one or more of a ratio of PUFAs in the subject, a relative abundance of one or more cell types, a relative abundance of one or more DEGs (or gene signatures, such as RNA, for such DEGs), and/or a relative abundance of one or more metabolites. In several embodiments, the method further comprises administering to the subject a FADS1 inhibitor compound.

Several embodiments disclosed herein provide a method of identifying a subject in need of treatment with a FADS1 inhibitor compound. In several embodiments, the method comprises measuring one or more biological indicators of FADS1-mediated disease or disorder in the subject. In several embodiments, the one or more biological indicators comprise, consist of, or consist essentially of one or more of a ratio of PUFAs in the subject, a relative abundance of one or more cell types, a relative abundance of one or more DEGs or gene signatures, and/or a relative abundance of one or more metabolites. In several embodiments, the method further comprises administering to the subject a FADS1 inhibitor compound.

Several embodiments disclosed herein provide a method of reducing body weight, reducing body-mass-index, treating obesity, treating a metabolic disorder, treating a cardiovascular disorder, treating diabetes, treating dyslipidemia, and/or treating non-alcoholic steatohepatitis (“NASH”) in a subject. In several embodiments, the method comprises measuring one or more biological indicators of FADS1-mediated disease or disorder in the subject. In several embodiments, the one or more biological indicators comprise, consist of, or consist essentially of one or more of a ratio of PUFAs in the subject, a relative abundance of one or more cell types, a relative abundance of one or more DEGs or gene signatures, and/or a relative abundance of one or more metabolites. In several embodiments, the method comprises administering to the subject a FADS1 inhibitor compound.

In several embodiments, as disclosed herein, the methods disclosed herein further comprise administering to the subject a FADS1 inhibitor compound. In several embodiments, the methods disclosed herein further comprise administering a dose of labeled DGLA to the subject and thereafter measuring a ratio of labeled AA to labeled DGLA. In several embodiments, the ratio of labeled AA to labeled DGLA allows calculation of the inhibitory concentration achieved using a FADS1 inhibitor. In several embodiments, the inhibitory concentration achieved using the FADS1 inhibitor is used to calculate the dose of inhibitor appropriate for the subject.

Another aspect of the disclosure provides a method of measuring a ratio of AA and DGLA in a subject; wherein the AA is isotopically labeled and the DGLA is isotopically labeled; wherein the ratio of AA to DGLA is measured by administering a dose of labeled DGLA to the subject and thereafter measuring a ratio of labeled AA to labeled DGLA.

Another aspect of the disclosure provides a compound represented by the following structure:

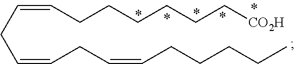 [SMILES: CCCCC/C=C\C/C=C\C/C=C\CCCCCCC(=O)O]

where: each “*” symbol indicates a position that may be isotopically enriched with ¹³C; and at least one “*” position is isotopically enriched with ¹³C.

Another aspect of the disclosure provides a compound represented by the following structure:

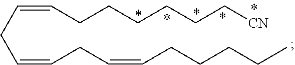 [SMILES: CCCCC/C=C\C/C=C\C/C=C\CCCCCCC#N]

where: each “*” symbol indicates a position that may be isotopically enriched with ¹³C; and at least one “*” position is isotopically enriched with ¹³C.

Another aspect of the disclosure provides a compound represented by the following structure:

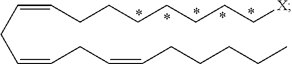

where: X is a halogen; each “*” symbol indicates a position that may be isotopically enriched with ¹³C; and at least one “*” position is isotopically enriched with ¹³C.

Another aspect of the disclosure provides a method of manufacturing a compound represented by the following structure:

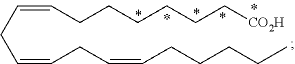 [SMILES: CCCCC/C=C\C/C=C\C/C=C\CCCCCCC(=O)O]

the method comprising reacting the following compound

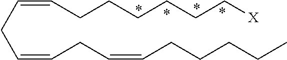

with KC*N; where: each “*” symbol indicates a position that may be isotopically enriched with ¹³C and at least one “*” position is isotopically enriched with ¹³C; and wherein X is an appropriate leaving group.

### BRIEF DESCRIPTION OF THE DRAWINGS

FIGS. 1(a)-(c) provide data indicating that FADS1 activity was increased in obese humans and rodent models. FIG. 1(a) provides flow charts showing n-3 PUFA and n-6 PUFA pathways. As shown, FADS1 mediates D5D of n-6 PUFA, DGLA to AA and n-3 PUFA, eicosatetraenoic acid (“ETA”) to eicosapentaenoic acid (“EPA”). FIG. 1(b) provides a comparison of the lean, overweight, and obese male human plasma AA/DGLA ratios as a surrogate indicator of FADS1 activity; n=8-41/group; data are presented as minimum to maximum. FIG. 1(c) provides a comparison of the AA/DGLA ratio as a surrogate of FADS1 activity in lean and high-fat diet (“HFD”)-induced obese (“DIO”) male C57Bl/6 mice on 12 weeks of HFD. n=8/group. FIG. 1(b,c) t-test. *P<0.05, ****P<0.00001, respectively.

FIGS. 2(a)-(h) provide data supporting that Fads1 KO mice are resistant to HFD-induced obesity and have improved metabolic profiles. FIG. 2(a) shows a comparison of age-matched WT and Fads1 KO mice on 12 weeks of HFD. The data demonstrate that Fads1 KO are resistant to HFD-induced body weight gain. FIG. 2(b) shows that Fads1 KO mice have lower fat mass and FIG. 2(c) shows that Fads1 KO mice lower lean mass relative to their WT littermates. FIG. 2(d) shows that food intake measured at 7 weeks was not significantly altered in the Fads1 KO mice relative to WT mice. FIG. 2(e) shows that the Fads1 KO mice have lower insulin relative to WT mice. FIG. 2(f) shows that Fads1 KO mice improved glucose tolerance during GTT relative to WT mice. FIG. 2(g) shows that Fads1 KO mice lower cholesterol relative to WT mice. FIG. 2(h) shows that no difference in plasma triglyceride levels was found; n=8-9/group. Data are presented as mean±SEM. (a-c) Two-way ANOVA with Sidak's test for multiple comparisons; (d-h) t-test. *P<0.05, **P<0.01, ***P<0.001, ****P<0.00001.

FIGS. 3(a)-(l) provide data supporting that HFD-induced obese Fads1 KO mice have lower respiratory exchange ratio (“RER”) and increased dark cycle energy expenditure. Fads1 KO and WT mice were placed on an HFD for 12 weeks; n=24 mice/group. FIG. 3(a,b) show oxygen consumption in the two groups, FIG. 3(c,d) shows carbon dioxide production in the two groups, FIG. 3(e,f) shows RER measurement in the two groups, FIG. 3(g,h) shows activity measurements taken at 0, 6, and 12 weeks of HFD feeding in the two groups. In FIGS. 3(a, c, e, g) each data point represents a rolling average of six time points where dark cycles (6:00 p.m. to 6:00 a.m.) are shown by a outlined box. In FIGS. 3(b, d, f, h) measurements were averaged and displayed as mean±SEM for each day or night over time. Two-way ANOVA with Sidak's test for multiple comparisons; *P<0.05, **P<0.01, ***P<0.001, ****P<0.00001. FIGS. 3(i-l) provide plots of light and dark cycle energy expenditures in kcal/hour versus body weight after 6 and 12 weeks of HFD feeding; least squares multiple linear regression model including body weight and genotype.

FIGS. 4(a)-(d) show RNA-seq analysis of liver, epididymal (“EPI”) white adipose tissue (“WAT”), and inguinal (“ING”) WAT of Fads1 KO mice versus WT on 12 weeks of HFD feeding. FIG. 4(a) provides volcano plots that represent DEGs in Fads1 KO versus WT of liver, EPI WAT, and ING WAT: genes with Benjamini-Hochberg (BH)-adjusted P value <0.01 and fold change ≥2 or ≤0.5 are color marked. FIG. 4(b) show averaged cellular composition in liver, EPI WAT and ING WAT of Fads1 KO and WT mice analyzed by SCDC using scRNA-Seq of corresponding mouse organs as a reference. Cell types are abbreviated as follows: adipocytes (“Adipo”), B cell (“Bcell”), endothelial (“Endo”), hepatocytes (“Hep”), kupffer (“Kupff”), myeloid (“Myel”), natural killer (“NK”), T cell (“Tcell”). Kyoto Encyclopedia of Genes and Genomes (“KEGG”) analysis of differentially altered metabolic pathways in the liver is provided in FIG. 4(c) and EPI WAT in FIG. 4(d) for Fads1 KO mice relative to WT mice; number of unique genes with significantly altered expression in each pathway are shown; n=4-8/group.

FIGS. 5(a)-(b) demonstrate that Fads1 knockout mice on high-fat diet have altered expression of metabolic genes, increased adiponectin levels and decreased inflammatory markers in adipose tissue. FIG. 5(a) provides a comparison of metabolic gene expression changes in EPI WAT, ING WAT, and liver of Fads1 KO mice relative to WT littermates; genes with Benjamini-Hochberg-adjusted P value <0.01 and fold change ≥2 or ≤0.5 are color-marked. FIG. 5(b) provides a comparison of plasma, EPI WAT and ING WAT of adiponectin, leptin. PAI-1, and MCP-1 levels in WT versus Fads1 KO mice; (a-b) data are shown as minimum to maximum. n=3-8/group. t-test *P<0.05, **P<0.01, ***P<0.001.

FIGS. 6(a)-(b) demonstrate that Fads1 KO mice have altered fatty acid composition in plasma lipid subfractions. FIG. 6(a) provides date for the fatty acid concentration of total plasma and each of the 4 major lipid subfractions (phospholipids, free fatty acids, cholesterol ester, and triglycerides) in HFD WT and Fads1 KO mice. FIG. 6(b) provides fatty acid composition of total plasma and each of the four lipid subfractions in the plasma of WT and Fads1 KO mice. (a-b) n=3 samples per genotype (each sample pooled from 2 mice). (a) Data are shown as minimum to maximum. t-test *P<0.05, **P<0.01, ***P<0.001.

FIGS. 7(a)-(c) show metabolomic analysis of Fads1 KO mice relative to WT on HFD. FIG. 7(a) provides a comparison of the plasma metabolite profile in Fads1 KO versus WT mice after 11 weeks of HFD feeding. The ratio of Fads1 KO metabolite levels relative to WT are expressed. Only metabolites with ratio of ≥1.15 or ≤0.85 and P-values ≤0.05 are shown; n=4-6/group. t-test. *P<0.05, **P<0.01, ***P<0.001, ****P<0.00001. FIG. 7(b) provides a heatmap of the top IPA Disease and Biofunctions affected based on the metabolite profile of Fads1 KO mice relative to WT mice on HFD. FIG. 7(c) provides a heatmap comparing the common and differential Disease and Biofunctions altered in the plasma metabolome and transcriptome of liver, EPI WAT, and ING WAT of Fads1 KO mice relative to WT mice. In FIGS. 7(b-c), only Disease and Biofunctions with P-values <0.05 are shown.

FIGS. 8(a)-(i) provide data demonstrating that a FADS1 small molecule inhibitor lowered body weight and improved metabolic parameters in DIO mice. Compound A FIG. 8(a) is a FADS1 inhibitor that, as shown in FIG. 8(b), has >1200× selectivity for human FADS1 over human FADS2 as determined in FADS1 and FADS2 cell-based assays. Similar potency was observed in FIG. 8(b) mouse FADS1 cell-based assay and FIG. 8(c), which provides mouse in vivo FADS1 potency assay. Treatment with 10 mg/kg and 30 mg/kg of Compound A once a day for 54 days in DIO mice lowered body weight, as shown in FIG. 8(d), and in vivo residual liver FADS1 activity, as shown in FIG. 8(e), without affecting food intake, as shown in FIG. 8(f). Compound A treatment lowered insulin (FIG. 8(g)), cholesterol (FIG. 8(h)), and triglyceride levels (FIG. 8(i)) (n=7-10/group). Data are presented as mean±SEM. FIG. 8(d) Two-way ANOVA with Sidak's test for multiple comparisons; FIG. 8(e-i) One-way ANOVA with Dunnett's test for multiple comparison. *P<0.05, **P<0.01, ***P<0.001, ****P<0.00001. ANOVA, analysis of variance; DIO, diet-induced obese; FADS1, fatty acid desaturase 1; FADS2, fatty acid desaturase 2; MW, molecular weight; POC, percent of control; SEM, standard error of the mean.

FIGS. 9(a)-(j) provide data for the indirect calorimetry measurement of Compound A-treated DIO mice or vehicle treated mice. FIG. 9(a) provides a comparison of body weight, FIG. 9(b) provides light and dark cycle energy expenditure versus body weight, FIGS. 9(c,d) provide oxygen consumption, FIGS. 9(e,f) provide carbon dioxide production, FIGS. 9(g,h) provide RERs, and FIGS. 9(i,j) provide activity. DIO mice were treated with vehicle or Compound A (30 mg/kg) for 42 days with measurement taken at the indicated times. In FIGS. 9(c, e, g, i), each data point represents a rolling average of 3 days, and dark cycles (6:00 p.m. to 6:00 a.m.) are shown by an outlined box. FIG. 9(d, f, h, j) measurements were averaged and displayed as mean±SEM for each day or night over time. FIG. 9(b) Least squares multiple linear regression model including body weight and treatment. FIG. 9(a, d, f, h, j) Two-way ANOVA with Sidak's test for multiple comparisons; *P<0.05, **P<0.01, ***P<0.001, ****P<0.00001. n=10-11 mice/group.

FIGS. 10(a)-(d) provide RNA-seq analysis of aged-matched Fads1 KO and WT DIO mice treated with either vehicle or 30 mg/kg of Compound A for 54 days. FIG. 10(a) provides radar charts of commonly altered genes in liver, EPI WAT and ING WAT of Fads1 KO mice in Compound A-treated mice relative to WT vehicle-treated mice. FIG. 10(b) provides averaged cellular composition in the liver, EPI and ING WAT of Fads1 KO, WT+ Compound A and WT mice by SCDC analysis using scRNA-Seq of corresponding mouse organs as a reference. Cell types are abbreviated as follows: Adipo, Bcell, Endo, Hep, Kupff, Myel, NK, Tcell. Ingenuity Pathway Analysis (“IPA”) analysis identified the common and unique pathways affected by Fads1 KO or inhibition in FIG. 10(c) EPI WAT and FIG. 10(d) liver; n=6-9/group.

FIGS. 11(a)-(c) provide data showing that no difference in FADS2 activity was observed in obese human and mice. Higher FADS1 but not FADS2 activity was observed in HFD-induced obese mice. FIG. 11(a) shows that no difference in plasma GLA/LA ratio, a surrogate marker of FADS2 n-6 activity, was observed in overweight or obese human males relative to lean subjects; n=8-41/group. FIG. 11(b) similarly shows, no difference in plasma GLA/LA ratio were observed in DIO mice fed with HFD for 12 weeks (n=7-8/group). In FIG. 11(c), SC-26196 is a FADS2 specific inhibitor that does not affect FADS1 activity as shown in vitro human FADS1, FADS2 and mouse FADS1 activity assays. FIG. 11(a,b) Data are presented as minimum to maximum; t-test. FIG. 11(c-d) n=4-8/group; Data shown as mean±SEM.

FIG. 12 shows that inhibition of FADS2 activity by SC-26196 affects not only the plasma GLA/LA ratio but also the plasma AA/DGLA ratio in DIO mice. FIG. 12 One-way ANOVA with Dunnett's test for multiple comparisons, *P<0.05, **P<0.01, ***P<0.001, ****P<0.0001.

FIG. 13(a)-(h) demonstrate that Compound A-treated DIO mice showed altered expression of metabolic genes, increased adiponectin levels, and decreased inflammatory markers. A comparison of metabolic gene expression changes is provided in the FIG. 13(a) liver, FIG. 13(b) EPI WAT, FIG. 13(c) ING WAT of vehicle-treated WT DIO mice (WT), Compound A (30 mg/kg) treated WT DIO mice (WT+A), and vehicle-treated Fads1 KO DIO mice (KO). n=6-9/group. IPA analysis of top predicted upstream regulators is provided in FIG. 13(d) liver and 13(e) EPI WAT of Compound A-treated WT DIO mice (WT+A) and Fads1 KO DIO mice (KO) relative to vehicle-treated WT DIO mice (WT); predicted upstream regulators with P value of overlap <0.01 and activation z-score are shaded. FIG. 13(f) provides a comparison of adiponectin, leptin, PAI, and MCP-1 levels in plasma. EPI WAT, and ING WAT in WT, Compound A-treated WT, and Fads1 KO DIO mice; n=7-9/group. FIG. 13(a-c, f) Data are presented as minimum to maximum. One-way ANOVA with Dunnett's test for multiple comparison relative to vehicle-treated WT. *P<0.05, **P<0.01, ***P<0.001, ****P<0.00001. FIG. 13(g) provides volcano plots representing DEGs in Fads1 KO mice versus WT and in 30 mg/kg Compound A-treated (WT+A) group versus vehicle-treated WT (WT) in liver, EPI and ING WAT of diet-induced obese mice; the number of genes with Benjamini-Hochberg-adjusted P value <0.01 and fold change ≥2 or ≤0.5 are color-marked and provided in a Venn diagram format that represents the number of uniquely or similarly altered genes in the liver and fat tissues in Fads1 KO versus WT or WT+A versus WT cohorts. FIG. 13(h) provides a comparison of liver PAI-1 and MCP-1 levels in WT, Compound A-treated WT, and Fads1 KO DIO mice.

FIGS. 14(a) and (b) provide data showing that FADS1 inhibition decreased endocannabinoid levels in the liver and adipose tissue. Comparison of endocannabinoids, 2-arachidonyl-glycerol (“2-AG”) and anandamide (“AEA”), levels in liver (FIG. 14(a)) and ING WAT (FIG. 14(b)) of vehicle-treated WT DIO mice (WT), Compound A (30 mg/kg) treated WT DIO mice (WT+A), and vehicle-treated Fads1 KO DIO mice (KO); data are presented as minimum to maximum; n=8-6/group. One-way analysis of variance with Dunnett's test for multiple comparison. *P<0.05, **P<0.01, ***P<0.001.

FIGS. 15(a)-(f) show that FADS1 inhibition increased expression of PPARα target genes with no significant effect on hepatic triglyceride content and steatosis-related genes. A comparison of liver triglyceride levels in WT and Fads1 KO mice on chow diet (FIG. 15(a)) or 12 weeks of HFD (FIG. 15(b)) is provided; n=4-8/group. A comparison of liver triglyceride level in vehicle-treated Fads1 KO mice and WT DIO mice after 8 weeks of vehicle or 10 or 30 mg/kg Compound A treatment is provided (FIG. 15(c)); data are presented as minimum to maximum; n=8/group. FIG. 15(d) provides a liver histology assessment of WT DIO mice treated with vehicle or Compound A for 24 days, and age-matched vehicle-treated Fads1 KO DIO mice. Compound-A (10 mg/kg) treated WT DIO mice and Fads1 KO DIO mice have similar or smaller sized vacuoles (H&E staining) and reduced lipid content (Oil-red-O staining) when compared to WT vehicle DIO mice suggesting no worsening of hepatic steatosis with Compound A treatment or loss of Fads1 expression; n=3/group. Representative images shown. Hepatic gene expression of steatosis-related genes are shown in FIG. 15(e) and PPARα target genes in Fads1 KO mice relative to WT littermates after 8 weeks of HFD are shown in FIG. 15(f); genes with BH-adjusted P value <0.01 and fold change ≥2 or ≤0.5 are color-marked. n=4-6/group. FIG. 15(a,b) t-test; FIG. 15(c) One-way ANOVA with Dunnett's test for multiple comparison.

FIGS. 16(a)-(b) demonstrate that Compound A-treated DIO mice have altered fatty acid composition in plasma lipid subfractions relative to vehicle-treated DIO mice. FIG. 16(a) provides fatty acid concentration of the total plasma and each of the 4 major lipid subfractions (phospholipids, free fatty acids, cholesterol ester, and triglycerides) of WT DIO mice that were treated with either vehicle (WT) or 30 mg/kg of Compound A (WT+A) for 54 days. FIG. 16(b) provides fatty acid composition of total plasma and each of the 4 plasma lipid subfractions of vehicle treated (WT) and 30 mg/kg Compound A-treated WT DIO mice (WT+A). (a-b) n=3 samples per group (each sample pooled from 2 mice). (a) Data are shown as minimum to maximum. t-test *P<0.05, **P<0.01, ***P<0.001.

FIG. 17a provides data for the % conversion of ¹³C₅-DGLA to ¹³C₅-AA for each of Compounds B-E as measured using ¹³C₅-DGLA and ¹³C₅-AA from blood plasma. FIG. 17b provides the % inhibition for each of Compounds B-E as measured using ¹³C₅-DGLA and ¹³C₅-AA collected from blood plasma. A dose response curve was generated for Compound F, which is shown in FIG. 17c.

### DETAILED DESCRIPTION

Chronic and persistent inflammation contributes to disease pathology of obesity and its comorbidities. FADS1 is a key enzyme for the synthesis of AA from DGLA. AA is a precursor of many pro-inflammatory eicosanoids. DGLA, on the other hand, is a precursor to several anti-inflammatory eicosanoids. Several embodiments, disclosed herein pertain to the treatment of FADS1 mediated diseases, disease states, and/or disorders. In several embodiments, the method includes selecting a subject suffering from a FADS1-mediated disease or disorder. In several embodiments, the subject is selected for treatment based on the level of FADS1 activity in the subject. In several embodiments, the subject has elevated FADS1 activity. In several embodiments, where the subject has elevated FADS1 activity, the method includes administering a FADS1 inhibitor to the subject. In several embodiments, the FADS1 activity of a subject is measured before, during, or after treatment. In several embodiments, the FADS1 activity of a subject is indicated by the AA/DGLA ratio in the subject (or by other biological indicators as disclosed elsewhere herein). In several embodiments, the FADS1 activity level in the subject is compared to the FADS1 activity level of a subject (or a population of subjects) not having a FADS1-mediated disease or disorder. In several embodiments, the FADS1 activity, as indicated by the AA/DGLA ratio (or by other biological indicators as disclosed elsewhere herein), is elevated in obese subjects (e.g., humans). Inhibition of FADS1 activity may alleviate obesity and its metabolic comorbidities. U.S. Application Publication Nos. US2021/0171529 and US2021/0188874 describe families of FADS1 inhibitor compounds as agents for treating metabolic or cardiovascular disorders.

The section headings used herein are for organizational purposes only and are not to be construed as limiting the subject matter described. Features disclosed under one heading (such as a composition) can be used in combination with features disclosed under a different heading (a method of manufacture or treating).

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

The term “pharmaceutically acceptable” as used herein refers to generally recognized for use in subjects, particularly in humans.

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherents, glidants, disintegrants, flavoring agents, coatings, binders, sweeteners, lubricants, sorbents, preservatives, and the like.

The term “pharmaceutically acceptable salt” refers to a salt of a compound that possesses the desired pharmacological activity of the parent compound and that is not biologically or otherwise undesirable for its end use. Pharmaceutically acceptable salts include, for example, acid addition salts formed with inorganic acids (e.g., hydrochloric acid, hydrobromic acid, sulfuric acid, nitric acid, phosphoric acid) or formed with organic acids (e.g., acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid). Pharmaceutically acceptable salts also include, for example, salts formed when an acidic proton present in the parent compound either is replaced by a metal ion (e.g., an alkali metal ion, an alkaline earth ion, or an aluminum ion) or associates with an organic base (e.g., ethanolamine, diethanolamine, triethanolamine, N-methylglucamine, dicyclohexylamine). Additionally, the salts of the compounds described herein, can exist in either hydrated or anhydrous form or as solvates with other solvent molecules.

The term “patient” or “subject” as used herein refers to humans and mammals, including, but not limited to, primates, cows, sheep, goats, horses, dogs, cats, rabbits, rats, and mice. In one embodiment, the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

As used herein, the term “relative abundance” is a comparison between the abundance of a particular feature in a test subject (e.g., a subject in need of treatment) relative to the abundance of that same feature in a non-test subject or population of non-test subjects. A non-test subject or population of non-test subjects may include healthy subjects and/or those that are not in need of treatment.

As used herein, the term “healthy subject” is a person having average characteristics of a population of individuals that are healthy. Healthy is given its plain and ordinary meaning and includes subjects who lack one or more or all FADS1-mediated diseases and/or disorders. A healthy population may be one including individuals having a body mass index below about 25, about 22.5, or about 20. A healthy population may be one including individuals having a body mass index above about 18.5. A healthy population may be one including individuals not suffering from obesity, a metabolic disorder, a cardiovascular disorder, diabetes, dyslipidemia, and/or NASH.

The term “isotopologue” refers to a species in which the chemical structure differs from a specific compound of this invention only in the isotopic composition thereof.

Disclosed herein are isotopically enriched compounds. It will be recognized that some variation of natural isotopic abundance occurs in a synthesized labeled compound as disclosed herein depending upon the origin of chemical materials used in the synthesis. Thus, a preparation of a labeled compound will contain small amounts of labeled isotopologues. In the labeled compounds disclosed herein any atom not specifically designated as a particular isotope is meant to represent any stable isotope of that atom. Unless otherwise stated, when a position is designated specifically as “C” or “carbon” or is undesignated, the position is understood to have carbon at its natural abundance isotopic composition. Also, unless otherwise stated, when a position is designated specifically as “¹³C” or as being “isotopically enriched,” the position is understood to have ¹³C at an abundance that is at least 100 times greater than the natural abundance of ¹³C.

The term “isotopic enrichment factor” as used herein means the ratio between the isotopic abundance and the natural abundance of a specified isotope. In several embodiments, a compound of this invention has an isotopic enrichment factor for each designated ¹³C atom is equal to or greater than about: 100, 500, 1000, 1500, 2000, 2500, 3000, 4000, 5000, or ranges including and/or spanning the aforementioned values. In several embodiments, an isotopically enriched compound of this invention has an isotopic enrichment factor for each designated ¹³C atom is equal to or greater than about: 100, 500, 1000, 1500, 2000, 2500, 3000, 4000, 5000, or ranges including and/or spanning the aforementioned values.

The term “halogen” as used herein refers to —F, —Cl, —Br, or —I.

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula.

When referring to numerical values, the terms “or ranges including and/or spanning the aforementioned values” (and variations thereof) is meant to include any range that includes or spans the aforementioned values. To illustrate, when a value is expressed as “20, 30, 40, 50, or ranges including and/or spanning the aforementioned values,” this includes each particular value provided (e.g., 20, 30, 40, and/or 50) or any ranges spanning or including any two values provided (e.g., 20 to 50, 20 to 40, 20 to 30, 30 to 50, 30 to 40, or 40 to 50).

The following description provides context and examples, but should not be interpreted to limit the scope of the inventions covered by the claims that follow in this specification or in any other application that claims priority to this specification. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

### INTRODUCTION

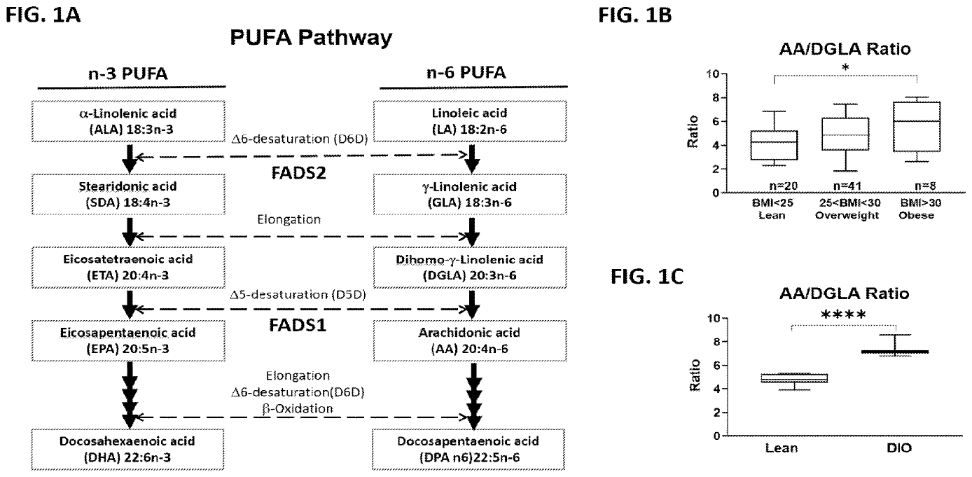

Dietary intake and endogenous synthesis of PUFAs and their physiological regulation impact human health and disease. Two main species of PUFAs, the omega-3 (n-3) and omega-6 (n-6) fatty acids (“FA”) can be consumed or synthesized endogenously from primary precursors, n-3 alpha-linolenic acid (“ALA”) or n-6 LA, which are essential FAs that cannot be synthesized by mammals. These FAs are metabolized by fatty acid desaturases and fatty acid elongases sequentially. The first and rate-limiting enzyme is FADS2, also known as D6D as it desaturates at the sixth carbon in LA or ALA, converting it to gamma-linolenic acid (“GLA”) or stearidonic acid, respectively (FIG. 1(a)). Subsequent elongation of GLA and stearidonic acid generates DGLA and ETA, respectively (FIG. 1(a)). DGLA and ETA are desaturated by FADS1, also known as D5D, as it desaturates at the fifth carbon in the fatty acid chain, and the respective products of the FADS1 enzymatic action are n-6 AA and n-3 EPA acid (FIG. 1(a)). Both AA and EPA are metabolized extensively by enzymes, including cyclooxygenases and lipoxygenases, to form bioactive eicosanoids, such as prostaglandins, thromboxanes, and leukotrienes. These eicosanoids play important roles in metabolism and inflammation, with the AA-derived eicosanoids being predominantly pro-inflammatory and the EPA-derived eicosanoids being anti-inflammatory.

Obesity and its associated comorbidities have become a worldwide public health concern. Increased rates of obesity over the past several decades have been partly attributed to the westernized diet, which have a high n-6 to n-3 ratio (˜10:1 to 20:1). This high ratio may contribute to the development of inflammation, cardiovascular diseases, cancer, and autoimmune disease. Excess n-6 PUFAs, such as AA, can lead to excess pro-inflammatory eicosanoids and other oxylipins. Obesity and metabolic disorders may in part be a consequence of the excess eicosanoid-mediated inflammatory damage. Humans depend on the consumption and generation of PUFAs by FADS2 and FADS1, encoded by their respective genes FADS2 and FADS1. The differences in the expression of FADS1 by the different alleles at the FADS locus has allowed the identification of many associations with traits and diseases from genome-wide association studies (GWAS). Notably, the highest association of the FADS locus to date is with AA concentrations (P=3×10⁻⁹⁷¹), and more specifically AA/DGLA concentrations (P=2×10⁻³⁶¹). The minor C alleles of FADS1 single nucleotide polymorphisms (“SNPs”) rs174556 and rs174547 are associated with reduced FADS1 activity and lower body weight or waist circumference. Additionally, improved metabolic phenotypes have also been observed in Fads1 KO mice when fed a normal chow diet or a HFD. Together, the phenotypes associated with FADS1 in both humans and mice suggest that reducing FADS1 activity could present therapeutic value for the treatment of obesity and associated metabolic comorbidities.

In view of the foregoing, several embodiments provided herein pertain to a method of determining if a subject is a candidate for treatment with a FADS1 inhibitor compound. In several embodiments, a candidate for treatment with a FADS1 inhibitor compound may be a subject who is suffering from or is at risk of suffering from a FADS1 mediated disorder or disease. In several embodiments, the method comprises determining the level of a biological indicator in a biological sample from the subject. In several embodiments, the level of the biological indicator provides information regarding whether the patient suffers from or is at risk of suffering from a FADS1-mediated disease or disorder. In several embodiments, the level of biological indicator is compared to a reference level of biological indicator from a different subject (or a population of subjects) who does not suffer from (or is not at risk of developing) a FADS1-mediated disease or condition. In several embodiments, if the level of the biological indicator in the subject indicates the subject is a candidate for treatment with a FADS1 inhibitor compound (e.g., suffers from or is at risk of suffering from a FADS1-mediated condition), a FADS1 inhibitor compound is administered to the subject. In several embodiments, the biological indicator is one or more PUFAs (or a ratio of PUFAs). In several embodiments, the biological indicator is a relative abundance of one or more cell types in the subject. In several embodiments, the biological indicator is a relative abundance of one or more DEGs. In several embodiments, the biological indicator is a relative abundance of one or more metabolites. In several embodiments, the FADS1 inhibitor compound is a FADS1 inhibiting small molecule. In several embodiments, by inhibiting FADS1, the availability of AA and production of the pro-inflammatory eicosanoids is limited and/or reduced.

### Methods of Treating and FADS1 Inhibitor Compounds for Use in Treating

Provided herein as Embodiment 1 is a method of treating a FADS1-mediated disease or disorder in a subject in need of treatment, the method comprising:
receiving information that compares a level of a biological indicator of FADS1-mediated disease from the subject to a reference level of the biological indicator;
wherein the biological indicator is a ratio arachidonic acid (AA) to dihomo-gamma-linolenic acid (DGLA); and administering to the subject a therapeutically effective amount of a FADS1 inhibitor compound if the level of biological indicator in the subject is greater than the reference level of biological indicator.

Provided herein as Embodiment 2 is a FADS1 inhibitor compound for use in a method of treating a subject in need of treatment who has a FADS1-mediated disease or disorder, the method comprising:
receiving information that compares a level of a biological indicator of FADS1-mediated disease from the subject to a reference level of the biological indicator;
wherein the biological indicator is a ratio arachidonic acid (AA) to dihomo-gamma-linolenic acid (DGLA); and administering to the subject a therapeutically effective amount of the FADS1 inhibitor compound if the level of biological indicator in the subject is greater than the reference level of biological indicator.

Provided herein as Embodiment 3 is the method or compound of Embodiment 1 or 2, wherein the reference level of the biological indicator is an AA to DGLA ratio that is equal to or at least about 5:1.

Provided herein as Embodiment 4 is the method or compound of Embodiment 1 or 2, wherein the reference level of the biological indicator is an AA to DGLA ratio that is equal to or at least about 6:1.

Provided herein as Embodiment 5 is the method or compound of Embodiment 1 or 2, wherein the reference level of the biological indicator is an AA to DGLA ratio that is equal to or at least about 7:1.

Provided herein as Embodiment 6 is the method or compound of Embodiment 1 or 2, wherein the reference level of the biological indicator is an AA to DGLA ratio that is equal to or at least about 15:2.

Provided herein as Embodiment 7 is the method or compound of Embodiment 1 or 2, wherein the reference level of the biological indicator is an AA to DGLA ratio that is equal to or at least about 8:1.

Provided herein as Embodiment 8 is the method or compound of Embodiment 1 or 2, wherein the reference level of the biological indicator is an AA to DGLA ratio that is equal to or at least about 17:2.

Provided herein as Embodiment 9 is the method or compound of Embodiment 1 or 2, wherein the reference level of the biological indicator is an AA to DGLA ratio that is equal to or at least about 9:1.

Provided herein as Embodiment 10 is a method of treating a FADS1-mediated disease or disorder in a subject in need of treatment, the method comprising:
receiving information that compares a level of a biological indicator of FADS1-mediated disease from the subject to a reference level of the biological indicator;
   wherein the biological indicator is a measured level of linoleic acid, gamma-linoleic acid, adrenic acid, docosapenatenoic acid n-6, alpha-linolenic acid, eicosatetraenoic acid, eicosapentaenoic acid, docosahexaenoic acid, or a combination of the foregoing; and
administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator.

Provided herein as Embodiment 11 is a FADS1 inhibitor compound for use in a method of treating a subject in need of treatment who has a FADS1-mediated disease or disorder, the method comprising:
receiving information that compares a level of a biological indicator of FADS1-mediated disease from the subject to a reference level of the biological indicator;
   wherein the biological indicator is a measured level of linoleic acid, gamma-linoleic acid, adrenic acid, docosapenatenoic acid n-6, alpha-linolenic acid, eicosatetraenoic acid, cicosapentacnoic acid, docosahexacnoic acid, or a combination of the foregoing; and
administering to the subject a therapeutically effective amount of the FADS1 inhibitor compound if the subject is determined to be one who would benefit from treatment with the FADS1 inhibitor compound as determined by the subject's level of the biological indicator compared to the reference level for the biological indicator.

Provided herein as Embodiment 12 is the method or compound of Embodiment 10 or 11, wherein the subject is determined to be one who would benefit from treatment with the FADS1 inhibitor compound where the subject's level of the biological indicator is higher than the reference level for the biological indicator.

Provided herein as Embodiment 13 is the method or compound of Embodiment 10 or 11, wherein the subject is determined to be one who would benefit from treatment with the FADS1 inhibitor compound where the subject's level of the biological indicator is lower than the reference level for the biological indicator.

Provided herein as Embodiment 14 is a method of treating a FADS1-mediated disease or disorder in a subject in need of treatment, the method comprising:
receiving information that compares a level of a biological indicator of FADS1-mediated disease from the subject to a reference level of the biological indicator;
   wherein the biological indicator is a measured level of plasma cholesterol, free cholesterol, total cholesterol, cholesterylester C20:4, malate, alpha-ketoglutarate, mannose, glucose, erythro-dihydrosphingosine (d18:0), 5-O-methylsphingosine (d18:1), erythro-sphingosine (d18:1), 3-O-methylsphingosine (d18:1), threo-sphingosine (d18:1), 1-hydroxy-2-amino-(cis,trans)-3,5-octadecadiene, 4-hydroxysphinganine (t18:0, phytosphingosine), sphingomyelin (d18:1,C23:0), sphingomyelin (d18:1,C24:0), ceramide (d18:1, C24:0), thromboxane B2, delta-12-prostaglandin D2, prostaglandin E2, prostaglandin D2, 12-hydroxyheptadecatrienoic acid (C17:[5,8,10]3), 14,15-dihydroxyeicosatrienoic acid (C20:cis[5,8,11]3), 11-hydroxyeicosatetraenoic acid (C20:cis[5,8,12,14]4), 13-hydroxyoctadecadienoic acid (13-HODE) (C18:cis[9]trans[11]2), arachidonic acid (C20:cis[5,8,11,14]4), docosahexaenoic acid (C22:cis[4,7,10,13,16,19]6), dihomo-gamma-linolenic acid (C20:cis[8,11,14]3), gamma-linolenic acid (C18:cis[6,9,12]3), docosapentaenoic acid (C22:cis[7,10,13,16,19]5), eicosapentaenoic acid (C20:cis[5,8,11,14,17]5), docosatetraenoic acid (C22:cis[7,10,13,16]4), stearic acid (C18:0), tryptophan, kynurenic acid, xanthurenic acid, histidine, leucine, isoleucine, valine, 3-hydroxyisobutyrate, glutamate, threonine, cysteine, sarcosine, plasma triglycerides, taurochenodeoxycholic acid, taurocholic acid, lysophosphatidylcholine (C18:0), lysophosphatidylcholine (C20:4), lysophosphatidylcholine (C17:0), lysophosphatidylethanolamine (C22:5), phosphatidylcholine (C18:0,C22:6), phosphatidylcholine (C18:0,C20:3), phosphatidylcholine (C18:1,C18:2), phosphatidylcholine (C16:1,C18:2), phosphatidylcholine (C18:0,C18:2), phosphatidylcholine (C16:0,C20:5), phosphatidylcholine (C16:0,C16:0), glycerol-3 phosphate, choline plasmalogen (C18,C20:4), myo-inositol, myo-inositolphospholipids, glycerol phosphate, phosphate lipid fraction, or a combination of the foregoing; and
administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator.

Provided herein as Embodiment 15 is a FADS1 inhibitor compound for use in a method of treating a FADS1-mediated disease or disorder in a subject in need of treatment, the method comprising:
receiving information that compares a level of a biological indicator of FADS1-mediated disease from the subject to a reference level of the biological indicator:
   wherein the biological indicator is a measured level of plasma cholesterol, free cholesterol, total cholesterol, cholesterylester C20:4, malate, alpha-ketoglutarate, mannose, glucose, erythro-dihydrosphingosine (d18:0), 5-O-methylsphingosine (d18:1), erythro-sphingosine (d18:1), 3-O-methylsphingosine (d18:1), threo-sphingosine (d18:1), 1-hydroxy-2-amino-(cis,trans)-3,5-octadecadiene, 4-hydroxysphinganine (t18:0, phytosphingosine), sphingomyelin (d18:1,C23:0), sphingomyelin (d18:1,C24:0), ceramide (d18:1, C24:0), thromboxane B2, delta-12-prostaglandin D2, prostaglandin E2, prostaglandin D2, 12-hydroxyheptadecatrienoic acid (C17:[5,8,10]3), 14,15-dihydroxyeicosatrienoic acid (C20:cis[5,8,11]3), 11-hydroxyeicosatetraenoic acid (C20:cis[5,8,12,14]4), 13-hydroxyoctadecadienoic acid (13-HODE) (C18:cis[9]trans[11]2), arachidonic acid (C20:cis[5,8,11,14]4), docosahexaenoic acid (C22:cis[4,7,10,13,16,19]6), dihomo-gamma-linolenic acid (C20:cis[8,11,14]3), gamma-linolenic acid (C18:cis[6,9,12]3), docosapentaenoic acid (C22:cis[7,10,13,16,19]5), eicosapentaenoic acid (C20:cis[5,8,11,14,17]5), docosatetraenoic acid (C22:cis[7,10,13,16]4), stearic acid (C18:0), tryptophan, kynurenic acid, xanthurenic acid, histidine, leucine, isoleucine, valine, 3-hydroxyisobutyrate, glutamate, threonine, cysteine, sarcosine, plasma triglycerides, taurochenodeoxycholic acid, taurocholic acid, lysophosphatidylcholine (C18:0), lysophosphatidylcholine (C20:4), lysophosphatidylcholine (C17:0), lysophosphatidylethanolamine (C22:5), phosphatidylcholine (C18:0,C22:6), phosphatidylcholine (C18:0,C20:3), phosphatidylcholine (C18:1,C18:2), phosphatidylcholine (C16:1,C18:2), phosphatidylcholine (C18:0,C18:2), phosphatidylcholine (C16:0,C20:5), phosphatidylcholine (C16:0,C16:0), glycerol-3 phosphate, choline plasmalogen (C18,C20:4), myo-inositol, myo-inositolphospholipids, glycerol phosphate, phosphate lipid fraction, or a combination of the foregoing; and
administering to the subject a therapeutically effective amount of the FADS1 inhibitor compound if the subject is determined to be one who would benefit from treatment with the FADS1 inhibitor compound as determined by the subject's level of the biological indicator compared to the reference level for the biological indicator.

Provided herein as Embodiment 16 is the method or compound of Embodiment 14 or 15, wherein the subject is determined to be one who would benefit from treatment with the FADS1 inhibitor compound where the subject's level of the biological indicator is higher than the reference level for the biological indicator.

Provided herein as Embodiment 17 is the method or compound of any one of Embodiments 14 to 16, wherein the biological indicator is plasma cholesterol, free cholesterol, total cholesterol, cholesterylester C20:4, mannose, glucose, erythro-dihydrosphingosine (d18:0), 5-O-methylsphingosine (d18:1), erythro-sphingosine (d18:1), 3-O-methylsphingosine (d18:1), threo-sphingosine (d18:1), 1-hydroxy-2-amino-(cis,trans)-3,5-octadecadiene, 4-hydroxysphinganine (t18:0, phytosphingosine), sphingomyelin (d18:1,C23:0), sphingomyelin (d18:1,C24:0), ceramide (d18:1, C24:0), thromboxane B2, delta-12-prostaglandin D2, prostaglandin E2, prostaglandin D2, 12-hydroxyheptadecatrienoic acid (C17:[5,8,10]3), 14,15-dihydroxyeicosatrienoic acid (C20:cis[5,8,11]3), 11-hydroxyeicosatetraenoic acid (C20:cis[5,8,12,14]4), 13-hydroxyoctadecadienoic acid (13-HODE) (C18:cis[9]trans[11]2), arachidonic acid (C20:cis[5,8,11,14]4), docosahexaenoic acid (C22:cis[4,7,10,13,16,19]6), docosapentaenoic acid (C22:cis[7,10,13,16,19]5), eicosapentaenoic acid (C20:cis[5,8,11,14,17]5), docosatetraenoic acid (C22:cis[7,10,13,16]4), stearic acid (C18:0), cysteine, plasma triglycerides, taurochenodeoxycholic acid, taurocholic acid, lysophosphatidylcholine (C18:0), lysophosphatidylcholine (C20:4), lysophosphatidylcholine (C17:0), phosphatidylcholine (C18:0,C22:6), phosphatidylcholine (C18:0,C20:3), phosphatidylcholine (C18:0,C18:2), phosphatidylcholine (C16:0,C20:5), choline plasmalogen (C18,C20:4), myo-inositol, myo-inositolphospholipids, glycerol phosphate, phosphate lipid fraction, or a combination of the foregoing.

Provided herein as Embodiment 18 is the method or compound of Embodiment 14 or 15, wherein the subject is determined to be one who would benefit from treatment with the FADS1 inhibitor compound where the subject's level of the biological indicator is lower than the reference level for the biological indicator.

Provided herein as Embodiment 19 is the method or compound of any one of Embodiments 14 to 15 and 18, wherein the biological indicator is malate, alpha-ketoglutarate, dihomo-gamma-linolenic acid (C20:cis[8,11,14]3), gamma-linolenic acid (C18:cis[6,9,12]3), tryptophan, kynurenic acid, xanthurenic acid, histidine, leucine, isoleucine, valine, 3-hydroxyisobutyrate, glutamate, threonine, sarcosine, lysophosphatidylethanolamine (C22:5), phosphatidylcholine (C18:1,C18:2), phosphatidylcholine (C16:1,C18:2), phosphatidylcholine (C16:0,C16:0), glycerol-3 phosphate, or a combination of the foregoing.

Provided herein as Embodiment 20 is a method of treating a FADS1-mediated disease or disorder in a subject in need of treatment, the method comprising:
receiving information that compares a level of a biological indicator of FADS1-mediated disease from the subject to a reference level of the biological indicator;
wherein the biological indicator is a measured level of a differentially expressed gene (DEG);
   wherein the DEG is Serpinb1a, Gna14, Serpina3m, Hsd11b1, Cyp2c29, Akr1d1, Aldh1a1, Mmp19, Gyp27a1, Tymp, Elov12, Chkb, H2afj, Tnfaip811, Tmem86a, Sel113, Agap2, 4833411C07Rik, Elov4, Fat3, Eef1a2, Atp7a, Rgs2, Cenpe, Mfap2, Mlki67, Ctsh, Trub2, Ubc, H6pd, Eepd1, Acss2, Aacs, Gm36827, Man2a2, Nudt18, Plagl1, TM4sf19, Atp6v0d2, Gm20056, Trem2, Il1rn, Mmp12, Cdk18, Efr3b, Tag1n2, Lurap1, Cp, I17rb, B230303O12Rik, Cfd, Sult1e1, Tdo2, Cyp2b9, Hao2, Cyp2b13, Cyp2a22, Acnat2, Ildr2, Rpl10a-ps1, Tm6sf2, Fitm1, Lpar1, C6, Cmah, Lbp, Arsg, Glra3, Lad1, A730063M14Rik. Ly6f, Foxi1, Crygc, Defb28, Wfdc9, Ph1da2, Aqp6, Gm16411, Adam7, Ppp2r5b, Slc6a7, Gpr50, Ahnak2, S100a6, Mmp14, Htr2b, Hpgds, Gm18537, Pclo, Adrb3, Gm38394, AC154232.2, Cadps, Adgrb2, Gn45470, Sdr9c7, Dsg1c, Slc17a1, Ces1c, Gss, 1810008I18Rik, Tlcd1, Snrk, Akr1c20, Gm19950, Ttr, Cbfa2t3, Acat2, Pmvk, Abcd3, Acacb, Arhgap27, Rnase9, Wfdc8, Ighv9-2, DerI3, Acap1, Ccl19, Tcf7, Xkrx, Trim46, Zfp369, Zfp871, Pcdhb21, Gm14288, Uprt, Atm, Dchs2, Cped1, Gm38357, Cck, Ckap2, Gm4419, 1600015I10Rik, Sez6I2, Prnd, Gm16702, S100a8, Pcdh12, Malat1, Kcnqlot1, ArI4c, Gm42549, Gm37310, Gm37776, Atp2a1, Ckm, Tnnt3, Adipoq, Fabp4, Lep, Rem, Hoxc8, Hoxc9, Cebpa, Dgat1, Dgat2, Elov13, Fas, Scd1, Srebf1, Hilpda, Lipe, Mg11, Plint, Plin4, Pnpla2, Pnpla3, Ldah, Cs, Gckr, Me1, Pck1, Pdk4, Irs1, Acaa1a, Acads, Acox1, Cpt1a, Cpt1b, Hadhb, Ehhadh, Ppara, Ppargc1a, Cyp4a12a, Cyp4a12b, Cyp2e1, Adgre1, Agtr1a, Cc12, Cd14, Cd68, Il1b, Tnf, or a combination of the foregoing; and
administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator.

Provided herein as Embodiment 21 is a FADS1 inhibitor compound for use in a method of treating a subject in need of treatment who has a FADS1-mediated disease or disorder, the method comprising:
receiving information that compares a level of a biological indicator of FADS1-mediated disease from the subject to a reference level of the biological indicator;
wherein the biological indicator is a measured level of a differentially expressed gene (DEG);
   wherein the DEG is Serpinb1a, Gna14, Serpina3m, Hsd11b1, Cyp2c29, Akr1d1, Aldh1a1, Mmp19, Gyp27a1, Tymp, Elov12, Chkb, H2afj, Tnfaip811, Tmem86a, Sel13, Agap2, 4833411C07Rik, Elov4, Fat3, Eef1a2, Atp7a, Rgs2, Cenpe, Mfap2, Mlki67, Ctsh, Trub2, Ubc, H6pd, Ecpd1, Acss2, Aacs, Gm36827, Man2a2, Nudt18, Plagl1, TM4sf19, Atp6v0d2, Gm20056, Trem2, I11rn, Mmp12, Cdk18, Efr3b, Tag1n2, Lurap1, Cp, I17rb, B230303O12Rik, Cfd, Sult1e1, Tdo2, Cyp2b9, Hao2, Cyp2b13, Cyp2a22, Acnat2, Ildr2, Rpl10a-ps1, Tm6sf2, Fitm1, Lpar1, C6, Cmah, Lbp, Arsg, Glra3, Lad1, A730063M14Rik, Ly6f, Foxi1, Crygc, Defb28, Wfdc9, Ph1da2, Aqp6, Gm16411, Adam7, Ppp2r5b, Slc6a7, Gpr50, Ahnak2, S100a6, Mmp14, Htr2b, Hpgds, Gm18537, Pclo, Adrb3, Gm38394, AC154232.2, Cadps, Adgrb2, Gm45470, Sdr9c7, Dsg1c, Slc17a1, Ces1c, Gss, 1810008I18Rik, Tlcd1, Snrk, Akr1c20, Gm19950, Ttr, Cbfa2t3, Acat2, Pmvk, Abcd3, Acacb, Arhgap27, Rnase9, Wfdc8, Ighv9-2, DerI3, Acap1, Ccl19, Tcf7, Xkrx, Trim46, Zfp369, Zfp871, Pcdhb21, Gm14288, Uprt, Atm, Dchs2, Cped1, Gm38357, Cck, Ckap2, Gm4419, 1600015I10Rik, Sez6I2, Prnd, Gm16702, S100a8, Pcdh12, Malat1, Kcng1ot1, ArI4c, Gm42549, Gm37310, Gm37776, Atp2a1, Ckm, Tnnt3, Adipoq, Fabp4, Lep, Rem, Hoxc8, Hoxc9, Cebpa, Dgat1, Dgat2, Elov13, Fas, Scd1, Srebf1, Hilpda, Lipe, Mg11, Plin1, Plin4, Pnpla2, Pnpla3, Ldah, Cs, Gckr, Me1, Pck1, Pdk4, Irs1, Acaa1a, Acads, Acox1, Cpt1a, Cpt1b, Hadhb, Ehhadh, Ppara, Ppargc1a, Cyp4a12a, Cyp4a12b, Cyp2e1, Adgre1, Agtr1a, Cc12, Cd14, Cd68, Il1b, Tnf, or a combination of the foregoing; and
administering to the subject a therapeutically effective amount of the FADS1 inhibitor compound if the subject is determined to be one who would benefit from treatment with the FADS1 inhibitor compound as determined by the subject's level of the biological indicator compared to the reference level for the biological indicator.

Provided herein as Embodiment 22 is the method or compound of Embodiment 20 or 21, wherein the subject is determined to be one who would benefit from treatment with the FADS1 inhibitor compound where the subject's level of the biological indicator is higher than the reference level for the biological indicator.

Provided herein as Embodiment 23 is the method or compound of any one of Embodiments 20 to 22, wherein the DEG is Serpinb1a, Gna14, Serpina3m, Hsd11b1, Cyp2c29, Hsd11b1, Akr1d1, Aldh1a1, or a combination of the foregoing.

Provided herein as Embodiment 24 is the method or compound of any one of Embodiments 20 to 23, wherein the DEG is Elov4, Fat3, Eef1a2, Atp7a, Rgs2, Cenpe, Mfap2, Mlki67, Ctsh, or a combination of the foregoing.

Provided herein as Embodiment 25 is the method or compound of any one of Embodiments 20 to 24, wherein the DEG is TM4sf19, Atp6v0d2, Gm20056, Trem2, I11rn, Mmp12, Cdk18, Efr3b, Tag1n2, or a combination of the foregoing.

Provided herein as Embodiment 26 is the method or compound of any one of Embodiments 20 to 25, wherein the DEG is Cyp2b9, Fads1, Hao2, Cyp2b13, Cyp2a22, Acnat2, Ildr2, Rpl10a-ps1, Tm6sf2, or a combination of the foregoing.

Provided herein as Embodiment 27 is the method or compound of any one of Embodiments 20 to 26, wherein the DEG is Ly6f, Foxi1, Crygc, Defb28, Wfdc9, Ph1da2, Aqp6, Gm16411, Adam7, or a combination of the foregoing.

Provided herein as Embodiment 28 is the method or compound of any one of Embodiments 20 to 27, wherein the DEG is Slc6a7, Gpr50, Ahnak2, S100a6, Mmp14, Htr2b, Hpgds, Mfap2, or a combination of the foregoing.

Provided herein as Embodiment 29 is the method or compound of any one of Embodiments 20 to 28, wherein the DEG is Sdr9c7, Dsg1c, Slc17a1, Acnat2, Ces1c, Gss, Hsd11b1, or a combination of the foregoing.

Provided herein as Embodiment 30 is the method or compound of any one of Embodiments 20 to 29, wherein the DEG is Adam7, Rnase9, Wfdc8. Ighv9-2, DerI3, Acap1, Ccl19, Tcf7, Xkrx, Trim46, or a combination of the foregoing.

Provided herein as Embodiment 31 is the method or compound of any one of Embodiments 20 to 30, wherein the DEG is Cck, Ckap2, Gm4419, 1600015I10Rik, Sez6I2, Prnd, Gm16702, S100a8, or a combination of the foregoing.

Provided herein as Embodiment 32 is the method or compound of Embodiment 20 or 21, wherein the subject is determined to be one who would benefit from treatment with the FADS1 inhibitor compound where the subject's level of the biological indicator is lower than the reference level for the biological indicator.

Provided herein as Embodiment 33 is the method or compound of any one of Embodiments 20 to 21 and 32, wherein the DEG is Tmem86a, Elov12, Agap2, Chkb, Tnfaip811, H2afj, Sel113, 4833411C07Rik, or a combination of the foregoing.

Provided herein as Embodiment 34 is the method or compound of any one of Embodiments 20 to 21 and 32 to 33, wherein the DEG is Trub2, Ube, H6pd, Eepd1, Acss2, Aacs, Gm36827, Man2a2, Nudt18. Plagl1, or a combination of the foregoing.

Provided herein as Embodiment 35 is the method or compound of any one of Embodiments 20 to 21 and 32 to 34, wherein the DEG is Lurap1, Cp, I17rb. B230303O12Rik, Cfd. Sult1e1, Tdo2, or a combination of the foregoing.

Provided herein as Embodiment 36 is the method or compound of any one of Embodiments 20 to 21 and 32 to 34, wherein the DEG is Fitm1, Tmem86a, Agap2, Lpar1, C6, Cmah, Lbp, Arsg, Glra3, Lad1, A730063M14Rik, or a combination of the foregoing.

Provided herein as Embodiment 37 is the method or compound of any one of Embodiments 20 to 21 and 32 to 34, wherein the DEG is Ppp2r5b.

Provided herein as Embodiment 38 is the method or compound of any one of Embodiments 20 to 21 and 32 to 34, wherein the DEG is Gm18537, Aacs, Pclo, Adrb3, Gm38394, AC154232.2, Cadps, Adgrb2, Gm45470, or a combination of the foregoing.

Provided herein as Embodiment 39 is the method or compound of any one of Embodiments 20 to 21 and 32 to 34, wherein the DEG is 1810008I18Rik, Tlcd1, Snrk, Akr1c20, Gm19950, Ttr, Cbfa2t3, Acat2, Pmvk, Abcd3, Acss2, Acacb, Arhgap27, or a combination of the foregoing.

Provided herein as Embodiment 40 is the method or compound of any one of Embodiments 20 to 21 and 32 to 34, wherein the DEG is Zfp369, Zfp871, Pcdhb21, Adrb3, Gm14288, Uprt, Atm, Dchs2, Cped1, Gm38357, or a combination of the foregoing.

Provided herein as Embodiment 41 is the method or compound of any one of Embodiments 20 to 21 and 32 to 34, wherein the DEG is Pcdh12, Malat1, Kcnqlot1, ArI4c, Gm38394, Gm42549, AC154232.2, Gm37310, Gm37776, Atp2a1, Ckm, Tnnt3, or a combination of the foregoing.

Provided herein as Embodiment 42 is the method or compound of any one of Embodiments 20 to 41, wherein the DEG is Mmp19, Gyp27a1, Tymp, or a combination of the foregoing.

Provided herein as Embodiment 43 is the method or compound of any one of Embodiments 20 to 42, wherein the DEG is Adipoq, Fabp4, Lep, Retn, Hoxc8, Hoxc9, Cebpa, Dgat1, Dgat2, Elov13, Fas, Scd1, Srebf1, Adrb3, Hilpda, Lipe, Mg11, Plin1, Plin4, Pnpla2, Pnpla3, Ldah, Cs, Gckr, Me1, Pck1, Pdk4, Irs1, Acaa1a, Acads, Acox1, Cpt1a, Cpt1b, Hadhb, Ehhadh, Ppara, Ppargc1a, Cyp4a12a, Cyp4a12b, Cyp2e1, Adgre1, Agtr1a, Cc12, Cd14, Cd68, Il1b, Tm6sf2, Tnf, or a combination of the foregoing.

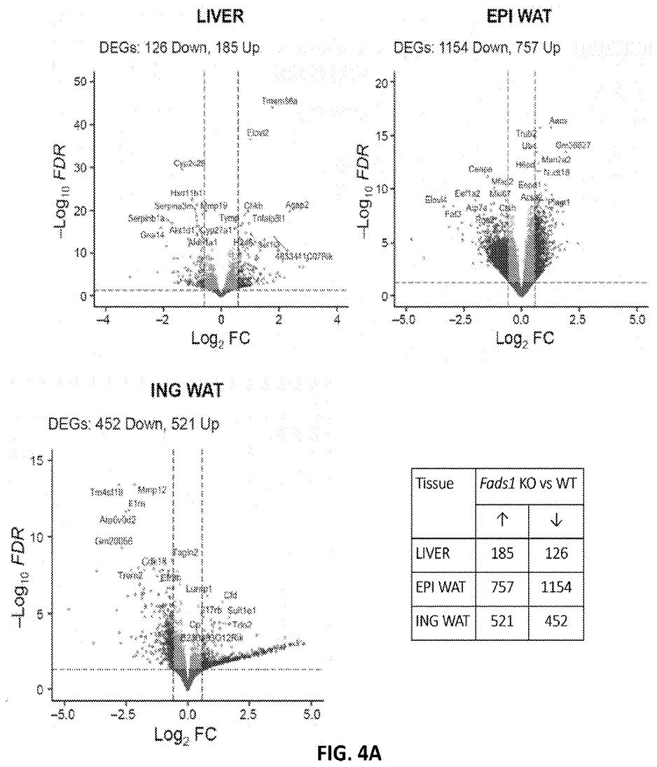

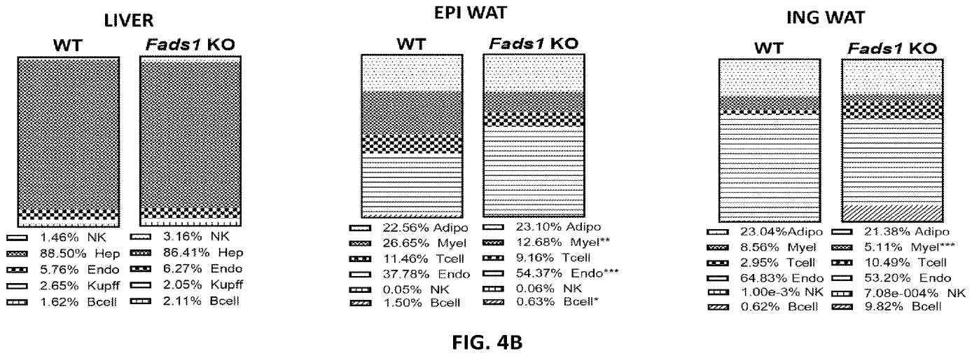

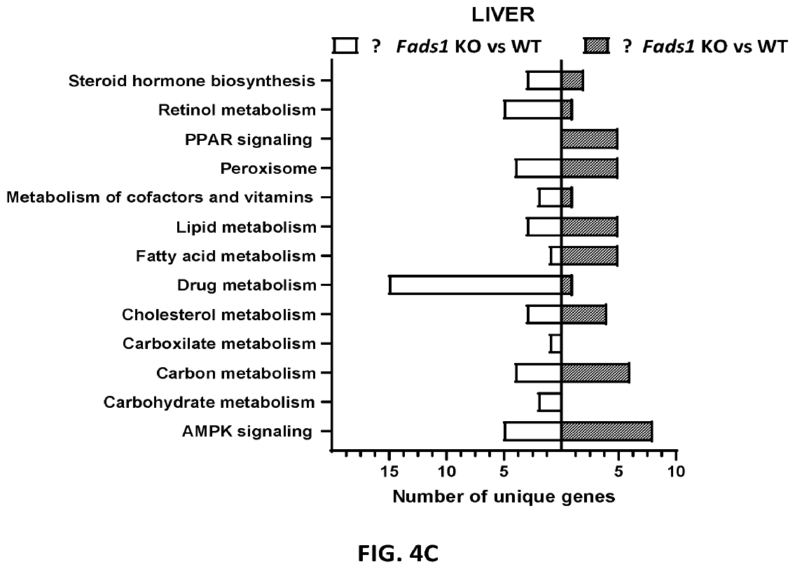

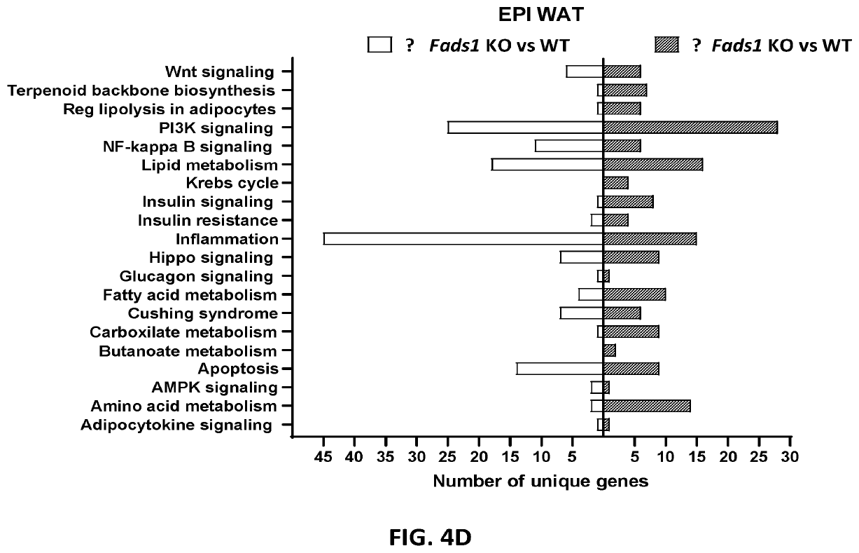

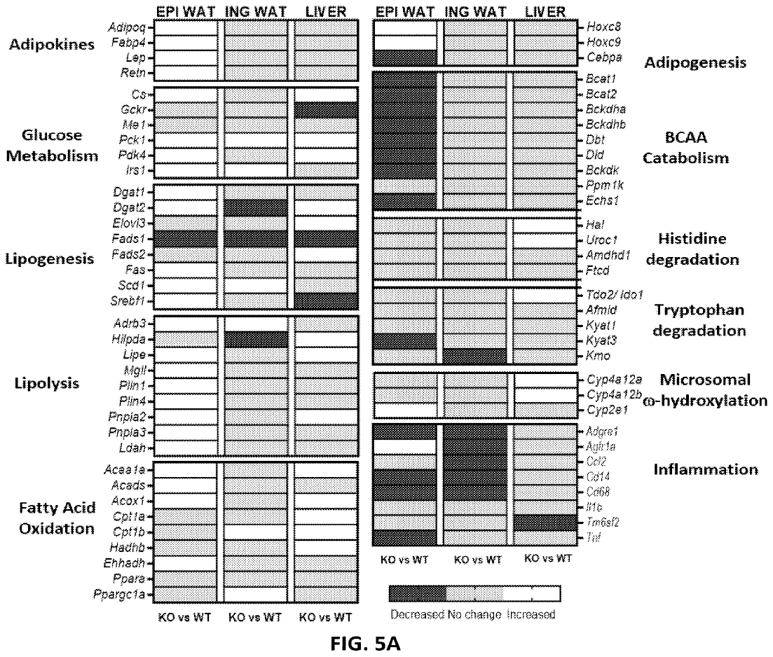

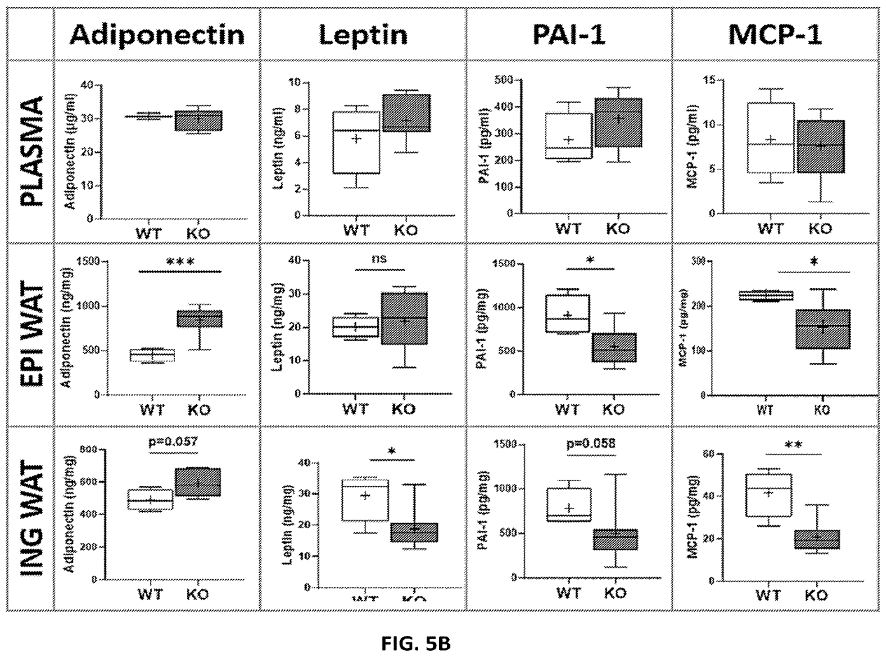

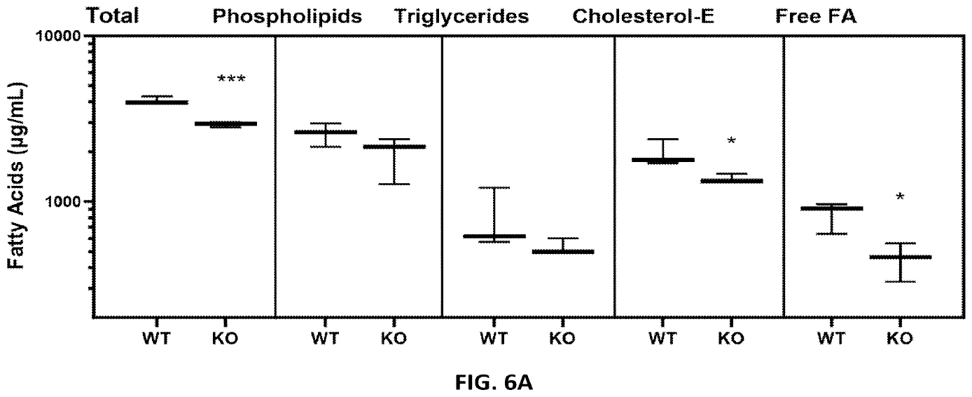

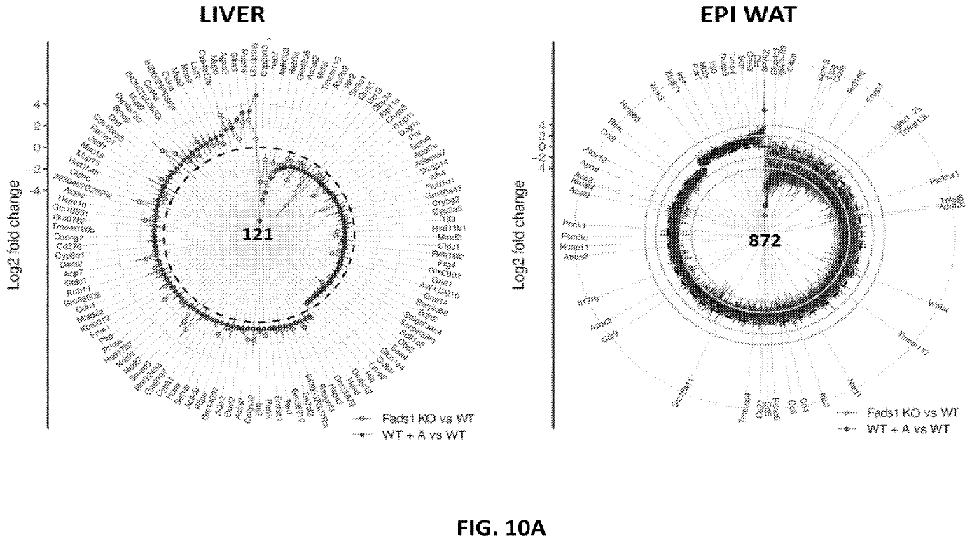

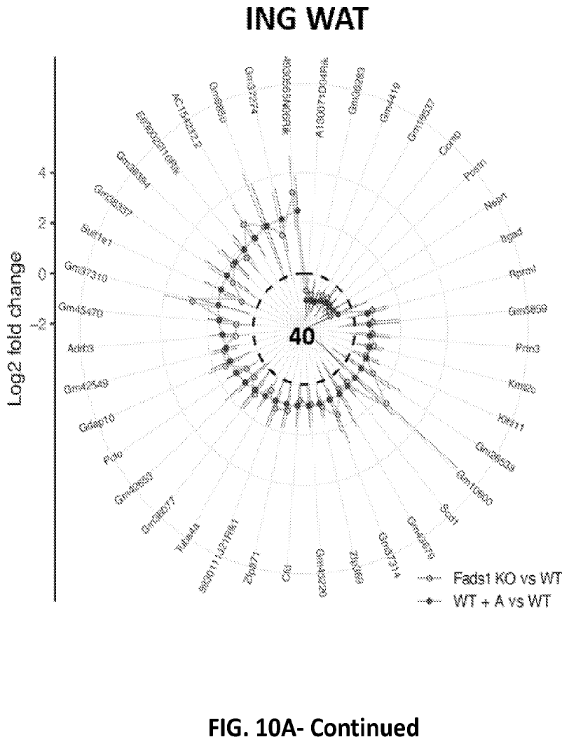

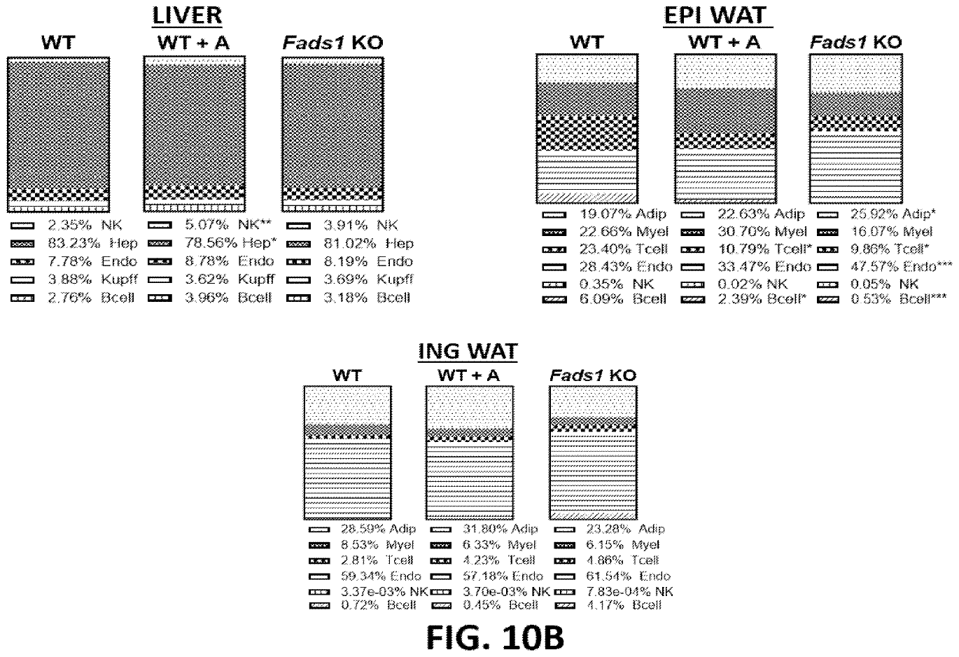

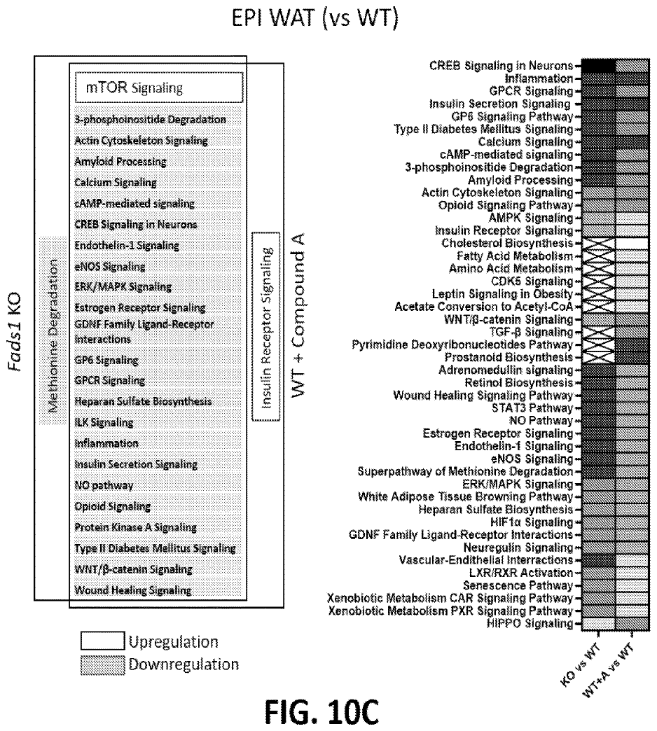

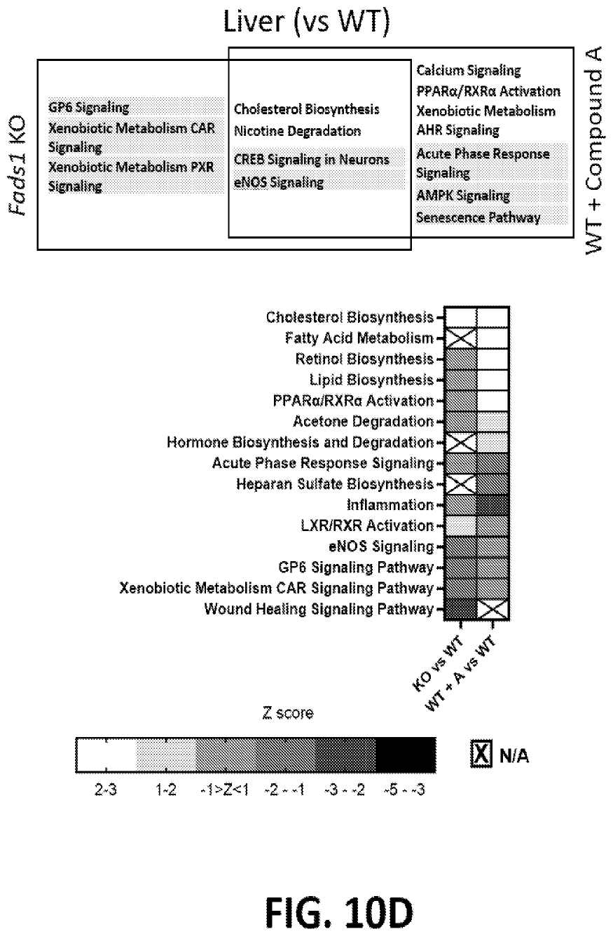

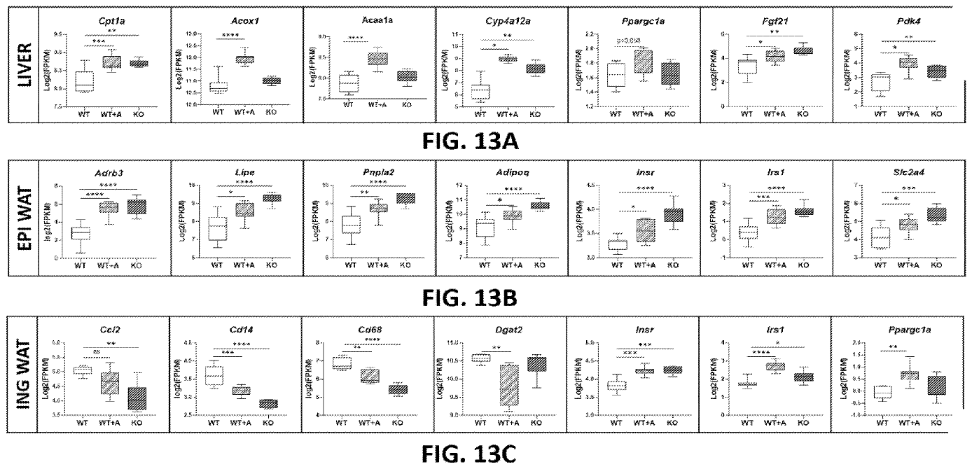

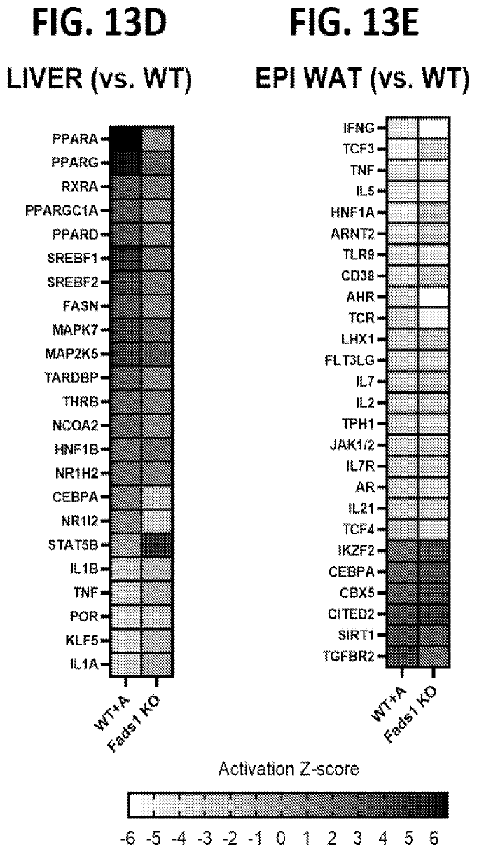

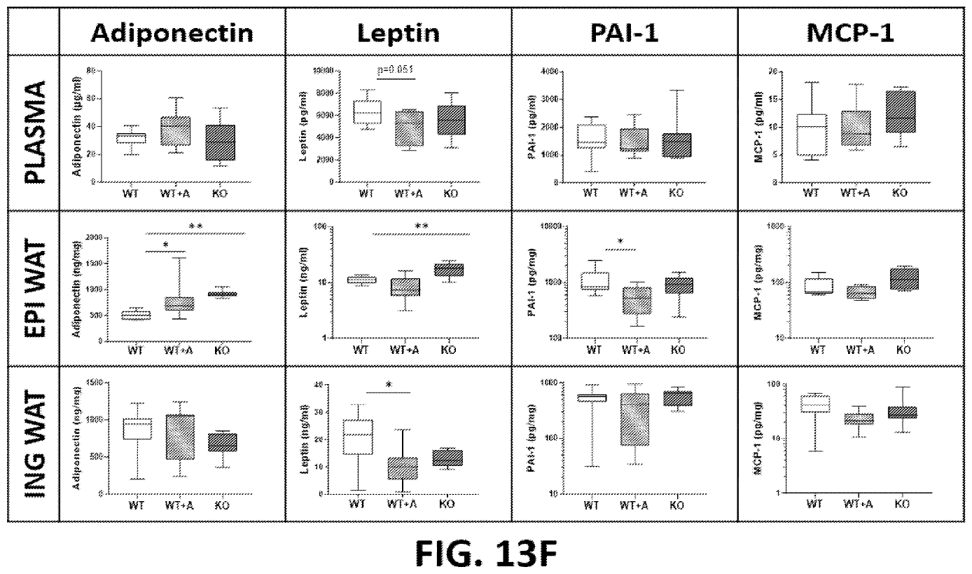

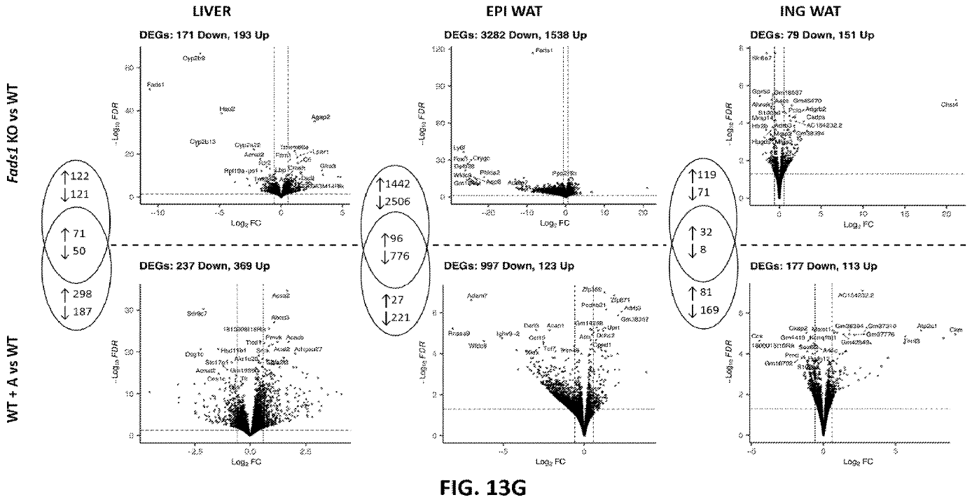

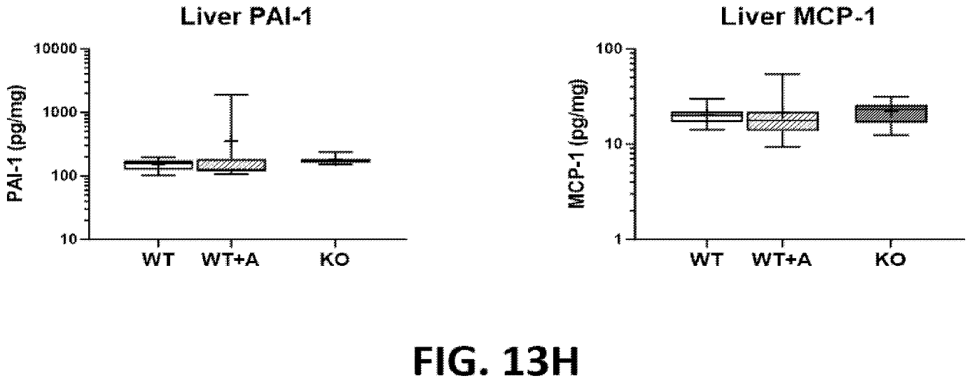

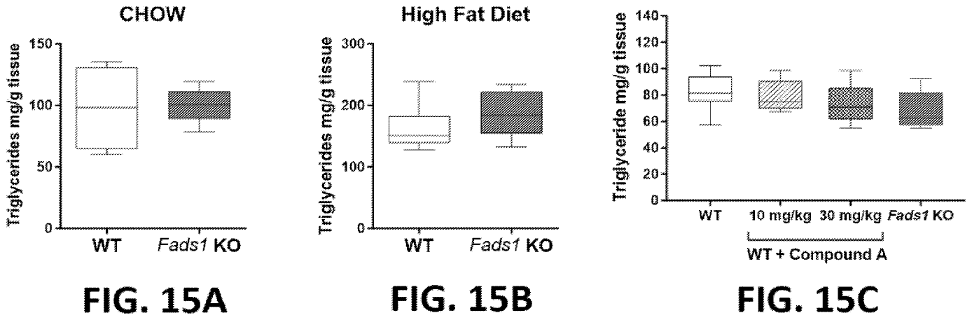

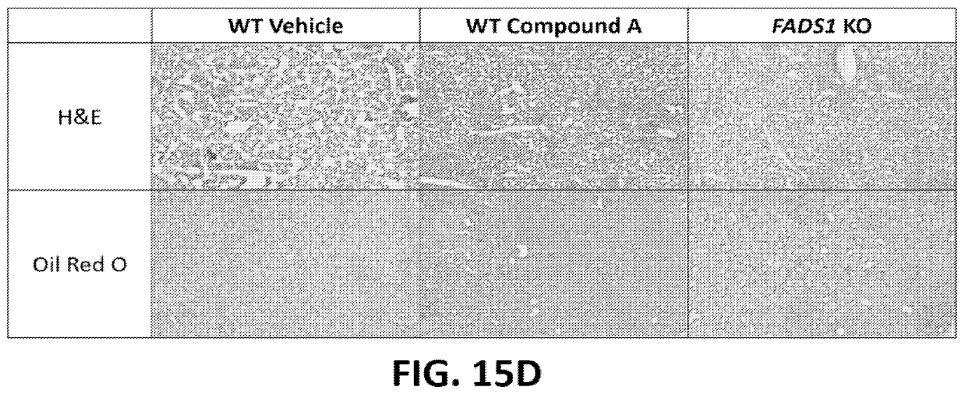

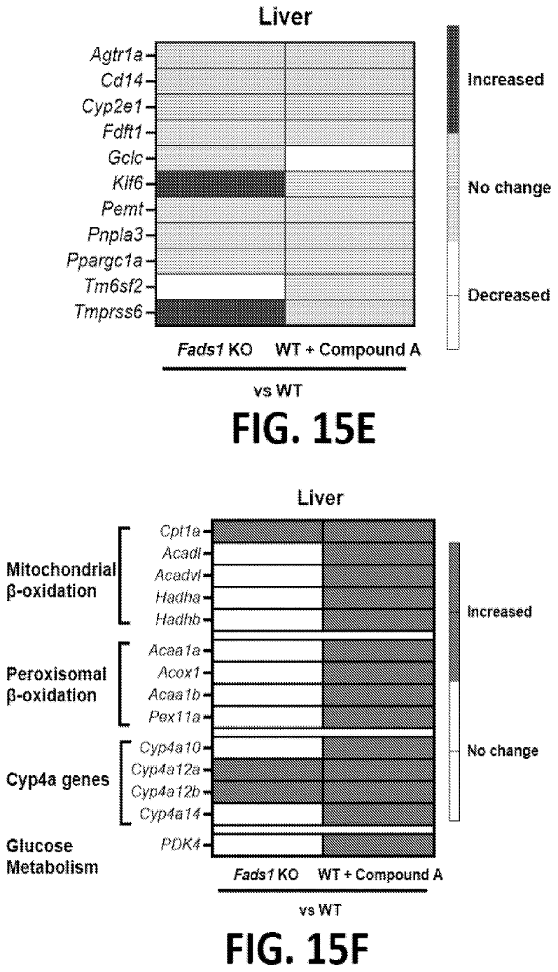

Provided herein as Embodiment 44 is the method or compound of any one of Embodiments 20 to 43, wherein the DEG is one disclosed in FIG. 4a. FIG. 5a. FIG. 10a, FIG. 13a, FIG. 13g. FIG. 15e, FIG. 15f, or a combination of DEGs disclosed in the foregoing. For example, in several embodiments, the DEG is one disclosed in FIG. 4a. In several embodiments, the DEG is one disclosed in FIG. 5a. In several embodiments, the DEG is one disclosed in FIG. 10a. In several embodiments, the DEG is one disclosed in FIG. 13a. In several embodiments, the DEG is one disclosed in FIG. 13g. In several embodiments, the DEG is one disclosed in FIG. 15e. In several embodiments, the DEG is one disclosed in FIG. 15f. In several embodiments, the DEG is one disclosed in or a combination of DEGs disclosed in the foregoing. In several embodiments, the DEG is one disclosed in any other figure provided herein.

Provided herein as Embodiment 45 is a method of treating a FADS1-mediated disease or disorder in a subject in need of treatment, the method comprising:
receiving information that compares a level of a biological indicator of FADS1-mediated disease from the subject to a reference level of the biological indicator;
   wherein the biological indicator is a measured level of one or more cell types in the subject, wherein the cell type is an adipocyte (Adipo), B cell (Bcell), endothelial cell (Endo), hepatocyte (Hep), kupffer cell (Kupff), myeloid cell (Myel), natural killer cell (NK), T cell (Tcell), or a combination of the foregoing; and administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator.

Provided herein as Embodiment 46 is a FADS1 inhibitor compound for use in a method of treating a subject in need of treatment who has a FADS1-mediated disease or disorder, the method comprising:
receiving information that compares a level of a biological indicator of FADS1-mediated disease from the subject to a reference level of the biological indicator:
   wherein the biological indicator is a measured level of one or more cell types in the subject, wherein the cell type is an adipocyte (Adipo), B cell (Bcell), endothelial cell (Endo), hepatocyte (Hep), kupffer cell (Kupff), myeloid cell (Myel), natural killer cell (NK), T cell (Tcell), or a combination of the foregoing; and
administering to the subject a therapeutically effective amount of the FADS1 inhibitor compound if the subject is determined to be one who would benefit from treatment with the FADS1 inhibitor compound as determined by the subject's level of the biological indicator compared to the reference level for the biological indicator.

Provided herein as Embodiment 47 is the method or compound of Embodiment 45 or 46, wherein the subject is determined to be one who would benefit from treatment with the FADS1 inhibitor compound where the subject's level of the biological indicator is higher than the reference level for the biological indicator.

Provided herein as Embodiment 48 is the method or compound of any one of Embodiments 45 to 46 and 47, wherein the cell type is Adipo, Myel, or a combination thereof.

Provided herein as Embodiment 49 is the method or compound of Embodiment 45 or 46, wherein the subject is determined to be one who would benefit from treatment with the FADS1 inhibitor compound where the subject's level of the biological indicator is lower than the reference level for the biological indicator.

Provided herein as Embodiment 50 is the method or compound of any one of Embodiments 45 to 46 and 49, wherein the cell type is Bcell, Endo, NK, Tcell, or a combination thereof.

Provided herein as Embodiment 51 is the method or compound of any one of Embodiments 1 to 50, wherein the FADS1-mediated disease or disorder is obesity, a metabolic disorder, a cardiovascular disorder, diabetes, dyslipidemia, non-alcoholic steatohepatitis (NASH), or a combination of any of the foregoing. For example, in several embodiments, the FADS1-mediated disease or disorder is obesity. In several embodiments, the FADS1-mediated disease or disorder is a metabolic disorder. In several embodiments, the FADS1-mediated disease or disorder is a cardiovascular disorder. In several embodiments, the FADS1-mediated disease or disorder is diabetes. In several embodiments, the FADS1-mediated disease or disorder is dyslipidemia. In several embodiments, the FADS1-mediated disease or disorder is non-alcoholic steatohepatitis (NASH).

Provided herein as Embodiment 52 is the method or compound of any one of Embodiments 1 to 51, wherein the reference level of biological indicator is an average amount of biological indicator in a population of healthy subjects.

Provided herein as Embodiment 53 is the method or compound of any one of Embodiments 1 to 52, wherein the reference level of biological indicator is an average amount of biological indicator in a population of subjects not having a FADS1-mediated disease.

Provided herein as Embodiment 54 is the method or compound of any one of Embodiments 1 to 53, wherein the reference level of biological indicator is an average amount of biological indicator in a population of subjects having a Body Mass Index (BMI) greater than or equal to 25.0.

Provided herein as Embodiment 55 is the method or compound of any one of Embodiments 1 to 53, wherein the reference level of biological indicator is an average amount of biological indicator in a population of subjects having a Body Mass Index (BMI) of greater than or equal to 30.0.

Provided herein as Embodiment 56 is the method or compound of any one of Embodiments 1 to 55, wherein the reference level of biological indicator is an average amount of biological indicator in a population of subjects having a body fat percentage of equal to or less than 19%.

Provided herein as Embodiment 57 is the method or compound of Embodiment 56, wherein the reference level of biological indicator is from a population of males and wherein the subject is a male.

Provided herein as Embodiment 58 is the method or compound of any one of Embodiments 1 to 55, wherein the reference level of biological indicator is an average amount of biological indicator in a population of subjects having a body fat percentage of equal to or less than 32%.

Provided herein as Embodiment 59 is the method or compound of Embodiment 58, wherein the reference level of biological indicator is from a population of females and wherein the subject is a female.

Provided herein as Embodiment 60 is the method or compound of any one of Embodiments 1 to 59, wherein the level of biological indicator of FADS1-mediated disease from the subject is quantified using a sample collected from the subject; wherein the sample is of blood, plasma, or a tissue biopsy. For example, in several embodiments, the level of a biological indicator of FADS1-mediated disease from the subject is determined using a blood sample from the subject. In several embodiments, the level of a biological indicator of FADS1-mediated disease from the subject is determined using a plasma sample from the subject. In several embodiments, the level of a biological indicator of FADS1-mediated disease from the subject is determined using a tissue sample from the subject. In several embodiments, the tissue sample is collected as a biopsy. In several embodiments, the blood or plasma sample is collected using a needle.

Provided herein as Embodiment 61 is the method or compound of Embodiment 60, wherein the tissue is fat tissue or organ tissue. For example, in several embodiments, a fat tissue sample is taken from the subject.

As disclosed elsewhere herein, several embodiments pertain to methods of determining and/or predicting whether a subject is a candidate for (e.g., would respond to) treatment with a FADS1 inhibitor compound. In several embodiments, whether a subject is a candidate for treatment is related to the activity of the FADS1 enzyme in the subject. As disclosed elsewhere herein, where FADS1 activity is increased in a subject relative to a healthy subject and/or a relative to a subject lacking a FADS1-mediated condition (e.g., a disease or disorder), treatment with a FADS1 inhibitor may be beneficial. In several embodiments, methods of identifying a subject having FADS1 activity are provided. In several embodiments, methods of measuring or approximating a subject's level of FADS1 activity are provided. In several embodiments, as disclosed elsewhere herein, methods of determining if a subject is in need of treatment with a FADS1 inhibitor are provided. In several embodiments, the identification of a subject suffering from a FADS1-mediated condition, the identification of a subject having increased FADS1 activity (relative to a healthy subject), and/or the identification of a subject in need of treatment with a FADS1 inhibitor compound can be performed by analyzing the relative abundance of and/or the ratio of one or more biological indicators of FADS1-mediated diseases or disorders (e.g., biological markers) in the subject. Thus, biological markers (e.g., biomarkers) to be measured and/or compared are provided herein. Several embodiments disclosed herein pertain to methods of selecting a subject for treatment with a FADS1 inhibitor compound based on the presence or relative abundance of one or more biological indicators.

Advantageously, depending on individual patient information and characteristics, treatment regimens can be tailored to that individual. For instance, administration of a FADS1 inhibitor compound may or may not be initiated based on biological indicator levels in the patient. Because a healthy subject has one or more biological indicators with relative abundances that are different (e.g., decreased or increased) when compared to a subject in need of treatment with a FADS1 inhibitor compound (e.g., a treatment candidate), by measuring biological indicators in a candidate for treatment, a patient selection step can be performed. During a selection step, biological indicator levels in the treatment candidate may be compared with those from healthy subjects or with those from other successfully treated subjects (e.g., prior to their treatment). Where the relative abundances of these biological indicators show that the subject might or would benefit from treatment (e.g., with a FADS1 inhibitor compound) or where a FADS1 disease or disorder is present or may occur, the subject can be selected for treatment.

Once selected for treatment, as disclosed elsewhere herein, a FADS1 inhibitor compound may be administered to the patient. If these biological indicator levels indicate that the subject would not benefit from treatment or is unlikely to be successfully treated, the subject can be excluded from treatment.

As disclosed elsewhere herein, in several embodiments, biological indicators may be extracted from or measured in a sample collected from a subject before, during, and/or after treatment. In several embodiments, the sample is a tissue sample (e.g., from the liver, an organ, or the blood). In several embodiments, the sample is collected through a tissue biopsy or using a syringe (e.g., to collect a blood or plasma sample). In several embodiments, the sample is a body fluid (e.g., urine, saliva, plasma, etc.).

In several embodiments, the relative abundance of one, two, three, four, five, six, seven, eight, or more biological indicators can be used to assess whether a subject has increased FADS1 activity and/or is a candidate for treatment with a FADS1 inhibitor compound. Any combination of different biological indicators disclosed herein may be used (e.g., in a patient selection step).

In several embodiments, the biological indicator may include one or more SNPs in the FADS1 gene. In several embodiments, a subject in need of treatment is one lacking a rs174556 and/or rs7115739 SNPs of FADS1.

As disclosed elsewhere herein, in several embodiments, the biological indicator or at least one of the biological indicators is a ratio of PUFAs in the subject. In several embodiments, the ratio of PUFAs comprises or consists of a ratio of AA to DGLA. In several embodiments, for a subject in need of treatment, the ratio of AA to DGLA is equal to or at least about: 4:1, 9:2, 5:1, 11:2, 6:1, 7:1, 8:1, 10:1, or ranges including and/or spanning the aforementioned values. In several embodiments, for a subject in need of treatment, the ratio of AA to DGLA is greater than that for a healthy patient by equal to or at least about: 10%, 20%, 30%, 40%, 50%, or ranges including and/or spanning the aforementioned values. To illustrate, where a ratio of AA to DGLA for a healthy individual is 4, a value that is 10% greater is 4.4.

As disclosed elsewhere herein, in several embodiments, the biological indicator or at least one of the biological indicators is one or more cell types. In several embodiments, the cell types include one or more of adipocytes (Adipo), B cell (Bcell), endothelial (Endo), hepatocytes (Hep), kupffer (Kupff), myeloid (Myel), natural killer (NK), T cell (Tcell), or combinations of any of the foregoing. In several embodiments, the relative abundance of at least one cell type of a subject in need of treatment is increased (e.g., relative to a healthy subject and/or a subject not in need of treatment). In several embodiments, the relative abundance of the cell type (e.g., from a sample) is increased by equal to or at least about: 10%, 20%, 30%, 50%, 75%, 100%, 150%, 200%, 250%, 300%, 350%, 400%, 500%, or ranges including and/or spanning the aforementioned values. To illustrate, where the relative abundance is increased by 200%, this is 2 fold increase where the relative abundance is two times as high. In several embodiments, the relative abundance of the cell type (e.g., from a sample) is increased by equal to or at least about: 1.1 fold, 1.2 fold, 1.3 fold, 1.5 fold, 1.75 fold, 2 fold, 2.5 fold, 3 fold, 3.5 fold, 4 fold, 5 fold, 10 fold, 15 fold, 20 fold, 40 fold, or ranges including and/or spanning the aforementioned values. In several embodiments, the relative abundance of at least one cell type of the subject is decreased. In several embodiments, the relative abundance of the cell type (e.g., from a sample) is decreased by equal to or at least about: 2.5%, 5%, 10%, 20%, 30%, 50%, 75%, 90%, 92.5%, 95%, 97.5%, 99%, 99.5%, or ranges including and/or spanning the aforementioned values. In several embodiments, the relative abundance of the cell type (e.g., from a sample) is decreased by equal to or at least about: 1.5 fold, 2 fold, 4 fold, 5 fold, 10 fold, 15 fold, 20 fold, 40 fold, or ranges including and/or spanning the aforementioned values. To illustrate, where the relative abundance is decreased by 90%, this is a 10 fold decrease where the relative abundance is ten times less (because the relative abundance is 10% of that of the reference).

As disclosed elsewhere herein, in several embodiments, the biological indicator or at least one of the biological indicators is one or more metabolites. In several embodiments, the metabolites include one or more of plasma cholesterol, free cholesterol, total cholesterol, cholesterylester, malate, alpha-ketoglutarate, mannose, glucose, erythron-dihydrosphingosine, 5-O-methylsphingosine, threo-sphinogosine, 1-hydroxy-2-amino-(cis,trans)-3,5-octadecadiene, 4-hydroxysphinganine, thromboxane B2, delta-12-prostaglandin D2, prostaglandin E2, prostaglandin D2, 12-hydroxyheptadecatrienoic acid, 14,15-dihydroxyeicosatrienoic acid, 11-hydroxyeicosatetraenoic acid, 13-hydroxyoctadecadienoic acid, arachidonic acid, docosahexaenoic acid, dihomo-gamma-linolenic acid, gamma-linolenic acid, docosapentaenoic acid, EPA acid, docosatetraenoic acid, stearic acid, tryptophan, histidine, valine, threonine, cysteine, kynurenic acid, taurochenodeoxycholic acid, taurocholic acid, plasma triglycerides, plasmalogens, choline plasmalogen, myo-inositol phospholipids, glycerol phosphate, phosphate, lysophosphatidylcholine, lysophosphatidylethanolamine, and/or phosphatidylcholine. In several embodiments, the relative abundance of at least one metabolite of the subject is increased (e.g., relative to a healthy subject and/or a subject not in need of treatment). In several embodiments, the relative abundance of the metabolite (e.g., from a sample) is increased by equal to or at least about: 10%, 20%, 30%, 50%, 75%, 100%, 200%, 300%, 500%, 700%, 1000%, or ranges including and/or spanning the aforementioned values. In several embodiments, the relative abundance of the metabolite (e.g., from a sample) is increased by equal to or at least about: 1.1 fold, 1.2 fold, 1.3 fold, 1.5 fold, 1.75 fold, 2 fold, 2.5 fold, 3 fold, 4 fold, 5 fold, 7 fold, 10 fold, 20 fold, or ranges including and/or spanning the aforementioned values. In several embodiments, the relative abundance of at least one metabolite of the subject is decreased. In several embodiments, the relative abundance of the metabolite (e.g., from a sample) is decreased by equal to or at least about: 10%, 20%, 30%, 50%, 75%, 80%, 90%, 95%, or ranges including and/or spanning the aforementioned values. In several embodiments, the relative abundance of the metabolite (e.g., from a sample) is decreased by equal to or at least about: 1.5 fold, 2 fold, 4 fold, 5 fold, 8 fold, 10 fold, 20 fold, or ranges including and/or spanning the aforementioned values.

As disclosed elsewhere herein, in several embodiments, the biological indicator or at least one of the biological indicators is one or more DEGs. In several embodiments, the DEGs include one or more of Serpinb1a, Gna14, Serpina3m, Hsd11b1, Cyp2c29, Hsd11b1, Akr1d1, Aldh1a1, Mmp19, Gyp27a1, Tymp, Elov12, Chkb, H2afj, Tnfaip811, Tmem86a, Sel113, Agap2, 4833411C07Rik, Elov4, Fat3, Eef1a2, Atp7a, Rgs2, Cenpe, Mfap2, Mlki67, Ctsh, Trub2, Ube, H6pd, Eepd1, Acss2, Aacs, Gm36827, Man2a2, Nudt18, Plagl1, TM4sf19, Atp6v0d2, Gm20056, Trem2, I11rn, Mmp12, Cdk18, Efr3b, Tag1n2, Lurap1, Cp, I17rb, B230303O12Rik, Cfd, Sult1e1, Tdo2, Cyp2b9, Fads1, Hao2, Cyp2b13, Cyp2a22, Acnat2, Ildr2, Rpl10a-ps1, Tm6sf2, Fitm1, Tmem86a, Agap2, Lpar1, C6, Cmah, Lbp, Arsg, Glra3, Lad1, A730063M14Rik, Ly6f, Foxi1, Crygc, Defb28, Wfdc9, Ph1da2, Aqp6, Gm16411, Adam7, Ppp2r5b, Slc6a7, Gpr50, Ahnak2, S100a6, Mmp14, Htr2b, Hpgds, Mfap2, Gm18537, Aacs, Pclo, Adrb3, Gm38394, AC154232.2, Cadps, Adgrb2, Gm45470, Sdr9c7, Dsg1c, Slc17a1, Acnat2, Ces1c, Gss, Hsd11b1, 1810008118Rik, Tlcd1, Snrk, Akr1c20, Gm19950, Ttr, Cbfa2t3, Acat2, Pmvk, Abcd3, Acss2, Acacb, Arhgap27, Adam7, Rnase9, Wfdc8, Ighv9-2, DerI3, Acap1, Ccl19, Tcf7, Xkrx, Trim46, Zfp369, Zfp871, Pcdhb21, Adrb3, Gm14288, Uprt, Atm, Dchs2, Cped1, Gm38357, Cck, Ckap2, Gm4419, 1600015110Rik, Sez6I2, Prnd, Gm16702, S100a8, Pcdh12, Malat1, Kcnqlot1, ArI4c, Gm38394, Gm42549, AC154232.2, Gm37310, Gm37776, Atp2a1, Ckm, Tnnt3, Serpinb1a, Gna14, Serpina3m, Hsd11b1, Cyp2c29, Hsd11b1, Akr1d1, Aldh1a1, Mmp19, Adipoq, Fabp4, Lep, Rem, Hoxc8, Hoxc9, Cebpa, Dgat1, Dgat2, Elov13, Fads1, Fads2, Fas, Scd1, Srebf1, Adrb3, Hilpda, Lipe, Mg11, Plin1, Plin4, Pnpla2, Pnpla3, Ldah, Cs, Gckr, Me1, Pck1, Pdk4, Irs1, Acaa1a, Acads, Acox1, Cpt1a, Cpt1b, Hadhb, Ehhadh, Ppara, Ppargc1a, Cyp4a12a, Cyp4a12b, Cyp2e1, Adgre1, Agtr1a, Cc12, Cd14, Cd68, Il1b, Tm6sf2, Tnf, or combinations of any of the foregoing. In several embodiments, the DEGs include one or more of those shown in any one or more of FIG. 4a, FIG. 5a, FIG. 10a. FIG. 13a. FIG. 13g, FIG. 15e, FIG. 15f, or a combination of DEGs disclosed in the foregoing. In several embodiments, the DEG is measured using RNA sequence analysis as disclosed elsewhere herein.

In several embodiments, the relative abundance of at least one DEG of the subject is increased (e.g., relative to a healthy subject and/or a subject not in need of treatment). In several embodiments, the relative abundance of the DEG (e.g., from a sample) is increased by a factor of equal to or at least about: 2 fold, 3 fold, 4 fold, 5 fold, 10 fold, 20 fold, 30 fold, 40 fold, 50 fold, 75 fold, 100 fold, 150 fold, 200 fold, 1000 fold, 100,000 fold, 200,000 fold, or ranges including and/or spanning the aforementioned values. In several embodiments, the relative abundance of at least one DEG of the subject is decreased. In several embodiments, the relative abundance of the DEG (e.g., from a sample) is decreased by equal to or at least about: 2 fold, 3 fold, 4 fold, 5 fold, 10 fold, 20 fold, 30 fold, 40 fold, 50 fold, 75 fold, 100 fold, 150 fold, 200 fold, 1000 fold, 100,000 fold, 200,000 fold, or ranges including and/or spanning the aforementioned values.

As disclosed elsewhere herein, several embodiments provide methods of treatment. In several embodiments, the subject to be treated suffers from a FADS1 mediated disease or disorder (e.g., a disease or disorder that can be treated through modulation of the FADS1 enzyme). In several embodiments, before, concurrent with, or after administration a FADS1 inhibitor compound, a biological indicator for FADS1 activity is measured in the subject. In several embodiments, the FADS1 mediated disease or disorder is one or more of increased body-mass-index, obesity, a metabolic disorder, cardiovascular disorder, diabetes, dyslipidemia, and/or non-alcoholic steatohepatitis (NASH). The scope of the methods and uses provided in the instant disclosure is to be understood to encompass methods and uses employing all compounds disclosed herein. Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

In several embodiments, a compound as disclosed herein or a pharmaceutical composition comprising such compound is provided for use in reducing the body weight of a subject. In several embodiments, the compound or a pharmaceutical composition comprising compound is provided for use in reducing the body-mass-index of a subject. In several embodiments, the compound or a pharmaceutical composition comprising such compound is provided for use in treating a metabolic disorder. In several embodiments, the compound or a pharmaceutical composition comprising such compound is provided for use in treating a cardiovascular disorder. In several embodiments, the compound or a pharmaceutical composition comprising such compound is provided for use in treating diabetes. In several embodiments, the compound or a pharmaceutical composition comprising such compound is provided for use in treating obesity. In several embodiments, the compound or a pharmaceutical composition comprising such compound is provided for use in treating dyslipidemia. In several embodiments, the compound or a pharmaceutical composition comprising such compound is provided for use in treating non-alcoholic steatohepatitis (NASH).

In several embodiments, the compound or a pharmaceutical composition comprising such compound is used in the preparation of a medicament for reducing the body weight or the body-mass-index of a subject. In several embodiments, the compound or a pharmaceutical composition comprising such compound is used in the preparation of a medicament for treating a metabolic or a cardiovascular disorder. In several embodiments, the compound or a pharmaceutical composition comprising such compound is used in the preparation of a medicament for treating diabetes, obesity, dyslipidemia, or non-alcoholic steatohepatitis (NASH).

In several embodiments, the compound or a pharmaceutical composition comprising such compound is used in a method of reducing the body weight and/or the body-mass-index of a subject in need thereof. In several embodiments, the compound or a pharmaceutical composition comprising such compound is used in a method of treating a metabolic and/or a cardiovascular disorder in a subject in need thereof. In several embodiments, the compound or a pharmaceutical composition comprising such compound is used in a method of treating diabetes, obesity, dyslipidemia, and/or non-alcoholic steatohepatitis (NASH) in a subject in need thereof. In several embodiments, the compound or a pharmaceutical composition comprising such compound is used in a method of reducing the waist-to-hip ratio (WHR) of a subject in need thereof. In several embodiments, the method comprises administering to the subject a therapeutically effective amount of the compound or composition.

Provided herein as a further embodiment is a method of lowering blood glucose in a subject in need thereof, the method comprising administering to the subject a therapeutically effective amount of the compound or a pharmaceutical composition comprising such compound. In several embodiments, the method lowers blood glucose 10% or greater. In several embodiments, the method lowers blood glucose 15% or greater. In several embodiments, the method lowers blood glucose 20% or greater. In several embodiments, the method lowers blood glucose 25% or greater. In several embodiments, the method lowers blood glucose 30% or greater. In several embodiments, the method lowers blood glucose 35% or greater. In several embodiments, the method lowers blood glucose 40% or greater. In several embodiments, the method lowers blood glucose 50% or greater. In several embodiments, the method lowers blood glucose while having minimal effect on food intake/appetite. In several embodiments, the method lowers blood glucose while having no effect on food intake/appetite.

Provided herein as a further embodiment is a method of lowering insulin in a subject in need thereof, the method comprising administering to the subject the compound or a pharmaceutical composition comprising such compound. In several embodiments, the method lowers insulin 50% or greater. In several embodiments, the method lowers insulin 60% or greater. In several embodiments, the method lowers insulin 70% or greater. In several embodiments, the method lowers insulin 80% or greater. In several embodiments, the method lowers blood insulin 85% or greater. In several embodiments, the method lowers insulin 86% or greater. In several embodiments, the method lowers insulin 87% or greater. In several embodiments, the method lowers insulin 88% or greater. In several embodiments, the method lowers insulin 89% or greater. In several embodiments, the method lowers insulin 90% or greater. In several embodiments, the method lowers insulin 91% or greater. In several embodiments, the method lowers insulin while having minimal effect on food intake/appetite. In several embodiments, the method lowers insulin while having no effect on food intake/appetite.

Provided herein as a further embodiment is a method of lowering cholesterol in a subject in need thereof, the method comprising administering to the subject a therapeutically effective amount of the compound or a pharmaceutical composition comprising such compound. In several embodiments, the method lowers cholesterol 10% or greater. In several embodiments, the method lowers cholesterol 15% or greater. In several embodiments, the method lowers cholesterol 20% or greater. In several embodiments, the method lowers cholesterol 30% or greater. In several embodiments, the method lowers cholesterol 31% or greater. In several embodiments, the method lowers cholesterol 32% or greater. In several embodiments, the method lowers cholesterol 33% or greater. In several embodiments, the method lowers cholesterol 34% or greater. In several embodiments, the method lowers cholesterol 35% or greater. In several embodiments, the method lowers blood cholesterol 36% or greater. In several embodiments, the method lowers cholesterol 37% or greater. In several embodiments, the method lowers cholesterol 38% or greater. In several embodiments, the method lowers cholesterol 39% or greater. In several embodiments, the method lowers cholesterol while having minimal effect on food intake/appetite. In several embodiments, the method lowers cholesterol while having no effect on food intake/appetite.

Provided herein as a further embodiment is a method of lowering LDL in a subject in need thereof, the method comprising administering to the subject a therapeutically effective amount of the compound or a pharmaceutical composition comprising such compound. In several embodiments, the method lowers low-density lipoproteins (LDL) 10% or greater. In several embodiments, the method lowers LDL 20% or greater. In several embodiments, the method lowers LDL 21% or greater. In several embodiments, the method lowers LDL 22% or greater. In several embodiments, the method lowers LDL 23% or greater. In several embodiments, the method lowers LDL 24% or greater. In several embodiments, the method lowers LDL 25% or greater. In several embodiments, the method lowers LDL 26% or greater. In several embodiments, the method lowers blood LDL 27% or greater. In several embodiments, the method lowers LDL while having minimal effect on food intake/appetite. In several embodiments, the method lowers LDL while having no effect on food intake/appetite.

Provided herein as a further embodiment is a method of lowering triglycerides in a subject in need thereof, the method comprising administering to the subject a therapeutically effective amount of the compound or a pharmaceutical composition comprising such compound. In several embodiments, the method lowers triglycerides 30% or greater. In several embodiments, the method lowers triglycerides 40% or greater. In several embodiments, the method lowers triglycerides 50% or greater. In several embodiments, the method lowers triglycerides 51% or greater. In several embodiments, the method lowers triglycerides 52% or greater. In several embodiments, the method lowers triglycerides 53% or greater. In several embodiments, the method lowers triglycerides 54% or greater. In several embodiments, the method lowers triglycerides 55% or greater. In several embodiments, the method lowers blood triglycerides 56% or greater. In several embodiments, the method lowers triglycerides 57% or greater. In several embodiments, the method lowers triglycerides while having minimal effect on food intake/appetite. In several embodiments, the method lowers triglycerides while having no effect on food intake/appetite

Provided herein as a further embodiment is a method of lowering fat mass in a subject in need thereof, the method comprising administering to the subject a therapeutically effective amount of the compound or a pharmaceutical composition comprising such compound. In several embodiments, the method lowers fat mass of a subject 30% or greater. In several embodiments, the method lowers fat mass of a subject 40% or greater. In several embodiments, the method lowers fat mass of a subject 45% or greater. In several embodiments, the method lowers fat mass of a subject 50% or greater. In several embodiments, the method lowers fat mass of a subject 55% or greater. In several embodiments, the method lowers blood fat mass of a subject 60% or greater. In several embodiments, the method lowers fat mass of a subject 65% or greater. In several embodiments, the method lowers fat mass of a subject 70% or greater. In several embodiments, the method lowers fat mass of a subject 75% or greater. In several embodiments, the method lowers fat mass of a subject while having minimal effect on food intake/appetite. In several embodiments, the method lowers fat mass of a subject while having no effect on food intake/appetite.

Provided herein as a further embodiment is a method of raising adiponectin in a subject in need thereof, the method comprising administering to the subject a therapeutically effective amount of the compound or a pharmaceutical composition comprising such compound.

Provided herein as a further embodiment is a method of lowering leptin in a subject in need thereof, the method comprising administering to the subject a therapeutically effective amount of the compound or a pharmaceutical composition comprising such compound.

Provided herein as a further embodiment is a method of lowering resistance in a subject in need thereof, the method comprising administering to the subject a therapeutically effective amount of the compound or a pharmaceutical composition comprising such compound.

The various method steps described above may be performed in an alternate sequence or order to give desired results.

Additionally, results of treatment in the methods disclosed herein provide further opportunities to tailor dosing regimens for patients. For example, results of treatment (e.g., amount of weight loss, reduction in dyslipidemia, etc.) after a period of treatment (e.g., equal to or greater than 2 months, 6 months, etc.) is lower than expected, the dosing of a FADS1 inhibitor may be increased. Where downstream results of treatment (e.g., amount of weight loss, reduction in dyslipidemia, etc.) after a period of treatment (e.g., equal to or greater than 2 months, 6 months, etc.) is higher than expected or desired, the dosing of a FADS1 inhibitor may be maintained or decreased.

### FADS1 Inhibitor Compounds

In several embodiments, the method of treating FADS1 mediated diseases or disorders is performed by administering to a subject in need of treatment a compound as disclosed herein (e.g., a FADS1 inhibiting compound), a tautomer thereof, or a pharmaceutically acceptable salt of said compound or said tautomer.

Provided herein as Embodiment 62 is the method or compound of any one of Embodiments 1 to 61, wherein the FADS1 inhibitor compound is:
6-[1-(2,2,3,3,3-pentafluoropropyl)-1H-pyrazol-4-yl]-7-(trifluoromethyl)-5H-[1,3]thiazolo[3,2-a]pyrimidin-5-one;
2-methyl-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
1-methyl-6-[1-(2,2,3,3,3-pentafluoropropyl)-1H-pyrazol-4-yl]-7-(trifluoromethyl)-1H,2H,3H,5H-imidazo[1,2-a]pyrimidine-2,5-dione;
2-methyl-6-[1-(2,2,3,3,3-pentafluoropropyl)-1H-pyrazol-4-yl]-7-(trifluoromethyl)-5H-[1,3]thiazolo[3,2-a]pyrimidin-5-one;
3-methyl-6-[1-(2,2,3,3,3-pentafluoropropyl)-1H-pyrazol-4-yl]-7-(trifluoromethyl)-5H-[1,3]thiazolo[3,2-a]pyrimidin-5-one;
6-[1-(2,2,3,3,3-pentafluoropropyl)-1H-pyrazol-4-yl]-3,7-bis(trifluoromethyl)-5H-[1,3]thiazolo[3,2-a]pyrimidin-5-one;
2-fluoro-6-[1-(2,2,3,3,3-pentafluoropropyl)-1H-pyrazol-4-yl]-7-(trifluoromethyl)-5H-[1,3]thiazolo[3,2-a]pyrimidin-5-one;
7-(trifluoromethyl)-6-[1-(3,3,3-trifluoropropyl)-1H-pyrazol-4-yl]-5H-[1,3]thiazolo[3,2-a]pyrimidin-5-one;
2-chloro-6-[1-(2,2,3,3,3-pentafluoropropyl)-1H-pyrazol-4-yl]-7-(trifluoromethyl)-5H-[1,3]thiazolo[3,2-a]pyrimidin-5-one;
2-(methoxymethyl)-7-(trifluoromethyl)-6-[1-(3,3,3-trifluoropropyl)-1H-pyrazol-4-yl]-5H-[1,3,4]thiadiazolo[3,2-a]pyrimidin-5-one:
2-cyclopropyl-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one:
2-cyclopropyl-7-(trifluoromethyl)-6-[1-(3,3,3-trifluoropropyl)-1H-pyrazol-4-yl]-5H-[1,3,4]thiadiazolo[3,2-a]pyrimidin-5-one;
2,3-dimethyl-6-[1-(2,2,3,3,3-pentafluoropropyl)-1H-pyrazol-4-yl]-5-(trifluoromethyl)-3H,7H-[1,2,4]triazolo[1,5-a]pyrimidin-7-one;
2,3-dimethyl-5-(trifluoromethyl)-6-[1-(3,3,3-trifluoropropyl)-1H-pyrazol-4-yl]-3H,7H-[1,2,4]triazolo[1,5-a]pyrimidin-7-one:
7-ethyl-2-methyl-6-[1-(2,2,3,3,3-pentafluoropropyl)-1H-pyrazol-4-yl]-5H-[1,3,4]thiadiazolo[3,2-a]pyrimidin-5-one;
1,2-dimethyl-6-[1-(2,2,3,3,3-pentafluoropropyl)-1H-pyrazol-4-yl]-5-(trifluoromethyl)-1H,7H-pyrazolo[1,5-a]pyrimidin-7-one;
1-methyl-6-[1-(2,2,3,3,3-pentafluoropropyl)-1H-pyrazol-4-yl]-5-(trifluoromethyl)-1H,7H-pyrazolo[1,5-a]pyrimidin-7-one;
1,3-dimethyl-6-[1-(2,2,3,3,3-pentafluoropropyl)-1H-pyrazol-4-yl]-5-(trifluoromethyl)-1H,7H-pyrazolo[1,5-a]pyrimidin-7-one;
3-[1-(2,2,3,3,3-pentafluoropropyl)-1H-pyrazol-4-yl]-2-(trifluoromethyl)-4H,6H,7H,8H-pyrrolo[1,2-a]pyrimidin-4-one;
2-methyl-7-(trifluoromethyl)-6-[1-(3,3,3-trifluoropropyl)-1H-pyrazol-4-yl]-5H-[1,3,4]thiadiazolo[3,2-a]pyrimidin-5-one;
2-(trifluoromethyl)-3-[1-(3,3,3-trifluoropropyl)-1H-pyrazol-4-yl]-4H,6H,7H,8H-pyrrolo[1,2-a]pyrimidin-4-one;
6-[1-(2,2,3,3,3-pentafluoropropyl)-1H-pyrazol-4-yl]-7-(trifluoromethyl)-2H,3H,5H-[1,3]thiazolo[3,2-a]pyrimidin-5-one;
6-{1-[(2,2-difluorocyclopropyl)methyl]-1H-pyrazol-4-yl}-2-methyl-7-(trifluoromethyl)-5H-[1,3,4]thiadiazolo[3,2-a]pyrimidin-5-one:
2-methyl-6-[1-(2,2,3,3,3-pentafluoropropyl)-1H-pyrazol-4-yl]-7-(trifluoromethyl)-2H,3H,5H-[1,3]thiazolo[3,2-a]pyrimidin-5-one;
8-[1-(2,2,3,3,3-pentafluoropropyl)-1H-pyrazol-4-yl]-9-(trifluoromethyl)-6,10-diazatricyclo[4.4.0.0^{2,4}]deca-1(10),8-dien-7-one;
6-{1-[(3,3-difluorocyclobutyl)methyl]-1H-pyrazol-4-yl}-2-methyl-7-(trifluoromethyl)-5H-[1,3,4]thiadiazolo[3,2-a]pyrimidin-5-one;
1-methyl-6-[1-(2,2,3,3,3-pentafluoropropyl)-1H-pyrazol-4-yl]-7-(trifluoromethyl)-1H,5H-[1,2,4]triazolo[4,3-a]pyrimidin-5-one;
3-methyl-6-[1-(2,2,3,3,3-pentafluoropropyl)-1H-pyrazol-4-yl]-5-(trifluoromethyl)-3H,7H-[1,2,3,4]tetrazolo[1,5-a]pyrimidin-7-one;
2-methyl-6-{1-[(oxetan-3-yl)methyl]-1H-pyrazol-4-yl}-7-(trifluoromethyl)-5H-[1,3,4]thiadiazolo[3,2-a]pyrimidin-5-one;
2-methyl-6-[1-(2,2,3,3,3-pentafluoropropyl)-1H-pyrazol-3-yl]-7-(trifluoromethyl)-5H-[1,3,4]thiadiazolo[3,2-a]pyrimidin-5-one;
6-[4-(2,2,2-trifluoroethoxy)phenyl]-7-(trifluoromethyl)-5H-[1,3]thiazolo[3,2-a]pyrimidin-5-one;
2-(hydroxymethyl)-6-[1-(2,2,3,3,3-pentafluoropropyl)-1H-pyrazol-4-yl]-7-(trifluoromethyl)-5H-[1,3]thiazolo[3,2-a]pyrimidin-5-one;
2-(hydroxymethyl)-6-[4-(2,2,2-trifluoroethoxy)phenyl]-7-(trifluoromethyl)-5H-[1,3]thiazolo[3,2-a]pyrimidin-5-one;
2-chloro-6-[4-(2,2,2-trifluoroethoxy)phenyl]-7-(trifluoromethyl)-5H-[1,3]thiazolo[3,2-a]pyrimidin-5-one;
6-[1-(2,2,3,3,3-pentafluoropropyl)-1H-pyrazol-4-yl]-1-(propan-2-yl)-7-(trifluoromethyl)-1H,2H,3H,5H-imidazo[1,2-a]pyrimidine-2,5-dione;
1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
3-methyl-6-[1-(2,2,3,3,3-pentafluoropropyl)-1H-pyrazol-4-yl]-5-(trifluoromethyl)-3H,7H-[1,2,4]triazolo[1,5-a]pyrimidin-7-one;
2-methyl-6-[1-(2,2,3,3,3-pentafluoropropyl)-1H-pyrazol-4-yl]-7-(trifluoromethyl)-5H-[1,3]oxazolo[3,2-a]pyrimidin-5-one;
2-methyl-6-[4-(2,2,2-trifluoroethoxy)phenyl]-7-(trifluoromethyl)-5H-[1,3,4]thiadiazolo[3,2-a]pyrimidin-5-one;
7-ethoxy-2-methyl-6-[1-(2,2,3,3,3-pentafluoropropyl)-1H-pyrazol-4-yl]-5H-[1,3,4]thiadiazolo[3,2-a]pyrimidin-5-one;
2-(methoxymethyl)-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one:
2-methoxy-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
3-chloro-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-(hydroxymethyl)-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
2-(hydroxymethyl)-7-(trifluoromethyl)-6-[1-(3,3,3-trifluoropropyl)-1H-pyrazol-4-yl]-5H-[1,3,4]thiadiazolo[3,2-a]pyrimidin-5-one;
6-[1-(2,2,3,3,3-pentafluoropropyl)-1H-pyrazol-4-yl]-7-(trifluoromethyl)-1H,2H,3H,5H-imidazo[1,2-a]pyrimidine-2,5-dione;
2-chloro-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-chloro-6-[1-(2,2,3,3,3-pentafluoropropyl)-1H-pyrazol-4-yl]-7-(trifluoromethyl)-1H,5H-imidazo[1,2-a]pyrimidin-5-one;
2-cyclopropyl-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-chloro-1-methyl-7-(trifluoromethyl)-6-[1-(3,3,3-trifluoropropyl)-1H-pyrazol-4-yl]-1H,5H-imidazo[1,2-a]pyrimidin-5-one;
1,2-dimethyl-6-[1-(2,2,3,3,3-pentafluoropropyl)-1H-pyrazol-4-yl]-7-(trifluoromethyl)-1H,5H-imidazo[1,2-a]pyrimidin-5-one;
1,2-dimethyl-7-(trifluoromethyl)-6-[1-(3,3,3-trifluoropropyl)-1H-pyrazol-4-yl]-1H,5H-imidazo[1,2-a]pyrimidin-5-one;
1,2-dimethyl-6-[4-(2,2,2-trifluoroethoxy)phenyl]-7-(trifluoromethyl)-1H,5H-imidazo[1,2-a]pyrimidin-5-one;
2-(methoxymethyl)-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1-ethyl-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1-(2-methoxyethyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(propan-2-yl)-7-(trifluoromethyl)-1H,5H-imidazo[1,2-a]pyrimidin-5-one;
6-{I-[(2,2-difluorocyclopropyl)methyl]-1H-pyrazol-4-yl}-1,2-dimethyl-7-(trifluoromethyl)-1H,5H-imidazo[1,2-a]pyrimidin-5-one;
6-{1-[(3,3-difluorocyclobutyl)methyl]-1H-pyrazol-4-yl}-1,2-dimethyl-7-(trifluoromethyl)-1H,5H-imidazo[1,2-a]pyrimidin-5-one:
6-[1-(cyclopropylmethyl)-1H-pyrazol-4-yl]-1,2-dimethyl-7-(trifluoromethyl)-1H,5H-imidazo[1,2-a]pyrimidin-5-one;
1-(cyclopropylmethyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-(methoxymethyl)-1-methyl-7-(trifluoromethyl)-6-[1-(3,3,3-trifluoropropyl)-1H-pyrazol-4-yl]-1H,5H-imidazo[1,2-a]pyrimidin-5-one;
1-(2-hydroxypropyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1,2-dimethyl-6-{1-[(oxetan-3-yl)methyl]-1H-pyrazol-4-yl}-7-(trifluoromethyl)-1H,5H-imidazo[1,2-a]pyrimidin-5-one;
1-(cyclopropylmethyl)-2-methyl-7-(trifluoromethyl)-6-[1-(3,3,3-trifluoropropyl)-1H-pyrazol-4-yl]-1H,5H-imidazo[1,2-a]pyrimidin-5-one;
1-[2-(dimethylamino)ethyl]-2-methyl-7-(trifluoromethyl)-6-[1-(3,3,3-trifluoropropyl)-1H-pyrazol-4-yl]-1H,5H-imidazo[1,2-a]pyrimidin-5-one;
1-(cyclopropylmethyl)-2-methyl-6-[4-(2,2,2-trifluoroethoxy)phenyl]-7-(trifluoromethyl)-1H,5H-imidazo[1,2-a]pyrimidin-5-one;
1-[2-(dimethylamino)ethyl]-2-methyl-6-[4-(2,2,2-trifluoroethoxy)phenyl]-7-(trifluoromethyl)-1H,5H-imidazo[1,2-a]pyrimidin-5-one;
1,2-dimethyl-6-[1-(2,2,3,3,3-pentafluoropropyl)-1H-pyrazol-3-yl]-7-(trifluoromethyl)-1H,5H-imidazo[1,2-a]pyrimidin-5-one;
2-methoxy-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methoxy-1-methyl-7-(trifluoromethyl)-6-[1-(3,3,3-trifluoropropyl)-1H-pyrazol-4-yl]-1H,5H-imidazo[1,2-a]pyrimidin-5-one;
2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-7-(trifluoromethyl)-6-[1-(3,3,3-trifluoropropyl)-1H-pyrazol-4-yl]-1H,5H-imidazo[1,2-a]pyrimidin-5-one;
2-methyl-6-[4-(2,2,2-trifluoroethoxy)phenyl]-7-(trifluoromethyl)-1H,5H-imidazo[1,2-a]pyrimidin-5-one;
6-{1-[(2,2-difluorocyclopropyl)methyl]-1H-pyrazol-4-yl}-2-methyl-7-(trifluoromethyl)-1H,5H-imidazo[1,2-a]pyrimidin-5-one;
1-(²H₃)methyl-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1-(²H₃)methyl-2-methyl-7-(trifluoromethyl)-6-[1-(3,3,3-trifluoropropyl)-1H-pyrazol-4-yl]-1H,5H-imidazo[1,2-a]pyrimidin-5-one:
1-(2-hydroxyethyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
methyl 2-methyl-5-oxo-6-[1-(2,2,3,3,3-pentafluoropropyl)-1H-pyrazol-4-yl]-7-(trifluoromethyl)-1H,5H-imidazo[1,2-a]pyrimidine-1-carboxylate;
1-[(2,2-difluorocyclopropyl)methyl]-2-methyl-7-(trifluoromethyl)-6-[1-(3,3,3-trifluoropropyl)-1H-pyrazol-4-yl]-1H,5H-imidazo[1,2-a]pyrimidin-5-one;
1-[(3,3-difluorocyclobutyl)methyl]-2-methyl-7-(trifluoromethyl)-6-[1-(3,3,3-trifluoropropyl)-1H-pyrazol-4-yl]-1H,5H-imidazo[1,2-a]pyrimidin-5-one;
1-(2-hydroxyethyl)-2-methyl-7-(trifluoromethyl)-6-[1-(3,3,3-trifluoropropyl)-1H-pyrazol-4-yl]-1H,5H-imidazo[1,2-a]pyrimidin-5-one;
1-[2-(dimethylamino)ethyl]-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(prop-2-yn-1-yl)-7-(trifluoromethyl)-1H,5H-imidazo[1,2-a]pyrimidin-5-one;
2-{2-methyl-5-oxo-6-[1-(2,2,3,3,3-pentafluoropropyl)-1H-pyrazol-4-yl]-7-(trifluoromethyl)-1H,5H-imidazo[1,2-a]pyrimidin-1-yl}acetonitrile;
2-[2-methyl-5-oxo-7-(trifluoromethyl)-6-[1-(3,3,3-trifluoropropyl)-1H-pyrazol-4-yl]-1H,5H-imidazo[1,2-a]pyrimidin-1-yl]acetonitrile;
1-(2-hydroxy-2-methylpropyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1-[2-(1-hydroxycyclopropyl)ethyl]-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-1-[(oxetan-3-yl)methyl]-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-1-(oxetan-3-yl)-6-[1-(2,2,3,3,3-pentafluoropropyl)-1H-pyrazol-4-yl]-7-(trifluoromethyl)-1H,5H-imidazo[1,2-a]pyrimidin-5-one;
1,2-dimethyl-6-[1-(2,2,3,3,3-pentafluoropropyl)-1H-pyrazol-4-yl]-7-(trifluoromethyl)-1H,5H-imidazo[1,2-a]pyrimidine-5-thione;
2-methyl-6-[1-(2,2,3,3,3-pentafluoropropyl)-1H-pyrazol-4-yl]-1-(pyridin-2-yl)-7-(trifluoromethyl)-1H,5H-imidazo[1,2-a]pyrimidin-5-one;
2-methyl-1-(pyridin-2-yl)-7-(trifluoromethyl)-6-[1-(3,3,3-trifluoropropyl)-1H-pyrazol-4-yl]-1H,5H-imidazo[1,2-a]pyrimidin-5-one;
2-methyl-6-[1-(2,2,3,3,3-pentafluoropropyl)-1H-pyrazol-4-yl]-1-(pyrazin-2-yl)-7-(trifluoromethyl)-1H,5H-imidazo[1,2-a]pyrimidin-5-one;
2-methyl-1-(6-methylpyridin-2-yl)-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-1-(1-methyl-1H-pyrazol-4-yl)-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(pyridin-3-yl)-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-phenyl-7-(trifluoromethyl)-1H,5H-imidazo[1,2-a]pyrimidin-5-one;
1-(6-chloropyridin-2-yl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(pyridin-4-yl)-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(1H-pyrazol-4-yl)-7-(trifluoromethyl)-1H,5H-imidazo[1,2-a]pyrimidin-5-one;
2-(fluoromethyl)-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
2-[(dimethylamino)methyl]-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
6-(1-{[(1R)-2,2-difluorocyclopropyl]methyl}-1H-pyrazol-4-yl)-2-methyl-7-(trifluoromethyl)-1H,5H-imidazo[1,2-a]pyrimidin-5-one;
6-(1-{[(1S)-2,2-difluorocyclopropyl]methyl}-1H-pyrazol-4-yl)-2-methyl-7-(trifluoromethyl)-1H,5H-imidazo[1,2-a]pyrimidin-5-one;
6-(1-{[(1R)-2,2-difluorocyclopropyl]methyl}-1H-pyrazol-4-yl)-1,2-dimethyl-7-(trifluoromethyl)-1H,5H-imidazo[1,2-a]pyrimidin-5-one;
6-(1-{[(1S)-2,2-difluorocyclopropyl]methyl}-1H-pyrazol-4-yl)-1,2-dimethyl-7-(trifluoromethyl)-1H,5H-imidazo[1,2-a]pyrimidin-5-one;
6-(1-{[(1R)-2,2-difluorocyclopropyl]methyl}-1H-pyrazol-4-yl)-2-methyl-7-(trifluoromethyl)-5H-[1,3,4]thiadiazolo[3,2-a]pyrimidin-5-one;
6-(1-{[(1S)-2,2-difluorocyclopropyl]methyl}-1H-pyrazol-4-yl)-2-methyl-7-(trifluoromethyl)-5H-[1,3,4]thiadiazolo[3,2-a]pyrimidin-5-one;
(2R)-2-methyl-6-[1-(2,2,3,3,3-pentafluoropropyl)-1H-pyrazol-4-yl]-7-(trifluoromethyl)-2H,3H,5H-[1,3]thiazolo[3,2-a]pyrimidin-5-one;
(2S)-2-methyl-6-[1-(2,2,3,3,3-pentafluoropropyl)-1H-pyrazol-4-yl]-7-(trifluoromethyl)-2H,3H,5H,6H,7H-[1,3]thiazolo[3,2-a]pyrimidin-5-one;
1-{[(1R)-2,2-difluorocyclopropyl]methyl}-2-methyl-7-(trifluoromethyl)-6-[1-(3,3,3-trifluoropropyl)-1H-pyrazol-4-yl]-1H,5H-imidazo[1,2-a]pyrimidin-5-one;
1-{[(1S)-2,2-difluorocyclopropyl]methyl}-2-methyl-7-(trifluoromethyl)-6-[1-(3,3,3-trifluoropropyl)-1H-pyrazol-4-yl]-1H,5H-imidazo[1,2-a]pyrimidin-5-one;
(2R)-2-(4-(8-methoxy-4-oxo-2-(trifluoromethyl)-4H-pyrido[1,2-a]pyrimidin-3-yl)phenoxy)propanenitrile;
(2R)-2-(4-(8-methoxy-4-oxo-2-(trifluoromethyl)-4H-pyrimido[1,2-a]pyrimidin-3-yl)phenoxy)propanenitrile;
(2S)-2-(4-(8-methoxy-4-oxo-2-(trifluoromethyl)-4H-pyrido[1,2-a]pyrimidin-3-yl)phenoxy)propanenitrile;
(2S)-2-(4-(8-methoxy-4-oxo-2-(trifluoromethyl)-4H-pyrimido[1,2-a]pyrimidin-3-yl)phenoxy)propanenitrile:
(4-(8-methoxy-4-oxo-2-(trifluoromethyl)-4H-pyrido[1,2-a]pyrimidin-3-yl)-1H-pyrazol-1-yl)acetonitrile;
(4-(8-methoxy-4-oxo-2-(trifluoromethyl)-4H-pyrido[1,2-a]pyrimidin-3-yl)phenoxy)acetonitrile;
(4-(8-methoxy-4-oxo-2-(trifluoromethyl)-4H-pyrido[1,2-a]pyrimidin-3-yl)phenyl)acetonitrile;
(4-(8-methoxy-4-oxo-2-(trifluoromethyl)-4H-pyrimido[1,2-a]pyrimidin-3-yl)phenoxy)acetonitrile;
(4-oxo-3-(1-(2,2,3,3,3-pentafluoropropyl)-1H-pyrazol-4-yl)-2-(trifluoromethyl)-4H-pyrido[1,2-a]pyrimidin-8-yl)acetonitrile;
1-(chloromethyl)-7-[1-(2,2,3,3,3-pentafluoropropyl)-1H-pyrazol-4-yl]-8-(trifluoromethyl)-1H,2H,6H-pyrimido[1,2-a][1,3]diazine-2,6-dione;
1-(fluoromethyl)-7-[1-(2,2,3,3,3-pentafluoropropyl)-1H-pyrazol-4-yl]-8-(trifluoromethyl)-1H,2H,6H-[1,3]diazino[1,2-a]pyrimidine-2,6-dione;
1-(methyl-d₃)-7-(4-(2,2,2-trifluoroethoxy)phenyl)-8-(trifluoromethyl)-2H-pyrimido[1,2-a]pyrimidine-2,6(1H)-dione;
1-methyl-7-[1-(2,2,3,3,3-pentafluoropropyl)-1H-pyrazol-4-yl]-8-(trifluoromethyl)-1H,2H,6H-[1,3]diazino[1,2-a]pyrimidine-2,6-dione;
2-(4-(8-methoxy-4-oxo-2-(trifluoromethyl)-4H-pyrido[1,2-a]pyrimidin-3-yl)phenoxy)-2-methylpropanenitrile;
2-(4-(8-methoxy-4-oxo-2-(trifluoromethyl)-4H-pyrido[1,2-a]pyrimidin-3-yl)phenoxy)propanenitrile;
2-(4-(8-methoxy-4-oxo-2-(trifluoromethyl)-4H-pyrimido[1,2-a]pyrimidin-3-yl)phenoxy)propanenitrile;
2-(difluoromethyl)-3-(3-fluoro-4-(2,2,2-trifluoroethoxy)phenyl)-8-methoxy-4H-pyrido[1,2-a]pyrimidin-4-one;
2-(difluoromethyl)-4-oxo-3-(1-(2,2,3,3,3-pentafluoropropyl)-1H-pyrazol-4-yl)-4H-pyrido[1,2-a]pyrimidine-8-carbonitrile;
2-(difluoromethyl)-8-methoxy-3-(1-(3,3,3-trifluoropropyl)-1H-pyrazol-4-yl)-4H-pyrido[1,2-a]pyrimidin-4-one;
2-(difluoromethyl)-8-methoxy-3-(1-(4,4,4-trifluorobutyl)-1H-pyrazol-4-yl)-4H-pyrido[1,2-a]pyrimidin-4-one;
2-(difluoromethyl)-8-methoxy-3-(4-(2,2,2-trifluoroethoxy)phenyl)-4H-pyrido[1,2-a]pyrimidin-4-one;
2-(difluoromethyl)-8-methoxy-3-(6-(2,2,2-trifluoroethoxy)-3-pyridinyl)-4H-pyrido[1,2-a]pyrimidin-4-one;
2-(fluoromethyl)-4-oxo-3-(1-(2,2,3,3,3-pentafluoropropyl)-1H-pyrazol-4-yl)-4H-pyrido[1,2-a]pyrimidine-8-carbonitrile; 2-(fluoromethyl)-8-methoxy-3-(4-(2,2,2-trifluoroethoxy)phenyl)-4H-pyrido[1,2-a]pyrimidin-4-one;
2-(trifluoromethyl)-3-[1-(3,3,3-trifluoropropyl)-1H-pyrazol-4-yl]-4H,6H,7H,9H-pyrimido[2,1-c][1,4]oxazin-4-one;
2,8-dimethoxy-3-(4-(2,2,2-trifluoroethoxy)phenyl)-4H-pyrido[1,2-a]pyrimidin-4-one;
2-cyclopropyl-8-methoxy-3-(4-(2,2,2-trifluoroethoxy)phenyl)-4H-pyrido[1,2-a]pyrimidin-4-one;
2-ethoxy-8-methoxy-3-(4-(2,2,2-trifluoroethoxy)phenyl)-4H-pyrido[1,2-a]pyrimidin-4-one;
2-ethoxy-8-methoxy-3-[1-(2,2,3,3,3-pentafluoropropyl)-1H-pyrazol-4-yl]-4H-pyrido[1,2-a]pyrimidin-4-one;
2-ethyl-8-methoxy-3-(1-(2,2,3,3,3-pentafluoropropyl)-1H-pyrazol-4-yl)-4H-pyrido[1,2-a]pyrimidin-4-one:
2-ethyl-8-methoxy-3-(4-(2,2,2-trifluoroethoxy)phenyl)-4H-pyrido[1,2-a]pyrimidin-4-one;
2-ethyl-8-methoxy-3-(6-(2,2,2-trifluoroethoxy)-3-pyridinyl)-4H-pyrido[1,2-a]pyrimidin-4-one;
3-(1-(2,2,3,3,3-pentafluoropropyl)-1H-pyrazol-4-yl)-2-(trifluoromethyl)-4H-pyrido[1,2-a]pyrimidine-4,8(1H)-dione;
3-(1-(2,2-difluoropropyl)-1H-pyrazol-4-yl)-8-methoxy-2-(trifluoromethyl)-4H-pyrido[1,2-a]pyrimidin-4-one;
3-(1-(3-fluorophenyl)-1H-pyrazol-4-yl)-8-methoxy-2-(trifluoromethyl)-4H-pyrido[1,2-a]pyrimidin-4-one;
3-(1-(4-fluorophenyl)-1H-pyrazol-4-yl)-2-(trifluoromethyl)-4H-pyrido[1,2-a]pyrimidin-4-one;
3-(1-(4-fluorophenyl)-1H-pyrazol-4-yl)-8-methoxy-2-(trifluoromethyl)-4H-pyrido[1,2-a]pyrimidin-4-one;
3-(1-{[(1R)-2,2-difluorocyclopropyl]methyl}-1H-pyrazol-4-yl)-8-methoxy-2-(trifluoromethyl)-4H-pyrido[1,2-a]pyrimidin-4-one;
3-(1-{[(1S)-2,2-difluorocyclopropyl]methyl}-1H-pyrazol-4-yl)-8-methoxy-2-(trifluoromethyl)-4H-pyrido[1,2-a]pyrimidin-4-one:
3-(1-benzofuran-2-yl)-8-methoxy-2-(trifluoromethyl)-4H-pyrido[1,2-a]pyrimidin-4-one;
3-(1-cyclopropyl-1H-pyrazol-4-yl)-2-(trifluoromethyl)-4H-pyrido[1,2-a]pyrimidin-4-one;
3-(1-cyclopropyl-1H-pyrazol-4-yl)-8-methoxy-2-(trifluoromethyl)-4H-pyrido[1,2-a]pyrimidin-4-one;
3-(1-phenyl-1H-pyrazol-4-yl)-2-(trifluoromethyl)-4H-pyrido[1,2-a]pyrimidin-4-one;
3-(1-propyl-1H-pyrazol-4-yl)-2-(trifluoromethyl)-4H-pyrido[1,2-a]pyrimidin-4-one;
3-(2-chloro-4-(2,2,2-trifluoroethoxy)phenyl)-8-methoxy-2-(trifluoromethyl)-4H-pyrido[1,2-a]pyrimidin-4-one;
3-(2-fluoro-4-(2,2,2-trifluoroethoxy)phenyl)-2-(trifluoromethyl)-4H-pyrido[1,2-a]pyrimidin-4-one;
3-(2-fluoro-4-(2,2,2-trifluoroethoxy)phenyl)-8-methoxy-2-(trifluoromethyl)-4H-pyrido[1,2-a]pyrimidin-4-one;
3-(2-fluoro-4-(2,2,2-trifluoroethoxy)phenyl)-8-methoxy-2-(trifluoromethyl)-4H-pyrimido[1,2-a]pyrimidin-4-one;
3-(2-fluoro-4-(trifluoromethoxy)phenyl)-8-methoxy-2-(trifluoromethyl)-4H-pyrido[1,2-a]pyrimidin-4-one:
3-(2-fluoro-6-(2,2,2-trifluoroethoxy)-3-pyridinyl)-8-methoxy-2-(trifluoromethyl)-4H-pyrido[1,2-a]pyrimidin-4-one;
3-(3-chloro-4-(2,2,2-trifluoroethoxy)phenyl)-2-(trifluoromethyl)-4H-pyrido[1,2-a]pyrimidin-4-one;
3-(3-fluoro-4-(2,2,2-trifluoroethoxy)phenyl)-8-methoxy-2-(trifluoromethyl)-4H-pyrido[1,2-a]pyrimidin-4-one;
3-(3-fluoro-4-(2,2,2-trifluoroethoxy)phenyl)-8-methoxy-2-(trifluoromethyl)-4H-pyrimido[1,2-a]pyrimidin-4-one;
3-(4-(((1R)-2,2-difluorocyclopropyl)methoxy)phenyl)-8-methoxy-2-(trifluoromethyl)-4H-pyrido[1,2-a]pyrimidin-4-one;
3-(4-(((1S)-2,2-difluorocyclopropyl)methoxy)phenyl)-8-methoxy-2-(trifluoromethyl)-4H-pyrido[1,2-a]pyrimidin-4-one;
3-(4-((2,2-difluorocyclopropyl)methoxy)phenyl)-8-methoxy-2-(trifluoromethyl)-4H-pyrido[1,2-a]pyrimidin-4-one;
3-(4-(2,2,2-trifluoroethoxy)phenyl)-2-(trifluoromethyl)-4H-pyrido[1,2-a]pyrimidin-4-one;
3-(4-(2,2,2-trifluoroethoxy)phenyl)-2-(trifluoromethyl)-4H-pyrido[1,2-a]pyrimidine-4,8(1H)-dione;
3-(4-(2,2,2-trifluoroethoxy)phenyl)-2,8-bis(trifluoromethyl)-4H-pyrido[1,2-a]pyrimidin-4-one;
3-(4-(2,2-difluoroethoxy)-2-fluorophenyl)-8-methoxy-2-(trifluoromethyl)-4H-pyrido[1,2-a]pyrimidin-4-one;
3-(4-(2,2-difluoroethoxy)phenyl)-8-(methyloxy-d₃)-2-(trifluoromethyl)-4H-pyrido[1,2-a]pyrimidin-4-one;
3-(4-(2,2-difluoroethoxy)phenyl)-8-methoxy-2-(trifluoromethyl)-4H-pyrido[1,2-a]pyrimidin-4-one;
3-(4-(2,2-difluoropropoxy)phenyl)-8-methoxy-2-(trifluoromethyl)-4H-pyrido[1,2-a]pyrimidin-4-one;
3-(4-(2-fluoroethoxy)phenyl)-8-(methyloxy-d₃)-2-(trifluoromethyl)-4H-pyrido[1,2-a]pyrimidin-4-one;
3-(4-(2-fluoroethoxy)phenyl)-8-(methyloxy-d₃)-2-(trifluoromethyl)-4H-pyrimido[1,2-a]pyrimidin-4-one;
3-(4-(2-fluoroethoxy)phenyl)-8-methoxy-2-(trifluoromethyl)-4H-pyrido[1,2-a]pyrimidin-4-one;
3-(4-(2-fluoroethoxy)phenyl)-8-methoxy-2-(trifluoromethyl)-4H-pyrimido[1,2-a]pyrimidin-4-one;
3-(4-(8-methoxy-4-oxo-2-(trifluoromethyl)-4H-pyrido[1,2-a]pyrimidin-3-yl)-1H-pyrazol-1-yl)propanenitrile:
3-(4-(8-methoxy-4-oxo-2-(trifluoromethyl)-4H-pyrido[1,2-a]pyrimidin-3-yl)phenyl)propanenitrile;
3-(4-(cyclopropylmethoxy)-2-fluorophenyl)-8-methoxy-2-(trifluoromethyl)-4H-pyrido[1,2-a]pyrimidin-4-one;
3-(4-(cyclopropylmethoxy)phenyl)-8-(methyloxy-d₃)-2-(trifluoromethyl)-4H-pyrido[1,2-a]pyrimidin-4-one;
3-(4-(cyclopropylmethoxy)phenyl)-8-methoxy-2-(trifluoromethyl)-4H-pyrido[1,2-a]pyrimidin-4-one;
3-(4-(cyclopropyhmethoxy)phenyl)-8-methoxy-2-(trifluoromethyl)-4H-pyrimido[1,2-a]pyrimidin-4-one;
3-(4-(difluoromethoxy)phenyl)-8-methoxy-2-(trifluoromethyl)-4H-pyrido[1,2-a]pyrimidin-4-one;
3-(5-(2,2,2-trifluoroethoxy)-2-pyridinyl)-2-(trifluoromethyl)-4H-pyrido[1,2-a]pyrimidin-4-one;
3-(5-fluoro-6-(2,2,2-trifluoroethoxy)-3-pyridinyl)-8-methoxy-2-(trifluoromethyl)-4H-pyrido[1,2-a]pyrimidin-4-one;
3-(6-(2,2,2-trifluoroethoxy)-3-pyridinyl)-2-(trifluoromethyl)-4H-pyrido[1,2-a]pyrimidin-4-one;
3-[1-(2,2,3,3,3-pentafluoropropyl)-1H-pyrazol-4-yl]-2-(trifluoromethyl)-4H,6H,7H,9H-pyrimido[2,1-c][1,4]oxazin-4-one;
3-[1-(2,2,3,3,3-pentafluoropropyl)-1H-pyrazol-4-yl]-2-(trifluoromethyl)-4H-pyrazino[1,2-a]pyrimidin-4-one;
3-[1-(2,2,3,3,3-pentafluoropropyl)-1H-pyrazol-4-yl]-2-(trifluoromethyl)-4H-pyrido[1,2-a]pyrimidin-4-one;
3-[1-(cyclopropylmethyl)-1H-pyrazol-4-yl]-8-methoxy-2-(trifluoromethyl)-4H-pyrido[1,2-a]pyrimidin-4-one;
3-[1-(cyclopropylmethyl)-1H-pyrazol-4-yl]-8-methyl-2-(trifluoromethyl)-4H-pyrimido[1,2-b]pyridazin-4-one;
3-[5-iodo-1-(2,2,3,3,3-pentafluoropropyl)-1H-1,2,3-triazol-4-yl]-8-methoxy-2-(trifluoromethyl)-4H-pyrido[1,2-a]pyrimidin-4-one;
3-{1-[(2,2-difluorocyclopropyl)methyl]-1H-pyrazol-4-yl}-7-fluoro-8-methoxy-2-(trifluoromethyl)-4H-pyrido[1,2-a]pyrimidin-4-one:
3-{1-[(2,2-difluorocyclopropyl)methyl]-1H-pyrazol-4-yl}-8-methoxy-2-(trifluoromethyl)-4H-pyrido[1,2-a]pyrimidin-4-one;
3-{1-[(2,2-difluorocyclopropyl)methyl]-1H-pyrazol-4-yl}-8-methyl-2-(trifluoromethyl)-4H-pyrimido[1,2-b]pyridazin-4-one;
3-{1-[(3,3-difluorocyclobutyl)methyl]-1H-pyrazol-4-yl}-8-methoxy-2-(trifluoromethyl)-4H-pyrido[1,2-a]pyrimidin-4-one;
3-{1-[(3,3-difluorocyclobutyl)methyl]-1H-pyrazol-4-yl}-8-methoxy-2-(trifluoromethyl)-4H-pyrimido[1,2-b]pyridazin-4-one;
3-{1-[(3,3-difluorocyclobutyl)methyl]-1H-pyrazol-4-yl}-8-methyl-2-(trifluoromethyl)-4H-pyrimido[1,2-b]pyridazin-4-one;
3-fluoro-1-methyl-7-[1-(2,2,3,3,3-pentafluoropropyl)-1H-pyrazol-4-yl]-8-(trifluoromethyl)-1H,2H,6H-[1,3]diazino[1,2-a]pyrimidine-2,6-dione;
4-oxo-3-(1-(2,2,3,3,3-pentafluoropropyl)-1H-pyrazol-4-yl)-2-(trifluoromethyl)-4H-pyrido[1,2-a]pyrimidine-8-carbonitrile;
4-oxo-3-(1-(2,2,3,3,3-pentafluoropropyl)-1H-pyrazol-4-yl)-2-(trifluoromethyl)-4H-pyrido[1,2-a]pyrimidine-8-carboxylic acid;
4-oxo-3-(4-(2,2,2-trifluoroethoxy)phenyl)-2-(trifluoromethyl)-4H-pyrido[1,2-a]pyrimidine-8-carbonitrile;
4-oxo-3-(4-(2,2,2-trifluoroethoxy)phenyl)-2-(trifluoromethyl)-4H-pyrido[1,2-a]pyrimidine-8-carboxamide;
4-oxo-3-(4-(2,2,2-trifluoroethoxy)phenyl)-2-(trifluoromethyl)-4H-pyrido[1,2-a]pyrimidine-8-carboxylic acid;
4-oxo-3-[1-(2,2,3,3,3-pentafluoropropyl)-1H-pyrazol-4-yl]-2-(trifluoromethyl)-4H-pyrido[1,2-a]pyrimidine-7-carbonitrile;
7-(1-(2,2,3,3,3-pentafluoropropyl)-1H-pyrazol-4-yl)-8-(trifluoromethyl)-2H-pyrimido[1,2-a]pyrimidine-2,6(1H)-dione;
7-(3-fluoro-4-(2,2,2-trifluoroethoxy)phenyl)-8-(trifluoromethyl)-2H-pyrimido[1,2-a]pyrimidine-2,6(1H)-dione;
7-(4-(2,2,2-trifluoroethoxy)phenyl)-8-(trifluoromethyl)-2H-pyrimido[1,2-a]pyrimidine-2,6(1H)-dione;
7-(4-(2-fluoroethoxy)phenyl)-8-(trifluoromethyl)-2H-pyrimido[1,2-a]pyrimidine-2,6(1H)-dione;
7-(methoxymethyl)-3-[1-(2,2,3,3,3-pentafluoropropyl)-1H-pyrazol-4-yl]-2-(trifluoromethyl)-4H-pyrido[1,2-a]pyrimidin-4-one;
7,8-dimethyl-2-(trifluoromethyl)-3-[1-(3,3,3-trifluoropropyl)-1H-pyrazol-4-yl]-4H-pyrimido[1,2-b]pyridazin-4-one;
7,8-dimethyl-3-[1-(2,2,3,3,3-pentafluoropropyl)-1H-pyrazol-4-yl]-2-(trifluoromethyl)-4H-pyrimido[1,2-b]pyridazin-4-one;
7,9-dimethyl-3-[1-(2,2,3,3,3-pentafluoropropyl)-1H-pyrazol-4-yl]-2-(trifluoromethyl)-4H-pyrazino[1,2-a]pyrimidin-4-one;
7-chloro-2-(trifluoromethyl)-3-[1-(3,3,3-trifluoropropyl)-1H-pyrazol-4-yl]-4H-pyrido[1,2-a]pyrimidin-4-one;
7-chloro-3-[1-(2,2,3,3,3-pentafluoropropyl)-1H-pyrazol-4-yl]-2-(trifluoromethyl)-4H-pyrazino[1,2-a]pyrimidin-4-one;
7-chloro-3-[1-(2,2,3,3,3-pentafluoropropyl)-1H-pyrazol-4-yl]-2-(trifluoromethyl)-4H-pyrido[1,2-a]pyrimidin-4-one;
7-chloro-8-methoxy-2-(trifluoromethyl)-3-[1-(3,3,3-trifluoropropyl)-1H-pyrazol-4-yl]-4H-pyrido[1,2-a]pyrimidin-4-one;
7-chloro-8-methoxy-3-[1-(2,2,3,3,3-pentafluoropropyl)-1H-pyrazol-4-yl]-2-(trifluoromethyl)-4H-[1,3]diazino[1,2-a]pyrimidin-4-one;
7-chloro-8-methoxy-3-[1-(2,2,3,3,3-pentafluoropropyl)-1H-pyrazol-4-yl]-2-(trifluoromethyl)-4H-pyrido[1,2-a]pyrimidin-4-one;
7-chloro-8-methyl-2-(trifluoromethyl)-3-[1-(3,3,3-trifluoropropyl)-1H-pyrazol-4-yl]-4H-pyrido[1,2-a]pyrimidin-4-one;
7-chloro-8-methyl-3-[1-(2,2,3,3,3-pentafluoropropyl)-1H-pyrazol-4-yl]-2-(trifluoromethyl)-4H-pyrido[1,2-a]pyrimidin-4-one;
7-chloro-8-methyl-3-[4-(2,2,2-trifluoroethoxy)phenyl]-2-(trifluoromethyl)-4H-pyrido[1,2-a]pyrimidin-4-one;
7-cyclopropyl-2-(trifluoromethyl)-3-[1-(3,3,3-trifluoropropyl)-1H-pyrazol-4-yl]-4H-pyrido[1,2-a]pyrimidin-4-one;
7-cyclopropyl-3-[1-(2,2,3,3,3-pentafluoropropyl)-1H-pyrazol-4-yl]-2-(trifluoromethyl)-4H-pyrido[1,2-a]pyrimidin-4-one;
7-fluoro-2-(trifluoromethyl)-3-[1-(3,3,3-trifluoropropyl)-1H-pyrazol-4-yl]-4H-pyrido[1,2-a]pyrimidin-4-one;
7-fluoro-3-[1-(2,2,3,3,3-pentafluoropropyl)-1H-pyrazol-4-yl]-2-(trifluoromethyl)-4H-pyrido[1,2-a]pyrimidin-4-one;
7-fluoro-8-hydroxy-3-[1-(2,2,3,3,3-pentafluoropropyl)-1H-pyrazol-4-yl]-2-(trifluoromethyl)-4H-pyrido[1,2-a]pyrimidin-4-one;
7-fluoro-8-methoxy-2-(trifluoromethyl)-3-[1-(3,3,3-trifluoropropyl)-1H-pyrazol-4-yl]-4H-pyrido[1,2-a]pyrimidin-4-one;
7-fluoro-8-methoxy-3-[1-(2,2,3,3,3-pentafluoropropyl)-1H-pyrazol-4-yl]-2-(trifluoromethyl)-4H-[1,3]diazino[1,2-a]pyrimidin-4-one:
7-fluoro-8-methoxy-3-[1-(2,2,3,3,3-pentafluoropropyl)-1H-pyrazol-4-yl]-2-(trifluoromethyl)-4H-pyrido[1,2-a]pyrimidin-4-one;
7-fluoro-8-methoxy-3-[4-(2,2,2-trifluoroethoxy)phenyl]-2-(trifluoromethyl)-4H-pyrido[1,2-a]pyrimidin-4-one;
7-fluoro-8-methyl-3-[1-(2,2,3,3,3-pentafluoropropyl)-1H-pyrazol-4-yl]-2-(trifluoromethyl)-4H-pyrido[1,2-a]pyrimidin-4-one;
7-methoxy-3-[1-(2,2,3,3,3-pentafluoropropyl)-1H-pyrazol-4-yl]-2-(trifluoromethyl)-4H-[1,3]diazino[1,2-a]pyrimidin-4-one:
7-methoxy-3-[1-(2,2,3,3,3-pentafluoropropyl)-1H-pyrazol-4-yl]-2-(trifluoromethyl)-4H-pyrazino[1,2-a]pyrimidin-4-one;
7-methoxy-3-[1-(2,2,3,3,3-pentafluoropropyl)-1H-pyrazol-4-yl]-2-(trifluoromethyl)-4H-pyrido[1,2-a]pyrimidin-4-one;
7-methyl-2-(trifluoromethyl)-3-[1-(3,3,3-trifluoropropyl)-1H-pyrazol-4-yl]-4H-pyrimido[1,2-b]pyridazin-4-one;
7-methyl-3-[1-(2,2,3,3,3-pentafluoropropyl)-1H-pyrazol-4-yl]-2-(trifluoromethyl)-4H-[1,3]diazino[1,2-a]pyrimidin-4-one;
7-methyl-3-[1-(2,2,3,3,3-pentafluoropropyl)-1H-pyrazol-4-yl]-2-(trifluoromethyl)-4H-pyrazino[1,2-a]pyrimidin-4-one;
7-methyl-3-[1-(2,2,3,3,3-pentafluoropropyl)-1H-pyrazol-4-yl]-2-(trifluoromethyl)-4H-pyrido[1,2-a]pyrimidin-4-one;
7-methyl-3-[1-(2,2,3,3,3-pentafluoropropyl)-1H-pyrazol-4-yl]-2-(trifluoromethyl)-4H-pyrimido[1,2-b]pyridazin-4-one;
8-((1R)-1-hydroxyethyl)-3-(1-(2,2,3,3,3-pentafluoropropyl)-1H-pyrazol-4-yl)-2-(trifluoromethyl)-4H-pyrido[1,2-a]pyrimidin-4-one:
8-((1R)-1-hydroxyethyl)-3-(4-(2,2,2-trifluoroethoxy)phenyl)-2-(trifluoromethyl)-4H-pyrido[1,2-a]pyrimidin-4-one;
8-((1S)-1-hydroxyethyl)-3-(1-(2,2,3,3,3-pentafluoropropyl)-1H-pyrazol-4-yl)-2-(trifluoromethyl)-4H-pyrido[1,2-a]pyrimidin-4-one;
8-((1S)-1-hydroxyethyl)-3-(4-(2,2,2-trifluoroethoxy)phenyl)-2-(trifluoromethyl)-4H-pyrido[1,2-a]pyrimidin-4-one;
8-((dimethylamino)methyl)-3-(1-(2,2,3,3,3-pentafluoropropyl)-1H-pyrazol-4-yl)-2-(trifluoromethyl)-4H-pyrido[1,2-a]pyrimidin-4-one;
8-(methyloxy-d₃)-2-(trifluoromethyl)-3-(1-(3,3,3-trifluoropropyl)-1H-pyrazol-4-yl)-4H-pyrido[1,2-a]pyrimidin-4-one;
8-((methylsulfanyl)methoxy)-3-(4-(2,2,2-trifluoroethoxy)phenyl)-2-(trifluoromethyl)-4H-pyrido[1,2-a]pyrimidin-4-one;
8-((R)-ethylsulfinyl)-3-(4-(2,2,2-trifluoroethoxy)phenyl)-2-(trifluoromethyl)-4H-pyrido[1,2-a]pyrimidin-4-one;
8-((R)-methylsulfinyl)-3-(1-(2,2,3,3,3-pentafluoropropyl)-1H-pyrazol-4-yl)-2-(trifluoromethyl)-4H-pyrido[1,2-a]pyrimidin-4-one;
8-((R)-methylsulfinyl)-3-(4-(2,2,2-trifluoroethoxy)phenyl)-2-(trifluoromethyl)-4H-pyrido[1,2-a]pyrimidin-4-one;
8-((S)-ethylsulfinyl)-3-(4-(2,2,2-trifluoroethoxy)phenyl)-2-(trifluoromethyl)-4H-pyrido[1,2-a]pyrimidin-4-one:
8-((S)-methylsulfinyl)-3-(1-(2,2,3,3,3-pentafluoropropyl)-1H-pyrazol-4-yl)-2-(trifluoromethyl)-4H-pyrido[1,2-a]pyrimidin-4-one;
8-((S)-methylsulfinyl)-3-(4-(2,2,2-trifluoroethoxy)phenyl)-2-(trifluoromethyl)-4H-pyrido[1,2-a]pyrimidin-4-one;
8-(1,3-oxazol-2-yl)-3-(4-(2,2,2-trifluoroethoxy)phenyl)-2-(trifluoromethyl)-4H-pyrido[1,2-a]pyrimidin-4-one;
8-(1-hydroxyethyl)-3-(1-(2,2,3,3,3-pentafluoropropyl)-1H-pyrazol-4-yl)-2-(trifluoromethyl)-4H-pyrido[1,2-a]pyrimidin-4-one;
8-(1-hydroxyethyl)-3-(4-(2,2,2-trifluoroethoxy)phenyl)-2-(trifluoromethyl)-4H-pyrido[1,2-a]pyrimidin-4-one;
8-(2-hydroxypropan-2-yl)-3-[1-(2,2,3,3,3-pentafluoropropyl)-1H-pyrazol-4-yl]-2-(trifluoromethyl)-4H-pyrido[1,2-a]pyrimidin-4-one;
8-(2-methyl-2-oxetanyl)-3-(4-(2,2,2-trifluoroethoxy)phenyl)-2-(trifluoromethyl)-4H-pyrido[1,2-a]pyrimidin-4-one;
8-(2-propanyl)-3-(4-(2,2,2-trifluoroethoxy)phenyl)-2-(trifluoromethyl)-4H-pyrido[1,2-a]pyrimidin-4-one;
8-(3-azetidinyl)-3-(4-(2,2,2-trifluoroethoxy)phenyl)-2-(trifluoromethyl)-4H-pyrido[1,2-a]pyrimidin-4-one;
8-(aminomethyl)-3-(1-(2,2,3,3,3-pentafluoropropyl)-1H-pyrazol-4-yl)-2-(trifluoromethyl)-4H-pyrido[1,2-a]pyrimidin-4-one;
8-(1-azetidinyl)-3-(1-(2,2,3,3,3-pentafluoropropyl)-1H-pyrazol-4-yl)-2-(trifluoromethyl)-4H-pyrido[1,2-a]pyrimidin-4-one;
8-(chloromethoxy)-3-[1-(2,2,3,3,3-pentafluoropropyl)-1H-pyrazol-4-yl]-2-(trifluoromethyl)-4H-pyrido[1,2-a]pyrimidin-4-one;
8-(chloromethoxy)-3-[1-(2,2,3,3,3-pentafluoropropyl)-1H-pyrazol-4-yl]-2-(trifluoromethyl)-4H-pyrimido[1,2-b]pyridazin-4-one;
8-(difluoromethoxy)-3-(4-(2,2,2-trifluoroethoxy)phenyl)-2-(trifluoromethyl)-4H-pyrido[1,2-a]pyrimidin-4-one;
8-(difluoromethyl)-3-(4-(2,2,2-trifluoroethoxy)phenyl)-2-(trifluoromethyl)-4H-pyrido[1,2-a]pyrimidin-4-one;
8-(dimethylamino)-3-(1-(2,2,3,3,3-pentafluoropropyl)-1H-pyrazol-4-yl)-2-(trifluoromethyl)-4H-pyrido[1,2-a]pyrimidin-4-one;
8-(dimethylamino)-3-(4-(2,2,2-trifluoroethoxy)phenyl)-2-(trifluoromethyl)-4H-pyrido[1,2-a]pyrimidin-4-one;
8-(ethylsulfinyl)-3-(4-(2,2,2-trifluoroethoxy)phenyl)-2-(trifluoromethyl)-4H-pyrido[1,2-a]pyrimidin-4-one;
8-(fluoromethoxy)-2-(trifluoromethyl)-3-(1-(3,3,3-trifluoropropyl)-1H-pyrazol-4-yl)-4H-pyrido[1,2-a]pyrimidin-4-one;
8-(fluoromethoxy)-3-(4-(2,2,2-trifluoroethoxy)phenyl)-2-(trifluoromethyl)-4H-pyrido[1,2-a]pyrimidin-4-one;
8-(fluoromethoxy)-3-[1-(2,2,3,3,3-pentafluoropropyl)-1H-pyrazol-4-yl]-2-(trifluoromethyl)-4H-pyrido[1,2-a]pyrimidin-4-one;
8-(fluoromethoxy)-3-[1-(2,2,3,3,3-pentafluoropropyl)-1H-pyrazol-4-yl]-2-(trifluoromethyl)-4H-pyrimido[1,2-b]pyridazin-4-one;
8-(fluoromethyl)-3-(1-(2,2,3,3,3-pentafluoropropyl)-1H-pyrazol-4-yl)-2-(trifluoromethyl)-4H-pyrido[1,2-a]pyrimidin-4-one;
8-(fluoromethyl)-3-(4-(2,2,2-trifluoroethoxy)phenyl)-2-(trifluoromethyl)-4H-pyrido[1,2-a]pyrimidin-4-one;
8-(hydroxymethyl)-3-(1-(2,2,3,3,3-pentafluoropropyl)-1H-pyrazol-4-yl)-2-(trifluoromethyl)-4H-pyrido[1,2-a]pyrimidin-4-one;
8-(hydroxymethyl)-3-(4-(2,2,2-trifluoroethoxy)phenyl)-2-(trifluoromethyl)-4H-pyrido[1,2-a]pyrimidin-4-one;
8-(methoxymethyl)-3-[1-(2,2,3,3,3-pentafluoropropyl)-1H-pyrazol-4-yl]-2-(trifluoromethyl)-4H-pyrido[1,2-a]pyrimidin-4-one;
8-(methylamino)-3-(1-(2,2,3,3,3-pentafluoropropyl)-1H-pyrazol-4-yl)-2-(trifluoromethyl)-4H-pyrido[1,2-a]pyrimidin-4-one;
8-(methylamino)-3-(4-(2,2,2-trifluoroethoxy)phenyl)-2-(trifluoromethyl)-4H-pyrido[1,2-a]pyrimidin-4-one;
8-(methylamino)-3-(4-(2,2,2-trifluoroethoxy)phenyl)-2-(trifluoromethyl)-4H-pyrimido[1,2-a]pyrimidin-4-one;
8-(methyl-d₃)-3-(4-(2,2,2-trifluoroethoxy)phenyl)-2-(trifluoromethyl)-4H-pyrido[1,2-a]pyrimidin-4-one;
8-(methyloxy-d₃)-3-(1-(2,2,3,3,3-pentafluoropropyl)-1H-pyrazol-4-yl)-2-(trifluoromethyl)-4H-pyrido[1,2-a]pyrimidin-4-one;
8-(methyloxy-d₃)-3-(1-(2,2,3,3,3-pentafluoropropyl)-1H-pyrazol-4-yl)-2-(trifluoromethyl)-4H-pyrimido[1,2-a]pyrimidin-4-one;
8-(methyloxy-d₃)-3-(1-(4,4,4-trifluorobutyl)-1H-pyrazol-4-yl)-2-(trifluoromethyl)-4H-pyrido[1,2-a]pyrimidin-4-one;
8-(methyloxy-d₃)-3-(4-(2,2,2-trifluoroethoxy)phenyl)-2-(trifluoromethyl)-4H-pyrido[1,2-a]pyrimidin-4-one;
8-(methyloxy-d₃)-3-(4-(2,2,2-trifluoroethoxy)phenyl)-2-(trifluoromethyl)-4H-pyrimido[1,2-a]pyrimidin-4-one;
8-(methyloxy-d₃)-3-(6-(2,2,2-trifluoroethoxy)-3-pyridinyl)-2-(trifluoromethyl)-4H-pyrido[1,2-a]pyrimidin-4-one;
8-(methylsulfanyl)-3-(1-(2,2,3,3,3-pentafluoropropyl)-1H-pyrazol-4-yl)-2-(trifluoromethyl)-4H-pyrido[1,2-a]pyrimidin-4-one;
8-(methylsulfanyl)-3-(4-(2,2,2-trifluoroethoxy)phenyl)-2-(trifluoromethyl)-4H-pyrido[1,2-a]pyrimidin-4-one;
8-(methylsulfinyl)-3-(1-(2,2,3,3,3-pentafluoropropyl)-1H-pyrazol-4-yl)-2-(trifluoromethyl)-4H-pyrido[1,2-a]pyrimidin-4-one;
8-(methylsulfinyl)-3-(4-(2,2,2-trifluoroethoxy)phenyl)-2-(trifluoromethyl)-4H-pyrido[1,2-a]pyrimidin-4-one;
8-(methylsulfonyl)-3-(4-(2,2,2-trifluoroethoxy)phenyl)-2-(trifluoromethyl)-4H-pyrido[1,2-a]pyrimidin-4-one;
8-acetyl-3-(4-(2,2,2-trifluoroethoxy)phenyl)-2-(trifluoromethyl)-4H-pyrido[1,2-a]pyrimidin-4-one;
8-amino-3-(1-(2,2,3,3,3-pentafluoropropyl)-1H-pyrazol-4-yl)-2-(trifluoromethyl)-4H-pyrido[1,2-a]pyrimidin-4-one;
8-amino-3-(4-(2,2,2-trifluoroethoxy)phenyl)-2-(trifluoromethyl)-4H-pyrido[1,2-a]pyrimidin-4-one;
8-amino-3-(4-(2,2,2-trifluoroethoxy)phenyl)-2-(trifluoromethyl)-4H-pyrimido[1,2-a]pyrimidin-4-one;
8-chloro-3-(1-(2,2,3,3,3-pentafluoropropyl)-1H-pyrazol-4-yl)-2-(trifluoromethyl)-4H-pyrido[1,2-a]pyrimidin-4-one;
8-chloro-3-(4-(2,2,2-trifluoroethoxy)phenyl)-2-(trifluoromethyl)-4H-pyrido[1,2-a]pyrimidin-4-one;
8-cyclopropyl-3-(4-(2,2,2-trifluoroethoxy)phenyl)-2-(trifluoromethyl)-4H-pyrido[1,2-a]pyrimidin-4-one;
8-cyclopropyl-3-(1-(2,2,3,3,3-pentafluoropropyl)-1H-pyrazol-4-yl)-2-(trifluoromethyl)-4H-pyrido[1,2-a]pyrimidin-4-one;
8-ethenyl-3-(4-(2,2,2-trifluoroethoxy)phenyl)-2-(trifluoromethyl)-4H-pyrido[1,2-a]pyrimidin-4-one;
8-ethoxy-3-(4-(2,2,2-trifluoroethoxy)phenyl)-2-(trifluoromethyl)-4H-pyrido[1,2-a]pyrimidin-4-one;
8-ethyl-3-(4-(2,2,2-trifluoroethoxy)phenyl)-2-(trifluoromethyl)-4H-pyrido[1,2-a]pyrimidin-4-one;
8-fluoro-3-(4-(2,2,2-trifluoroethoxy)phenyl)-2-(trifluoromethyl)-4H-pyrido[1,2-a]pyrimidin-4-one:
8-hydroxy-3-[1-(2,2,3,3,3-pentafluoropropyl)-1H-pyrazol-4-yl]-2-(trifluoromethyl)-4H-pyrimido[1,2-b]pyridazin-4-one;
8-methoxy-2-(trifluoromethyl)-3-(1-(3-(trifluoromethyl)phenyl)-1H-pyrazol-4-yl)-4H-pyrido[1,2-a]pyrimidin-4-one;
8-methoxy-2-(trifluoromethyl)-3-(1-(3,3,3-trifluoropropyl)-1H-imidazol-4-yl)-4H-pyrido[1,2-a]pyrimidin-4-one;
8-methoxy-2-(trifluoromethyl)-3-(1-(3,3,3-trifluoropropyl)-1H-pyrazol-4-yl)-4H-pyrido[1,2-a]pyrimidin-4-one;
8-methoxy-2-(trifluoromethyl)-3-(1-(3,3,3-trifluoropropyl)-1H-pyrazol-4-yl)-4H-pyrimido[1,2-a]pyrimidin-4-one;
8-methoxy-2-(trifluoromethyl)-3-(1-(4-(trifluoromethyl)phenyl)-1H-pyrazol-4-yl)-4H-pyrido[1,2-a]pyrimidin-4-one;
8-methoxy-2-(trifluoromethyl)-3-(4-(3,3,3-trifluoropropyl)phenyl)-4H-pyrido[1,2-a]pyrimidin-4-one;
8-methoxy-2-(trifluoromethyl)-3-[1-(3,3,3-trifluoropropyl)-1H-pyrazol-4-yl]-4H-pyrimido[1,2-b]pyridazin-4-one;
8-methoxy-2-(trifluoromethyl)-3-[3-(3,3,3-trifluoropropyl)-1,2-oxazol-5-yl]-4H-pyrido[1,2-a]pyrimidin-4-one;
8-methoxy-2-(trifluoromethyl)-3-[4-(3,3,3-trifluoropropyl)-1H-imidazol-1-yl]-4H-pyrido[1,2-a]pyrimidin-4-one;
8-methoxy-2-(trifluoromethyl)-3-[5-(3,3,3-trifluoropropyl)-1,3-thiazol-2-yl]-4H-pyrido[1,2-a]pyrimidin-4-one;
8-methoxy-2-methyl-3-(4-(2,2,2-trifluoroethoxy)phenyl)-4H-pyrido[1,2-a]pyrimidin-4-one;
8-methoxy-3-(1-(2,2,2-trifluoroethyl)-1H-pyrazol-4-yl)-2-(trifluoromethyl)-4H-pyrido[1,2-a]pyrimidin-4-one;
8-methoxy-3-(1-(2,2,3,3,3-pentafluoropropyl)-1H-pyrazol-4-yl)-2-(trifluoromethyl)-4H-pyrido[1,2-a]pyrimidin-4-one;
8-methoxy-3-(1-(2,2,3,3,3-pentafluoropropyl)-1H-pyrazol-4-yl)-2-(trifluoromethyl)-4H-pyrimido[1,2-a]pyrimidin-4-one;
8-methoxy-3-(1-(4,4,4-trifluorobutyl)-1H-pyrazol-4-yl)-2-(trifluoromethyl)-4H-pyrido[1,2-a]pyrimidin-4-one;
8-methoxy-3-(1-(4,4,4-trifluorobutyl)-1H-pyrazol-4-yl)-2-(trifluoromethyl)-4H-pyrimido[1,2-a]pyrimidin-4-one;
8-methoxy-3-(1-phenyl-1H-pyrazol-3-yl)-2-(trifluoromethyl)-4H-pyrido[1,2-a]pyrimidin-4-one;
8-methoxy-3-(1-phenyl-1H-pyrazol-4-yl)-2-(trifluoromethyl)-4H-pyrido[1,2-a]pyrimidin-4-one;
8-methoxy-3-(1-propyl-1H-pyrazol-4-yl)-2-(trifluoromethyl)-4H-pyrido[1,2-a]pyrimidin-4-one;
8-methoxy-3-(2-methyl-4-(2,2,2-trifluoroethoxy)phenyl)-2-(trifluoromethyl)-4H-pyrido[1,2-a]pyrimidin-4-one;
8-methoxy-3-(2-phenyl-1,3-oxazol-5-yl)-2-(trifluoromethyl)-4H-pyrido[1,2-a]pyrimidin-4-one;
8-methoxy-3-(3-phenyl-1,2-oxazol-5-yl)-2-(trifluoromethyl)-4H-pyrido[1,2-a]pyrimidin-4-one;
8-methoxy-3-(4-(2,2,2-trifluoroethoxy)-2-(trifluoromethyl)phenyl)-2-(trifluoromethyl)-4H-pyrido[1,2-a]pyrimidin-4-one;
8-methoxy-3-(4-(2,2,2-trifluoroethoxy)phenyl)-2-(trifluoromethyl)-4H-pyrido[1,2-a]pyrimidin-4-one;
8-methoxy-3-(4-(2,2,2-trifluoroethoxy)phenyl)-2-(trifluoromethyl)-4H-pyrimido[1,2-a]pyrimidin-4-one;
8-methoxy-3-(4-(2,2,3,3,3-pentafluoropropoxy)phenyl)-2-(trifluoromethyl)-4H-pyrido[1,2-a]pyrimidin-4-one;
8-methoxy-3-(4-(2,2,3,3-tetrafluoropropoxy)phenyl)-2-(trifluoromethyl)-4H-pyrido[1,2-a]pyrimidin-4-one;
8-methoxy-3-(4-(trifluoromethoxy)phenyl)-2-(trifluoromethyl)-4H-pyrido[1,2-a]pyrimidin-4-one;
8-methoxy-3-(4-(trifluoromethoxy)phenyl)-2-(trifluoromethyl)-4H-pyrimido[1,2-a]pyrimidin-4-one;
8-methoxy-3-(4-propylphenyl)-2-(trifluoromethyl)-4H-pyrido[1,2-a]pyrimidin-4-one;
8-methoxy-3-(5-propyl-1,2-oxazol-3-yl)-2-(trifluoromethyl)-4H-pyrido[1,2-a]pyrimidin-4-one;
8-methoxy-3-(6-(2,2,2-trifluoroethoxy)-3-pyridinyl)-2-(trifluoromethyl)-4H-pyrido[1,2-a]pyrimidin-4-one;
8-methoxy-3-(6-(2,2,2-trifluoroethoxy)-3-pyridinyl)-2-(trifluoromethyl)-4H-pyrimido[1,2-a]pyrimidin-4-one;
8-methoxy-3-(6-propyl-3-pyridinyl)-2-(trifluoromethyl)-4H-pyrido[1,2-a]pyrimidin-4-one;
8-methoxy-3-[1-(2,2,3,3,3-pentafluoropropyl)-1H-1,2,3-triazol-4-yl]-2-(trifluoromethyl)-4H-pyrido[1,2-a]pyrimidin-4-one;
8-methoxy-3-[1-(2,2,3,3,3-pentafluoropropyl)-1H-pyrazol-3-yl]-2-(trifluoromethyl)-4H-pyrido[1,2-a]pyrimidin-4-one;
8-methoxy-3-[1-(2,2,3,3,3-pentafluoropropyl)-1H-pyrazol-4-yl]-2-(trifluoromethyl)-4H-[1,3]diazino[1,6-a]pyrimidin-4-one:
8-methoxy-3-[1-(2,2,3,3,3-pentafluoropropyl)-1H-pyrazol-4-yl]-2-(trifluoromethyl)-4H-pyrimido[1,2-b]pyridazin-4-one;
8-methoxy-3-[2-(2,2,2-trifluoroethoxy)-1,3-thiazol-5-yl]-2-(trifluoromethyl)-4H-pyrido[1,2-a]pyrimidin-4-one;
8-methoxy-3-[2-(2,2,2-trifluoroethoxy)pyrimidin-5-yl]-2-(trifluoromethyl)-4H-[1,3]diazino[1,2-a]pyrimidin-4-one;
8-methoxy-3-[2-(2,2,2-trifluoroethoxy)pyrimidin-5-yl]-2-(trifluoromethyl)-4H-pyrido[1,2-a]pyrimidin-4-one;
8-methoxy-3-[2-(2,2,2-trifluoroethoxy)pyrimidin-5-yl]-2-(trifluoromethyl)-4H-pyrimido[1,2-b]pyridazin-4-one:
8-methoxy-3-[2-(2,2,3,3,3-pentafluoropropoxy)pyrimidin-5-yl]-2-(trifluoromethyl)-4H-pyrido[1,2-a]pyrimidin-4-one;
8-methoxy-3-[3-(2,2,3,3,3-pentafluoropropyl)-1,2-oxazol-5-yl]-2-(trifluoromethyl)-4H-pyrido[1,2-a]pyrimidin-4-one;
8-methoxy-3-[4-(2,2,2-trifluoroethoxy)-1,3-thiazol-2-yl]-2-(trifluoromethyl)-4H-pyrido[1,2-a]pyrimidin-4-one;
8-methoxy-3-[5-(2,2,2-trifluoroethoxy)-1,3-thiazol-2-yl]-2-(trifluoromethyl)-4H-pyrido[1,2-a]pyrimidin-4-one;
8-methoxy-3-{11-[(oxetan-3-yl)methyl]-1H-pyrazol-4-yl}-2-(trifluoromethyl)-4H-pyrido[1,2-a]pyrimidin-4-one;
8-methoxy-6-methyl-3-[1-(2,2,3,3,3-pentafluoropropyl)-1H-pyrazol-4-yl]-2-(trifluoromethyl)-4H-pyrido[1,2-a]pyrimidin-4-one;
8-methyl-2-(trifluoromethyl)-3-[1-(3,3,3-trifluoropropyl)-1H-pyrazol-4-yl]-4H-pyrimido[1,2-b]pyridazin-4-one:
8-methyl-2-(trifluoromethyl)-3-[5-(3,3,3-trifluoropropyl)-1,2,4-oxadiazol-3-yl]-4H-pyrido[1,2-a]pyrimidin-4-one;
8-methyl-3-(4-(2,2,2-trifluoroethoxy)phenyl)-2-(trifluoromethyl)-4H-pyrido[1,2-a]pyrimidin-4-one;
8-methyl-3-[1-(2,2,3,3,3-pentafluoropropyl)-1H-pyrazol-4-yl]-2-(trifluoromethyl)-4H,6H,7H,8H,9H-pyrimido[1,2-a]pyrazin-4-one;
8-methyl-3-[1-(2,2,3,3,3-pentafluoropropyl)-1H-pyrazol-4-yl]-2-(trifluoromethyl)-4H-pyrimido[1,2-b]pyridazin-4-one;
8-methyl-3-[2-(2,2,2-trifluoroethoxy)pyrimidin-5-yl]-2-(trifluoromethyl)-4H-pyrimido[1,2-b]pyridazin-4-one;
8-methyl-3-[4-(2,2,2-trifluoroethoxy)phenyl]-2-(trifluoromethyl)-4H-pyrimido[1,2-b]pyridazin-4-one;
9-chloro-8-methoxy-3-[1-(2,2,3,3,3-pentafluoropropyl)-1H-pyrazol-4-yl]-2-(trifluoromethyl)-4H-pyrido[1,2-a]pyrimidin-4-one;
9-fluoro-8-methoxy-3-[1-(2,2,3,3,3-pentafluoropropyl)-1H-pyrazol-4-yl]-2-(trifluoromethyl)-4H-pyrido[1,2-a]pyrimidin-4-one;
9-methyl-3-[1-(2,2,3,3,3-pentafluoropropyl)-1H-pyrazol-4-yl]-2-(trifluoromethyl)-4H-pyrazino[1,2-a]pyrimidin-4-one;
methyl 4-oxo-3-(4-(2,2,2-trifluoroethoxy)phenyl)-2-(trifluoromethyl)-4H-pyrido[1,2-a]pyrimidine-8-carboxylate;
methyl(4-oxo-3-(1-(2,2,3,3,3-pentafluoropropyl)-1H-pyrazol-4-yl)-2-(trifluoromethyl)-4H-pyrido[1,2-a]pyrimidin-8-yl)carbamyl fluoride;
N-(4-oxo-3-(1-(2,2,3,3,3-pentafluoropropyl)-1H-pyrazol-4-yl)-2-(trifluoromethyl)-4H-pyrido[1,2-a]pyrimidin-8-yl)acetamide:
N-(4-oxo-3-(4-(2,2,2-trifluoroethoxy)phenyl)-2-(trifluoromethyl)-4H-pyrido[1,2-a]pyrimidin-8-yl)acetamide;
N,N-dimethyl-4-oxo-3-(4-(2,2,2-trifluoroethoxy)phenyl)-2-(trifluoromethyl)-4H-pyrido[1,2-a]pyrimidine-8-carboxamide;
N-ethyl-4-oxo-3-(4-(2,2,2-trifluoroethoxy)phenyl)-2-(trifluoromethyl)-4H-pyrido[1,2-a]pyrimidine-8-carboxamide:
N-methyl-4-oxo-3-(1-(2,2,3,3,3-pentafluoropropyl)-1H-pyrazol-4-yl)-2-(trifluoromethyl)-4H-pyrido[1,2-a]pyrimidine-8-carboxamide; or
N-methyl-4-oxo-3-(4-(2,2,2-trifluoroethoxy)phenyl)-2-(trifluoromethyl)-4H-pyrido[1,2-a]pyrimidine-8-carboxamide; or
   a pharmaceutically acceptable salt of any of the foregoing.

Provided herein as Embodiment 63 is the method or compound of any one of Embodiments 1 to 61, wherein the FADS1 inhibitor compound is:

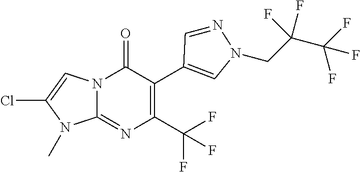 [SMILES: Cn1c(Cl)cn2c(=O)c(-c3cnn(CC(F)(F)C(F)(F)F)c3)c(C(F)(F)F)nc12]

or a pharmaceutically acceptable salt thereof.

Provided herein as Embodiment 64 is the method or compound of any one of Embodiments 1 to 61, wherein the FADS1 inhibitor compound is:

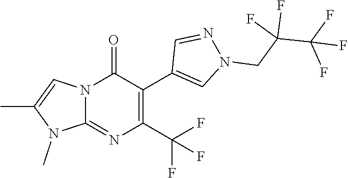 [SMILES: Cc1cn2c(=O)c(-c3cnn(CC(F)(F)C(F)(F)F)c3)c(C(F)(F)F)nc2n1C]

or a pharmaceutically acceptable salt thereof.

Provided herein as Embodiment 65 is the method or compound of any one of Embodiments 1 to 61, wherein the FADS1 inhibitor compound is:

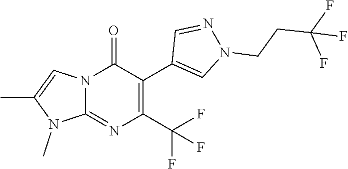 [SMILES: Cc1cn2c(=O)c(-c3cnn(CCC(F)(F)F)c3)c(C(F)(F)F)nc2n1C]

or a pharmaceutically acceptable salt thereof.

Provided herein as Embodiment 66 is the method or compound of any one of Embodiments 1 to 61, wherein the FADS1 inhibitor compound is:

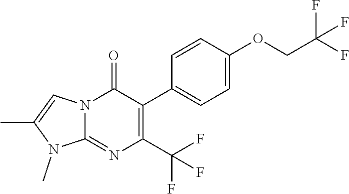 [SMILES: Cc1cn2c(=O)c(-c3ccc(OCC(F)(F)F)cc3)c(C(F)(F)F)nc2n1C]

or a pharmaceutically acceptable salt thereof.

Provided herein as Embodiment 67 is the method or compound of any one of Embodiments 1 to 61, wherein the FADS1 inhibitor compound is:

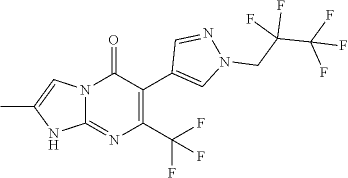 [SMILES: Cc1cn2c(=O)c(-c3cnn(CC(F)(F)C(F)(F)F)c3)c(C(F)(F)F)nc2[nH]1]

or a pharmaceutically acceptable salt thereof.

Provided herein as Embodiment 68 is the method or compound of any one of Embodiments 1 to 61, wherein the FADS1 inhibitor compound is:

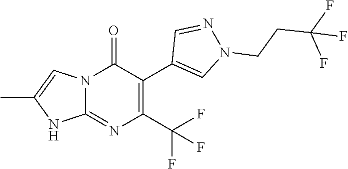 [SMILES: Cc1cn2c(=O)c(-c3cnn(CCC(F)(F)F)c3)c(C(F)(F)F)nc2[nH]1]

or a pharmaceutically acceptable salt thereof.

Provided herein as Embodiment 69 is the method or compound of any one of Embodiments 1 to 61, wherein the FADS1 inhibitor compound is:

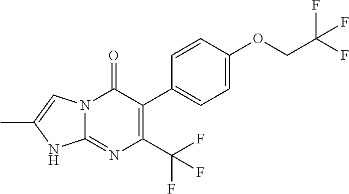 [SMILES: Cc1cn2c(=O)c(-c3ccc(OCC(F)(F)F)cc3)c(C(F)(F)F)nc2[nH]1]

or a pharmaceutically acceptable salt thereof.

Provided herein as Embodiment 70 is the method or compound of any one of Embodiments 1 to 61, wherein the FADS1 inhibitor compound is:

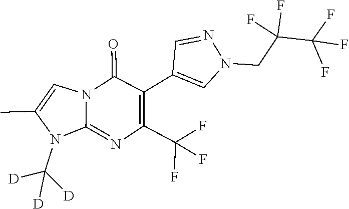 [SMILES: [2H]C([2H])([2H])n1c(C)cn2c(=O)c(-c3cnn(CC(F)(F)C(F)(F)F)c3)c(C(F)(F)F)nc12]

or a pharmaceutically acceptable salt thereof.

Provided herein as Embodiment 71 is the method or compound of any one of Embodiments 1 to 61, wherein the FADS1 inhibitor compound is:

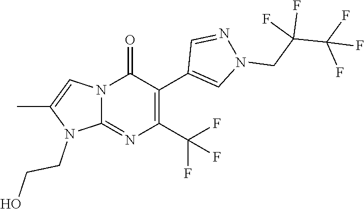 [SMILES: Cc1cn2c(=O)c(-c3cnn(CC(F)(F)C(F)(F)F)c3)c(C(F)(F)F)nc2n1CCO]

or a pharmaceutically acceptable salt thereof.

Provided herein as Embodiment 72 is the method or compound of any one of Embodiments 1 to 61, wherein the FADS1 inhibitor compound is:

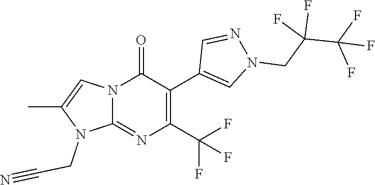 [SMILES: Cc1cn2c(=O)c(-c3cnn(CC(F)(F)C(F)(F)F)c3)c(C(F)(F)F)nc2n1CC#N]

or a pharmaceutically acceptable salt thereof.

Provided herein as Embodiment 73 is the method or compound of any one of Embodiments 1 to 61, wherein the FADS1 inhibitor compound is:

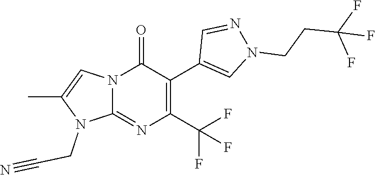 [SMILES: Cc1cn2c(=O)c(-c3cnn(CCC(F)(F)F)c3)c(C(F)(F)F)nc2n1CC#N]

or a pharmaceutically acceptable salt thereof.

Provided herein as Embodiment 74 is the method or compound of any one of Embodiments 1 to 61, wherein the FADS1 inhibitor compound is:

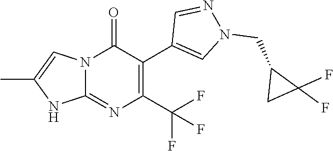 [SMILES: Cc1cn2c(=O)c(-c3cnn(C[C@H]4CC4(F)F)c3)c(C(F)(F)F)nc2[nH]1]

or a pharmaceutically acceptable salt thereof.

Provided herein as Embodiment 75 is the method or compound of any one of Embodiments 1 to 61, wherein the FADS1 inhibitor compound is:

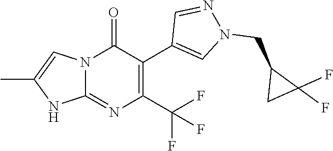 [SMILES: Cc1cn2c(=O)c(-c3cnn(C[C@@H]4CC4(F)F)c3)c(C(F)(F)F)nc2[nH]1]

or a pharmaceutically acceptable salt thereof.

Provided herein as Embodiment 76 is the method or compound of any one of Embodiments 1 to 61, wherein the FADS1 inhibitor compound is:

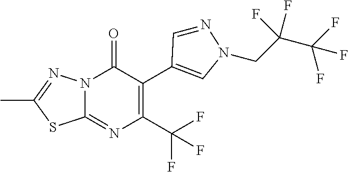 [SMILES: Cc1nn2c(=O)c(-c3cnn(CC(F)(F)C(F)(F)F)c3)c(C(F)(F)F)nc2s1]

or a pharmaceutically acceptable salt thereof.

Provided herein as Embodiment 77 is the method or compound of any one of Embodiments 1 to 61, wherein the FADS1 inhibitor compound is:

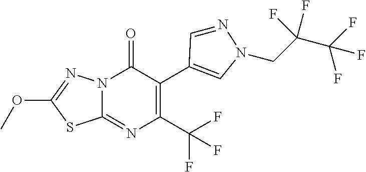 [SMILES: COc1nn2c(=O)c(-c3cnn(CC(F)(F)C(F)(F)F)c3)c(C(F)(F)F)nc2s1]

or a pharmaceutically acceptable salt thereof.

Provided herein as Embodiment 78 is the method or compound of any one of Embodiments 1 to 61, wherein the FADS1 inhibitor compound is:

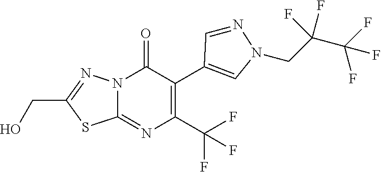 [SMILES: O=c1c(-c2cnn(CC(F)(F)C(F)(F)F)c2)c(C(F)(F)F)nc2sc(CO)nn12]

or a pharmaceutically acceptable salt thereof.

Provided herein as Embodiment 79 is the method or compound of any one of Embodiments 1 to 61, wherein the FADS1 inhibitor compound is:

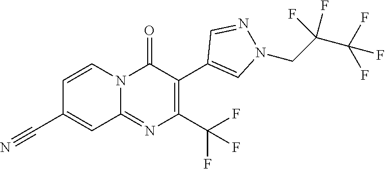 [SMILES: N#Cc1ccn2c(=O)c(-c3cnn(CC(F)(F)C(F)(F)F)c3)c(C(F)(F)F)nc2c1]

or a pharmaceutically acceptable salt thereof.

Provided herein as Embodiment 80 is the method or compound of any one of Embodiments 1 to 61, wherein the FADS1 inhibitor compound is:

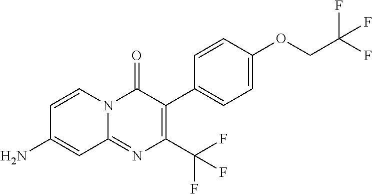 [SMILES: Nc1ccn2c(=O)c(-c3ccc(OCC(F)(F)F)cc3)c(C(F)(F)F)nc2c1]

or a pharmaceutically acceptable salt thereof.

Provided herein as Embodiment 81 is the method or compound of any one of Embodiments 1 to 61, wherein the FADS1 inhibitor compound is:

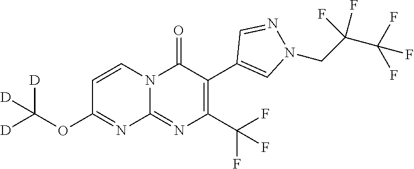 [SMILES: [2H]C([2H])([2H])Oc1ccn2c(=O)c(-c3cnn(CC(F)(F)C(F)(F)F)c3)c(C(F)(F)F)nc2n1]

or a pharmaceutically acceptable salt thereof.

Provided herein as Embodiment 82 is the method or compound of any one of Embodiments 1 to 61, wherein the FADS1 inhibitor compound is:

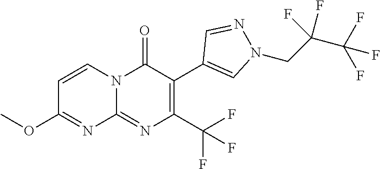 [SMILES: COc1ccn2c(=O)c(-c3cnn(CC(F)(F)C(F)(F)F)c3)c(C(F)(F)F)nc2n1]

or a pharmaceutically acceptable salt thereof.

Provided herein as Embodiment 83 is the method or compound of any one of Embodiments 1 to 61, wherein the FADS1 inhibitor compound is:

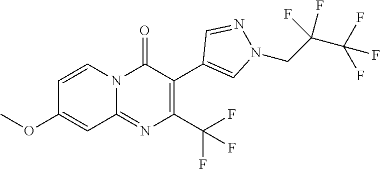 [SMILES: COc1ccn2c(=O)c(-c3cnn(CC(F)(F)C(F)(F)F)c3)c(C(F)(F)F)nc2c1]

or a pharmaceutically acceptable salt thereof.

Provided herein as Embodiment 84 is the method or compound of any one of Embodiments 1 to 61, wherein the FADS1 inhibitor compound is:

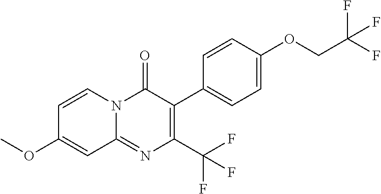 [SMILES: COc1ccn2c(=O)c(-c3ccc(OCC(F)(F)F)cc3)c(C(F)(F)F)nc2c1]

or a pharmaceutically acceptable salt thereof.

Provided herein as Embodiment 85 is the method or compound of any one of claims 1 to 84, wherein the FADS1 inhibitor compound is a free base.

### Patient Response and Labeled DGLA

Also disclosed herein are methods of assessing, analyzing, and/or adjusting dosing regimens for a patient who has undergone a treatment with a FADS1 inhibitor compound or who is in an ongoing treatment regimen. Using one or more methods disclosed herein, the level of inhibition occurring with a dose of a FADS1 inhibitor compound can be tested relative to the maximal level of inhibition (e.g., the IC level). Where the inhibitory concentration level is lower than target and/or desired levels, the dose can be adjusted (e.g., upwardly). Where the inhibitory concentration level is at or above target levels, the dose can be adjusted downwardly or maintained. To illustrate, where the target therapeutic dose is an inhibitory concentration of 90% of the maximal inhibitory concentration (IC90) or above and where the actual dose provides 50% inhibition (e.g., an IC50), the dose of FADS1 inhibitor can be increased (cither with larger doses or doses at increased frequency). In several embodiments, the target therapeutic inhibitory concentration is equal to or at least about: 70% (e.g., IC70), 80% (e.g., IC80), 90% (e.g., IC90), 95% (e.g., IC95), 97.5% (e.g., IC97.5), 99% (e.g., IC99), 99.9% (e.g., IC99.9), 100% (IC100), or ranges including and/or spanning the aforementioned values. In several embodiments, the measured and/or actual inhibitory concentration at an administered dose is equal to or less than about: 10% (e.g., IC10), 20% (e.g., IC20), 30% (e.g., IC30), 40% (e.g., IC40), 50% (e.g., IC50), 60% (e.g., IC60), 70% (e.g., IC70), 80% (e.g., IC80), 90% (e.g., IC90), or ranges including and/or spanning the aforementioned values. In several embodiments, where the actual inhibitory concentration is equal to or less than about: 10% (e.g., IC10), 20% (e.g., IC20), 30% (e.g., IC30), 50% (e.g., IC50), 60% (e.g., IC60), 70% (e.g., IC70), 80% (e.g., IC80), 90% (e.g., IC90), 95% (e.g., IC95), or ranges including and/or spanning the aforementioned values of the maximal inhibitory concentration, then the dose may be increased. In several embodiments, where the inhibitory concentration is below what is desired, the dose is increased by equal to or at least about: 5%, 10%, 15%, 20%, 30%, 50%, 75%, 100%, 200%, or ranges including and/or spanning the aforementioned values. In several embodiments, where the inhibitory concentration is below what is desired, the frequency of dosing is doubled. In several embodiments, both the dose and the dose frequency may be increased.

In several embodiments, to measure the inhibitory concentration achieved with a particular FADS1 inhibiting compound, a labeled DGLA molecule can be administered to a subject (e.g., orally, intravascularly, intravenously, intraarterial, intraperitoneally, subcutaneously, etc.). After a period of time, a sample is collected from the subject, as disclosed elsewhere herein. Thereafter, the amount of labeled AA and the amount of labeled DGLA in the sample can be determined. The values for the labeled AA and labeled DGLA may be used to calculate the inhibitory concentration (e.g., using methods as disclosed in the Examples Section). In several embodiments, the sample is a tissue biopsy or a blood sample (e.g., a plasma sample). In several embodiments, the period of time between administration of the labeled DGLA to sample collection is equal to or at least about: 30 minutes, 45 minutes, 1 hour, 2 hours, 3 hours, 4 hours, 6 hours, 8 hours, 10 hours, 12 hours, 16 hours, 18 hours, 24 hours, or ranges including and/or spanning the aforementioned values.

In several embodiments, the FADS1 inhibitor compound is administered to the subject prior to, concurrent with, or after administration of the labeled DGLA molecule (e.g., an isotopologue of DGLA). In several embodiments, the period of time between administration of the FADS1 inhibitor to administration of the labeled DGLA is equal to or at least about: 30 minutes, 45 minutes, 1 hour, 2 hours, 3 hours, 4 hours, 6 hours, 8 hours, 10 hours, 12 hours, 16 hours, 18 hours, 24 hours, or ranges including and/or spanning the aforementioned values.

In several embodiments, the labeled DGLA comprises DGLA isotopically enriched with ¹³C at one or more positions. In several embodiments, the labeled DGLA comprises DGLA with equal to or at least two positions that are isotopically enriched with ¹³C atoms. In several embodiments, the labeled DGLA comprises DGLA with equal to or at least three positions that are isotopically enriched with ¹³C atoms. In several embodiments, the labeled DGLA comprises DGLA with equal to or at least four positions that are isotopically enriched with ¹³C atoms. In several embodiments, the labeled DGLA comprises DGLA with equal to or at least five positions that are isotopically enriched with ¹³C atoms. In several embodiments, the labeled DGLA comprises DGLA that is isotopically enriched with ¹³C atoms at equal to or at least about: 1 position, 2 positions, 3 positions, 4 positions, 5 positions, 6 positions, 7 positions, 8 positions, 9 positions, 10 positions, 11 positions, 12 positions, 13 positions, 14 positions, 15 positions, 16 positions, 17 positions, 18 positions, 19 positions, 20 positions, or ranges including and/or spanning the aforementioned values. In several embodiments, the labeled DGLA comprises DGLA that is uniformly isotopically enriched with ¹³C atoms.

In several embodiments, the labeled DGLA comprises the following structure (of Formula):

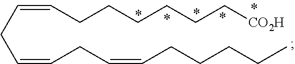 [SMILES: CCCCC/C=C\C/C=C\C/C=C\CCCCCCC(=O)O]

wherein each “*” symbol indicates a position that may be isotopically enriched with ¹³C and at least one “*” position is isotopically enriched with ¹³C. In several embodiments, this compound is referred to as ¹³C_{w}-DGLA, where “w” is the number of “*” positions that are isotopically enriched with ¹³C. In several embodiments, “w” is equal to or at least 1, 2, 3, 4, or 5. For example, where w is two, then two “*” symbols represent a position that is isotopically enriched with ¹³C. In several embodiments, equal to or at least two “*” symbols represent a position that is isotopically enriched with ¹³C. In several embodiments, equal to or at least three “*” symbols represent a position that is isotopically enriched with ¹³C. In several embodiments, equal to or at least four “*” symbols represent a position that is isotopically enriched with ¹³C. In several embodiments, equal to or at least five “*” symbols represent a position that is isotopically enriched with ¹³C.

In several embodiments, the measurement of biological indicators as disclosed elsewhere herein may be incorporated into methods of treatments and uses disclosed elsewhere herein. In several embodiments, the measurement of biological indicators and/or patient selection steps as disclosed elsewhere herein are used in methods of reducing body weight, reducing body-mass-index, treating obesity, treating a metabolic disorder, treating a cardiovascular disorder, treating diabetes, treating obesity, treating dyslipidemia, and/or treating non-alcoholic steatohepatitis (NASH) in a subject. The methods and uses that may be incorporated with the measurements and selection steps described in this section are described in elsewhere herein.

Provided herein as Embodiment 86 is a method of measuring a ratio of arachidonic acid (AA) and dihomo-gamma-linolenic acid (DGLA) in a subject;
wherein the AA is isotopically labeled and the DGLA is isotopically labeled;
wherein the ratio of AA to DGLA is measured by administering a dose of labeled DGLA to the subject and thereafter measuring a ratio of labeled AA to labeled DGLA.

Provided herein as Embodiment 87 is the method of Embodiment 86, wherein the labeled DGLA comprises DGLA that is isotopically enriched with ¹³C.

Provided herein as Embodiment 88 is the method of Embodiment 87, wherein the labeled DGLA comprises DGLA with equal to or at least two carbon positions that are isotopically enriched with ¹³C atoms. For example, in several embodiments, the labeled DGLA is DGLA with two carbon positions that are isotopically enriched with ¹³C atoms. In several embodiments, the labeled DGLA is DGLA with more than two carbon positions that are isotopically enriched with ¹³C atoms.

Provided herein as Embodiment 89 is the method of Embodiment 87, wherein the labeled DGLA comprises DGLA with equal to or at least three carbon positions that are isotopically enriched with ¹³C atoms. For example, in several embodiments, the labeled DGLA is DGLA with three carbon positions that are isotopically enriched with ¹³C atoms. In several embodiments, the labeled DGLA is DGLA with more than three carbon positions that are isotopically enriched with ¹³C atoms.

Provided herein as Embodiment 90 is the method of Embodiment 87, wherein the labeled DGLA comprises DGLA with equal to or at least four carbon positions that are isotopically enriched with ¹³C atoms. For example, in several embodiments, the labeled DGLA is DGLA with four carbon positions that are isotopically enriched with ¹³C atoms. In several embodiments, the labeled DGLA is DGLA with more than four carbon positions that are isotopically enriched with ¹³C atoms.

Provided herein as Embodiment 91 is the method of Embodiment 87, wherein the labeled DGLA comprises DGLA with equal to or at least five carbon positions that are isotopically enriched with ¹³C atoms. For example, in several embodiments, the labeled DGLA is DGLA with five carbon positions that are isotopically enriched with ¹³C atoms. In several embodiments, the labeled DGLA is DGLA with more than five carbon positions that are isotopically enriched with ¹³C atoms.

Provided herein as Embodiment 92 is the method of any one of Embodiments 86 to 91, wherein the labeled DGLA includes labels at one or more or all of the carbons indicated with an “*” below:

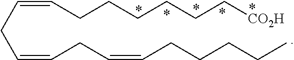 [SMILES: CCCCC/C=C\C/C=C\C/C=C\CCCCCCC(=O)O]

In several embodiments, equal to or at least two “*” symbols represent a position that is isotopically enriched with ¹³C. In several embodiments, equal to or at least three “*” symbols represent a position that is isotopically enriched with ¹³C. In several embodiments, equal to or at least four “*” symbols represent a position that is isotopically enriched with ¹³C. In several embodiments, each of the five “*” symbols represent a position that is isotopically enriched with ¹³C.

Provided herein as Embodiment 93 is the method of any one of Embodiments 86 to 92, wherein a dose of a FADS1 inhibitor compound is administered to the subject prior to, concurrently with, or after the administration of the labeled dose of DGLA to the subject.

Provided herein as Embodiment 94 is the method of Embodiment 92, wherein the conversion of labeled DGLA to labeled AA is used to measure and/or calculate a level of inhibition of the FADS1 enzyme by the FADS1 inhibitor compound at the dose provided to the patient.

Provided herein as Embodiment 95 is the method of Embodiment 94, further comprising comparing the measured level of inhibition by the FADS1 inhibitor compound at the dose provided to the patient to a desired level of inhibition FADS1.

Provided herein as Embodiment 96 is the method of Embodiment 95, further comprising determining an adjusted dose of FADS1 inhibitor for the subject based on the comparison of the level of measured level and desired level of inhibition of FADS1.

Provided herein as Embodiment 97 is the method of Embodiment 96, further comprising administering the adjusted dose of FADS1 inhibitor to the subject.

Provided herein as Embodiment 98 is a compound as represented the following structure:

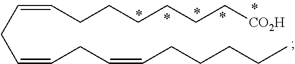 [SMILES: CCCCC/C=C\C/C=C\C/C=C\CCCCCCC(=O)O]

where
each “*” symbol indicates a position that may be isotopically enriched with ¹³C; and
at least one “*” position is isotopically enriched with ¹³C.

Provided herein as Embodiment 99 is a compound as represented the following structure:

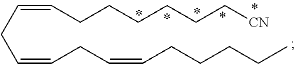 [SMILES: CCCCC/C=C\C/C=C\C/C=C\CCCCCCC#N]

where
each “*” symbol indicates a position that may be isotopically enriched with ¹³C; and
at least one “*” position is isotopically enriched with ¹³C.

Provided herein as Embodiment 100 is a compound as represented the following structure:

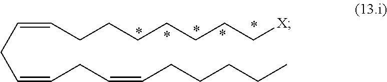

where
X is a halogen;
each “*” symbol indicates a position that may be isotopically enriched with ¹³C; and
at least one “*” position is isotopically enriched with ¹³C.

Provided herein as Embodiment 101 is the compound of any one of Embodiments 98 to 100, wherein at least two “*” positions are isotopically enriched with ¹³C.

Provided herein as Embodiment 102 is the compound of any one of Embodiments 98 to 100, wherein at least three “*” positions are isotopically enriched with ¹³C.

Provided herein as Embodiment 103 is the compound of any one of Embodiments 98 to 100, wherein at least four “*” positions are isotopically enriched with ¹³C.

Provided herein as Embodiment 104 is the compound of any one of Embodiments 98 to 100, wherein all five “*” positions are isotopically enriched with ¹³C.

Provided herein as Embodiment 105 is the compound of any one of Embodiments 98 to 104, wherein each position that is isotopically enriched is enriched by a factor of equal to or at least 100. For example, the isotopic enrichment factor at a labeled position is equal to or greater than 100.

Provided herein as Embodiment 106 is the compound of any one of Embodiments 98 to 104, wherein each position that is isotopically enriched is enriched by a factor of equal to or at least 500. For example, the isotopic enrichment factor at a labeled position is equal to or greater than 500.

Provided herein as Embodiment 107 is the compound of any one of Embodiments 98 to 104, wherein each position that is isotopically enriched is enriched by a factor of equal to or at least 1000. For example, the isotopic enrichment factor at a labeled position is equal to or greater than 1000.

### Methods of Making Labeled DGLA

Several embodiments disclosed herein provide methods of making labeled DGLA. In several embodiments, as disclosed elsewhere herein, the DGLA is labeled with ¹³C. DGLA labeled with too few ¹³C atoms was found to provide insufficient signal to measure labeled DGLA to labeled AA ratios (e.g., for pharmacodynamic analysis). Advantageously, DGLA having multiple (e.g., two, three, four, or five) ¹³C labels was found to provide sufficient signal to allow calculation of inhibitory concentration values (e.g., as a measure of direct response with differentiation from endogenous DGLA and AA levels). In several embodiments, DGLA labeled with five ¹³C labels was found to provide sufficient signal to allow calculation of inhibitory concentration values. In several embodiments. DGLA having five ¹³C labels (e.g., dihomo-gamma-linoleic acid 1,2,3,4,5-¹³C) was prepared.

The following scheme (Scheme 1) provides one exemplary route for synthesis of DGLA, though others will be readily understood based on this disclosure:

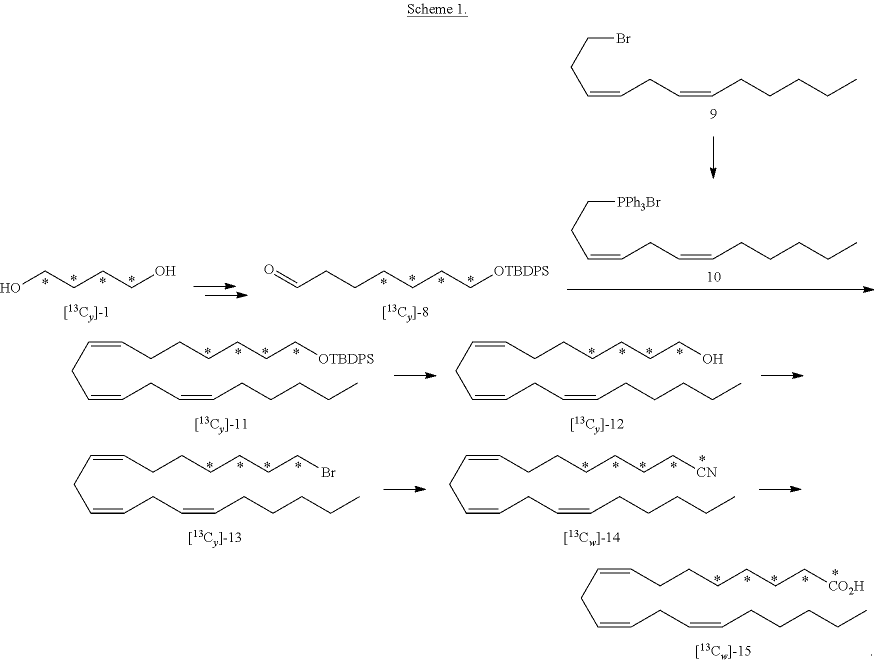

wherein each “*” symbol indicates a position that may be isotopically enriched with ¹³C and at least one “*” position is isotopically enriched with ¹³C. In several embodiments, y is equal to or at least 1, 2, 3, or 4. For example, where y is two, then two “*” symbols represent a position that is isotopically enriched with ¹³C (which can be indicated as [¹³C₂]). In several embodiments, at least two “*” symbols represent a position that is isotopically enriched with ¹³C. In several embodiments, at least three “*” symbols represent a position that is isotopically enriched with ¹³C. In several embodiments, at least four “*” symbols represent a position that is isotopically enriched with ¹³C. In several embodiments, for structures [¹³C_{w}]-14 and [¹³C_{w}]-15, each of the five “*” symbols represent a position that is isotopically enriched with ¹³C.

The following scheme (Scheme 2) provides another exemplary route for synthesis of DGLA, where each “*” is isotopically enriched with ¹³C:

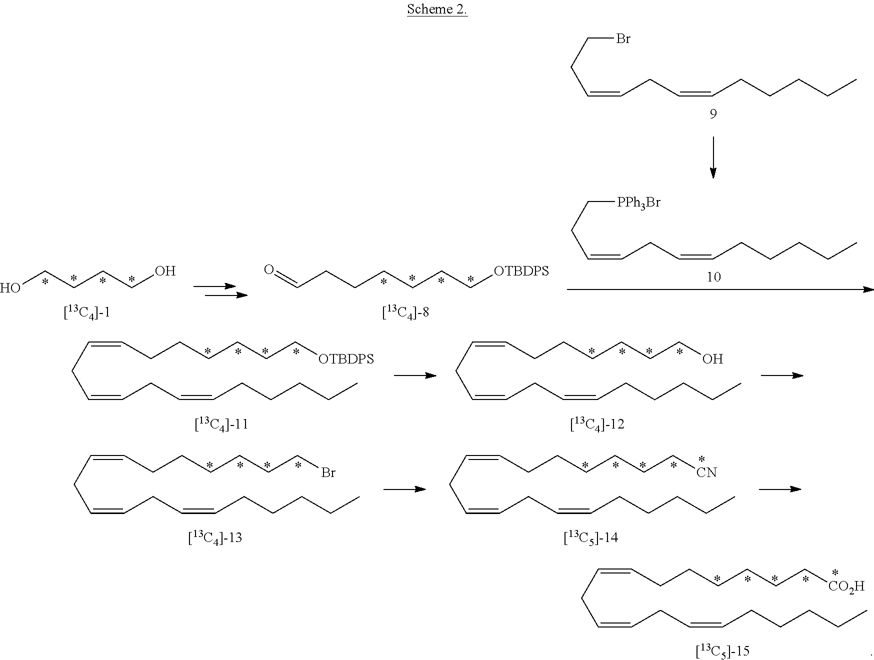

In several embodiments, the method of manufacturing ¹³C_{w}-DGLA (as represented by the following structure):

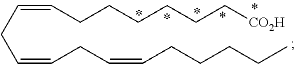 [SMILES: CCCCC/C=C\C/C=C\C/C=C\CCCCCCC(=O)O]

comprises reacting the following compound

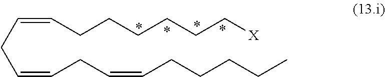

with KC*N, where each “*” symbol indicates a position that may be isotopically enriched with ¹³C and at least one “*” position is isotopically enriched with ¹³C; and wherein X is an appropriate leaving group. In several embodiments, X is a halogen. In several embodiments, X is Br. In several embodiments, at least two “*” symbols on ¹³C_{w}-DGLA represent a position that is isotopically enriched with ¹³C. In several embodiments, at least three “*” symbols on ¹³C_{w}-DGLA represent a position that is isotopically enriched with ¹³C. In several embodiments, at least four “*” symbols on ¹³C_{w}-DGLA represent a position that is isotopically enriched with ¹³C. In several embodiments, all five “*” symbols on ¹³C_{w}-DGLA represent a position that is isotopically enriched with ¹³C.

Labeled 1,4-butanediol (e.g., 1,4-butanediol-¹³C₄) is commercially available. It can be converted to compound [¹³C₄]-8 using the following synthetic scheme (Scheme 3):

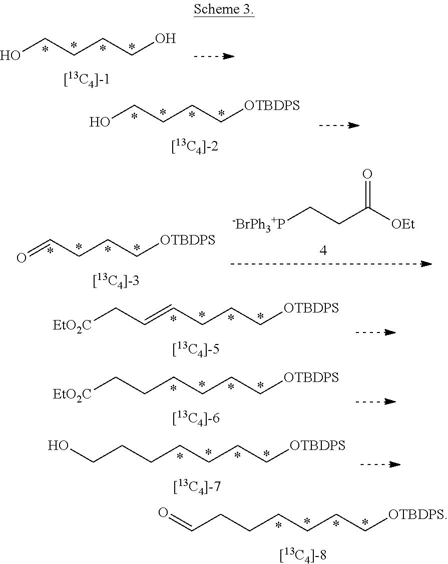

Provided herein as Embodiment 108 is a method of manufacturing a compound represented by the following structure:

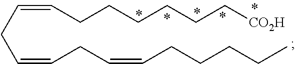 [SMILES: CCCCC/C=C\C/C=C\C/C=C\CCCCCCC(=O)O]

the method comprising reacting the following compound

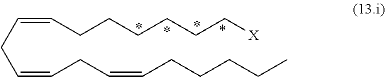

with KC*N;
where
each “*” symbol indicates a position that may be isotopically enriched with ¹³C and at least one “*” position is isotopically enriched with ¹³C; and
wherein X is an appropriate leaving group. In several embodiments, equal to or at least two “*” symbols represent a position that is isotopically enriched with ¹³C. In several embodiments, equal to or at least three “*” symbols represent a position that is isotopically enriched with ¹³C. In several embodiments, equal to or at least four “*” symbols represent a position that is isotopically enriched with ¹³C. In several embodiments, each of the five “*” symbols represent a position that is isotopically enriched with ¹³C.

Provided herein as Embodiment 109 is the method of Embodiment 108, wherein X is a halogen.

Provided herein as Embodiment 110 is the method of Embodiment 109, wherein X is Br.

### Formulation and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. In several embodiments, a pharmaceutical composition comprises a therapeutically effective amount of a compound disclosed herein (e.g., a FADS1 inhibitor compound).

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

### ENUMERATED EMBODIMENTS

Provided herein as Embodiment A.1 is a method of identifying a subject having increased fatty acid desaturase 1 (FADS1) activity, the method comprising:
measuring one or more biological indicators of FADS1-mediated disease or disorder in the subject;
wherein the one or more biological indicators comprise, consist of, or consist essentially of one or more of a ratio of polyunsaturated fatty acids (“PUFAs”) in the subject, a relative abundance of one or more cell types, a relative abundance of one or more differentially expressed genes (DEGs) or gene signatures, and/or a relative abundance of one or more metabolites.

Provided herein as Embodiment A.2 is a method of identifying a subject in need of treatment with a FADS1 inhibitor compound, the method comprising:
measuring one or more biological indicators of FADS1-mediated disease or disorder in the subject;
wherein the one or more biological indicators comprise, consist of, or consist essentially of one or more of a ratio of polyunsaturated fatty acids (“PUFAs”) in the subject, a relative abundance of one or more cell types, a relative abundance of one or more differentially expressed genes (DEGs) or gene signatures, and/or a relative abundance of one or more metabolites.

Provided herein as Embodiment A.3 is a method of reducing body weight, reducing body-mass-index, treating obesity, treating a metabolic disorder, treating a cardiovascular disorder, treating diabetes, treating dyslipidemia, and/or treating non-alcoholic steatohepatitis (NASH) in a subject, the method comprising:
measuring one or more biological indicators of FADS1-mediated disease or disorder in the subject;
wherein the one or more biological indicators comprise, consist of, or consist essentially of one or more of a ratio of polyunsaturated fatty acids (“PUFAs”) in the subject, a relative abundance of one or more cell types, a relative abundance of one or more differentially expressed genes (DEGs) or gene signatures, and/or a relative abundance of one or more metabolites.

Provided herein as Embodiment A.4 is the method of any one of Embodiments A.1 to A.3, further comprising administering to the subject a FADS1 inhibitor compound.

Provided herein as Embodiment A.5 is the method of any one of Embodiments A.1 to A.4, wherein the ratio of PUFAs comprises or consists of a ratio of arachidonic acid (AA) to dihomo-gamma-linolenic acid (DGLA).

Provided herein as Embodiment A.6 is the method of Embodiment A.5, wherein the ratio of AA to DGLA is equal to or at least about 5:1.

Provided herein as Embodiment A.7 is the method of Embodiment A.5, wherein the ratio of AA to DGLA is equal to or at least about 6:1.

Provided herein as Embodiment A.8 is the method of Embodiment A.5, wherein the ratio of AA to DGLA is equal to or at least about 7:1.

Provided herein as Embodiment A.9 is the method of any one of Embodiments A.1 to A.8, wherein the one or more cell types include one or more of adipocytes (Adipo), B cell (Bcell), endothelial (Endo), hepatocytes (Hep), kupffer (Kupff), myeloid (Myel), natural killer (NK). T cell (Tcell), or combinations of any of the foregoing.

Provided herein as Embodiment A.10 is the method of Embodiment A.9, wherein the relative abundance of at least one cell type of the subject is increased.

Provided herein as Embodiment A.11 is the method of any one of Embodiments A.9 or A.10, wherein the relative abundance of at least one cell type of the subject is decreased.

Provided herein as Embodiment A.12 is the method of any one of Embodiments A.1 to A.11, wherein the one or more metabolites comprise, consists of, or consist essentially of one or more of plasma cholesterol, free cholesterol, total cholesterol, cholesterylester, malate, alpha-ketoglutarate, mannose, glucose, erythron-dihydrosphingosine, 5-O-methylsphingosine, threo-sphinogosine, 1-hydroxy-2-amino-(cis,trans)-3,5-octadecadiene, 4-hydroxysphinganine, thromboxane B2, delta-12-prostaglandin D2, prostaglandin E2, prostaglandin D2, 12-hydroxyheptadecatrienoic acid, 14,15-dihydroxyeicosatrienoic acid, 11-hydroxyeicosatetraenoic acid, 13-hydroxyoctadecadienoic acid, arachidonic acid, docosahexaenoic acid, dihomo-gamma-linolenic acid, gamma-linolenic acid, docosapentaenoic acid, eicosapentaenoic acid, docosatetraenoic acid, stearic acid, tryptophan, histidine, valine, threonine, cysteine, kynurenic acid, taurochenodeoxycholic acid, taurocholic acid, plasma triglycerides, plasmalogens, choline plasmalogen, myo-inositol phospholipids, glycerol phosphate, phosphate, lysophosphatidylcholine, lysophosphatidylethanolamine, and/or phosphatidylcholine.

Provided herein as Embodiment A.13 is the method of Embodiment A.12, wherein the relative abundance of at least one metabolite of the subject is increased.

Provided herein as Embodiment A.14 is the method of Embodiment A.12 or A.13, wherein the relative abundance of at least one metabolite of the subject is decreased.

Provided herein as Embodiment A.15 is the method of any one of Embodiments A.1 to A.14, wherein the DEG include one or more of Serpinb1a, Gna14, Serpina3m, Hsd11b1, Cyp2c29, Hsd11b1, Akr1d1, Aldh1a1, Mmp19, or combinations of any of the foregoing.

Provided herein as Embodiment A.16 is the method of any one of Embodiments A.1 to A.15, wherein the DEG include one or more of Gyp27a1, Tymp, Elov12, Chkb, H2afj, Tnfaip811, Tmem86a, Sel113, Agap2, 4833411C07Rik, or combinations of any of the foregoing.

Provided herein as Embodiment A.17 is the method of any one of Embodiments A.1 to A.16, wherein the DEG include one or more of Elov4, Fat3, Eef1a2, Atp7a, Rgs2, Cenpe, Mfap2, Mlki67, Ctsh, or combinations of any of the foregoing.

Provided herein as Embodiment A.18 is the method of any one of Embodiments A.1 to A.17, wherein the DEG include one or more of Trub2, Ubc, H6pd, Eepd1, Acss2, Aacs, Gm36827, Man2a2, Nudt18, Plagl1, or combinations of any of the foregoing.

Provided herein as Embodiment A.19 is the method of any one of Embodiments A.1 to A.18, wherein the DEG include one or more of TM4sf19, Atp6v0d2, Gm20056, Trem2, I11rn, Mmp12, Cdk18, Efr3b, Tag1n2, or combinations of any of the foregoing.

Provided herein as Embodiment A.20 is the method of any one of Embodiments A.1 to A.19, wherein the DEG include one or more of Lurap1, Cp, I17rb, B230303O12Rik, Cfd, Sult1e1, Tdo2, or combinations of any of the foregoing.

Provided herein as Embodiment A.21 is the method of any one of Embodiments A.1 to A.20, wherein the DEG include one or more of Cyp2b9, Fads1, Hao2, Cyp2b13, Cyp2a22, Acnat2, Ildr2, Rpl10a-ps1, Tm6sf2, or combinations of any of the foregoing.

Provided herein as Embodiment A.22 is the method of any one of Embodiments A.1 to A.21, wherein the DEG include one or more of Fitm1, Tmem86a, Agap2, Lpar1, C6, Cmah, Lbp, Arsg, Glra3, Lad1, A730063M14Rik, or combinations of any of the foregoing.

Provided herein as Embodiment A.23 is the method of any one of Embodiments A.1 to A.22, wherein the DEG include one or more of Ly6f, Foxi1, Crygc, Defb28, Wfdc9, Ph1da2, Aqp6, Gm16411, Adam7, or combinations of any of the foregoing.

Provided herein as Embodiment A.24 is the method of any one of Embodiments A.1 to A.23, wherein the DEG include Ppp2r5b.

Provided herein as Embodiment A.25 is the method of any one of Embodiments A.1 to A.24, wherein the DEG include one or more of Slc6a7, Gpr50, Ahnak2, S100a6, Mmp14, Htr2b. Hpgds, Mfap2, or combinations of any of the foregoing.

Provided herein as Embodiment A.26 is the method of any one of Embodiments A.1 to A.25, wherein the DEG include one or more of Gm18537, Aacs, Pclo, Adrb3. Gm38394, AC154232.2, Cadps, Adgrb2, Gm45470, or combinations of any of the foregoing.

Provided herein as Embodiment A.27 is the method of any one of Embodiments A.1 to A.26, wherein the DEG include one or more of Sdr9c7, Dsg1c, Slc17a1, Acnat2, Ces1c. Gss, Hsd11b1, or combinations of any of the foregoing.

Provided herein as Embodiment A.28 is the method of any one of Embodiments A.1 to A.27, wherein the DEG include one or more of 1810008I18Rik, Tlcd1, Snrk, Akr1c20, Gm19950, Ttr, Cbfa2t3, Acat2, Pmvk, Abcd3, Acss2, Acacb, Arhgap27, or combinations of any of the foregoing.

Provided herein as Embodiment A.29 is the method of any one of Embodiments A.1 to A.28, wherein the DEG include one or more of Adam7, Rnase9, Wfdc8, Ighv9-2, DerI3, Acap1, Ccl19, Tcf7, Xkrx, Trim46, or combinations of any of the foregoing.

Provided herein as Embodiment A.30 is the method of any one of Embodiments A.1 to A.29, wherein the DEG include one or more of Zfp369, Zfp871, Pcdhb21, Adrb3, Gm14288, Uprt, Atm, Dchs2, Cped1, Gm38357, or combinations of any of the foregoing.

Provided herein as Embodiment A.31 is the method of any one of Embodiments A.1 to A.30, wherein the DEG include one or more of Cck, Ckap2, Gm4419, 1600015I10Rik, Sez6I2, Prnd, Gm16702, S100a8, or combinations of any of the foregoing.

Provided herein as Embodiment A.32 is the method of any one of Embodiments A.1 to A.31, wherein the DEG include one or more of Pcdh12, Malat1, Kcnqlot1, ArI4c, Gm38394, Gm42549, AC154232.2, Gm37310, Gm37776, Atp2a1, Ckm, Tnnt3, or combinations of any of the foregoing.

Provided herein as Embodiment A.33 is the method of any one of Embodiments A.1 to A.32, wherein the DEG include one or more of Serpinb1a, Gna14, Serpina3m, Hsd11b1, Cyp2c29. Hsd11b1, Akr1d1, Aldh1a1, Mmp19, or combinations of any of the foregoing Adipoq, Fabp4, Lep, Retn, Hoxc8, Hoxc9, Cebpa, Dgat1, Dgat2, Elov13, Fads1, Fads2, Fas, Scd1, Srebf1, Adrb3, Hilpda, Lipe, Mg11, Plin1, Plin4, Pnpla2, Pnpla3, Ldah, Cs, Gckr, Me1, Pck1, Pdk4, Irs1, Acaa1a, Acads, Acox1, Cpt1a, Cpt1b, Hadhb, Ehhadh, Ppara, Ppargc1a, Cyp4a12a, Cyp4a12b, Cyp2e1, Adgre1, Agtr1a, Cc12, Cd14, Cd68, Il1b, Tm6sf2, Tnf, or combinations of any of the foregoing.

Provided herein as Embodiment A.34 is the method of any one of Embodiments A.1 to A.33, wherein the DEG include one or more DEGs as disclosed in any one or more of FIG. 4a, FIG. 5a, FIG. 10a, and/or FIG. 13a.

Provided herein as Embodiment A.35 is the method of any one of Embodiments A.1 to A.34, wherein the relative abundance of at least one DEG of the subject is upregulated.

Provided herein as Embodiment A.36 is the method of Embodiment A.35, wherein the at least one upregulated DEG is upregulated by a factor of equal to or greater than 2.

Provided herein as Embodiment A.37 is the method of any one of Embodiments A.1 to A.36, wherein the relative abundance of at least one DEG of the subject is downregulated.

Provided herein as Embodiment A.38 is the method of Embodiment A.37, wherein the at least one downregulated DEG is downregulated by factor of equal to or greater than 2.

Provided herein as Embodiment A.39 is the method of any one of Embodiments A.1 to A.38, wherein the ratio of PUFAs comprises or consists of a ratio of labeled DGLA to labeled AA;
wherein the ratio of DGLA to AA is measured by administering a dose of labeled DGLA to the subject and thereafter measuring a ratio of labeled AA to labeled DGLA.

Provided herein as Embodiment A.40 is the method of Embodiment A.39, wherein the labeled DGLA comprises DGLA isotopically enriched with 13C labeling.

Provided herein as Embodiment A.41 is the method of Embodiment A.40, wherein the labeled DGLA comprises DGLA with equal to or at least two positions that are isotopically enriched with 13C atoms.

Provided herein as Embodiment A.42 is the method of Embodiment A.40, wherein the labeled DGLA comprises DGLA with equal to or at least three positions that are isotopically enriched with 13C atoms.

Provided herein as Embodiment A.43 is the method of Embodiment A.40, wherein the labeled DGLA comprises DGLA with equal to or at least four positions that are isotopically enriched with 13C atoms.

Provided herein as Embodiment A.44 is the method of Embodiment A.40, wherein the labeled DGLA comprises DGLA with equal to or at least five positions that are isotopically enriched with 13C atoms.

Provided herein as Embodiment A.45 is the method of Embodiment A.40, wherein the labeled DGLA comprises DGLA that is uniformly isotopically enriched with 13C atoms.

Provided herein as Embodiment A.46 is the method of any one of Embodiments A.39 to A.44, wherein the labeled DGLA includes labels at one or more or all of the indicated areas below:

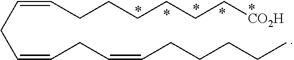 [SMILES: CCCCC/C=C\C/C=C\C/C=C\CCCCCCC(=O)O]

Provided herein as Embodiment A.47 is the method of any one of Embodiments A.39 to A.46, wherein a dose of a FADS1 inhibitor compound is administered to the subject prior to, concurrently with, or after the administration of the labeled dose of DGLA to the subject.

Provided herein as Embodiment A.48 is the method of Embodiment A.46, wherein the conversion of labeled DGLA to labeled AA is used to measure and/or calculate a level of inhibition of the FADS1 enzyme by the FADS1 inhibitor compound at the dose provided to the patient.

Provided herein as Embodiment A.49 is the method of Embodiment A.48, further comprising comparing the measured level of inhibition by the FADS1 inhibitor compound at the dose provided to the patient to a desired level of inhibition FADS1.

Provided herein as Embodiment A.50 is the method of Embodiment A.49, further comprising determining an adjusted dose of FADS1 inhibitor for the subject based on the comparison of the level of measured level and desired level of inhibition of FADS1.

Provided herein as Embodiment A.51 is the method of Embodiment A.50, further comprising administering the adjusted dose of FADS1 inhibitor to the subject.

Provided herein as Embodiment A.52 is a compound as represented by the following structure:

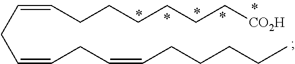 [SMILES: CCCCC/C=C\C/C=C\C/C=C\CCCCCCC(=O)O]

where
each “*” symbol indicates a position that may be isotopically enriched with 13C; and
at least one “*” position is isotopically enriched with 13C.

Provided herein as Embodiment A.53 is a compound as represented by the following structure:

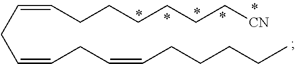 [SMILES: CCCCC/C=C\C/C=C\C/C=C\CCCCCCC#N]

where
each “*” symbol indicates a position that may be isotopically enriched with 13C; and
at least one “*” position is isotopically enriched with 13C.

Provided herein as Embodiment A.54 is a compound as represented by the following structure:

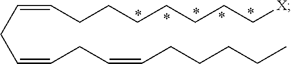

where
X is a halogen
each “*” symbol indicates a position that may be isotopically enriched with 13C; and
at least one “*” position is isotopically enriched with 13C.

Provided herein as Embodiment A.55 is the method of any one of Embodiments A.52 to A.54, wherein at least two “*” positions are isotopically enriched with 13C.

Provided herein as Embodiment A.56 is the method of any one of Embodiments A.52 to A.54, wherein at least three “*” positions are isotopically enriched with 13C.

Provided herein as Embodiment A.57 is the method of any one of Embodiments A.52 to A.54, wherein at least four “*” positions are isotopically enriched with 13C.

Provided herein as Embodiment A.58 is the method of any one of Embodiments A.52 to A.54, wherein at least five “*” positions are isotopically enriched with 13C.

Provided herein as Embodiment A.59 is a method of manufacturing a compound as represented by the following structure:

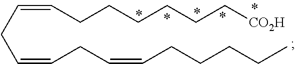 [SMILES: CCCCC/C=C\C/C=C\C/C=C\CCCCCCC(=O)O]

the method comprising reacting the following compound

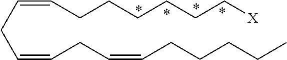

with KC*N;
where
each “*” symbol indicates a position that may be isotopically enriched with 13C and at least one “*” position is isotopically enriched with 13C; and
wherein X is an appropriate leaving group.

Provided herein as Embodiment A.60 is the method of Embodiment A.59, wherein X is a halogen.

Provided herein as Embodiment A.61 is the method of Embodiment A.60, wherein X is Br.

The following examples are given for the purpose of illustrating various embodiments of the disclosure and are not meant to limit the present disclosure in any fashion. One skilled in the art will appreciate readily that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### EXAMPLES

This section provides specific examples of compounds of Formula (I) and methods of making the same.

### Materials and Methods

Provided below are exemplary materials and methods used in the accompanying examples.

### Reagents

### List of Chemical Abbreviations

| | |
|---|---|
| Ac | acetyl |
| DCM | dichloromethane |
| DMSO | dimethyl sulfoxide |
| Et₂O | diethyl ether |
| EtOAc | ethyl acetate |
| EtOH | ethanol |
| KHMDS | potassium hexamethyldisilazide |
| NaHMDS | sodium hexamethylsilazide |
| NaHCO₃ | sodium bicarbonate |
| Na₂SO₄ | sodium sulfate |
| TsOH | toluenesulfonic acid |
| TBAF | tetra-n-butylammonium fluoride |
| TBDPS | tert-butyldiphenylsilyl |
| TBDPSCl | tert-butyldiphenylsilyl chloride |
| THP | tetrahydropyran |
| TEA or Et₃N | triethylamine |
| THF | tetrahydrofuran |

FADS1 (NM_013402) and FADS2 (NM_004265) BacMam reagents were generated by the Department of Biologics. Amgen (Thousand Oaks. CA). [¹³C₁-C₅] arachidonic acid (¹³C₅-AA), alpha-linolenic acid-d14 (ALA-d14), anandamide-d8 (AEA-d8) and polyunsaturated fatty acid standards were purchased from Cayman Chemical, (Ann Arbor, MI). |¹³C₁-C₅|8,11,14-eicosatrienoic acid (¹³C₅-DGLA) was custom synthesized by Curachem (Korea). [¹³C₁₈] Linoleic acid (¹³C₁₈-LA) was purchased from IsoSciences (Ambler, PA).

### Human Plasma Samples

All Human Specimens were Collected Under Site-Specific Institutional Review Board approval, with appropriate informed consent in compliance with all applicable laws and regulations. In all cases, materials obtained were surplus to standard clinical practice and standard of care. Patient identity and protected health information/identifying information were redacted from tissues and clinical data before submission. The inclusion criteria were set such that subjects did not have cancer, cardiovascular diseases, and autoimmune disorders.

### Compound A FADS1 Small Molecule Inhibitor

“Compound A,” 8-methoxy-3-(1-(2,2,3,3,3-pentafluoropropyl)-1H-pyrazol-4-yl)-2-(trifluoromethyl)-4H-pyrido[1,2-a]pyrimidin-4-one, was synthesized according to the reaction steps described in WO2021/108404A1. Compound A was dissolved in dimethyl sulfoxide (DMSO) for in vitro activity assays and formulated in 2% hydroxypropyl methylcellulose (HPMC), 1% Tween 80 for mouse studies.

### Mouse Models and Care

Mice were housed at an AAALAC, International accredited facility. Animals were cared for in accordance with the Guide for the Care and Use of Laboratory Animals, 8th Edition. Mice were housed in individual ventilated caging (IVC) system (Innorack, Innovive or Greenline, Tecniplast) on an irradiated corncob bedding (Envigo Teklad 7097). Lighting in animal holding rooms was maintained on 12:12 hr. light:dark cycle, and the ambient temperature and humidity range was at 68 to 79° F. and 30 to 70%, respectively. Animals had ad libitum access to food and reverse-osmosis (RO) chlorinated (2 to 3 ppm) water via an automatic watering system. Male AGN-rFADS1KO knockout (Fads1 KO; Fads1^{Gtr(IST11525H2)Tigm}, gene trap IST11525H2, Texas A&M Institute for Genomic Medicine) and WT C57BL/6 mice were bred at Charles River Laboratories (Hollister, CA) or Jackson Laboratories (Bar Harbor. Main). Only male mice were used in the studies to avoid the confounding factor of female estrous cycle. Mice were fed standard chow provided by Charles River (5066; LabDiet; St. Louis, MA) until 5-8 weeks of age. After arrival and 1-2 weeks of acclimation, DIO mice were placed on an HFD (60 kcal % fat, D12492 Research Diets, New Brunswick, NJ) for 12 weeks or until indicated time. Lean controls were fed standard chow diet (Envigo Teklad Global Soy Protein-Free Extruded Rodent Diet 2020X, Indianapolis, IN).

### Fads1 KO Mice Phenotyping Study

Fads1 KO and WT mice were fed HFD for 12 weeks. Weekly body weights and weekly fat and lean body composition measurements (EchoMRI, Houston, TX) were recorded throughout the duration of the study. Food intake was measured at 7 weeks on an HFD. An intraperitoneal (IP) GTT was performed following a 12-hour fast at 7 weeks on an HFD, using 2 g/kg glucose. Four-hour fasted retro-orbital blood was collected on the day the mice were switched to an HFD and at 6 and 12 weeks on the diet. At the conclusion of the study at 12 weeks, mice were euthanized by conscious decapitation, and plasma, liver, EPI, and ING WAT tissues were collected.

### Chronic FADS1 Inhibition Study in DIO Mice

WT DIO mice were randomized and administered daily PO gavage with vehicle (2% HPMC, 1% Tween 80) or a dose of Compound A (10 mg/kg or 30 mg/kg) for 54 days. Body weight was measured daily and used for dosing calculations. A 4-hour fasted retro-orbital blood sample was collected at baseline and day 46 and used for insulin, cholesterol, and triglyceride analysis. A 3-day average food consumption was measured on days 39-41. All animals received 0.1 ml of 2 mM ¹³C₅-DGLA by IP injection 23.5 hours after the last PO gavage with the different treatment to allow for 30 minutes of exposure prior to necropsy to determine the residual in vivo FADS1 activity.

### Indirect Calorimetry Studies

Twenty-four Fads1 KO and 24 WT mice entered the Comprehensive Laboratory Animal Monitoring System (CLAMS; Columbus Instruments, Columbus, Ohio—Oxymax Model 2018, 0233-004M, Oxymax for Windows v5.53 software, Hardware configuration 190395) before starting the HFD feeding phase, and again at 6 and 12 weeks on HFD. After 12 weeks of HFD, WT animals were randomized while in the system (after 96 hours for baseline measurements) by body weight, oxygen consumption, carbon dioxide production, age, and RER into two groups: vehicle (2% HPMC, 1% Tween 80) and Compound A at 30 mg/kg. Body weight was measured daily, and food consumption was measured biweekly. Animals reentered the CLAMS on days 17-21 and on days 38-42. The volume of oxygen consumption (VO₂), carbon dioxide production (VCO₂), RER, and energy expenditure (HEAT) were measured during that period. For simplicity, data are displayed in lights-on (light) and lights-off (dark) cycles (12 hours on and 12 hours off, respectively).

### Plasmid Lipid, Glucose, and Insulin Measurements

Mice were bled consciously from the retro-orbital sinus, and blood was collected into EDTA plasma tubes. Plasma lipids were measured using the Olympus AU400e Chemistry Analyzer (Olympus America, Center Valley, PA). Plasma insulin was measured using the ALPCO mouse high range insulin ELISA (Alpco, Salem, NH). Blood glucose was measured using an AlphaTRAK blood glucometer (Zoetis, Parsippany, NJ).

### RNA-Seq Analysis

RNA was extracted by using the Qiagen RNeasy 96 Universal kit (QIAGEN, Germantown, MD). The concentration and quality of the isolated RNA samples were assessed and determined on an Agilent 4200 TapeStation system and had RNA integrity numbers more than 9.1. Adipose or liver RNA (250 ng) was used to prepare a cDNA library by using a protocol modified from the Illumina TruSeq Stranded mRNA kit (Illumina, San Diego CA). Briefly, after polyA⁺ RNA selection, fragmentation, and priming, the fragmented RNA was transcribed to cDNA in the reaction of reverse transcription containing SuperScript™ II (Thermo Fisher Scientific—Invitrogen™, Waltham, MA) and RNase-out (Thermo Fisher Scientific—Invitrogen™, Waltham, MA) and, sequentially, the products of the first strand cDNA synthesis reaction were converted to double-stranded cDNAs and subjected to end repair, A-tailing, and adapter ligation by following the commercial instruction. The constructed libraries were amplified and barcoded using the PCR program: a denaturation step at 98° C. for 30 seconds, 15 cycles of 95° C. for 10 seconds, 60° C. for 30 seconds, and 72° C. for 30 seconds, and a 72° C. extension cycle for 5 minutes followed by a hold step at 4° C. Libraries were sequenced to a minimum depth of 30 million paired end reads on an Illumina HiSeq4000 with the 100 nt or 150 nt read length.

RNA-Seq sequencing reads of the mouse samples were aligned and processed using OSA aligner, which is implemented in the OmicSoft Array Suite (QIAGEN OmicSoft, Cary, NC), based on mouse genome version GRCm38 and gene model GENCODE v19. Read count quantification was performed to the gene level based on OmicSoft implementation of RSEM. Downstream analysis using the read count output was performed following the standard RNA-seq workflow of DESeq2 R/Bioconductor package. Briefly, raw read count was transformed and nonnalized using the Variance Stabilizing Transformation (VST) method. Principal component analysis (PCA) was carried out by the plotPCA function using top 1000 variable genes. Differential expression analysis based on the negative binomial distribution was performed using the DESeq function.

Gene expression was represented as normalized fragments per kilobase per million reads (FPKM). FPKM values were further normalized with a refinement of the commonly employed upper-quartile method that sets the FPKM to a value of 10 at the 70th percentile. Lowly expressed genes with fewer than five samples at FPKM≥1 were removed. Genes with Benjamini-Hochberg (BH)-adjusted P value <0.01 and fold change ≥2 or ≤0.5 were selected as DEGs. Volcano plots of differential expression analysis results were made using EnhancedVolcano R package (github.com/kevinblighe/EnhancedVolcano).

The selected DEGs were annotated by IPA (QIAGEN, Redwood City, CA) on canonical pathways and toxicity lists. Gene expression enrichment in Gene Ontology (GO), KEGG was analyzed by using Cluster Profiler R package with default parameters. Deconvolution analysis was performed using MuSiC software v0.2.0 to estimate relative cell type abundance in each mouse bulk tissue samples. The Tabula Muris single-cell RNA-seq data set was used as a reference for MuSiC algorithm. The analysis was done separately for each tissue types including EPI and ING WAT, and liver.

### Plasma PUFA Measurement

Ten microliter of plasma or PUFA standards diluted in surrogate matrix (60 g/l bovine serum albumin in Dulbecco's phosphate-buffered saline [DPBS]) were mixed with 10 μl of ALA-d₁₄ as the internal stand in a 96-well plate. 100 μl of 2N NaOH was added to the mixture for subsequent saponification at 65° C. for 1 hour. The mixture was then acidified with 50 μl of formic acid followed by two consecutive 500 μl hexane extractions. The organic extracts from the two extractions were combined and the solvent was evaporated under nitrogen gas. Extracts were then resuspended in 250 μl of 90% methanol and analyzed by liquid chromatography with tandem mass spectrometry (LC-MS/MS) for the following PUFAs: LA, GLA, DGLA, and AA, with ALA-d₁₄ as the internal standard. The PUFA peak areas were quantified by using the Analyst software (SCIEX, Framingham, MA).

### Tissue Protein Extraction and Biomarker Analysis of Tissue and Plasma Samples

Frozen liver and adipose tissue samples were powdered with stainless steel Bessman Tissue Pulverizers (Spectrum Laboratory Products, Gardena, CA) per the manufacturer's instructions and the resulting tissue powders were stored in cryotubes at −80° C. until extraction. A portion of each powdered tissue sample was extracted using an in-house protein extraction buffer (50-mM Tris buffer, pH 7.4, 0.1 M NaCl and 0.1% Triton X-100) containing a protease inhibitor cocktail (Millipore Sigma, Burlington, MA). Samples were homogenized with a MagNA Lyser (Roche Diagnostics, Indianapolis, IN) for 30 seconds at 6500 rpm, placed at 4° C. for 30 minutes and then spun in a refrigerated microcentrifuge at 10,000 rpm for 10 minutes. The resulting supernatants were removed to clean 1.5 ml microfuge tubes and spun again at 14,000 rpm for 15 minutes. The final supernatants were aliquoted into labeled cluster tubes and protein concentrations were determined using a standard BCA Protein Assay (Thermo Fisher Scientific, Waltham, MA) prior to storage at −80° C.

Separate aliquots of tissue protein extracts and terminal plasma samples were used to quantify levels of various analytes. Multiple adipokines and hormones (MCP-1, leptin, insulin, and PAI-1) were assessed using a multiplex mouse-specific Luminex assay while adiponectin was evaluated using a single-plex mouse-specific Luminex assay (Millipore Sigma, Burlington, MA). All the commercial assays were performed per the manufacturer's instructions. Data were acquired on a FlexMap 3D (Luminex Corporation, Austin. TX). Tissue protein expression levels were normalized per mg of total protein.

### Plasma Metabolomic Analysis

Plasma samples from male Fads1 KO mice (n=4) and their WT (n=6) littermates fed a HFD for 11 weeks were processed and analyzed for metabolomics analysis at Metanomics Health (Germany) using the MxP Broad Profiling and MxP Eicosanoid platform.

### Liver Triglyceride Measurement

Liver total lipids were homogenized and extracted using chloroform methanol solution at a ratio of 2:1. Extracted lipids were dried under nitrogen gas and suspended in 90% isopropanol, 10% Triton X-100 solution. Liver triglyceride levels in mg/g tissue were determined using total triglyceride measurement kits from Wako Diagnostics (Richmond, VA) and Sigma-Aldrich (Burlington, MA).

### Tissue Endocannabinoid Measurement

Approximately 200 mg of tissue per sample were used for endocannbabinoid extraction. To each frozen tissue sample, 10 μl of ice-cold 100 mM butylated hydroxytoluene (BHT; in methanol) and 0.5 ml of 100 nM AEA-d8 (in water) were added. Tissue sample solution with BHT were immediately homogenized using TissueLyser (QIAGEN, Germantown. MD) for 3 minutes at 30 Hz, followed by two consecutive 1.25 ml of ice-cold ethyl acetate:hexane (9:1) extraction. The pooled extracts were dried to complete dryness under nitrogen gas. Dried samples were redissolved in 80% acetonitrile and subjected to LC/MS analysis. 10 μl of sample was injected onto a Kinetex C18 2.1×100 mm, 2.6 μm column (Phenomenex, Torrance, CA). Mobile phase A is 0.2% acetic acid in water and mobile phase B is 0.1% formic acid in acetonitrile. The LC gradient was a 7-minute long method at a flow rate of 0.2 ml/minute consisting of an isocratic period from 0 to 1 minute at 75% B, followed by a gradient from 75 to 100% from 1 to 4 minutes, and 100 to 75% B from 4 to 7 minutes. The analyte peak areas were quantified using the Analyst software (SCIEX, Framingham MA).

Preparation of ¹³C₅-DGLA ([¹³C₅]-15)
Synthesis of [¹³C₄]-2

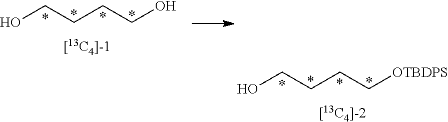

To a 500 mL flask was placed NaH (2.20 g, 55.0 mmol). The NaH was washed with hexane (decantation, two times), then to it was added THF (50 mL). The resulting suspension was cooled to 0° C. A solution of [¹³C₄]-1 (4.70 g, 50.0 mmol) in THE (15 mL) was added dropwise, and the reaction mixture was raised to room temperature. After 2 h of vigorous stirring at room temperature, the reaction mixture was cooled again to 0° C., and TBDPS-Cl (13.7 g, 50.0 mmol) was added dropwise. Then temperature was raised to room and stirred for 1 h. The reaction mixture was quenched with 10% K₂CO₃ aqueous solution (˜30 mL), extracted with Et₂O, dried with Na₂SO₄, filtered, and evaporated. The product was purified by a column chromatography on SiO₂ (EtOAc:hexane=1:4) to afford [¹³C₄]-2 (16.1 g, 97%). ¹H NMR (CHLOROFORM-d) δ: 7.67 (4H, br in), 7.4 (6H, br in), 4.12 (1H, br s), 3.85 (2H, br d), 3.49 (2H, br d), 1.81 (2H, br m), 1.50 (2H, br m), 1.06 (9H, s).

Synthesis of [¹³C₄]-3

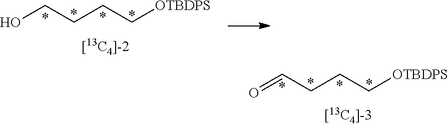

To a 500 mL flask purged with nitrogen was added a solution of oxalyl chloride (5.47 mL, 62.9 mmol) in DCM (15 mL). The flask was cooled to −78° C. A solution of DMSO (4.47 mL, 62.9 mmol) in DCM (20 mL) was added dropwise, and the resulting mixture was stirred at −78° C. for additional 30 min. Then a solution of [¹³C₄]-2 (16.1 g, 48.4 mmol) in DCM (50 mL) was added dropwise. The resulting mixture was stirred at −78° C. for additional 30 min. At that time, TEA (17.5 mL, 126 mmol) was added dropwise, and the reaction mixture was allowed to reach room temperature. After being stirred for overnight at room temperature, the reaction mixture was quenched by water (30 mL) addition, extracted with DCM, dried over Na₂SO₄ and filtered. The product was purified by a column chromatography on SiO₂ (5% EtOAc in Hex) to afford [¹³C₄]-3 (11.2 g, 70%). ¹H NMR (CHLOROFORM-d) δ: 9.75 (1H, dd), 7.67 (4H, br m), 7.4 (6H, br m), 3.86 (1H, br m), 3.50 (1H, br m), 2.70 (1H, br m), 2.38 (1H, br m), 2.04 (1H, br m), 1.72 (1H, br m), 1.06 (s, 9H).

Synthesis of [¹³C₄]-5.1

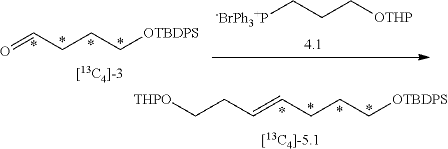

A solution of phosphonium 4.1 (17.5 g, 36.1 mmol) in THF (60 mL) was cooled to −78° C., and KHMDS (37.7 mL, 37.7 mmol) was added dropwise. After being stirred for 10 min at −78° C., the reaction mixture was raised to room temperature. After being stirred for 30 min, the reaction mixture was cooled down again to −78° C. A solution of [¹³C₄]-3 (11.2 g, 33.9 mmol) in THF (20 mL) was added dropwise, and the reaction mixture was allowed to room temperature. After being stirred for overnight at room temperature, the reaction mixture was quenched by water (30 mL) addition, stirred for 15 min, and then extracted with Et₂O, dried Na₂SO₄, and filtered. A column chromatography on SiO₂ (EtOAc:hexanes=1:9) afforded [¹³C₄]-5.1 (14.4 g, 93%). ¹H NMR (CHLOROFORM-d) δ: 7.66 (4H, br m), 7.40 (6H, br m), 6.62 (1H, br m), 5.40 (2H, br m), 5.15 (1H, br m), 4.58 (br m), 3.84 (2H, br m), 3.71 (1H, br m), 3.49 (br m), 3.38 (br m), 2.34 (br m), 1.98 (br m), 1.78-1.65 (br m), 1.60-1.21 (br m), 1.06 (s, 9H).

Synthesis of [¹³C₄]-6.1

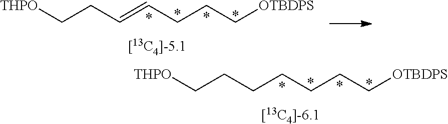

To a 500 mL flask were placed [¹³C₄]-5.1 (14.4 g, 31.4 mmol), 150 mL of EtOH, and 0.5 g of Pd/C. The reaction mixture was hydrogenated with 1 atm of H₂ (balloon) for overnight at room temperature. After a celite filtration followed by evaporation, the crude product was used in the next step without additional purification. [³C₄]-6.1: 13.6 g (94%). ¹H NMR (CHLOROFORM-d) δ: 7.66 (6H, br m), 7.38 (8H, br m), 4.57 (br m), 3.84 (2H, br m), 3.72 (11H, br m), 3.48 (br m), 3.37 (br m), 1.90-1.21 (br m), 1.06 (s, 9H).

Synthesis of [¹³C₄]-7

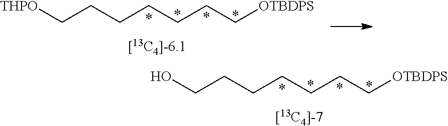

To a solution of crude [¹³C₄]-6.1 (13.6 g, 29.6 mmol) in EtOH (200 mL) was added TsOH (1.23 g, 6.47 mmol), and the reaction mixture was stirred at room temperature for 3 h. Then, NaHCO₃ saturated aqueous solution (50 mL) was added. The resulting mixture was extracted with Et₂O, dried with Na₂SO₄, and filtered. Column chromatography on SiO₂ (EtOAc:hexane=1:9) afforded [¹³C₄]-7 (6.53 g, 59%). ¹H NMR (CHLOROFORM-d) δ: 7.66 (4H, br m), 7.38 (6H, br m), 3.83 (1H, br m), 3.63 (2H, br m), 3.48 (1H, br m), 1.7-1.11 (11H, br m), 1.06 (s, 9H).

Synthesis of [¹³C₄]-8

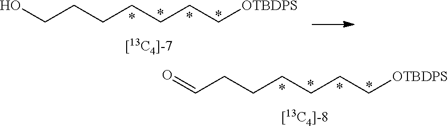

To a 500 mL flask purged with nitrogen was placed, a solution of oxalyl chloride (1.97 mL, 22.7 mmol) in DCM (15 mL). The flask was cooled to −78° C. A solution of DMSO (1.61 mL, 22.7 mmol) in DCM (10 mL) was added dropwise, and the resulting mixture was stirred at −78° C. for additional 30 min. Then, a solution of [¹³C₄]-7 (6.53 g, 17.4 mmol) in DCM (50 mL) was added dropwise, and the resulting mixture was stirred at −78° C. for additional 30 min. Finally. TEA (6.31 mL, 45.5 mmol) was added dropwise, and the reaction mixture was allowed to reach room temperature. After being stirred for 5 h at room temperature, the reaction mixture was quenched by water (30 mL) addition, extracted with DCM, dried Na₂SO₄, and filtered. Column chromatography on SiO₂ (EtOAc:hexanes=1:9) afforded [¹³C₄]-8 (5.73 g, 88%). ¹H NMR (CHLOROFORM-d) δ: 9.75 (1H, s), 7.66 (4H, br m), 7.38 (6H, br m), 3.83 (1H, br m), 3.47 (1H, br m), 2.40 (2H, br m), 1.7-1.11 (8H, br m), 1.06 (s, 9H).

Synthesis of A-[¹³C₄]-11

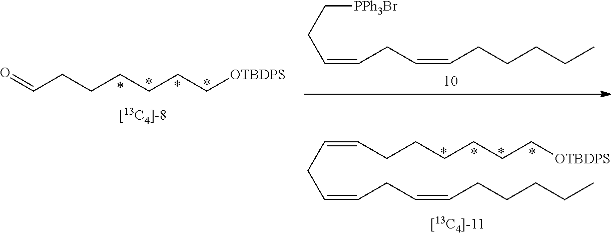

A solution of phosphonium 10 (9.99 g, 19.7 mmol) in THF (100 mL) was cooled to −78° C. and NaHMDS (19.7 mL, 19.7 mmol) was added dropwise. After being stirred for 1.5 h at −78° C., a solution of aldehyde [¹³C₄]-8 (5.73 g, 15.4 mmol) in 30 mL of dry THF (30 mL) was added dropwise, and the reaction mixture was allowed to reach room temperature. After being stirred for 2 h at room temperature, the reaction mixture was quenched by adding NH₄Cl aqueous solution, extracted with Et₂O, dried Na₂SO₄, filtered, and evaporated. Column chromatography on SiO₂ (1% EtOAc in hexane) afforded [¹³C₄]11 (4.63 g, 58%). ¹H NMR (CHLOROFORM-d) δ: 7.66 (4H, br m), 7.38 (6H, br m), 5.36 (5H, m), 3.82 (1H, br m), 3.47 (1H, br m), 2.80 (3H, br m), 2.04 (4H, br m), 1.7-1.11 (15H, br m), 1.04 (s, 9H), 0.88 (4H, br m).

Synthesis of [¹³C₄]-12

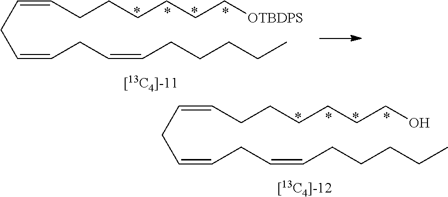

To a solution of [¹³C₄]-11 (4.63 g, 8.89 mmol) in THE (50 mL) was added a solution of TBAF in THF (13.3 mL, 13.3 mmol). After being stirred for 3 h at room temperature, the reaction mixture was quenched with water, extracted with Et₂O, dried over Na₂SO₄, filtered, and evaporated. Column chromatography on SiO₂ (EtOAc:hexanes=1:9) afforded [¹³C₄]-12 (2.36 g, 94%). ¹H NMR (CHLOROFORM-d) δ: 5.36 (5H, br in), 4.11 (1H, br s), 3.81 (1H, br m), 3.47 (1H, br m), 2.81 (3H, br in), 2.04 (4H, br m), 1.7-1.11 (16H, br m), 0.88 (3H, br m).

Synthesis of [¹³C₄]-13

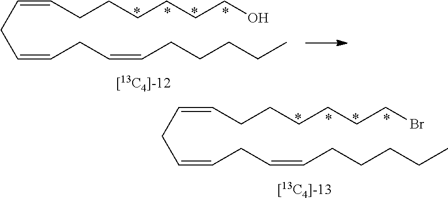 [SMILES: CCCCC/C=C\C/C=C\C/C=C\CCCCCCO ; CCCCC/C=C\C/C=C\C/C=C\CCCCCCBr]

A solution of [¹³C₄]-12 (2.36 g, 8.35 mmol) in DCM (30 mL) was cooled to 0° C. and CBr₄ (3.73 g, 11.3 mmol) was added in one portion. After 5 min at 0° C., a solution of PPh₃ (2.84 g, 10.8 mmol) in DCM (10 mL) was added dropwise. The reaction mixture was raised to room temperature, and stirred for 1 h. The reaction mixture was poured into a mixture of EtOAc:hexane (300 mL, 1:9), and the resulted suspension was celite filtered. Column chromatography on SiO₂ (Hex) afforded [¹³C₄]-13 (2.58 g, 89%). ¹H NMR (CHLOROFORM-d) δ: 5.36 (5H, br m), 3.59 (1H, br m), 3.21 (1H, br m), 2.81 (3H, br m), 2.05 (4H, br m), 1.7-1.11 (16H, br m), 0.88 (3H, br m).

Synthesis of [¹³C₅]-14

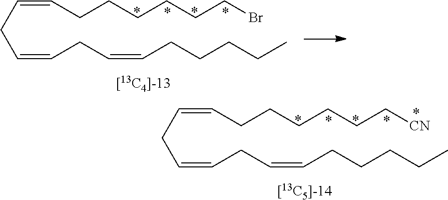 [SMILES: CCCCC/C=C\C/C=C\C/C=C\CCCCCCBr ; CCCCC/C=C\C/C=C\C/C=C\CCCCCCC#N]

A solution of [¹³C₄]-14 (2.58 g, 7.47 mmol) and [¹³C]KCN (0.64 g, 9.68 mmol) in DMSO (20 mL) was stirred at 50° C. for overnight. Then, the reaction mixture was poured into water and extracted with EtOAc. The combined organic layer was washed with water, dried over Na₂SO₄, and filtered. Column chromatography on SiO₂ (2% EtOAc in Hex) afforded [¹³C₅]-14 (2.08 g, 95%). ¹H NMR (CHLOROFORM-d) δ: 5.37 (5H, br m), 2.81 (3H, br m), 2.50 (1H, br s), 2.17-2.05 (5H, br m), 1.82 (l H, br m), 1.7-1.11 (15H, br m), 0.89 (3H, br m).

Synthesis of [¹³C₅]-15

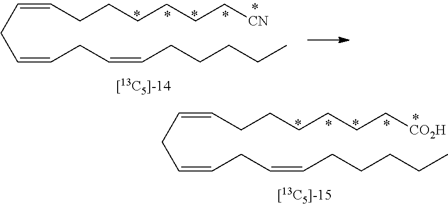 [SMILES: CCCCC/C=C\C/C=C\C/C=C\CCCCCCC#N ; CCCCC/C=C\C/C=C\C/C=C\CCCCCCC(=O)O]

A solution of [¹³C₅]-14 (2.08 g, 7.11 mmol) and NaOH (1.14 g, 28.50 mmol) in a mixture of EtOH/water mixture (30 mL, 4/1) was heated at 100° C. for 20 h. The reaction mixture was evaporated, dissolved in water, and 1N aqueous HCl was added to make acidic solution (pH˜2). The product was extracted with DCM, dried over Na₂SO₄, and filtered. Column chromatography on SiO₂ (EtOAc:Hex=3:7) afforded [³C₅]-15 (2.06 g, 93%). After two consecutive MPLC (medium pressure liquid chromatography) purifications (Wakogel, Mobile phase—water/acetonitrile/acetic acid=10/90/0.1, at RT), 300 mg of [¹³C₅]-15 was obtained. ¹H NMR (CHLOROFORM-d) δ: 5.97 (br m, 6H), 2.81 (br m, 4H), 2.51 (br m, 2H), 2.18 (br m, 2H), 2.05 (br m, 4H), 1.80 (br m, 1H), 1.54 (br m, 4H), 1.49 (br m, 4H), 1.30 (br m, 4H), 1.19 (br m, 2H), 0.89 (br m, 1H), m/z=310.39.

### Overview

The following examples provide additional details regarding the experiments performed herein. Briefly, to determine whether FADS1 activity is elevated with obesity, FADS1 activity was measured using AA/DGLA as a surrogate marker in obese male humans and diet-induced obese (DIO) mice. Fads1 KO mice fed with HFD were then characterized. Indirect calorimetry, transcriptome, and metabolomic analysis were used to understand the physiological and metabolic impact of FADS1 inhibition. Furthermore, the pharmacological inhibition of FADS1 in DIO mice was analyzed with Compound A, a novel, selective and potent small molecule inhibitor of FADS1. Overall, the following studies demonstrate that an FADS1 inhibitor recapitulates the phenotype observed in FADS1-deficient humans and KO mice, and such an inhibitor has the potential to become a therapeutic for the treatment of obesity and related comorbidities.

### Example 1: FADS1 Activity in Obese Humans and DIO Mice

The levels of n-3 and n-6 fatty acids (members of PUFA pathway shown in FIG. 1(a)) were measured in plasma from lean [body mass index (BMI)<25 kg/m², n=20], overweight (25<BMI<30 kg/mt n=41) and obese (BMI>30 kg/m², n=8) male humans (Tables 1.1 and 1.2).

TABLE 1.1
Summary of male blood donors

| Cohort | N | Age (Years) | BMI (Kg/m²) |
|---|---|---|---|
| Lean | 20 | 50.1 ± 12.1 | 23.7 ± 1.3 |
| Overweight | 41 | 54.6 ± 9.1 | 27.4 ± 1.4 |
| Obese | 8 | 52.6 ± 7.2 | 32.2 ± 1.0 |

Data are presented as mean ± SD.

TABLE 1.2
PUFA profile in human male plasma (n = 8-41/group)

| | | Shorthand | Plasma PUFA, μg/ml | | |
|---|---|---|---|---|---|
| Common name | PUFA | Notation | Lean | Overweight | Obese |
| Linoleic acid | LA | 18:2n-6 | 1051 ± 562 | 865 ± 420 | 754 ± 283 |
| γ-Linoleic acid | GLA | 18:3n-6 | 10.6 ± 9.4 | 5.6 ± 4.6 | 8.0 ± 5.5 |
| Dihomo-γ-Linolenic | DGLA | 20:3n-6 | 75.1 ± 41.2 | 52.8 ± 27.7 | 54.1 ± 19.4 |
| acid | | | | | |
| Arachidonic acid | AA | 20:4n-6 | 265 ± 102 | 226 ± 101 | 273 ± 66 |
| Adrenic Acid | AdA | 22:4n-6 | 12.0 ± 5.1 | 8.9 ± 3.7 | 11.1 ± 2.8 |
| Docosapentaenoic acid | DPA n-6 | 18:3n-3 | 8.2 ± 4.7 | 5.9 ± 3.5 | 6.8 ± 1.7 |
| n-6 | | | | | |
| α-Linolenic acid | ALA | 20:4n-3 | 38.1 ± 40.1 | 17.5 ± 20.6 | 29.0 ± 21.9 |
| Eicosapentacnoic acid | EPA | 20:5n-3 | 20.2 ± 16.0 | 10.4 ± 9.5 | 10.2 ± 6.0 |
| Decosahexaenoic acid | DHA | 22:6n-3 | 74.6 ± 27.5 | 62.2 ± 34.2 | 39.3 ± 11.4 |
| FADS2 activity | GLA/LA | 20:4n-6/ | 0.008 ± 0.004 | 0.007 ± 0.004 | 0.01 ± 0.004 |
| | | 20:3n-6 | | | |
| FADS1 activity | AA/DGLA | 18:3n-6/ | 4.1 ± 1.4 | 4.8 ± 1.6 | 5.6 ± 2.1* |
| | | 18:2n-6 | | | |

Data are presented as mean ± SD.

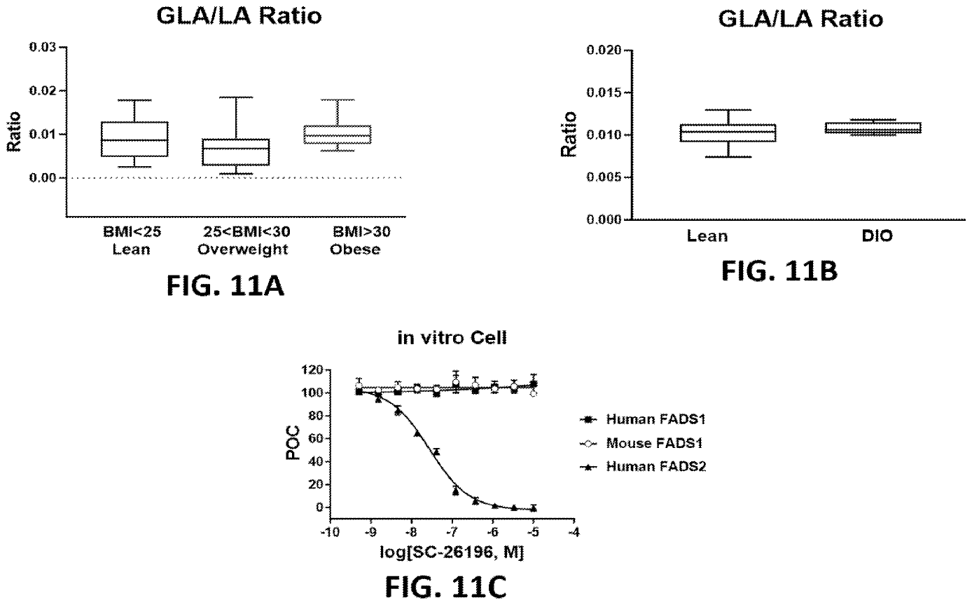

There were no differences in circulating levels of AA and DGLA between lean, overweight, and obese male subjects (Table 1.2); however. AA/DGLA ratio, a surrogate for n-6 FADS1 activity, was significantly increased in obese males relative to lean subjects (FIG. 1(b)). Similarly. PUFA measured in plasma of lean and DIG C57B/6 male mice (Table 1.3) demonstrated a significant increase in n-6 FADS1 activity (AA/DGLA ratio) in DIG mice relative to lean mice (Table 1.3 and FIG. 1(c)). No differences in GLA/LA ratio, a surrogate of n-6 FADS2 (D6D) activity, were observed between obese, overweight, and lean human males (FIG. 11(a)) and in DIG mice relative to lean mice (FIG. 11(b)). The observation of increased FADS1 activity in obese humans and DIG mice suggested that DIG mice ere suitable preclinical models to further understand the role of FADS1 at the molecular and pharmacological levels.

TABLE 1.3
PUFA profile in plasma of Lean and DIO male mice

| | | Shorthand | | | P-value* |
|---|---|---|---|---|---|
| Common Name | Abbreviation | Notation | Lean, μg/ml | DIO, μg/ml | (Lean vs DIO) |
| Linoleic acid | LA | 18:2 n-6 | 882 ± 184 | 926 ± 127 | ns |
| g-Linoleic acid | GLA | 18:3 n-6 | 9.1 ± 2.3 | 9.1 ± 3.6 | ns |
| Dihomo-g-Linolenic | DGLA | 20:3 n-6 | 85.4 ± 9.0 | 105.7 ± 19.3 | 0.0171 |
| acid | | | | | |
| Arachidonic acid | AA | 20:4 n-6 | 409 ± 55.9 | 926 ± 130 | <0.0001 |
| Adrenic acid | AdA | 22:4 n-6 | 16.8 ± 3.1 | 23.0 ± 2.6 | 0.0007 |
| a-Linolenic acid | ALA | 18:3 n-3 | 37.8 ± 8.5 | 28.0 ± 15.7 | ns |
| Eicosatetraenoic acid | ETA | 20:4 n-3 | 1.6 ± 0.6 | 0.9 ± 0.3 | ns |
| Eicosapentaenoic acid | EPA | 20:5 n-3 | 17.2 ± 3.9 | 14.6 ± 1.5 | ns |
| Decosahexaenoic acid | DHA | 22:6 n-3 | 251 ± 28 | 339 ± 58 | 0.0013 |
| FADS1 activity | AA/DGLA | 20:4n-6/20:3n-6 | 4.8 ± 0.5 | 7.3 ± 0.6 | <0.0001 |
| FADS2 activity | GLA/LA | 18:3n-6/18:2n-6 | 0.01 ± 0.002 | 0.010 ± 0.004 | ns |

### Example 2: Fads1 KO Mice are Resistant to HFD-Induced Obesity Due to Increased Energy Expenditure

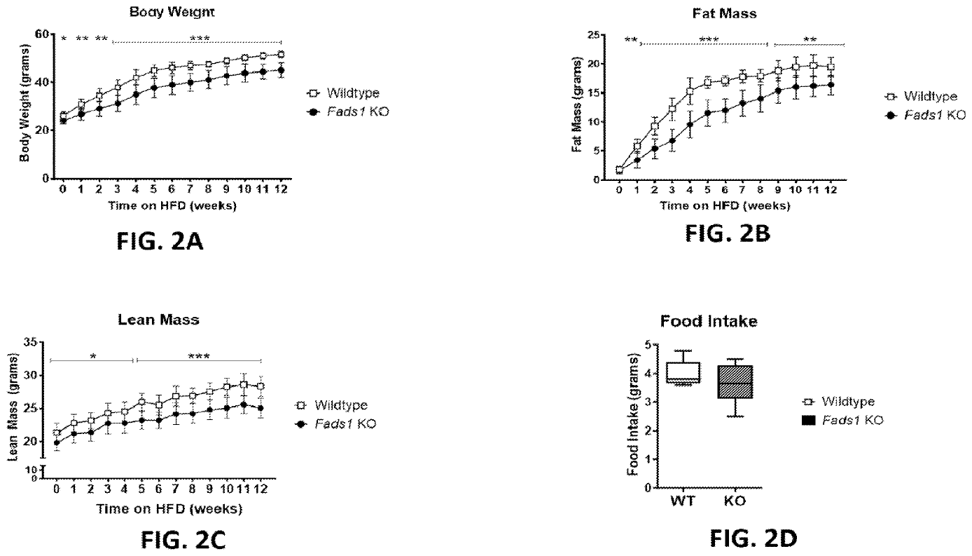

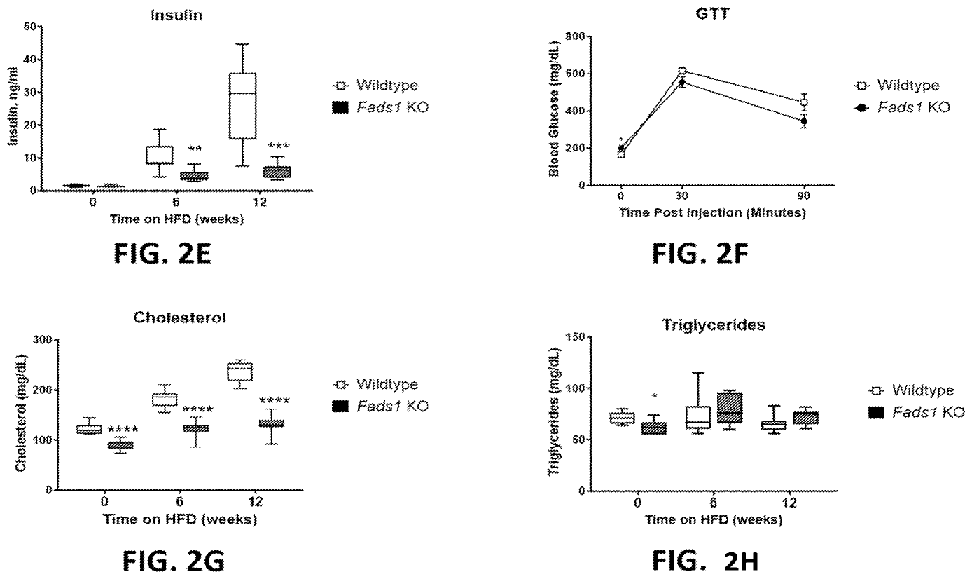

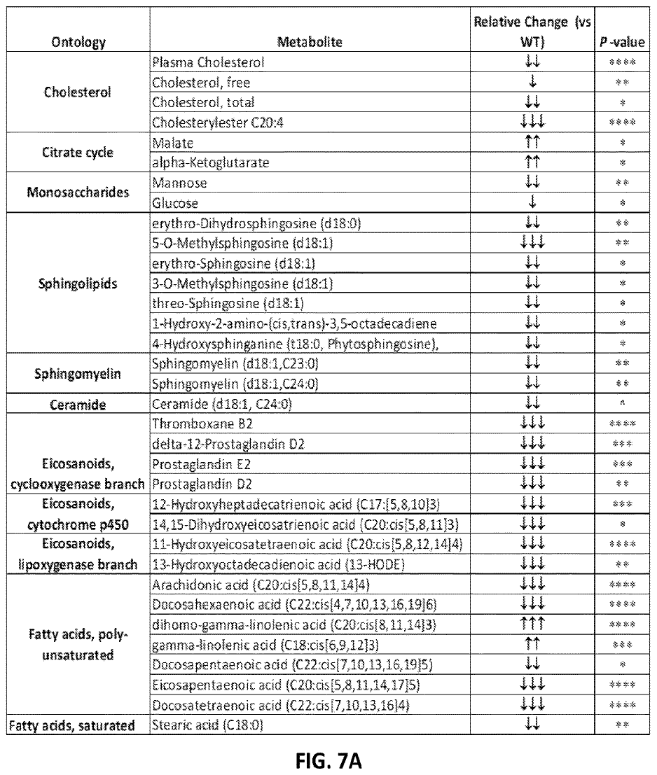

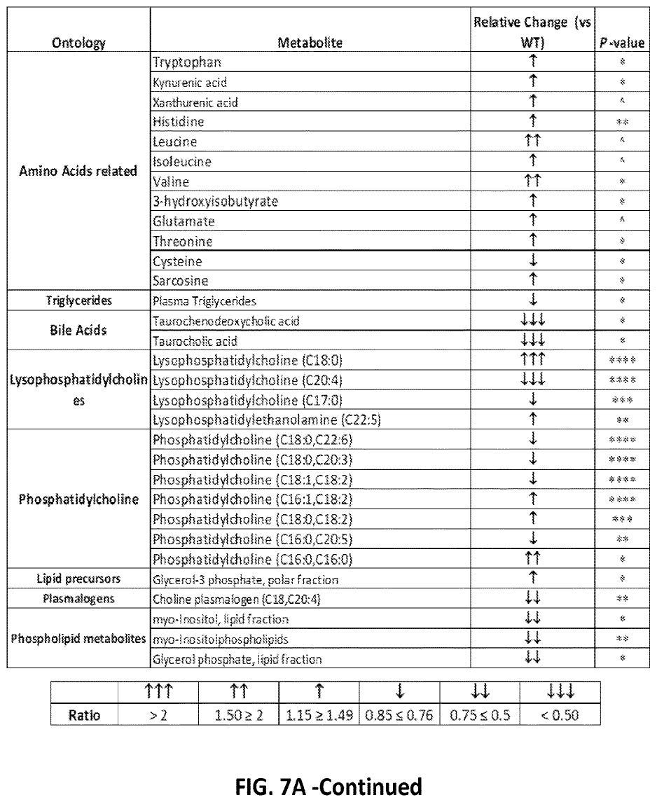

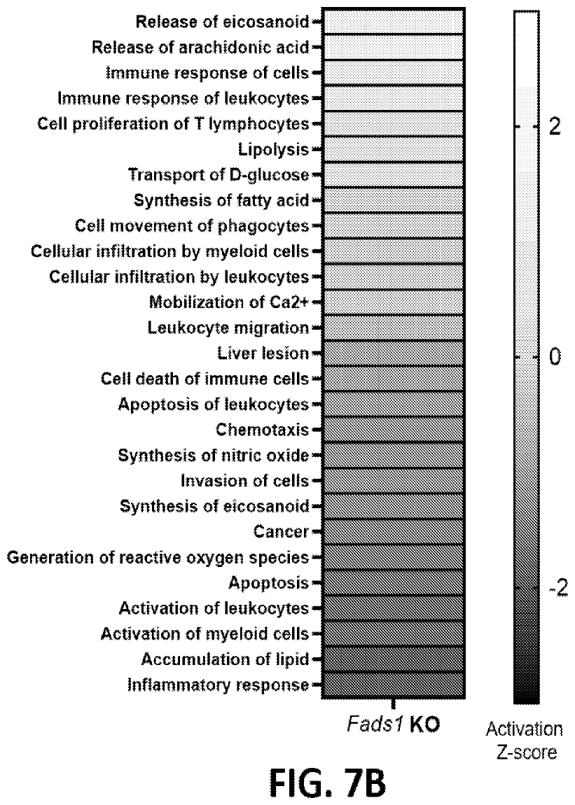

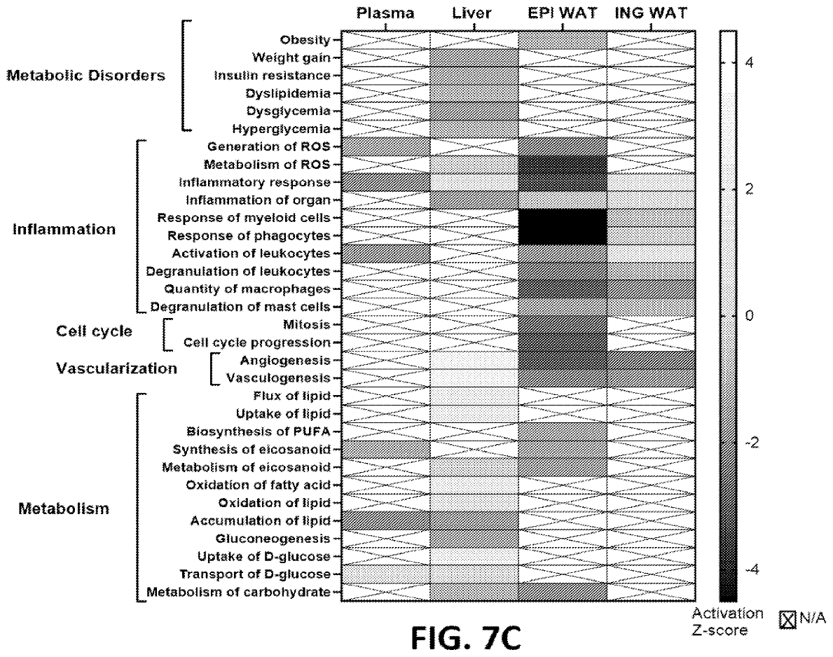

To investigate the impact of FADS1 inhibition on obesity, age-matched wild type (WT) and Fads1 KO male littermates were fed a HFD for 12 weeks. Due to previous reports on the dependence of Fads1 KO mice on AA supplementation for survival, the viability of Fads1 KO mice on HFD that contained 0.064% AA (wt/wt) was tested. Notably, Fads1 KO mice demonstrated complete viability when fed 0.064% AA and no health issues were observed after 32 weeks. At the end of the feeding period, Fads1 KO mice weighed 12.8% less than their WT littermates (FIG. 2(a)). Fads1 KO mice gained 15.8% less fat mass and 11.6% less lean mass relative to WT mice (FIG. 2(b)-(c)). Fads1 KO mice showed no statistical significance in comparison to the WT mice in food intake (FIG. 2(d)). Fads1 KO mice had lower insulin levels (FIG. 2(e)) and improved glucose tolerance during a glucose tolerance test (GTT) compared with their WT littermates (FIG. 2(f)). Fads1 KO mice also showed reduced plasma cholesterol levels before the initiation of HFD and maintained lower cholesterol levels throughout the HFD feeding period compared with their WT littermates (FIG. 2(g)). No differences (FIG. 2(h)) or slightly lower (FIG. 7(a)) triglyceride levels was observed between Fads1 KO and WT mice.

Since AA supplementation may impact the phenotype of Fads1 KO mice, WT and Fads1 KO mice were fed with HFD supplemented with or without 2% AA for 9 weeks. Supplementation of Fads1 KO with HFD containing 2% AA completely reversed the anti-obesogenic phenotype described above suggesting that the metabolic phenotype with Fads1 deletion is AA dependent and lack of additional AA supplementation is critical for the manifestation of the obesity-resistant phenotype of Fads1 KO on HFD.

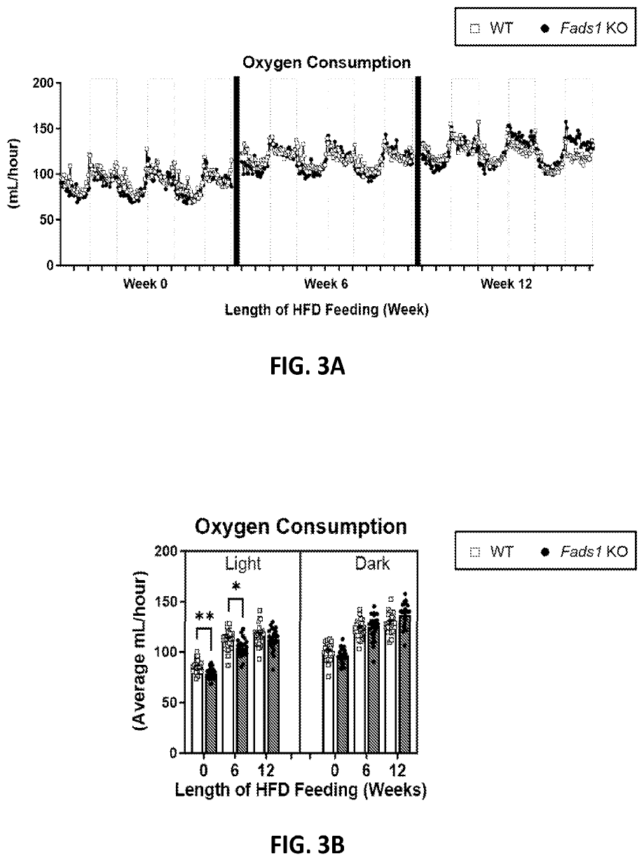

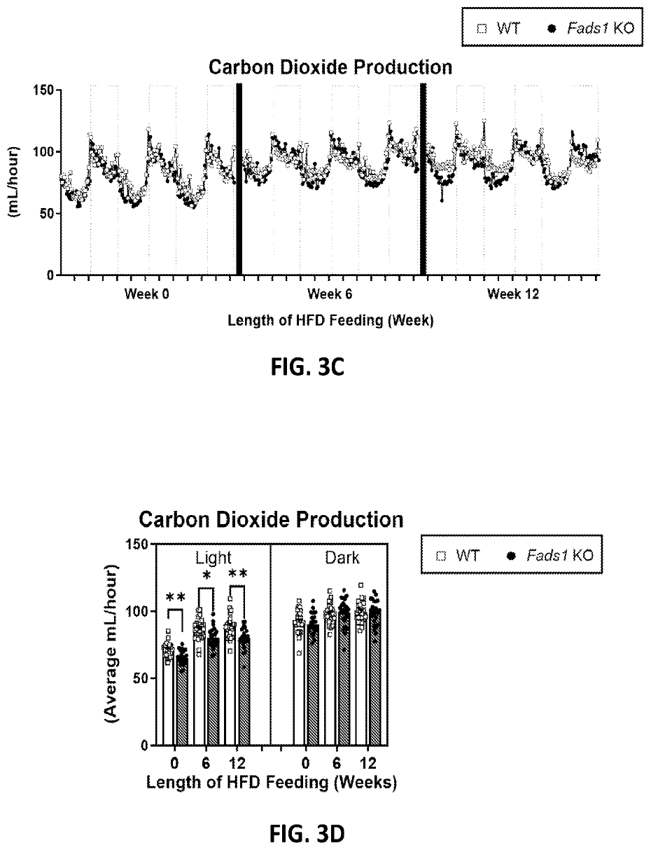

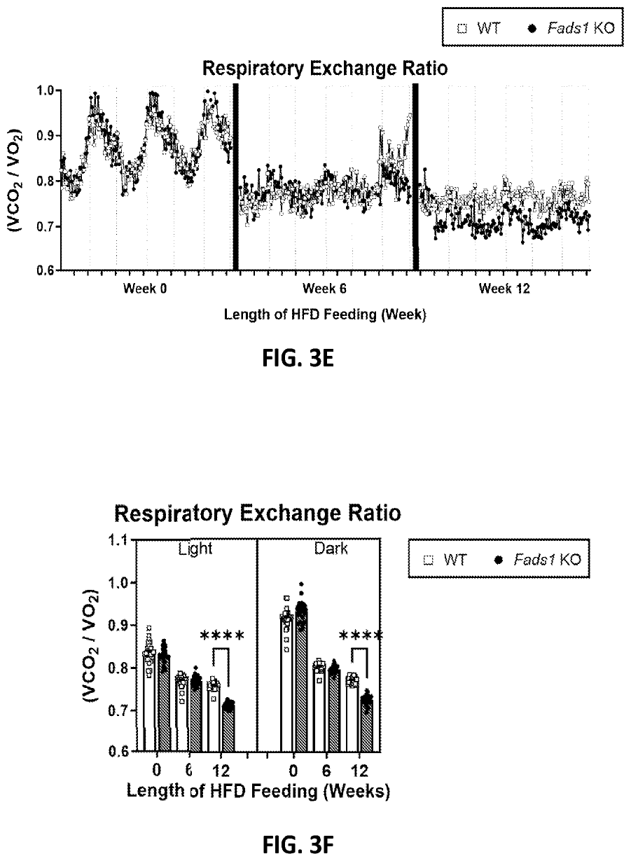

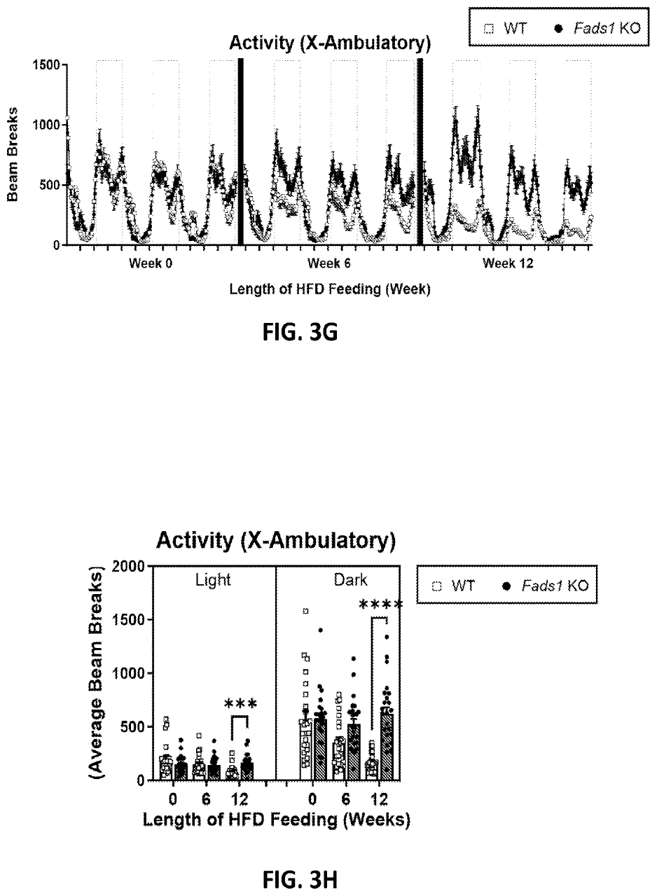

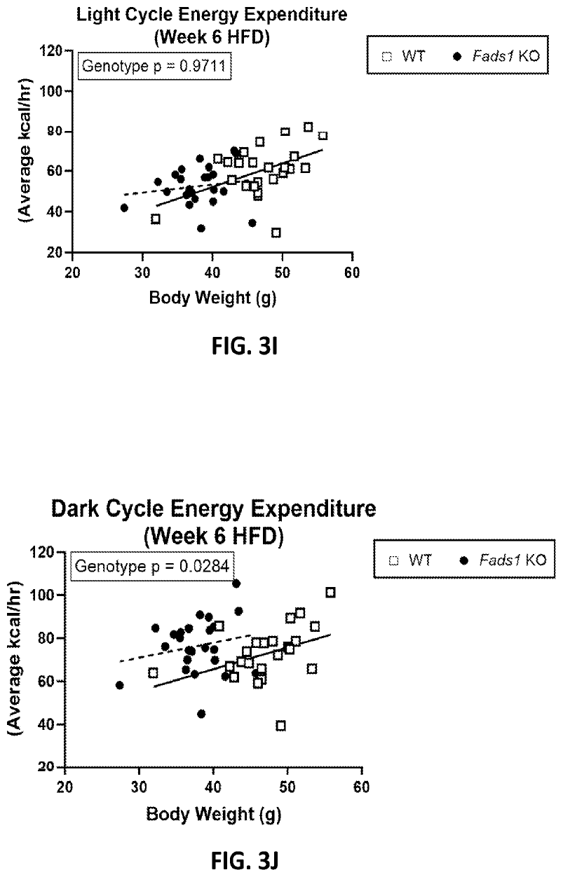

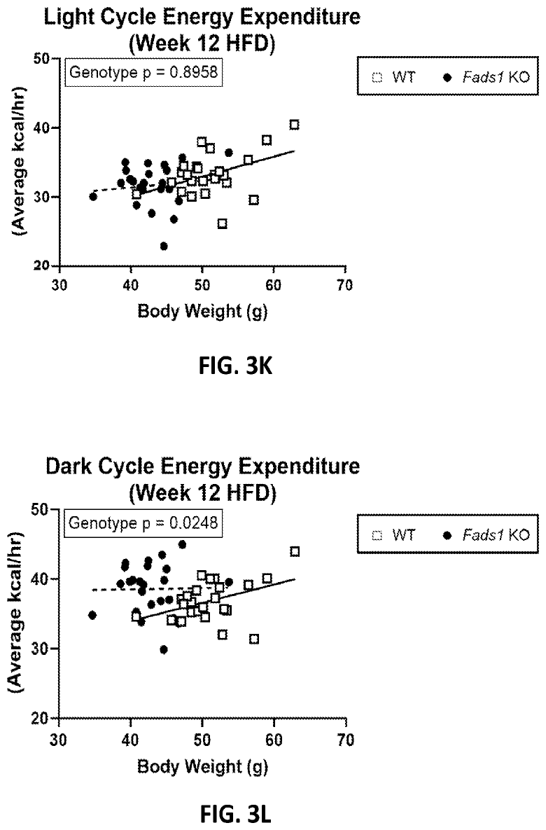

To investigate the physiological and metabolic impact of FADS1 deletion, indirect calorimetry measurement in Fads1 KO and WT mice was performed at the start of and after 6 and 12 weeks of HFD feeding (FIG. 3). Fads1 KO mice had significantly lower oxygen consumption and carbon dioxide production during the light cycle at 0 and 6 weeks (FIG. 3(a)-(b)) and at 0, 6, and 12 weeks (FIG. 3(c)-(d)), respectively. The RER of Fads1 KO mice at both light and dark cycles were significantly lower after 12 weeks of HFD feeding (FIG. 3(e)-(f)), demonstrating increased lipid oxidation in Fads1 KO mice relative to WT mice. Fads1 KO mice were more active than WT mice at 12 weeks in the dark cycle and demonstrated higher dark cycle energy expenditure relative to WT mice after 6 and 12 weeks of HFD feeding (FIG. 3(g)-(h)). FIGS. 3(i)-(l) provide plots of light and dark cycle energy expenditures in kcal/hour versus body weight after 6 and 12 weeks of HFD feeding. Due to the lack of a significant difference in food intake (FIG. 2(d)), increased energy expenditure helps elucidate the reason for resistant of Fads1 KO to DIO.

### Example 3: Transcriptome and Metabolome Changes in Liver, Fat, and Plasma of Fads1 KO Mice

Liver, EPI and ING WAT were collected from HFD-fed male Fads1 KO and WT mice and RNA-Seq was performed. First, the Fads1 KO versus WT RNA-Seq expression profile was characterized, with the goal of defining genotype-related expression differences and identifying genes that are relevant to FADS1 in relation to obesity. Liver and WAT exhibited distinct genotype-specific transcriptional profiles (FIG. 4(a), FIG. 5(a)). Fads1 KO compared to WT demonstrated the greatest transcriptome changes in EPI WAT in terms of number of affected genes and tissue cellular composition (FIG. 4(a)-(b), FIG. 5(a)). Cellular composition from EPI WAT was modified in Fads1 KO mice showing a larger population of endothelial cells (54% versus 38%) and less myeloid cells (13% versus 27%) relative to WT mice (FIG. 4(b)).

The gene expression changes observed in the liver from Fads1 KO mice were likely due to hepatic gene expression alterations and not due to changes in the cellular composition (FIG. 4(b)-(c), FIG. 5(a)). From the 185 higher-expressing and 126 lower-expressing genes in the liver of Fads1 KO mice compared to WT, some belonged to metabolic pathways including Lipid Metabolism (Dgat2, Elov13, Fads2, Lipe), Fatty Acid Metabolism (Acox1, Hadhb), and PPAR Signaling (Cpt1a, Cpt1b, Cyp4a12b) (FIG. 4(c), FIG. 5(a)). For EPI WAT, the metabolic pathways that show the most changes were Regulated Lipolysis in Adipocytes (Adrb3, Lipe, Mg11, Pnpla2, Pnpla3), Lipid Metabolism (Dgat1, Dgat2, Fas, Scd1, Srebf1), Fatty Acid Metabolism (Acaa1a, Acads, Acox1, Ehhadh), and Insulin Signaling (Irs1, Irs2) (FIG. 4(d), FIG. 5(a)). Significant changes in gene expression within PI3K signaling, Apoptosis, and Inflammation (Adgre1, Agtr1a, CD14, CD68, Fdft1) pathways coincided with the shift in cellular representation of subsets of immune cells from both myeloid and lymphoid lineage in the EPI WAT of Fads1 KO relative to WT mice (FIG. 4(b), FIG. 4(d), FIG. 5(a)).

Gene expression of adiponectin (Adipoq) was significantly upregulated in EPI WAT of Fads1 KO mice than in WT mice (FIG. 5(a)) and coincided with a higher protein concentration of adiponectin in EPI WAT (FIG. 5(b)). Protein concentration of pro-inflammatory markers such as PAI-1 and MCP-1 were decreased in EPI and ING WAT of Fads1 KO mice (FIG. 5(a)-(b)).

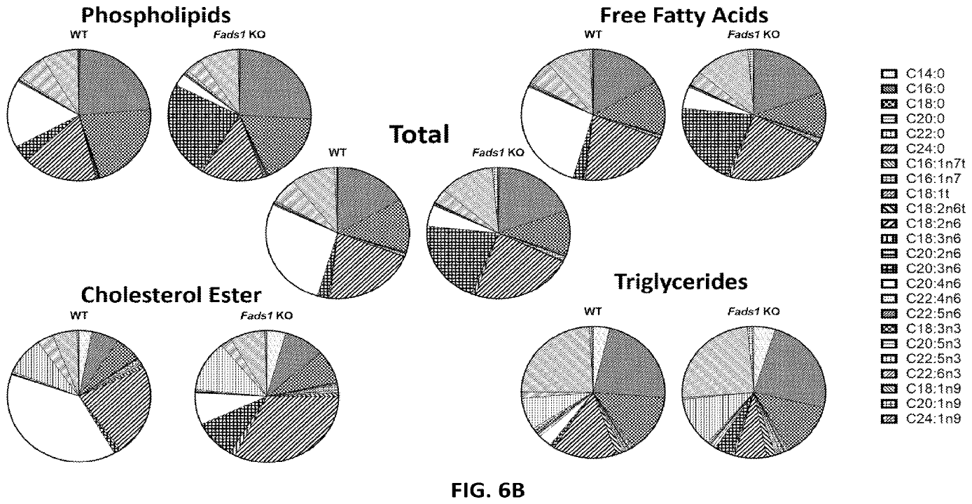

Plasma PUFA measurements and metabolomic analysis of HFD-fed Fads1 KO mice were significantly different compared to WT mice (FIG. 7(a)-(c), Table 3.1). FA concentration is decreased in the plasma and trends to decreased in all four lipid subfractions: phospholipids, triglycerides, cholesterol esters, and free fatty acids (FFAs) (FIG. 6(a)), with alteration of FA composition in each subfraction (FIG. 6(b)). Fads1 KO mice have decreased circulating palmitic acid and stearic acid levels compared to WT mice (FIG. 7(a), Table 3.1, Table 3,2). AA and EPA levels, as well as the downstream PUFAs levels such as adrenic acid, docosapentaenoic acid, and docosahexaenoic acid were significantly decreased in Fads1 KO mice (FIG. 7, Table 3.1). FADS1 substrate DGLA and upstream PUFA GLA were significantly elevated in Fads1 KO mice than in WT mice. All four eicosanoids measured in the cyclooxygenase (COX) branch (prostaglandin E2, prostaglandin D2, delta-12 prostaglandin D2, and thromboxane B2) were decreased (FIG. 7) in Fads1 KO mice compared with WT mice, whereas only a fraction of eicosanoids measured in each of the less inflammatory lipoxygenase (two of eight) and cytochrome P450 (CYP) epoxygenase (two of five) branches were significantly decreased in Fads1 KO mice compared with WT mice. Furthermore, Fads1 KG mice had elevated citrate cycle intermediates (malate and aipha-ketoghutarate), a signature of increased oxidation, and decreased taurine-bile acids (taurocholic acid and taurochenodeoxycholic acid) in the plasma in comparison to WT mice (FIG. 7). The levels of choline plasmalogen, sphingolipids, and phospholipid metabolites were also decreased in Fads1 KG mice relative to WI mice (FIG. 7).

TABLE 3.1
PUFA profile in plasma of Fads1 KO and WT male mice

| | | Shorthand | Wildtype, | Fads1 KO, | P-value* (KO |
|---|---|---|---|---|---|
| Common Name | Abbreviation | Notation | μg/ml | μg/ml | vs WT) |
| Linoleic acid | LA | 18:2 n-6 | 447.0 ± 28.2 | 286.2 ± 33.3 | 0.0021 |
| γ-Linoleic acid | GLA | 18:3 n-6 | 3.7 ± 0.5 | 6.0 ± 0.5 | <0.0001 |
| Dihomo-γ-Linolenic acid | DGLA | 20:3 n-6 | 51.9 ± 12.8 | 265.6 ± 51.2 | <0.0001 |
| Arachidonic acid | AA | 20:4 n-6 | 285.3 ± 36.2 | 71.2 ± 8.1 | <0.0001 |
| Adrenic acid | AdA | 22:4 n-6 | 7.6 ± 1.3 | 2.2 ± 0.3 | <0.0001 |
| α-Linolenic acid | ALA | 18:3 n-3 | 5.1 ± 1.9 | 3.7 ± 1.9 | 0.1498 |
| Eicosatetraenoic acid | ETA | 20:4 n-3 | 1.0 ± 0.2 | 6.7 ± 0.7 | <0.0001 |
| Eicosapentaenoic acid | EPA | 20:5 n-3 | 4.8 ± 0.6 | 0.5 ± 0.1 | <0.0001 |
| Decosahexaenoic acid | DHA | 22:6 n-3 | 145.5 ± 19.0 | 71.9 ± 12.7 | <0.0001 |
| FADS1 activity | AA/DGLA | 20:4n-6/ | 5.65 ± 0.8 | 0.3 ± 0.1 | <0.0001 |
| | | 20:3n-6 | | | |
| FADS2 activity | GLA/LA | 18:3n-6/ | 0.01 ± 0.00 | 0.02 ± 0.00 | <0.0001 |
| | | 18:2n-6 | | | |

TABLE 3.2
Fatty acid concentration of WT and Fads1 KO plasma lipid subfractions

| Fatty | Total | | Phospholipids | | Triglycerides | | Cholesterol ester | | Free fatty acids | |
|---|---|---|---|---|---|---|---|---|---|---|
| acids | | Fads1 | | Fads1 | | Fads1 | | Fads1 | | Fads1 |
| (μg mL⁻¹) | WT | KO | WT | KO | WT | KO | WT | KO | WT | KO |
| C14:0 | 7.1 | 7.0 | 4.6 | 5.2 | 27.4 | 25.9 | 58.8 | 58.9 | 40.5 | 21.9 |
| C16:0 | 668 | 560* | 605 | 496 | 177 | 127 | 121 | 124 | 258.4 | 142.7* |
| C18:0 | 533 | 318*** | 553 | 336* | 113 | 69.1 | 118 | 112 | 207.2 | 94.6* |
| C20:0 | 7.3 | 8.1 | 4.2 | 4.0 | 2.5 | 1.5 | 1.5 | 1.4 | 3.1 | 1.8 |
| C22:0 | 5.9 | 5.6 | 3.1 | 2.4 | 3.6 | 2.6 | 2.5 | 2.7 | 5.1 | 2.0 |
| C24:0 | 2.2 | 2.3 | 1.8 | 1.0 | 1.5 | 1.5 | 1.4 | 0.8 | 2.2 | 1.0 |
| C16:1n7t | 3.7 | 3.6 | 2.3 | 2.7 | 1.0 | 0.5 | 2.0 | 4.1** | 0.8 | 0.5 |
| C16:1n7 | 27.3 | 29.6 | 4.9 | 5.8 | 4.4 | 4.7 | 18.6 | 16.8 | 4.2 | 7.9* |
| C18:1t | 3.8 | 4.3 | 5.3 | 4.4 | 5.6 | 6.2 | 12.6 | 9.4 | 7.0 | 2.3 |
| C18:2n6t | 2.9 | 5.1* | 4.6 | 3.9 | 11.2 | 10.6 | 18.8 | 13.3 | 3.4 | 1.2 |
| C18:2n6 | 845 | 672** | 431 | 269 | 137 | 38.6 | 430 | 439 | 69.4 | 29.0 |
| C18:3n6 | 11.5 | 21.3*** | 2.1 | 3.9 | 1.0 | 2.7 | 9.3 | 18.2** | 0.5 | 0.8 |
| C20:2n6 | 9.5 | 9.5 | 7.7 | 7.5 | 2.9 | 1.9 | 0.8 | 0.8 | 0.9 | 1.1 |
| C20:3n6 | 96.3 | 596*** | 98.8 | 452* | 4.1 | 20.1*** | 20.8 | 129*** | 1.7 | 10.7*** |
| C20:4n6 | 1121 | 155*** | 425 | 58.7*** | 22.6 | 3.0*** | 748 | 108*** | 7.2 | 2.0 |
| C22:4n6 | 7.9 | 2.6*** | 5.1 | 2.1** | 4.9 | 3.5 | 1.6 | 1.2 | 1.8 | 0.9 |
| C22:5n6 | 7.6 | 8.8 | 5.4 | 5.9 | 2.3 | 1.2 | 1.4 | 1.9 | 0.8 | 0.4 |
| C18:3n3 | 6.2 | 4.9* | 0.8 | 0.6 | 9.1 | 1.5 | 3.0 | 2.2 | 5.6 | 1.3 |
| C20:5n3 | 13.5 | 1.7*** | 4.0 | 3.1 | 4.8 | 2.3* | 8.7 | 4.8 | 7.2 | 5.5 |
| C22:5n3 | 10.7 | 3.3*** | 14.6 | 15.2 | 56.3 | 64.0 | 193 | 174 | 108.7 | 36.7* |
| C22:6n3 | 249 | 105*** | 175 | 71.9** | 12.3 | 2.3** | 70.7 | 34.5*** | 3.6 | 2.6 |
| C18:1n9 | 403 | 362 | 208 | 173 | 190 | 133 | 108 | 107 | 94.6 | 79.8 |
| C20:1n9 | 26.8 | 34.2 | 6.4 | 6.1 | 3.9 | 5.5 | 7.3 | 4.6 | 1.9 | 3.4* |
| C24:1n9 | 4.9 | 5.9 | 2.4 | 1.7 | 2.3 | 2.4 | 3.1 | 2.5 | 1.8 | 1.0 |
| Total | 4074 | 2925*** | 2574 | 1933 | 801 | 531 | 1960 | 1371* | 838 | 451* |
| EPA/AA | 0.012 | 0.011 | 0.009 | 0.044 | 0.211 | 0.814* | 0.012 | 0.045* | 1.67 | 3.23 |

Interestingly, plasma levels of branched-chain amino acids (BCAAs; valine, leucine, and isoleucine) trended to be elevated despite increased plasma levels of 3-hydroxy-isobutyrate, a metabolite of valine degradation, and upregulation of BCAA catabolism genes (Bcat1, Bcat2, Bckdha, Bckdhb, Bckdk) in EPI WAT (FIG. 5(a), FIG. 7). Increased plasma levels of histidine and tryptophan were also observed and were likely the result of decreased hepatic expression of the rate-limiting enzymes in histidine degradation (Hal), and tryptophan degradation (Tdo2) (FIG. 5(a), FIG. 7(a)). Interestingly, although increase in plasma histidine level was accompanied by the expected increase in glutamate levels (FIG. 7(a)), increased tryptophan levels were not accompanied by decreases in downstream metabolites: instead, an increase in kynurenic acid and xanthurenic acid were observed (FIG. 7(a)). This may be due to upregulation of tryptophan degradation genes such as Kyat3 and Kno in the WAT (FIG. 5(a)). FIG. 5(b) provides a comparison of plasma, EPI WAT and ING WAT of adiponectin, leptin, PAI-1, and MCP-1 levels in WT versus Fads1 KO mice.

IPA analysis of the metabolome changes in the Fads1 KO mice revealed trends in downregulation of biofunctions such as Inflammatory Response, Accumulation of Lipids, Activation of Myeloid Cells and Leukocytes, and Generation of Reactive Oxygen Species (ROS) (FIG. 7(b)). These biofunctions were also similarly altered in the liver or EPI WAT of Fads1 KO mice when comparing the tissue transcriptome to the plasma metabolome of Fads1 KO mice relative to WT mice (FIG. 7(c)).

Fads1 KO mice have elevated circulating kynurenic acid (FIG. 7), which was shown to increase energy expenditure through activation of GPR35 in the adipose tissue to stimulate lipid oxidation, thermogenesis, and reduce inflammation, leading to reduced weight gain and improved insulin sensitivity. FIG. 6(b) provides fatty acid composition of total plasma and each of the four lipid subfractions in the plasma of WT and Fads1 KO mice.

### Example 4: FADS1 Inhibition Studies Using Small Molecule FADS1 Inhibitors

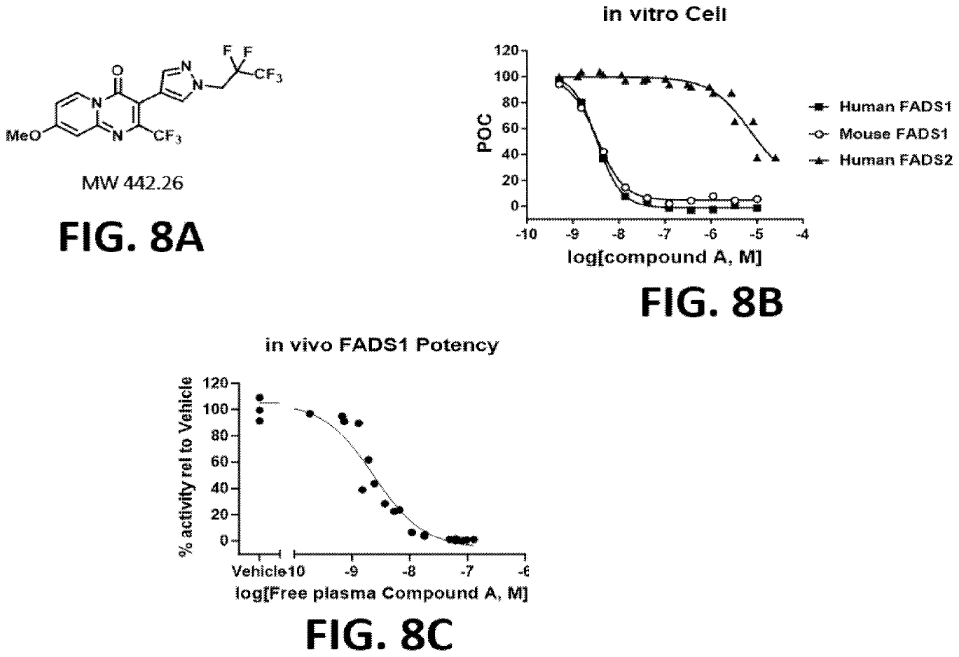

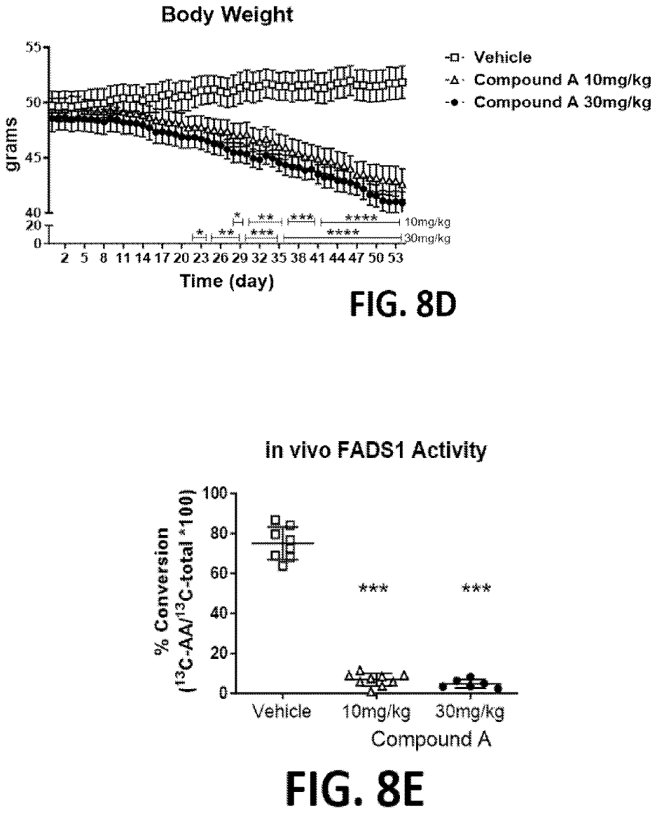

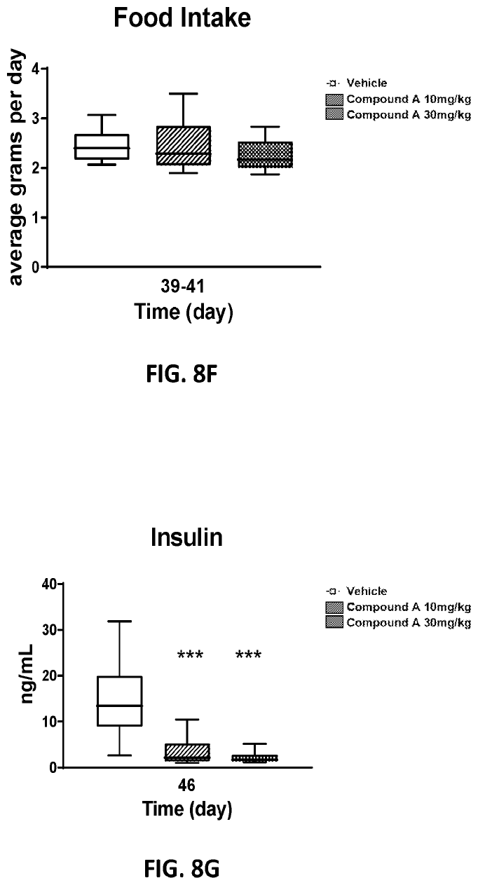

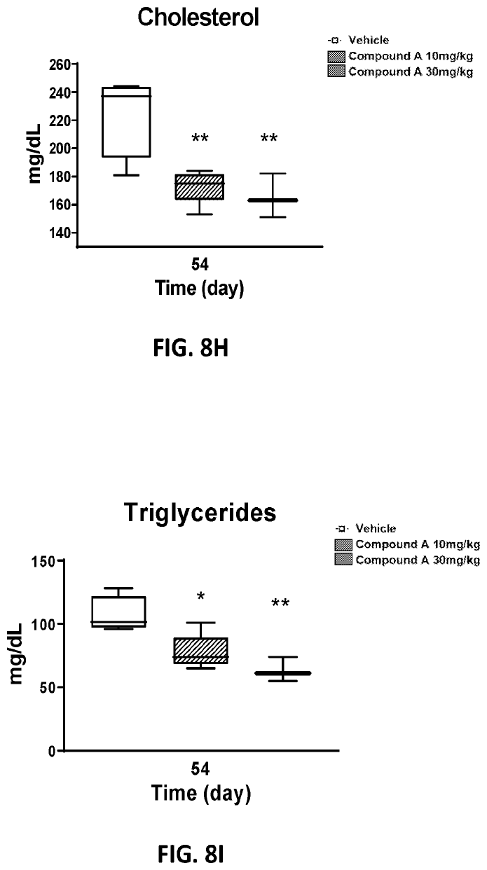

Based on the alteration in FADS1 activity in obese humans and mice and protection against diet-induced obesity in Fads1 KO mice, FADS1 inhibition was targeted for investigation. A potent, orally bioavailable molecule, Compound A (FIG. 8(a)) was identified. This compound inhibits FADS1 activity in vitro with a half-maximal inhibitory concentration (IC₅₀) of 0.003 μM and 0.004 μM against human and mouse FADS1, respectively, and shows selectivity over FADS2 (FIG. 8(b)).

### In Vitro FADS1 and FADS2 Activity Assays

HEK 293 cells in Dulbecco's Modified Eagle's medium (DMEM), 10% fetal bovine serum (FBS), 1× penicillin-streptomycin-glutamine (PSG) were mixed with FADS1 BacMam or FADS2 BacMam reagents at multiplicity of infection (MOI) ratio of 25 and plated in a in 96-well poly-D-lysine-coated plate. Plates were incubated in a 37° C., 5% CO₂ for 24 hours. Cells were washed with DPBS and cells were incubated with 90 ml of compound dilution in DPBS at 5% CO₂, 37° C. for 20 minutes. Ten L of the substrate, ¹³C₅-DGLA (FADS1), or 13C-LA (FADS2) diluted in DPBS were added to the cells and incubated at 5% CO₂, 37° C. for 30 minutes (FADS1) or 45 minutes (FADS2). Cells were then washed with DPBS and 50 μL of 2N NaOH were added to each well and incubated in a 65° C. oven for 1 hour for the saponification reaction. After cooling to room temperature, 22 μl of formic acid was added followed by the addition of 113 μl of acetonitrile with 1.5 μM ALA-d₁₄ as the internal standard. Samples were then subjected to LC-MS/MS detection for the following analytes for the FADS1 reaction: ¹³C₅-DGLA and ¹³C₅-AA or the following analytes for the FADS2 reaction: ¹³C₁₈-LA, ¹³C₁₈-GLA, ¹³C₁₈-DGLA and ¹³C₁₈-AA. Samples were injected onto a Poroshell 120 EC-C18 3.0×50 mm, 1.9 um id column (Agilent, Santa Clara, CA).

The analyte peak areas were quantified by using the Analyst software (SCIEX, Framingham, MA). The percent of substrate to product conversion relative to untreated control were calculated (percent of control [POC]). Non-linear regression of the POC versus the compound concentration was plotted using GraphPad Prism (GraphPad Software, San Diego, CA).

### In Vivo FADS1 Activity Measurement Using the Liver and Blood Plasma

To measure the in vivo activity of FADS1 using the liver, dose titrations of Compound A were orally administered in DIO C57Bl/6 mice for 23.5 hours. Mice were then dosed by intraperitoneal injection with a solution comprising of a mixture of 0.1 ml solution of ¹³C₅-DGLA (2 mM in 18.42 mM Na₂CO₃ buffer). Necropsy were performed at 30 minutes post ¹³C₅-DGLA dosing. Liver from ¹³C₅-DGLA dosed DIO mice was collected for pharmacodynamic (PD) analysis and EDTA plasma was collected for compound exposure determination. For PD analysis, 150 mg of frozen liver tissue were premixed with ALA-D₁₄ as an internal standard. Liver samples were homogenized and extracted twice with chloroform/methanol (2:1) solution. The organic phases for the two extractions were pooled and evaporated to dryness under nitrogen gas. The liver extract was resuspended in 200 μl 2N NaOH and saponified at 65° C. for 1 hour. After cooling to room temperature, 100 μl of formic acid was added and the mixture was subjected to two consecutive extractions with a chloroform:methanol (2:1) solution. The pooled organic phases were evaporated to dryness under nitrogen gas and resuspended in a solution containing acetonitrile and methanol at a ratio of 1:1. The extracts were then analyzed by LC/MS/MS for the following analytes: ¹³C₅-DGLA, ¹³C₅-AA, with ALA-d₁₄ as the internal standard. The sample (20 μl) was injected onto a Poroshell 120 EC-C18 3.0×50 mm, 1.9 μm id column (Agilent, Santa Clara, CA). The analyte peak areas were quantified using the Analyst software (SCIEX, Framingham, MT). The residual in vivo FADS1 activity was calculated by taking the percent of substrate to product conversion normalized to the vehicle group to determine the percent of control (POC) and the percent inhibition relative to control (vehicle).

To determine the degree of FADS1 inhibition, the in vivo FADS1 activity as the percent of conversion calculated using the following equation:

$$Percent Conversion = produce / ( substrate + product ) * 100 {=}^{13} {C}_{5 -} AA / {(}^{13} {C}_{5 -} DGLA {+}^{13} {C}_{5 -} AA ) * 100 was calculated .$$

The data were then normalized to the vehicle group to determine the percent of inhibition relative to control (vehicle) by the following equation:

$$Percent of inhibition = ( 1 - ( FADS 1 activity of each mouse / average FADS 1 activity of vehicle group ) ) * 100$$

To calculate the in vivo potency, dose response graphs were generated by plotting the plasma concentration of the test compound determined from the PK analysis on the abscissa, against the percent of control (POC, relative to vehicle) of the FADS1 activity for each mouse. The data points were then fitted by using the log(inhibitor) vs response nonlinear regression algorithm using GraphPad Prism (version 8.4.3, San Diego CA) to calculate the in vivo IC₅₀ (total).

Free (unbound) IC₅₀ and IC₉₀ values were calculated by first calculating the unbound compound concentration in the plasma by multiplying the plasma concentration of test compound with the fraction unbound (fu) value in mouse plasma determined in the plasma protein binding study. A dose response curve was used to determine the unbound IC₅₀ and IC₉₀ by plotting the unbound plasma concentration of the compound on the abscissa, against the percent of control (POC, relative to vehicle) of FADS1 activity for each mouse. The data points were then fitted with a log (inhibitor) versus response nonlinear regression algorithm using GraphPad Prism (version 8.4.3, San Diego, CA) to calculate the in vivo IC₅₀ (Free). In vivo IC₉₀ (Free) was calculated by using the generated curve to interpolate the concentration at which 90% of the in vivo FADS1 activity was inhibited with only a 10% activity observed.

FADS1 activity in the presence of Compounds B-E was also measured using blood plasma (in addition to using the liver). The above procedures were perforned to measure the in vivo activity of FADS1 using the liver. The following general procedures were used to measure FADS1 activity using blood plasma. Diet-induced obese (DIO) mice that were on 12 weeks of high fat diet were used to determine the pharmacokinetic (PK) and pharmacodynamic (PD) properties of test compounds. To determine the target coverage at 24 hours after dosing with a test compound (e.g., Compounds B-E, shown below), mice were IP dosed with 0.1 ml solution of ¹³C₅-DGLA (2 mM in 18.42 mM Na₂CO₃ buffer). Necropsy were performed at 30 minutes post ¹³C₅-DGLA dosing. EDTA Plasma was collected for PK and PD analysis.

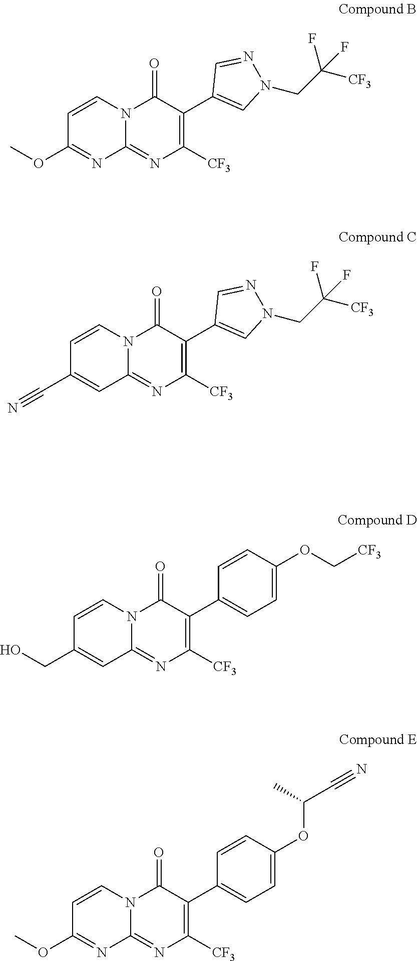 [SMILES: COc1ccn2c(=O)c(-c3cnn(CC(F)(F)C(F)(F)F)c3)c(C(F)(F)F)nc2n1 ; N#Cc1ccn2c(=O)c(-c3cnn(CC(F)(F)C(F)(F)F)c3)c(C(F)(F)F)nc2c1 ; O=c1c(-c2ccc(OCC(F)(F)F)cc2)c(C(F)(F)F)nc2cc(CO)ccn12 ; COc1ccn2c(=O)c(-c3ccc(O[C@H](C)C#N)cc3)c(C(F)(F)F)nc2n1]

For PD analysis, 50 μl of plasma or standards (pooled untreated mouse plasma with ¹³C₅-DGLA and ¹³C₅-AA added at concentration of 4.57 nM to 10 uM) were mixed with 10 μl of 90% methanol and 10 μl of 50 μg/mL of ALA-D14 as an internal standard (100 μM alpha-linolenic acid-d₁₄ (ALA-d₁₄, Cayman Chemical)) in a 96 well plate. 490 μl of chloroform:methanol (2:1) were added to each well. Plates were plated on a shaker for 5 min prior to adding 66.5 μl water. The plates were shaken for another 5 minutes, followed by centrifugation at 2000 RPM for 5 minutes. The bottom organic layer was transferred to a clean 96 well plate. 333 ul of chloroform:methanol (2:1) were added to the to the remaining liquid phase in the original plate. After 5 min of shaking, the plate was centrifuged at 2000 RPM for 5 minutes. The bottom layer was collected and combined with the first bottom layer. The combined layers were evaporated to dryness under a constant stream of nitrogen gas. After the plate has been dried, 100 μl of 2N NaOH was added to the mixture for subsequent saponification at 65° C. for 1 hour. The samples were then cooled to room temperature and the mixture was then acidified with 50 μl of formic acid followed by two consecutive chloroform:methanol (2:1) extractions. 500 μl of Chloroform:methanol (2:1) was added and the mixture thoroughly mixed by shaking. The plate was then centrifuged at 2,000 rpm for 5 min. The bottom chloroform phase was transferred to a new 2 mL 96 well plate and the remaining aqueous layer was extracted with another 250 μl of chloroform:methanol (2:1). The organic extracts were combined, and the solvent was evaporated to dryness by placing the plate under nitrogen. Extracts were then dissolved in 200 μl of acetonitrile/methanol (1:1). The plate was placed on a shaker for 30 minutes to ensure thorough mixing and dissolution of the extracted fatty acids. The plate was then centrifuged at 2000 RPM for 5 minutes. 80 μl of the samples were transferred to a new 96 well polypropylene plate for analysis on a LC-MS/MS for the following analytes: ¹³C₅-DGLA, ¹³C₅-AA, with ALA-d₁₄ as the internal standard. Briefly describing the LC-MS/MS method: 20 μl of sample was injected onto a Poroshell 120 EC-C18 3.0×50 mm, 1.9 μm id column. Mobile phases were 80% acetonitrile containing 5 mM ammonium acetate for mobile phase A and 99.5% acetonitrile containing 5 mM ammonium acetate for mobile phase B. The LC gradient was a 3 min long method at a flow rate of 0.4 mL/minute consisting of 45% B at 0 to 0.5 min, followed by a 45% B to 100% B from 0.5 to 2.0 min; the system was then maintained at 100% B from 2.0 min to 2.5 min and returned to 45% B at 2.6 min and maintained until end of the method. The analyte peak areas were quantified by using the SCIEX Analyst software. To determine the degree of FADS1 inhibition, the in vivo FADS1 activity, POC, percent of inhibition, and in vivo IC50 values are calculated as described above for liver FADS1 activity measurement.

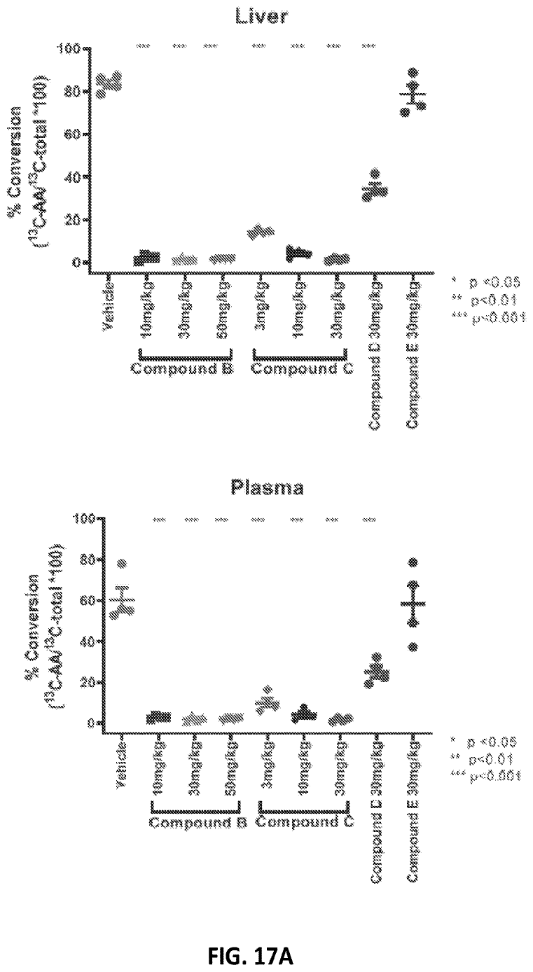

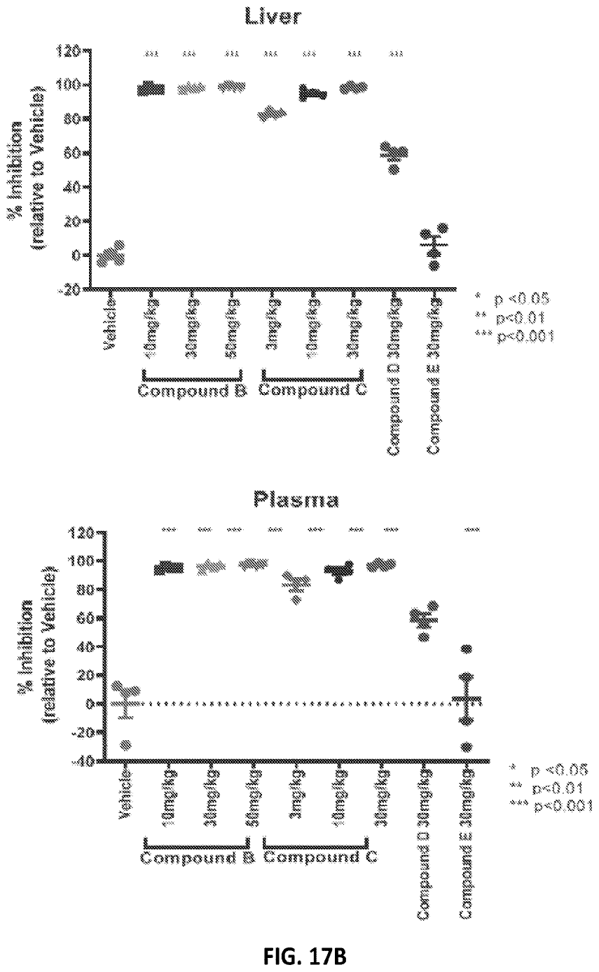

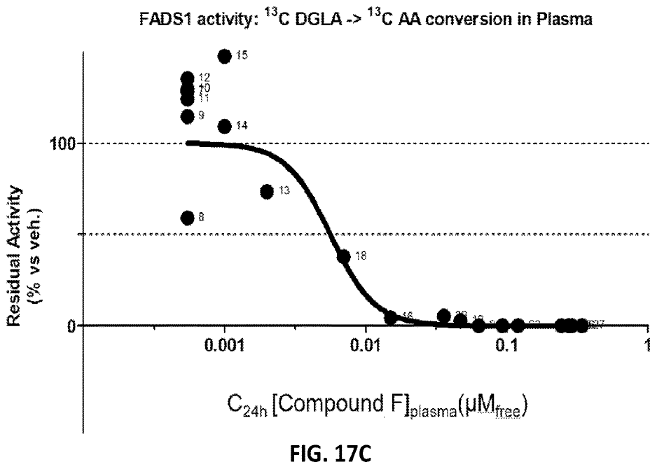

For each of Compounds B-E, % conversion data for ¹³C₅-DGLA to ¹³C₅-AA and % inhibition data was compiled using data from the liver and blood plasma. Table 4.1 and FIG. 17a provide data for the % conversion of ¹³C₅-DGLA to ¹³C₅-AA for each of Compounds B-E as measured using ¹³C₅-DGLA and ¹³C₅-AA from the liver and blood plasma, respectively.

TABLE 4.1

| | Liver | |
|---|---|---|
| | Avg % | Plasma Avg % |
| Group | Conversion | Conversion |
| Vehicle | 83.5 | 60.3 |
| Compound B 10 mg/kg | 2.2 | 2.9 |
| Compound B 30 mg/kg | 1.5 | 2.2 |
| Compound B 50 mg/kg | 1.4 | 1.9 |
| Compound C 3 mg/kg | 14.2 | 10.1 |
| Compound C 10 mg/kg | 4.3 | 4.3 |
| Compound C 30 mg/kg | 1.5 | 1.7 |
| Compound D 30 mg/kg | 34.5 | 25.1 |
| Compound E 30 mg/kg | 78.7 | 58.1 |

Table 4.2 and FIG. 17b provide the % inhibition for each of Compounds B-E as measured using ¹³C₅-DGLA and ¹³C₅-AA collected from the liver and blood plasma, respectively.

TABLE 4.2

| | Liver | Plasma Avg % |
|---|---|---|
| Group | Avg % Inhibition | Inhibition |
| Vehicle | 0.0 | 0.0 |
| Compound B 10 mg/kg | 97.4 | 95.2 |
| Compound B 30 mg/kg | 98.2 | 96.3 |
| Compound B 50 mg/kg | 98.3 | 96.9 |
| Compound C 3 mg/kg | 83.0 | 83.3 |
| Compound C 10 mg/kg | 94.8 | 92.8 |
| Compound C 30 mg/kg | 98.2 | 97.1 |
| Compound D 30 mg/kg | 58.7 | 58.3 |
| Compound E 30 mg/kg | 5.8 | 3.7 |

Comparing FADS1 activity in liver and plasma of DIO mice at 24 hours after oral administration of one single dose of each of Compounds B, C, D, and E, percent inhibition values were found to be highly similar between the two tissues despite differences in the percent conversion detected, suggesting plasma FADS1 activity measurement is reflective of liver FADS1 activity. In other experiments (not shown), target coverage at 1, 2, 4, 6, 8, or 16 hours after dosing with a test compound (or other time points) is measured (in, e.g., a time course study).

The FADS1 unbound in vivo IC50 was calculated for Compounds A, B, and C by measuring ¹³C₅-DGLA and ¹³C₅-AA in the liver of DIO mice for different doses of the respective compound. The calculated IC50 values are shown in Table 4.3. An exemplary dose response curve for Compound A is provided in FIG. 8(c).

TABLE 4.3

| | Free (unbound) |
|---|---|
| Compound | in vivo IC₅₀ [μM] |
| Compound A | 0.002 |
| Compound B | 0.004 |
| Compound C | 0.004 |

Using procedures similar to those described above for Compounds A-E, an additional in vivo FADS1 potency determination was made by measuring ¹³C₅-DGLA and ¹³C₅-AA in blood plasma for various doses of Compound F (shown below). A dose response curve was generated, which is shown in FIG. 17c.

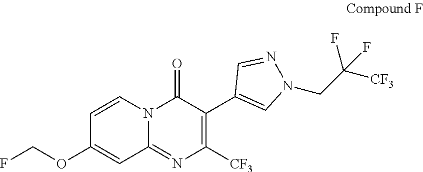 [SMILES: O=c1c(-c2cnn(CC(F)(F)C(F)(F)F)c2)c(C(F)(F)F)nc2cc(OCF)ccn12]

Inhibitory concentration values for Compound F using ¹³C₅-DGLA and ¹³C₅-AA collected from plasma were as follows (Table 4.4):

TABLE 4.4

| Free (unbound) in vivo ICx [μM] | Compound F |
|---|---|
| IC₅₀ | 0.00562 |
| IC₉₀ | 0.0124 |
| IC₉₅ | 0.0164 |

### Example 5: Compound a Treatment Reduced Body Weight and Improved Metabolic Profile in DIO Mice

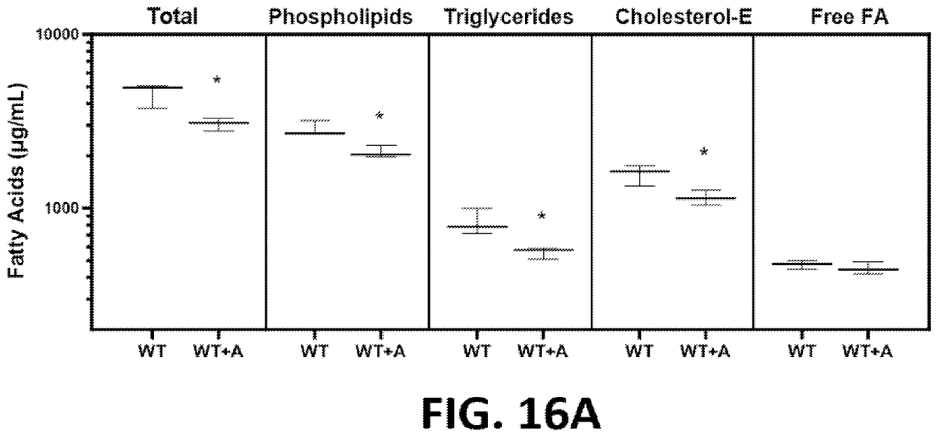

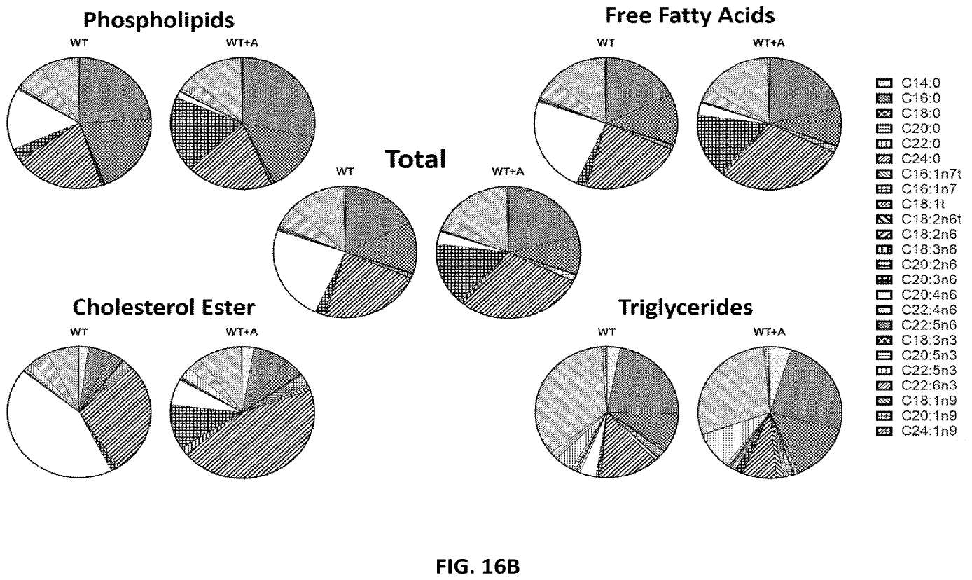

The physiological and metabolic impact of chronic pharmacological inhibition of FADS1 by Compound A was assessed in DIO mice. Chronic FADS1 inhibition by daily oral administration of Compound A at 10 mg/kg and 30 mg/kg for 54 days resulted in 17.9% and 21.1% lower body weight, respectively, relative to the vehicle-treated mice (FIG. 8(d)). FADS1 activity (measured after 54 days of treatment) was inhibited by 90.0% and 93.6% in the 10 mg/kg and 30 mg/kg Compound A treated groups, respectively (FIG. 8(e)). No significant differences in food intake were observed (FIG. 8(f)). In comparison to vehicle-treated mice, plasma insulin levels of mice treated with 10 mg/kg and 30 mg/kg of Compound A were significantly decreased by 74.9% and 84.7%, respectively (FIG. 8(g)). Plasma cholesterol trended lower (FIG. 8(h)) and dose-dependent decreases in plasma triglycerides were observed, with a 41.6% lower level in mice treated with 30 mg/kg of Compound A compared with vehicle-treated mice (FIG. 8(i)). Similar changes in plasma FAs and lipid subfraction composition were observed in the Compound A-treated DIG mice (FIG. 16(a), Tables 5.4, 5.5, 5.6) as seen in the Fads1 KO mice (FIG. 6(a), Tables 5.1, 5.2, 5.3). Interestingly, the ratio of the two products of FADS11 reaction, EPA/AA ratio, a marker that is inversely correlated with inflammation and metabolic diseases, are increased in all four lipid subfractions (Table 5.5); a similar trend was also observed in the Fads1 KG mice (Table 5.2) suggesting differential metabolism of EPA and AA.

TABLE 5.1
Plasma PUFA and metabolic profile of Fads1 KO and
WT male mice after 12 weeks of high fat diet.

| | | Shorthand | | | P-value\hat{ } |
|---|---|---|---|---|---|
| Common name | Abbreviation | notation | Wild-type | Fads1 KO | (KO vs WT) |
| Linoleic acid (μg mL⁻¹) | LA | 18:2 n-6 | 447.0 ± 28.2 | 286.2 ± 33.3 | ** |
| Gamma-Linoleic acid | GLA | 18:3 n-6 | 3.7 ± 0.5 | 6.0 ± 0.5 | *** |
| (μg mL⁻¹) | | | | | |
| Dihomo-gamma- | DGLA | 20:3 n-6 | 51.9 ± 12.8 | 265.6 ± 51.2 | *** |
| linolenic acid (μg mL⁻¹) | | | | | |
| Arachidonic acid (μg mL⁻¹) | AA | 20:4 n-6 | 285.3 ± 36.2 | 71.2 ± 8.1 | *** |
| Adrenic acid (μg mL⁻¹) | AdA | 22:4 n-6 | 7.6 ± 1.3 | 2.2 ± 0.3 | *** |
| Alpha-Linolenic acid | ALA | 18:3 n-3 | 5.1 ± 1.9 | 3.7 ± 1.9 | ns |
| (μg mL⁻¹) | | | | | |
| Eicosatetraenoic acid | ETA | 20:4 n-3 | 1.0 ± 0.2 | 6.7 ± 0.7 | *** |
| (μg mL⁻¹) | | | | | |
| Eicosapentaenoic acid | EPA | 20:5 n-3 | 4.8 ± 0.6 | 0.5 ± 0.1 | *** |
| (μg mL⁻¹) | | | | | |
| Docosahexaenoic acid | DHA | 22:6 n-3 | 145.5 ± 19.0 | 71.9 ± 12.7 | *** |
| (μg mL⁻¹) | | | | | |
| FADS1 activity | AA/DGLA | 20:4n-6/20:3n-6 | 5.65 ± 0.8 | 0.3 ± 0.1 | *** |
| FADS2 activity | GLA/LA | 18:3n-6/18:2n-6 | 0.01 ± 0.00 | 0.02 ± 0.00 | *** |
| Aspartate | AST | | 138.1 ± 31.8 | 115.7 ± 20.2 | ns |
| Aminotransferase, | | | | | |
| (U L⁻¹) | | | | | |
| Alanine | ALT | | 187.4 ± 46.0 | 106.0 ± 42.1 | ** |
| Aminotransferase (U | | | | | |
| L⁻¹) | | | | | |
| Glucose (mg dL⁻¹) | | | 198.7 ± 8.0 | 233.2 ± 25.8 | ** |
| Insulin (ng mL⁻¹) | | | 26.5 ± 12.3 | 6.27 ± 2.4 | *** |
| Total cholesterol (mg | | | 236.9 ± 19.9 | 130.9 ± 21.0 | *** |
| dL⁻¹) | | | | | |
| Triglycerides (mg dL⁻¹) | | | 65.9 ± 8.2 | 72.0 ± 7.3 | ns |
| HDL-Cholesterol (mg | HDL-C | | 81.2 ± 4.4 | 79.4 ± 3.3 | ns |
| dL⁻¹) | | | | | |
| LDL-Cholesterol (mg | LDL-C | | 21.1 ± 3.5 | 12 ± 1.4 | *** |
| dL⁻¹) | | | | | |
| Non-esterified fatty | NEFA | | 0.59 ± 0.06 | 0.73 ± 0.08 | ** |
| acid (mEq L⁻¹) | | | | | |

Polyunsaturated fatty acids (PUFA); wild-type (WT); knockout (KO); Fatty acids measured by LC/MS-MS; n = 8-9/genotype; data are presented as mean ± standard deviation;
\hat{ }unpaired t-test,
*P < 0.05,
**P < 0.01,
***P < 0.001, ns = not significant.

TABLE 5.2
Fatty acid concentration of WT and Fads1 KO plasma lipid subfractions

| Fatty | Total | | Phospholipids | | Triglycerides | | Cholesterol ester | | Free fatty acids | |
|---|---|---|---|---|---|---|---|---|---|---|
| acids | | Fads1 | | Fads1 | | Fads1 | | Fads1 | | Fads1 |
| (μg mL⁻¹) | WT | KO | WT | KO | WT | KO | WT | KO | WT | KO |
| C14:0 | 7.1 | 7.0 | 4.6 | 5.2 | 27.4 | 25.9 | 58.8 | 58.9 | 40.5 | 21.9 |
| C16:0 | 668 | 560* | 605 | 496 | 177 | 127 | 121 | 124 | 258.4 | 142.7* |
| C18:0 | 533 | 318*** | 553 | 336* | 113 | 69.1 | 118 | 112 | 207.2 | 94.6* |
| C20:0 | 7.3 | 8.1 | 4.2 | 4.0 | 2.5 | 1.5 | 1.5 | 1.4 | 3.1 | 1.8 |
| C22:0 | 5.9 | 5.6 | 3.1 | 2.4 | 3.6 | 2.6 | 2.5 | 2.7 | 5.1 | 2.0 |
| C24:0 | 2.2 | 2.3 | 1.8 | 1.0 | 1.5 | 1.5 | 1.4 | 0.8 | 2.2 | 1.0 |
| C16:1n7t | 3.7 | 3.6 | 2.3 | 2.7 | 1.0 | 0.5 | 2.0 | 4.1** | 0.8 | 0.5 |
| C16:1n7 | 27.3 | 29.6 | 4.9 | 5.8 | 4.4 | 4.7 | 18.6 | 16.8 | 4.2 | 7.9* |
| C18:1t | 3.8 | 4.3 | 5.3 | 4.4 | 5.6 | 6.2 | 12.6 | 9.4 | 7.0 | 2.3 |
| C18:2n6t | 2.9 | 5.1* | 4.6 | 3.9 | 11.2 | 10.6 | 18.8 | 13.3 | 3.4 | 1.2 |
| C18:2n6 | 845 | 672** | 431 | 269 | 137 | 38.6 | 430 | 439 | 69.4 | 29.0 |
| C18:3n6 | 11.5 | 21.3*** | 2.1 | 3.9 | 1.0 | 2.7 | 9.3 | 18.2** | 0.5 | 0.8 |
| C20:2n6 | 9.5 | 9.5 | 7.7 | 7.5 | 2.9 | 1.9 | 0.8 | 0.8 | 0.9 | 1.1 |
| C20:3n6 | 96.3 | 596*** | 98.8 | 452* | 4.1 | 20.1*** | 20.8 | 129*** | 1.7 | 10.7*** |
| C20:4n6 | 1121 | 155*** | 425 | 58.7*** | 22.6 | 3.0*** | 748 | 108*** | 7.2 | 2.0 |
| C22:4n6 | 7.9 | 2.6*** | 5.1 | 2.1** | 4.9 | 3.5 | 1.6 | 1.2 | 1.8 | 0.9 |
| C22:5n6 | 7.6 | 8.8 | 5.4 | 5.9 | 2.3 | 1.2 | 1.4 | 1.9 | 0.8 | 0.4 |
| C18:3n3 | 6.2 | 4.9* | 0.8 | 0.6 | 9.1 | 1.5 | 3.0 | 2.2 | 5.6 | 1.3 |
| C20:5n3 | 13.5 | 1.7*** | 4.0 | 3.1 | 4.8 | 2.3* | 8.7 | 4.8 | 7.2 | 5.5 |
| C22:5n3 | 10.7 | 3.3*** | 14.6 | 15.2 | 56.3 | 64.0 | 193 | 174 | 108.7 | 36.7* |
| C22:6n3 | 249 | 105*** | 175 | 71.9** | 12.3 | 2.3** | 70.7 | 34.5*** | 3.6 | 2.6 |
| C18:1n9 | 403 | 362 | 208 | 173 | 190 | 133 | 108 | 107 | 94.6 | 79.8 |
| C20:1n9 | 26.8 | 34.2 | 6.4 | 6.1 | 3.9 | 5.5 | 7.3 | 4.6 | 1.9 | 3.4* |
| C24:1n9 | 4.9 | 5.9 | 2.4 | 1.7 | 2.3 | 2.4 | 3.1 | 2.5 | 1.8 | 1.0 |
| Total | 4074 | 2925*** | 2574 | 1933 | 801 | 531 | 1960 | 1371* | 838 | 451* |
| EPA/AA | 0.012 | 0.011 | 0.009 | 0.044 | 0.211 | 0.814* | 0.012 | 0.045* | 1.67 | 3.23 |

Wild-type (WT); knockout (KO); n = 3 pooled samples per group; each sample pooled from 2 mice after 12 weeks of high fat diet; Data are presented as mean values in μg mL⁻¹; Measured by GC (OmegaQuant, Sioux Falls, SD); Statistics: unpaired t- test relative to WT for each subfraction;
*P < 0.05,
**P < 0.01,
***P < 0.001.

TABLE 5.3
Fatty acid composition of WT and Fads1 KO plasma lipid subfractions

| Fatty | Total | | Phospholipids | | Triglycerides | | Cholesterol ester | | Free fatty acids | |
|---|---|---|---|---|---|---|---|---|---|---|
| acids | | Fads1 | | Fads1 | | Fads1 | | Fads1 | | Fads1 |
| (%) | WT | KO | WT | KO | WT | KO | WT | KO | WT | KO |
| C14:0 | 0.17 | 0.24* | 0.18 | 0.26 | 3.76 | 4.84 | 2.92 | 4.27* | 4.8 | 5.0 |
| C16:0 | 16.4 | 19.1*** | 23.5 | 25.7** | 22.6 | 23.9 | 6.16 | 9.08** | 30.8 | 31.5* |
| C18:0 | 13.1 | 10.9*** | 21.5 | 17.4*** | 14.4 | 13.2 | 5.94 | 8.16* | 24.4 | 21.1* |
| C20:0 | 0.18 | 0.28* | 0.16 | 0.21* | 0.33 | 0.3 | 0.08 | 0.10* | 0.4 | 0.4 |
| C22:0 | 0.14 | 0.19* | 0.12 | 0.13 | 0.47 | 0.49 | 0.13 | 0.2 | 0.6 | 0.4 |
| C24:0 | 0.05 | 0.08** | 0.07 | 0.05 | 0.19 | 0.26 | 0.08 | 0.06 | 0.2 | 0.2 |
| C16:1n7t | 0.09 | 0.12** | 0.09 | 0.14** | 0.14 | 0.09 | 0.11 | 0.30** | 0.1 | 0.1 |
| C16:1n7 | 0.67 | 1.01 | 0.19 | 0.3 | 0.62 | 0.86 | 0.97 | 1.24 | 0.5 | 1.8* |
| C18:1t | 0.09 | 0.15* | 0.2 | 0.23 | 0.72 | 1.19 | 0.64 | 0.68 | 0.8 | 0.6 |
| C18:2n6t | 0.07 | 0.17** | 0.18 | 0.2 | 1.58 | 2.01 | 0.91 | 0.97 | 0.4 | 0.3 |
| C18:2n6 | 20.7 | 23.0* | 16.7 | 14.0** | 14.3 | 7.21 | 22.3 | 32.2* | 8.2 | 6.1 |
| C18:3n6 | 0.28 | 0.73*** | 0.08 | 0.2** | 0.15 | 0.49* | 0.48 | 1.33*** | 0.1 | 0.2 |
| C20:2n6 | 0.23 | 0.32*** | 0.30 | 0.39* | 0.41 | 0.36 | 0.04 | 0.06 | 0.1 | 0.2 |
| C20:3n6 | 2.36 | 20.4*** | 3.76 | 23.3*** | 0.6 | 3.84** | 1.07 | 9.44*** | 0.2 | 2.2* |
| C20:4n6 | 27.5 | 5.32*** | 16.6 | 3.05*** | 3.09 | 0.58** | 38.6 | 7.87*** | 1.0 | 0.4 |
| C22:4n6 | 0.19 | 0.09 | 0.2 | 0.12* | 0.65 | 0.67 | 0.08 | 0.09 | 0.2 | 0.2 |
| C22:5n6 | 0.19 | 0.3 | 0.21 | 0.32* | 0.32 | 0.21 | 0.07 | 0.14 | 0.1 | 0.1 |
| C18:3n3 | 0.15 | 0.17 | 0.03 | 0.03 | 0.83 | 0.28 | 0.16 | 0.16 | 0.7 | 0.3 |
| C20:5n3 | 0.33 | 0.06 | 0.16 | 0.14 | 0.64 | 0.43 | 0.46 | 0.35 | 0.9 | 1.3 |
| C22:5n3 | 0.26 | 0.11 | 0.57 | 0.76 | 7.74 | 12.1* | 9.21 | 12.5 | 13.0 | 8.4 |
| C22:6n3 | 6.11 | 3.58 | 6.78 | 3.72*** | 1.64 | 0.42** | 3.64 | 2.53** | 0.5 | 0.6 |
| C18:1n9 | 9.91 | 12.4** | 8.1* | 8.86 | 24.0 | 24.9 | 5.56 | 7.81* | 11.7 | 17.5 |
| C20:1n9 | 0.66 | 1.17* | 0.25 | 0.32 | 0.49 | 1.03 | 0.32 | 0.32 | 0.2 | 0.8** |
| C24:1n9 | 0.12 | 0.2 | 0.09 | 0.09 | 0.35 | 0.44 | 0.16 | 0.19 | 0.2 | 0.2 |
| EPA/AA | 0.012 | 0.011 | 0.010 | 0.044 | 0.211 | 0.814 | 0.012 | 0.045* | 1.67 | 3.23 |

Wild-type (WT); knockout (KO); n = 3 pooled samples per group: each sample pooled from 2 mice after 12 weeks of high fat diet; Data are presented as mean % composition of each lipid subfraction; Measured by GC (OmegaQuant, Sioux Falls, SD); Statistics: unpaired t- test relative to WT for each fraction;
*P < 0.05,
**P < 0.01,
***P < 0.001

TABLE 5.4
Plasma PUFA and metabolic profile of Compound A-treated WT DIO mice

| | Shorthand | | 10 mg kg⁻¹ | 30 mg kg⁻¹ |
|---|---|---|---|---|
| Common name | notation | Vehicle | Compound A | Compound A |
| Linoleic acid (μg | 18:2 n-6 | 296.5 ± 25.5 | 198.5 ± 10.5*** | 196.5 ± 24.6*** |
| mL⁻¹) | | | | |
| Gamma-Linoleic | 18:3 n-6 | 2.2 ± 0.6 | 3.4 ± 0.4*** | 2.8 ± 0.5* |
| acid (μg mL⁻¹) | | | | |
| Dihomo-gamma- | 20:3 n-6 | 58.6 ± 9.0 | 281.3 ± 34.6*** | 242.3 ± 23.4*** |
| Linolenic acid | | | | |
| (μg mL⁻¹) | | | | |
| Arachidonic acid | 20:4 n-6 | 135.5 ± 10.6 | 16.1 ± 3.2*** | 12.5 ± 3.0*** |
| (μg ml⁻¹) | | | | |
| Adrenic acid | 22:4 n-6 | 5.5 ± 0.5 | 1.0 ± 0.1*** | 0.8 ± 0.1*** |
| (μg mL⁻¹) | | | | |
| Alpha-Linolenic | 18:3 n-3 | 1.8 ± 0.6 | 0.9 ± 0.3*** | 0.7 ± 0.2*** |
| acid (μg mL⁻¹) | | | | |
| Eicosatetraenoic | 20:4 n-3 | 0.6 ± 0.1 | 2.5 ± 0.5*** | 1.9 ± 0.4*** |
| acid (μg mL⁻¹) | | | | |
| Eicosapentaenoic | 20:5 n-3 | 3.9 ± 0.6 | 0.3 ± 0.1*** | 0.3 ± 0.1*** |
| acid (μg mL⁻¹) | | | | |
| Docosahexaenoic | 22:6 n-3 | 172.5 ± 19.5 | 93.5 ± 11.8*** | 78.2 ± 11.6*** |
| acid (μg mL⁻¹) | | | | |
| FADS1 activity | 20:4n-6/20:3n-6 | 2.35 ± 0.3 | 0.06 ± 0.01*** | 0.05 ± 0.01*** |
| FADS2 activity | 18:3n-6/18:2n-6 | 0.008 ± 0.001 | 0.018 ± 0.002*** | 0.014 ± 0.003*** |
| Alanine | | 178.0 ± 45.6 | 142.8 ± 26.6 | 102.0 ± 32.4* |
| Aminotransferase | | | | |
| (U L⁻¹) | | | | |
| Glucose (mg dL⁻¹) | | 212.2 ± 20.5 | 170.3 ± 22.3*** | 168.6 ± 23.2*** |
| Insulin (ng mL⁻¹) | | 14.6 ± 8.9 | 3.7 ± 3.2*** | 2.2 ± 1.3*** |
| Cholesterol (mg | | 244.8 ± 29.8 | 173.0 ± 11.9** | 165.3 ± 15.6** |
| dL⁻¹) | | | | |
| Triglycerides (mg | | 106.8 ± 14.4 | 77.8 ± 13.7* | 63.3 ± 9.7** |
| dL⁻¹) | | | | |
| HDL-Cholesterol | | 85.0 ± 8.0 | 86.4 ± 8.0 | 85.7 ± 5.5 |
| (mg dL⁻¹) | | | | |
| LDL-Cholesterol | | 29.8 ± 4.3 | 22.4 ± 2.3* | 19.7 ± 2.5** |
| (mg dL⁻¹) | | | | |
| Non-esterified fatty | | 1.1 ± 0.07 | 0.9 ± 0.1** | 0.7 ± 0.1*** |
| acids (mEq L⁻¹) | | | | |
| Serum Amyloid A | | 23.0 ± 9.9 | 5.2 ± 1.3*** | 4.8 ± 1.8*** |
| (μg mL⁻¹) | | | | |
| C-reactive Protein | | 47.5 ± 11.1 | 43.6 ± 9.7 | 37.1 ± 6.5* |
| (μg mL⁻¹) | | | | |

Polyunsaturated fatty acids (PUFA); wild-type (WT); diet induced obese (DIO); n = 3-10/ group, after 46 or 54 days of treatment; Data are presented as mean ± SD; fatty acids measured by LC-MS/MS; Statistics: one-way ANOVA with Dunnett's multiple comparison test relative to vehicle control;
*P < 0.05,
**P < 0.01,
***P < 0.001.

TABLE 5.5
Fatty acid concentration of Compound A-treated DIO mouse plasma lipid subfractions

Fatty

| acids | Total | | Phospholipids | | Triglycerides | | Cholesterol ester | | Free fatty acids | |
|---|---|---|---|---|---|---|---|---|---|---|
| (μg mL⁻¹) | WT | WT + A | WT | WT + A | WT | WT + A | WT | WT + A | WT | WT + A |
| C14:0 | 9.5 | 7.7 | 4.3 | 5.3 | 24.8 | 26.7 | 33.2 | 29.9 | 17.2 | 18.8 |
| C16:0 | 807 | 635 | 682 | 591 | 187 | 135 | 83.8 | 90.5 | 148.5 | 132.7 |
| C18:0 | 568 | 285** | 573 | 302*** | 74.6 | 80.4 | 45.4 | 50.3 | 98.6 | 76.8 |
| C20:0 | 5.1 | 2.3** | 3.5 | 1.4** | 1.8 | 1.6 | 0.4 | 1.2 | 1.5 | 1.8 |
| C22:0 | 5.8 | 3.3** | 2.4 | 1.8 | 2.9 | 3.5 | 2.2 | 1.3 | 1.8 | 1.4 |
| C24:0 | 2.3 | 1.9 | 1.2 | 1.0 | 1.2 | 1.2 | 0.7 | 1.2 | 1.2 | 1.0 |
| C16:1n7t | 3.8 | 3.0 | 3.0 | 1.2 | 0.9 | 0.7 | 1.9 | 2.6 | 0.2 | 0.5 |
| C16:1n7 | 39.4 | 41.4 | 6.5 | 7.7 | 12.0 | 6.2 | 22.6 | 27.7 | 4.9 | 8.7 |
| C18:1t | 6.2 | 3.6 | 4.7 | 3.9 | 3.8 | 6.1 | 6.2 | 8.1 | 3.0 | 2.5 |
| C18:2n6t | 4.0 | 6.0 | 3.6 | 4.5 | 8.7 | 9.0 | 6.9 | 10.4* | 1.9 | 3.3 |
| C18:2n6 | 1038 | 870 | 584 | 400** | 108 | 42.6** | 434 | 516 | 45.9 | 38.5 |
| C18:3n6 | 13.7 | 23.3** | 3.8 | 4.3 | 2.2 | 0.7 | 9.4 | 20.4* | 0.4 | 1.2 |
| C20:2n6 | 12.9 | 9.9 | 9.8 | 9.0 | 3.3 | 1.7 | 1.4 | 1.4 | 1.3 | 1.1 |
| C20:3n6 | 88.5 | 465*** | 74.2 | 382*** | 4.7 | 8.3 | 18.0 | 124*** | 2.6 | 8.1** |
| C20:4n6 | 1081 | 92.3** | 432 | 31.2*** | 30.4 | 2.5*** | 681 | 72.4*** | 17.1 | 2.9*** |
| C22:4n6 | 9.9 | 2.1*** | 5.1 | 2.1*** | 4.9 | 2.5* | 1.9 | 1.5 | 1.6 | 1.1* |
| C22:5n6 | 10.2 | 2.4*** | 6.2 | 1.8*** | 1.7 | 2.5 | 1.8 | 1.7 | 0.6 | 0.4 |
| C18:3n3 | 9.2 | 5.9** | 1.6 | 1.0 | 4.1 | 1.8 | 3.1 | 3.0 | 2.4 | 1.6 |
| C20:5n3 | 14.1 | 2.7** | 4.3 | 1.2* | 6.5 | 3.7** | 7.5 | 2.8* | 3.0 | 8.3 |
| C22:5n3 | 11.0 | 2.8*** | 20.5 | 12.8 | 33.7 | 49.9* | 30.5 | 31.0 | 23.2 | 32.0 |
| C22:6n3 | 247 | 98.5** | 174.5 | 70.2** | 16.5 | 0.6** | 64.9 | 37.4* | 7.4 | 5.3 |
| C18:1n9 | 585 | 478 | 249 | 260 | 286 | 161* | 111 | 113 | 85.6 | 98.1* |
| C20:1n9 | 11.6 | 8.4 | 6.2 | 5.8 | 7.8 | 5.6* | 0.7 | 0.6 | 2.2 | 2.8 |
| C24:1n9 | 6.2 | 6.9 | 2.5 | 3.3 | 2.9 | 2.3 | 2.3 | 2.6 | 1.2 | 1.8 |
| Total | 4589 | 3058* | 2858 | 2105* | 831 | 556* | 1570 | 1150* | 473 | 451 |
| EPA/AA | 0.013 | 0.030** | 0.010 | 0.038* | 0.22 | 1.51** | 0.011 | 0.039* | 0.179 | 2.94* |

Wild-type (WT); diet-induced obese (DIO); WT vehicle treated (WT); 30 mg/kg treated WT (WT + A); n = 3 pooled samples per group; each sample pooled from 2 WT DIO mice; Data are presented as mean values in μg mL⁻¹; Measured by GC (OmegaQuant, Sioux Falls, SD); Statistics: Unpaired t- test relative to WT, vehicle treated group for each subfraction;
*P < 0.05,
**P < 0.01,
***P < 0.001.

TABLE 5.6
Fatty acid composition of Compound A-treated DIO mouse plasma lipid subfractions

| Fatty | Total | | Phospholipids | | Triglycerides | | Cholesterol ester | | Free fatty acids | |
|---|---|---|---|---|---|---|---|---|---|---|
| acids (%) | WT | WT + A | WT | WT + A | WT | WT + A | WT | WT + A | WT | WT + A |
| C14:0 | 0.21 | 0.25 | 0.15 | 0.25* | 3.01 | 4.77* | 2.12 | 2.61* | 3.64 | 4.15 |
| C16:0 | 17.6 | 20.8 | 23.8 | 28.1** | 22.5 | 24.2 | 5.36 | 7.89*** | 31.3 | 29.5 |
| C18:0 | 12.4 | 9.3 | 20.1 | 14.4*** | 9.0 | 14.5 | 2.91 | 4.41* | 20.7 | 17.1* |
| C20:0 | 0.11 | 0.08 | 0.12 | 0.07* | 0.22 | 0.28 | 0.03 | 0.1 | 0.32 | 0.39 |
| C22:0 | 0.13 | 0.11 | 0.08 | 0.08 | 0.35 | 0.63 | 0.14 | 0.12 | 0.37 | 0.31 |
| C24:0 | 0.05 | 0.06 | 0.04 | 0.04 | 0.15 | 0.23 | 0.04 | 0.11 | 0.26 | 0.22 |
| C16:1n7t | 0.08 | 0.1 | 0.11 | 0.05 | 0.11 | 0.12 | 0.12 | 0.23** | 0.03 | 0.11 |
| C16:1n7 | 0.86 | 1.35 | 0.23 | 0.36** | 1.44 | 1.10 | 1.44 | 2.39* | 1.05 | 1.91 |
| C18:1t | 0.13 | 0.12 | 0.17 | 0.18 | 0.47 | 1.09 | 0.39 | 0.7*** | 0.65 | 0.57 |
| C18:2n6t | 0.09 | 0.2 | 0.13 | 0.22** | 1.06 | 1.64* | 0.44 | 0.9*** | 0.39 | 0.76 |
| C18:2n6 | 22.7 | 28.5 | 20.5 | 19.0 | 13.0 | 7.68** | 27.7 | 44.7*** | 9.76 | 8.61 |
| C18:3n6 | 0.3 | 0.76 | 0.13 | 0.2* | 0.26 | 0.13 | 0.6 | 1.76*** | 0.09 | 0.26* |
| C20:2n6 | 0.28 | 0.32 | 0.34 | 0.43* | 0.39 | 0.31 | 0.09 | 0.12 | 0.27 | 0.25 |
| C20:3n6 | 1.93 | 15.2 | 2.59 | 18.2*** | 0.55 | 1.5 | 1.14 | 10.8*** | 0.56 | 1.82 |
| C20:4n6 | 23.5 | 3.01 | 15.12 | 1.49*** | 3.71 | 0.46*** | 43.2 | 6.34*** | 3.60 | 0.64*** |
| C22:4n6 | 0.22 | 0.07*** | 0.18 | 0.10*** | 0.59 | 0.45 | 0.12 | 0.13 | 0.34 | 0.25* |
| C22:5n6 | 0.22 | 0.08*** | 0.22 | 0.09** | 0.2 | 0.45* | 0.11 | 0.14 | 0.13 | 0.10 |
| C18:3n3 | 0.2 | 0.19 | 0.06 | 0.05 | 0.48 | 0.33 | 0.2 | 0.27 | 0.51 | 0.34 |
| C20:5n3 | 0.31 | 0.09*** | 0.15 | 0.06* | 0.8 | 0.67 | 0.48 | 0.25 | 0.63 | 1.75 |
| C22:5n3 | 0.24 | 0.09*** | 0.7 | 0.61 | 4.12 | 8.98*** | 1.95 | 2.71* | 4.90 | 7.14 |
| C22:6n3 | 5.37 | 3.22 | 6.09 | 3.33*** | 1.97 | 0.12*** | 4.12 | 3.26** | 1.58 | 1.15 |
| C18:1n9 | 12.7 | 15.7 | 8.69 | 12.3** | 34.3 | 29.0 | 7.09 | 9.73* | 18.2 | 21.8 |
| C20:1n9 | 0.25 | 0.27 | 0.22 | 0.28 | 0.95 | 1.01 | 0.04 | 0.05 | 0.46 | 0.61 |
| C24:1n9 | 0.13 | 0.23** | 0.09 | 0.16 | 0.37 | 0.41 | 0.15 | 0.23 | 0.25 | 0.39 |
| EPA/AA | 0.013 | 0.030** | 0.010 | 0.038 | 0.216 | 1.51** | 0.011 | 0.039* | 0.18 | 2.94* |

Wild-type (WT); diet-induced obese (DIO); WT vehicle treated (WT); 30 mg/kg treated WT (WT + A); n = 3 pooled samples per group; each sample pooled from 2 WT DIO mice; Data are presented as mean % composition of each lipid subfraction; Measured by GC (OmegaQuant, Sioux Falls, SD); Statistics: Unpaired t- test relative to WT, vehicle treated group for each subfraction;
*P < 0.05,
**P < 0.01,
***P < 0.001.

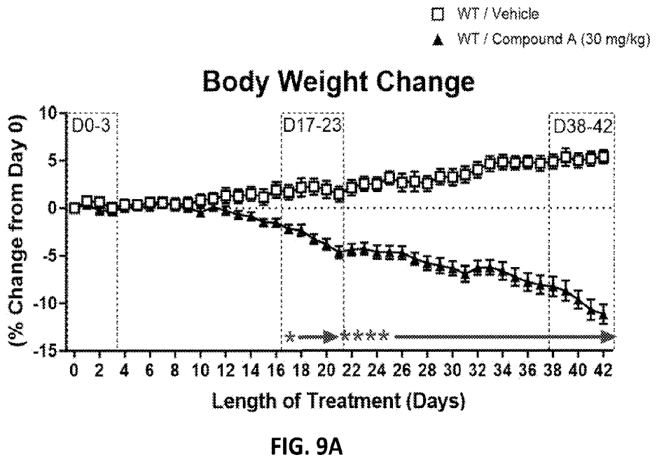

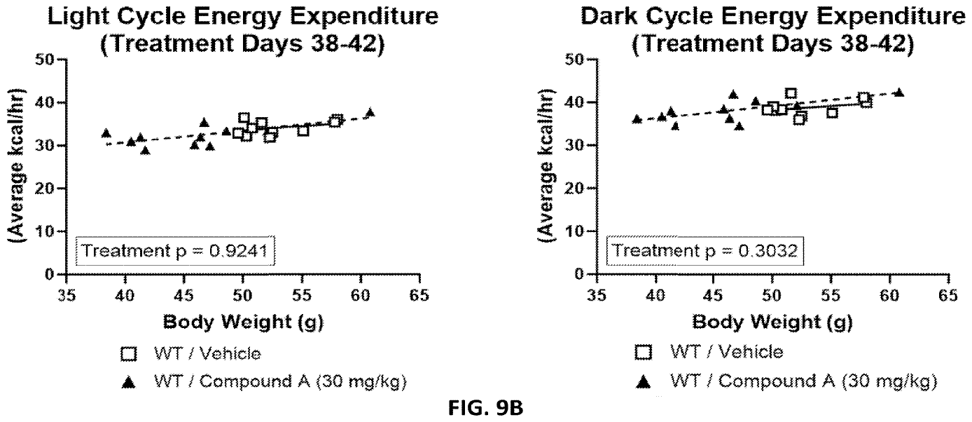

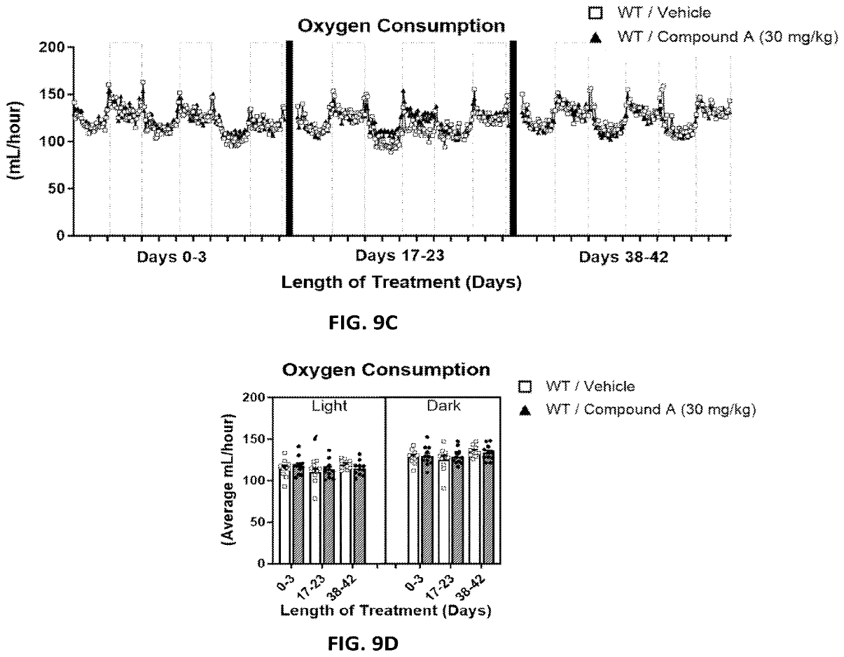

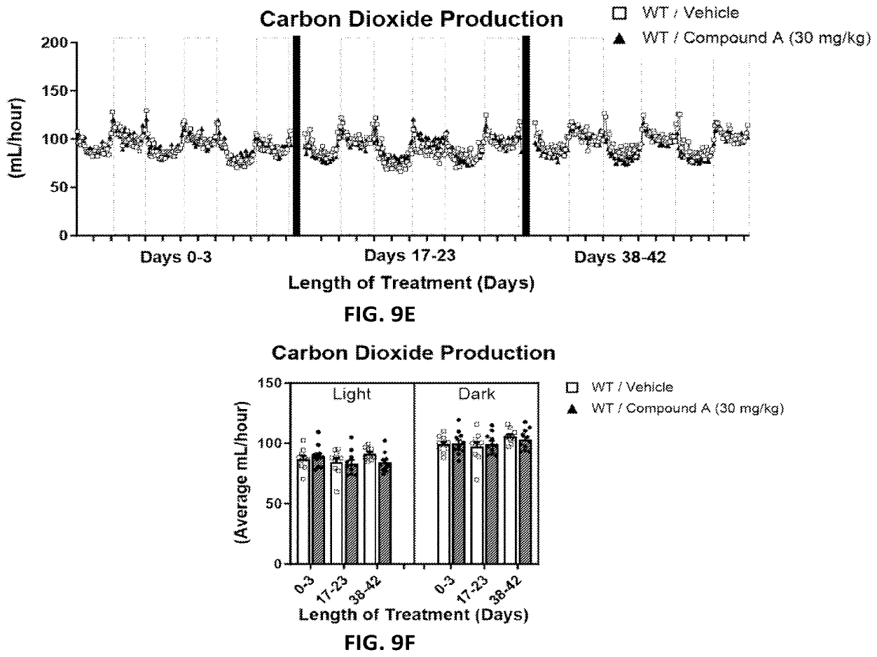

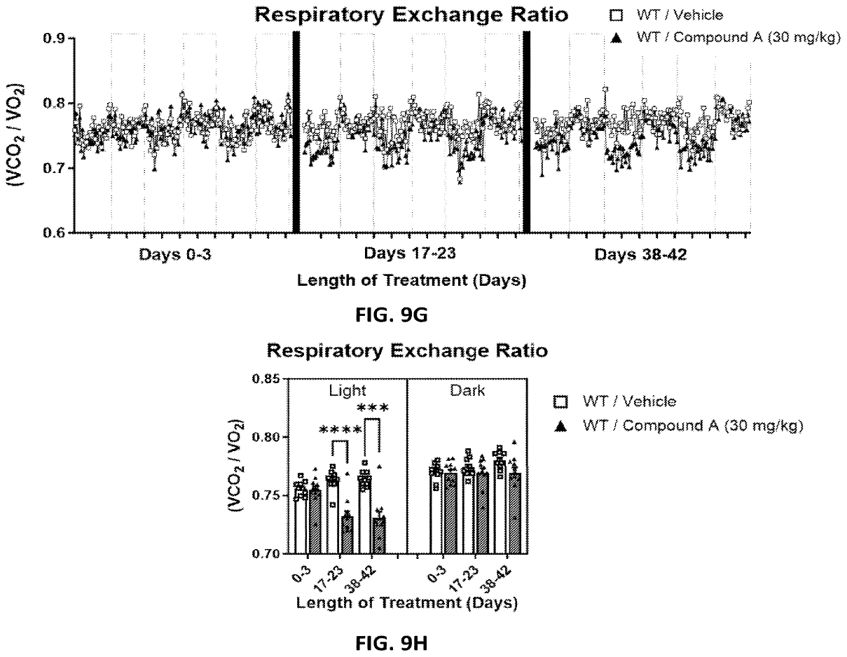

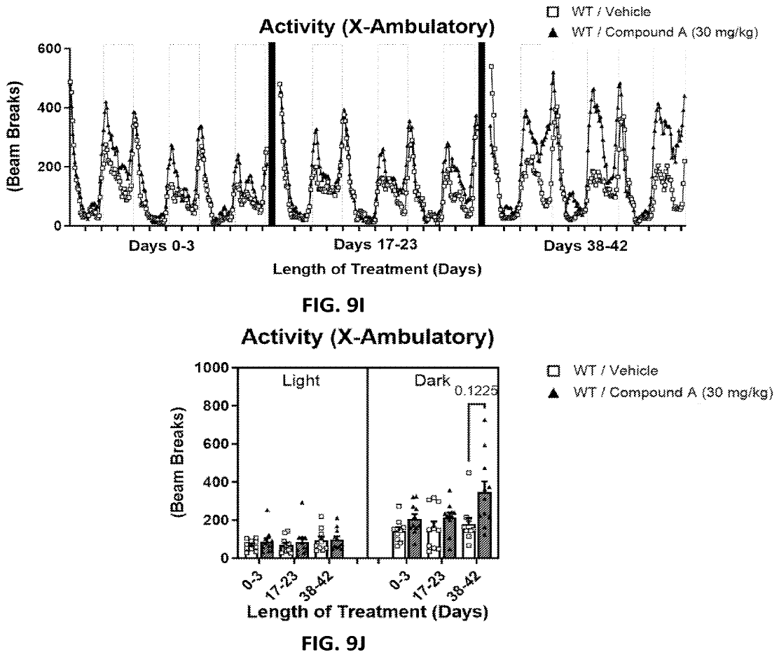

Indirect calorimetry measurements in DIO mice treated with either vehicle or 30 mg/kg of Compound A for 42 days again significantly reduced body weight as expected (FIG. 9(a)); however, no differences in energy expenditure, oxygen consumption and carbon dioxide production were observed (FIG. 9(b)-FIG. 9(f)). Nevertheless, Compound A-treated mice had decreased light cycle RER at both measured periods: 17-23 days and 38-42 days (FIG. 9(g), FIG. 9(h)), indicating increased resting lipid oxidation. Additionally, Compound A-treated mice trended to be more active in the dark cycle (FIG. 9(i)-FIG. 9(j)) than vehicle-treated mice.

### Example 6: RNA-Seg Analysis with Compound a Treatment in DIO Mice

Next, RNA-seq analysis was performed to compare the transcriptome changes induced by either genetic deletion or pharmacological inhibition of FADS1. Comparison of transcriptome changes in DIO mice treated with 30 mg/kg of Compound A (WT+A) for 54 days and age-matched vehicle-treated DIO Fads1 KO relative to vehicle-treated WT mice revealed similarities and differences in the pathways affected (FIG. 10). FIG. 10(a) provides radar charts of commonly altered genes in liver, EPI WAT and ING WAT of Fads1 KO mice in Compound A-treated mice relative to WT vehicle-treated mice. Genetic and pharmacological inhibition of FADS1 activity by Compound A led to a greater number of transcriptome changes in EPI WAT in comparison to WT. The number of DEGs was modest in the liver and minimal in ING WAT (FIG. 13(g)). In addition, inhibition of FADS1 activity genetically or pharmacologically led to transcriptome changes of 122 common genes in the liver, 872 in EPI WAT, and 40 in ING WAT (FIG. 13(g)). Cellular compositions were moderately affected in the liver and ING WAT of Compound A-treated mice relative to WT mice. The greatest change observed in cellular composition of EPI WAT after treatment with Compound A was in the immune cell population, with decreases in T cells, B cells, and natural killer cells.

Ingenuity Pathway Analysis (IPA) of transcriptome changes achieved with genetical or pharmacological inhibition of Compound A of FADS1 in DIO mice revealed some common pathways in EPI WAT: upregulation of the mTOR Signaling pathway and downregulation of 23 pathways (FIG. 10(c)). Less pathways were affected in the liver, and commonly affected pathways shared by Fads1 KO and Compound A-treated mice included upregulation of Cholesterol Biosynthesis and Nicotine Degradation pathways and decrease in CREB Signaling in Neurons and eNOS Signaling pathways (FIG. 10(d)). The unique pathways affected by genetic deletion of Fads1 included Methionine Degradation in EPI WAT (FIG. 10(c)) and three signaling pathways in the liver (GP6 Signaling, Xenobiotic Metabolism CAR Signaling, and PXR signaling) (FIG. 10(d)). In EPI WAT, the inhibition of FADS1 by Compound A increased Insulin Receptor Signaling (FIG. 10(c)). In the liver of Compound A-treated WT mice (FIG. 10(d)), upregulation of Calcium Signaling, PPARα/RXRα Activation, and Xenobiotic metabolism AHR Signaling pathways and downregulation of Acute Phase Response. AMPK Signaling, and Senescence pathways were observed. Disease and Biofunction analysis of the common DEGs of Fads1 KO mice and Compound A-treated mice revealed common and differential biofunctions affected in the liver, EPI WAT, and ING WAT, with Diabetes mellitus, Diabetic nephropathy, and Inflammation of Absolute Anatomical Region commonly altered in all three tissues.

Gene expression profiling of the liver, EPI WAT, and ING WAT of Compound A-treated mice showed similar trends in gene expression as Fads1 KO mice: increased fatty acid oxidation-related genes (Cpt1a, Acox1, Acaa1a, Fgf21) in the liver; increased lipolysis (Lipe, Mg11, Pnpla2) and insulin-signaling genes (Insr. Irs1, Slc2a4) in EPI WAT; and decreased inflammation-related genes (Cc12, Cd14, Cd68) and upregulation of insulin-signaling genes (Insr, Irs1) in ING WAT (FIG. 13(a)-(c)). Upstream analysis by IPA showed PPARA, PPARG, RXRA, and PPPARGC1A as the top predicted upstream regulators that were activated in the liver (FIG. 13(d)). Many inflammation-related upstream regulators, including IFNG, TCF3, and TNF, were predicted to be inhibited in the EPI WAT of Fads1 KO and Compound A-treated mice (FIG. 13(e)).

Compound A treatment decreased leptin levels in the plasma and in both EPI and ING WAT (FIG. 13(f)). This differs from Fads1 KO mice, which have slightly higher leptin levels than WT in EPI WAT (FIG. 13(f)). As observed in Fads1 KO mice, adiponectin levels were elevated in the EPI WAT of Compound A-treated WT mice. WT mice treated with Compound A show trends towards lower PAI-1 and MCP-1 levels in EPI and ING WAT (FIG. 13(d)-(f)), agreeing with the decreased expression of inflammation-related genes in the respective tissue (FIG. 10, FIG. 13(c)).

### Discussion

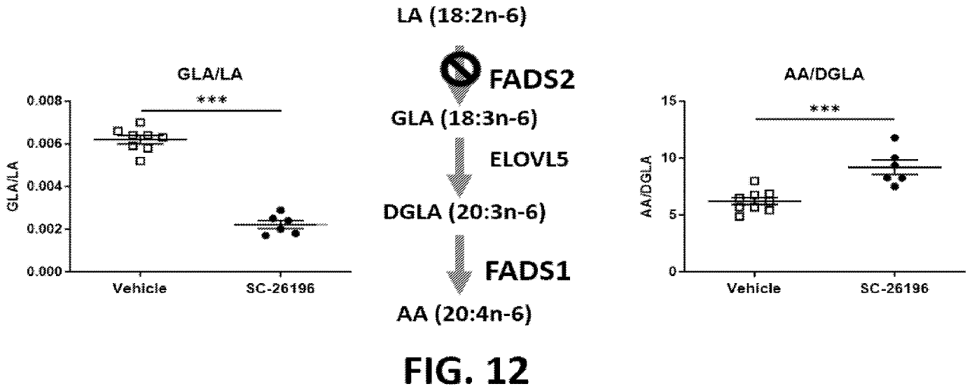

Obesity is associated with chronic low-grade inflammation and FADS1 synthesizes AA, a precursor of many pro-inflammatory eicosanoids. As demonstrated in the Examples herein, it was found that FADS1 activity, as indicated by the plasma AA/DGLA ratio, was elevated in obese male humans and mice, suggesting that FADS1 activity is elevated in the obese state (FIG. 1(b)-(c)). Similarly, increased FADS1 (or D5D) activity is found in HFD-fed E3L mice. However, the data provided herein conflict with other human studies that have reported either a lack of association or a negative correlation of FADS1 activity with obesity or insulin resistance. The discrepancy in FADS1 activity found between the different reports likely reflects differences in the population studied (i.e., the disease states), the type of diets the individuals were ingesting, and other confounders. These differences may influence PUFA levels and fatty acid desaturase expression and activity. For instance, a negative correlation for FADS1 activity and BMI was often detected when a positive correlation for FADS2 activity and BMI was found. Since FADS2 is upstream of FADS1 and the rate-limiting enzyme of the pathway, inhibition of FADS2 activity with SC-26196, a FADS2 specific inhibitor, increased downstream AA/DGLA ratio (FIG. 11(c)) resulting in a false appearance of increased FADS1 activity. FIG. 12 shows that inhibition of FADS2 activity by SC-26196 affects not only the plasma GLA/LA ratio but also the plasma AA/DGLA ratio in DIO mice. Increased FADS2 activity may have the reverse effect. Therefore, it is hypothesized that in human population studies, true effect of FADS1 may be confounded by changes in FADS2 activity: the AA/DGLA ratio may be lower in obese subjects with elevated FADS2 activity resulting in an apparent negative correlation of FADS1 with obesity. Importantly, the FADS locus is associated with body weight, type 2 diabetes, dyslipidemia, and other cardiometabolic traits from GWAS, implicating FADS1 involvement in body weight and metabolic diseases. Minor alleles of FADS1 SNP rs174556 and rs7115739 that are linked with reduced FADS1 activity may be associated with lower body weight. Additionally, the carriers of the minor C allele of FADS1 SNP rs174547, which may be associated with reduced FADS1 activity, may have lower odds of developing metabolic syndrome and larger waist circumference relative to the carriers of TT genotype. Together, these support the concept that FADS1 activity and obesity are positively correlated.

Here, abrogation of FADS1 activity either by genetic deletion or by pharmacological inhibition reduced body weight and metabolic profiles with minimal impact on food intake. By using the untargeted transcriptomics and metabolomics approach, that data provided herein shed light on the possible mechanisms of action such as decreased inflammation and increased lipid oxidation by PPARα activation.

Improvement in metabolic parameters by inhibition of FADS1 activity by genetic deletion or pharmacological inhibition are likely in part mediated by dampening inflammation via limiting the availability of AA (Tables 3.1, 6.1) and AA-derived pro-inflammatory eicosanoids, particularly COX-derived eicosanoids (FIG. 7(a)). COX has been implicated in obesity-associated metabolic syndrome. Administration of indomethacin, a general COX inhibitor, was shown to reduce body weight and improve insulin sensitivity in HFD-fed DIO C57BL/6J mice. Therefore, lowering of COX-derived eicosanoids may contribute in part to the anti-obesogenic effect of FADS1 inhibition.

The reduction of inflammation is particularly evident in the adipose tissue of the Fads1 KO and Compound A-treated mice. Analysis of EPI and ING WAT showed downregulation of inflammatory pathways and biofunctions including monocyte/macrophage markers (Cd14 and Cd68) (FIGS. 4(d), 5(a), 7(c), 10(c), 13(c)-(d)), alteration of immune cell population (FIGS. 4(b), 10(b)), and decreased PAI-1 and MCP-1 protein levels (FIGS. 5(b), 13(d)) suggesting decreased adipose macrophage infiltration and inflammation with FADS1 inhibition. This result is consistent with previous report of downregulation of inflammatory genes in the adipose of FADS1-inhibited mice. Obesity induces a drastic increase in macrophages in adipose tissue in both humans and mice, which results in increased inflammatory cytokines that alter insulin signaling and contribute to insulin resistance in obesity: hence, reducing adipose macrophage infiltration and inflammation by FADS1 inhibition may ameliorate insulin resistance in DIO mice. Improved insulin sensitivity is suggested in Fads1 KO and Compound A-treated mice as evidenced by improved glucose tolerance, lower glucose, and insulin levels (FIGS. 2(e)-(f), 7, 8(g)), and upregulation of insulin-signaling genes (Insr, Irs1, Slc2a4) in EPI and ING WAT (FIGS. 4(d), 10(c), 5(a)). Corroborating data have also been reported in humans; carriers of the minor allele genotype (CC) for FADS1 SNP rs174550 are suggested to be protected against LA-induced adipose tissue inflammation and have improved insulin sensitivity with lower FADS1 activity. Together, these data suggest that FADS1 inhibition protects from obesity-induced adipose tissue inflammation and improves insulin sensitivity.

The reduction of inflammation is particularly evident in the adipose tissue of the FADS1-inhibited mice. Transcriptomic profiling of EPI and ING WAT of Fads1 KO and Compound A-treated mice showed downregulation of inflammatory pathways (FIGS. 4(d), 10(c), 5(a), 13(b)-(c)) and alteration of immune cell populations (FIGS. 4(b), 8(b)). Decreased PAI-1 and MCP-1 protein levels and monocyte/macrophage markers (Cd14 and Cd68) in the adipose tissue of Fads1 KO and Compound A-treated mice suggest decreased macrophage infiltration and inflammation with FADS1 inhibition (FIGS. 5(a)-(b), 13(b)-(c), 13(f)). This implies that FADS1 inhibition protects from obesity-induced adipose tissue inflammation.

Obesity induces a drastic increase in macrophages present in adipose tissue in both humans and mice, and an excess in the number of macrophages increase secreted inflammatory cytokines that alter insulin signaling and contribute to insulin resistance in obesity. Hence, reducing macrophage infiltration and subsequent inflammation by FADS1 inhibition may ameliorate insulin resistance in DIO mice. Improved insulin sensitivity is suggested in Fads1 KO and Compound A treated mice as evidenced by the improved glucose tolerance and lower glucose and insulin levels (FIGS. 2(e)-(f), 7, 8(g)) and upregulation of insulin-signaling genes (Insr, Irs1, Slc2a4) in the EPI and ING WAT (FIGS. 4(d), 14(a), 15(b)-(c)). Decreased inflammation and improved insulin sensitivity by FADS1 inhibition may help alleviate the dysregulation of energy metabolism in the obese state.

Body weight reduction associated with abrogation of FADS1 activity as shown herein was not attributed to a decrease in food intake, but rather to a shift towards increased lipid oxidation as suggested by the decrease in RER by both genetic and pharmacological FADS1 inhibition (FIGS. 3(e)-(f), 9(g)-(h)). In addition to decreased RER, other signatures of increased lipid oxidation were also observed, including increased levels of citrate cycle intermediates in the plasma of Fads1 KG mice (FIG. 7). Transcriptomic analysis suggested activation of the PPARα pathway as the mediator of the increased lipid oxidation; increased hepatic PPAR Signaling pathway/PPARα/RXRα Activation (FIGS. 4(c), 10(d)) and increased hepatic expression of fatty oxidation genes (Cpt1a, Acox1, Fgf21) were observed when FADS1 activity was abrogated either by genetic deletion or by pharmacological inhibition (FIGS. 5(a), 13(a)). In particular, upregulation of hepatic expression of Cyp4a12 genes (FIGS. 5(a), 13(a)) strongly suggests activation of PPARα since studies using Ppara KG mice demonstrated that hepatic expression of Cyp4a2 genes is dependent on PPARα, and Cyp4a expression is extremely sensitive to ligand activation and serves as a marker for PPAR, activation. Although the mechanism of PPARα activation is unclear, alterations of PUFA levels and downstream metabolites from FADS1 inhibition may be involved. For instance, a high AA/DHA ratio decreases mitochondrial function and decreases PPARα expression, resulting in decreased fatty acid oxidation in hepatocytes. The decreased AA/DHA ratios (by two-fold and five-fold in the Fads1 KG mice and Compound A-treated mice, respectively) along with FADS1 inhibition may contribute to increased fatty acid oxidation (Tables 3.1, 6.1).

TABLE 6.1
PUFA profile in plasma of Compound A-treated mice

| | | | | 10 mg/kg | 30 mg/kg |
|---|---|---|---|---|---|
| | | Shorthand | | Compound A, | Compound A, |
| Common Name | Abbreviation | Notation | Vehicle, μg/ml | μg/ml | μg/ml |
| Linoleic acid | LA | 18:2 n-6 | 296.5 ± 25.5 | 198.5 ± 10.5 | 196.5 ± 24.6 |
| γ-Linoleic acid | GLA | 18:3 n-6 | 2.2 ± 0.6 | 3.4 ± 0.4* | 2.8 ± 0.5* |
| Dihomo-γ-Linolenic acid | DGLA | 20:3 n-6 | 58.6 ± 9.0 | 281.3 ± 34.6* | 242.3 ± 23.4* |
| Arachidonic acid | AA | 20:4 n-6 | 135.5 ± 10.6 | 16.1 ± 3.2* | 12.5 ± 3.0* |
| Adrenic acid | AdA | 22:4 n-6 | 5.5 ± 0.5 | 1.0 ± 0.1* | 0.8 ± 0.1* |
| α-Linolenic acid | ALA | 18:3 n-3 | 1.8 ± 0.6 | 0.9 ± 0.3 | 0.7 ± 0.2 |
| Eicosatetraenoic acid | ETA | 20:4 n-3 | 0.6 ± 0.1 | 2.5 ± 0.5* | 1.9 ± 0.4* |
| Eicosapentaenoic acid | EPA | 20:5 n-3 | 3.9 ± 0.6 | 0.3 ± 0.1* | 0.3 ± 0.1* |
| Decosahexaenoic acid | DHA | 22:6 n-3 | 172.5 ± 19.5 | 93.5 ± 11.8* | 78.2 ± 11.6* |
| FADS1 activity | AA/DGLA | 20:4n-6/20:3n-6 | 2.35 ± 0.3 | 0.06 ± 0.01* | 0.05 ± 0.01* |
| FADS2 activity | GLA/LA | 18:3n-6/18:2n-6 | 0.008 ± 0.001 | 0.018 ± 0.002* | 0.014 ± 0.003* |

In addition to increased fatty acid oxidation, lipolytic signatures (increased Lipe, Mg11, Pnpla2 expression) were also observed in EPI WAT of Fads1 KO mice and Compound A-treated mice (FIGS. 4(d), 5(a), 13(b)). Isolated adipocytes from Fads1 KO mice also have increased isoproterenol stimulated lipolysis relative to WT adipocytes. Decreased insulin and Prostaglandin E2 (PGE2) levels (FIGS. 2(e), 7, 8(g)), known anti-lipolytic agents may have contributed to the apparent upregulation of lipolytic genes. Since decreased lipolysis per kilogram of fat mass and decreased FA oxidation has been observed with obesity, concomitant upregulation of adipose lipolysis and hepatic fatty acid oxidation may have enhanced mobilization of lipids from WAT to the liver (and possibly other tissues) for oxidation as a potential mechanism for the anti-obesogenic effect with FADS1 inhibition. Here, only liver and two adipose tissue depots were the focus, with EPI WAT representing the visceral adipose and ING WAT representing subcutaneous adipose tissue. However, since FADS1 is ubiquitously expressed, FADS1 inhibition may impact other metabolically relevant tissues.

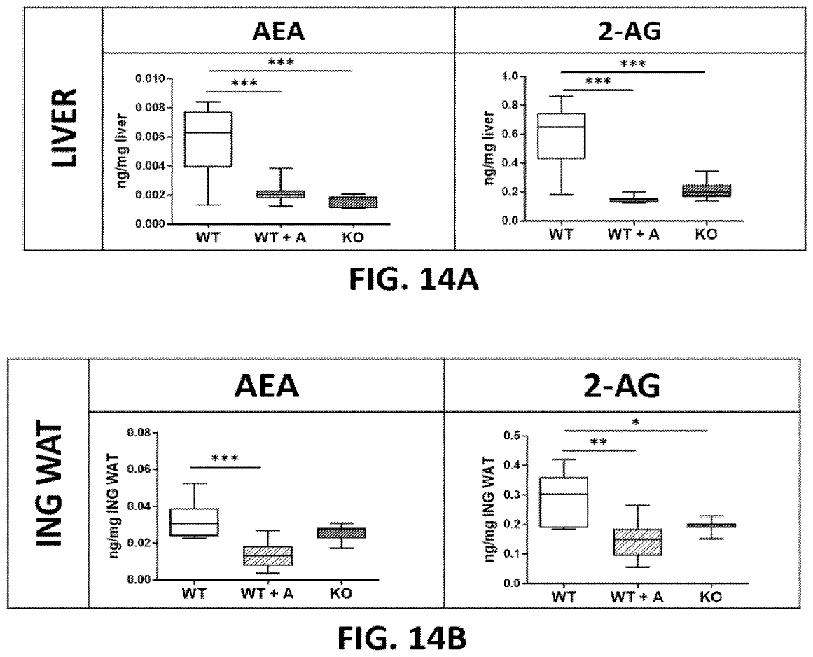

Since AA is a precursor for the endocannabinoids 2-AG and AEA, downregulation of endocannabinoid tone was predicted when AA availability is limited by FADS1 inhibition. This effect was observed in Fads1 KO and Compound A-treated mice (FIGS. 14(a)-14(b)). Decreased levels of endocannabinoids may contribute to the metabolic phenotype observed in the Fads1 KO and Compound A-treated mice, since rimonabant, a selective inverse agonist of cannabinoid receptor 1 (CB1), the receptor for 2-AG and AEA, has been shown to reduce body weight with a similar phenotype in C57BL/6 DIO mice, including reduced adipose tissue inflammation. However, despite similarities, differences were also noted. Rimonabant treatment increased physical activity only in the light cycle, whereas either FADS1 inhibition or deletion increased physical activity only during the dark cycle (FIGS. 3(g)-(h), 9(i)-(j)), when the mice are more active. Therefore, although reduction in endocannabinoid tone may contribute in part to the phenotype observed in Fads1 KO and Compound A-treated mice, the data provided herein suggest differences in phenotype between rimonabant- and FADS1 inhibitor-treated mice, suggesting that additional mechanisms may be involved in driving the improved metabolic phenotype when FADS1 activity has been abrogated.

Monitoring was performed for hepatic PPARα target gene expression and hepatic triglyceride content in Fads1 KO mice fed a chow or HFD (FIGS. 15 (a)-(b)) and in DIO mice treated with Compound A (FIG. 15 (c)). No apparent increase in hepatic triglyceride content was observed. Some groups have reported low liver fat content in Fads1 KO mice. A general increase in expression of hepatic PPARα target genes such as Cpt1a and downregulation of lipogenic gene Srebf1 in liver of FADS1-inhibited mice was observed, with a more pronounced effect in Compound A-treated mice relative to Fads1 KO mice (FIGS. 4(c), 5(a), 10(d), 13(a), 15(e)). No significant increase in hepatic expression of hepatic steatosis markers were observed in the Fads1 KO mice (FIG. 15(d)). Plasma liver enzyme alanine aminotransferase (ALT) was decreased in both Fads1 KO and Compound A-treated mice. Additionally, CP-24879, a dual inhibitor of FADS1/FADS2 (D5D/D6D), demonstrated anti-steatotic effects in hepatocytes. Together, these data suggest that hepatic steatosis is likely not a significant concern for FADS1 inhibition.

In summary, inhibition of FADS1 activity in DIO mice alleviates obesity and improves the metabolic profile of these mice by decreasing inflammation and increasing insulin sensitivity and lipid oxidation. These improvements are partially explained by decreased RER and increased physical activity as measured by indirect calorimetry. Transcriptomic and metabolomic analysis shed light on the metabolic state of FADS1-inhibited mice. Observed signatures of decreased inflammation and increased adiponectin levels in EPI WAT, and upregulation of PPARα activated fatty acid oxidation genes in the liver of Fads1 KO and Compound A-treated mice implicate the pathways involved in FADS1 inhibition-mediated weight loss and improvement of metabolic profiles. Furthermore, downregulation of endocannabinoids levels may contribute partially to the anti-obesogenic mechanism of FADS1 inhibition. Together, these data suggest that FADS1 inhibitors may have therapeutic potential for the treatment of obesity and related comorbidities.

FADS1 activity is elevated in obese male mice and humans. Fads1 KO mice are protected from HFD-induced obesity and show improved metabolic parameters. Similarly, chronic administration of a FADS1 inhibitor, Compound A, to DIO mice significantly reduced body weight and improved metabolic profile while exerting minimal impact on food consumption. Genetic deletion or inhibition of FADS1 leads to alteration of lipid profile, increased lipid oxidation and decreased inflammation. A multi-omics approach reveals insights into the potential underlying mechanisms, highlighting varied involvement across metabolic tissues. The mechanism by which FADS1 inhibition combats obesity may involve concurrent rise in adipose lipolysis and hepatic lipid oxidation to reduce adiposity.

1. A method of treating a FADS1-mediated disease or disorder in a subject in need of treatment, the method comprising:
receiving information that compares a level of a biological indicator of FADS1-mediated disease from the subject to a reference level of the biological indicator;
wherein the biological indicator is a ratio arachidonic acid (AA) to dihomo-gamma-linolenic acid (DGLA); and
administering to the subject a therapeutically effective amount of a FADS1 inhibitor compound if the level of biological indicator in the subject is greater than the reference level of biological indicator.

2. The method of claim 1, wherein the reference level of the biological indicator is an AA to DGLA ratio that is equal to or at least about 5:1.

3.-4. (canceled)

5. A method of treating a FADS1-mediated disease or disorder in a subject in need of treatment, the method comprising:
receiving information that compares a level of a biological indicator of FADS1-mediated disease from the subject to a reference level of the biological indicator;
wherein the biological indicator is a measured level of linoleic acid, gamma-linoleic acid, adrenic acid, docosapenatenoic acid n-6, alpha-linolenic acid, eicosatetraenoic acid, eicosapentaenoic acid, docosahexaenoic acid, plasma cholesterol, free cholesterol, total cholesterol, cholesterylester C20:4, malate, alpha-ketoglutarate, mannose, glucose, erythro-dihydrosphingosine (d18:0), 5-O-methylsphingosine (d18:1), erythro-sphingosine (d18:1), 3-O-methylsphingosine (d18:1), threo-sphingosine (d18:1), 1-hydroxy-2-amino-(cis,trans)-3,5-octadecadiene, 4-hydroxysphinganine (t18:0, phytosphingosine), sphingomyelin (d18:1,C23:0), sphingomyelin (d18:1,C24:0), ceramide (d18:1, C24:0), thromboxane B2, delta-12-prostaglandin D2, prostaglandin E2, prostaglandin D2, 12-hydroxyheptadecatrienoic acid (C17:[5,8,10]3), 14,15-dihydroxyeicosatrienoic acid (C20:cis[5,8,11]3), 11-hydroxyeicosatetraenoic acid (C20:cis[5,8,12,14]4), 13-hydroxyoctadecadienoic acid (13-HODE) (C18:cis[9]trans[11]2), arachidonic acid (C20:cis[5,8,11,14]4), docosahexaenoic acid (C22:cis[4,7,10,13,16,19]6), dihomo-gamma-linolenic acid (C20:cis[8,11,14]3), gamma-linolenic acid (C18:cis[6,9,12]3), docosapentaenoic acid (C22:cis[7,10,13,16,19]5), eicosapentaenoic acid (C20:cis[5,8,11,14,17]5), docosatetraenoic acid (C22:cis[7,10,13,16]4), stearic acid (C18:0), tryptophan, kynurenic acid, xanthurenic acid, histidine, leucine, isoleucine, valine, 3-hydroxyisobutyrate, glutamate, threonine, cysteine, sarcosine, plasma triglycerides, taurochenodeoxycholic acid, taurocholic acid, lysophosphatidylcholine (C18:0), lysophosphatidylcholine (C20:4), lysophosphatidylcholine (C17:0), lysophosphatidylethanolamine (C22:5), phosphatidylcholine (C18:0,C22:6), phosphatidylcholine (C18:0,C20:3), phosphatidylcholine (C18:1,C18:2), phosphatidylcholine (C16:1,C18:2), phosphatidylcholine (C18:0,C18:2), phosphatidylcholine (C16:0,C20:5), phosphatidylcholine (C16:0,C16:0), glycerol-3 phosphate, choline plasmalogen (C18,C20:4), myo-inositol, myo-inositolphospholipids, glycerol phosphate, phosphate lipid fraction, or a combination of the foregoing; and
administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator.

6. The method of claim 5, wherein the subject is determined to be one who would benefit from treatment with the FADS1 inhibitor compound where the subject's level of the biological indicator is higher than the reference level for the biological indicator.

7. The method of claim 5, wherein the subject is determined to be one who would benefit from treatment with the FADS1 inhibitor compound where the subject's level of the biological indicator is lower than the reference level for the biological indicator.

8.-10. (canceled)

11. A method of treating a FADS1-mediated disease or disorder in a subject in need of treatment, the method comprising:
receiving information that compares a level of a biological indicator of FADS1-mediated disease from the subject to a reference level of the biological indicator;
wherein the biological indicator is a measured level of a differentially expressed gene (DEG);
wherein the DEG is Serpinb1a, Gna14, Serpina3m, Hsd11b1, Cyp2c29, Akr1d1, Aldh1a1, Mmp19, Gyp27a1, Tymp, Elov12, Chkb, H2afj, Tnfaip811, Tmem86a, Sel113, Agap2, 4833411C07Rik, Elov4, Fat3, Eef1a2, Atp7a, Rgs2, Cenpe, Mfap2, Mlki67, Ctsh, Trub2, Ubc, H6pd, Eepd1, Acss2, Aacs, Gm36827, Man2a2, Nudt18, Plagl1, TM4sf19, Atp6v0d2, Gm20056, Trem2, I11rn, Mmp12, Cdk18, Efr3b, Tag1n2, Lurap1, Cp, I17rb, B230303O12Rik, Cfd, Sult1e1, Tdo2, Cyp2b9, Hao2, Cyp2b13, Cyp2a22, Acnat2, Ildr2, Rpl10a-ps1, Tm6sf2, Fitm1, Lpar1, C6, Cmah, Lbp, Arsg, Glra3, Lad1, A730063M14Rik, Ly6f, Foxi1, Crygc, Defb28, Wfdc9, Ph1da2, Aqp6, Gm16411, Adam7, Ppp2r5b, Slc6a7, Gpr50, Ahnak2, S100a6, Mmp14, Htr2b, Hpgds, Gm18537, Pclo, Adrb3, Gm38394, AC154232.2, Cadps, Adgrb2, Gm45470, Sdr9c7, Dsg1c, Slc17a1, Ces1c, Gss, 1810008118Rik, Tlcd1, Snrk, Akr1c20, Gm19950, Ttr, Cbfa2t3, Acat2, Pmvk, Abcd3, Acacb, Arhgap27, Rnase9, Wfdc8, Ighv9-2, DerI3, Acap1, Ccl19, Tcf7, Xkrx, Trim46, Zfp369, Zfp871, Pcdhb21, Gm14288, Uprt, Atm, Dchs2, Cped1, Gm38357, Cck, Ckap2, Gm4419, 1600015I10Rik, Sez6I2, Prnd, Gm16702, S100a8, Pcdh12, Malat1, Kcnqlot1, ArI4c, Gm42549, Gm37310, Gm37776, Atp2a1, Ckm, Tnnt3, Adipoq, Fabp4, Lep, Retn, Hoxc8, Hoxc9, Cebpa, Dgat1, Dgat2, Elov13, Fas, Scd1, Srebf1, Hilpda, Lipe, Mg11, Plin1, Plin4, Pnpla2, Pnpla3, Ldah, Cs, Gckr, Me1, Pck1, Pdk4, Irs1, Acaa1a, Acads, Acox1, Cpt1a, Cpt1b, Hadhb, Ehhadh, Ppara, Ppargc1a, Cyp4a12a, Cyp4a12b, Cyp2e1, Adgre1, Agtr1a, Cc12, Cd14, Cd68, Il1b, Tnf, or a combination of the foregoing; and
administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator.

12. The method of claim 11, wherein the subject is determined to be one who would benefit from treatment with the FADS1 inhibitor compound where the subject's level of the biological indicator is higher than the reference level for the biological indicator.

13. The method of claim 11, wherein the subject is determined to be one who would benefit from treatment with the FADS1 inhibitor compound where the subject's level of the biological indicator is lower than the reference level for the biological indicator.

14. (canceled)

15. A method of treating a FADS1-mediated disease or disorder in a subject in need of treatment, the method comprising:
receiving information that compares a level of a biological indicator of FADS1-mediated disease from the subject to a reference level of the biological indicator;
wherein the biological indicator is a measured level of a cell type in the subject, wherein the cell type is an adipocyte (Adipo), B cell (Bcell), endothelial cell (Endo), hepatocyte (Hep), kupffer cell (Kupff), myeloid cell (Myel), natural killer cell (NK), T cell (Tcell), or a combination of the foregoing; and
administering to the subject a therapeutically effective amount of a FADS1 inhibitor compound if the subject is determined to be one who would benefit from treatment with the FADS1 inhibitor compound as determined by the subject's level of the biological indicator compared to the reference level for the biological indicator.

16. The method of claim 15, wherein the subject is determined to be one who would benefit from treatment with the FADS1 inhibitor compound where the subject's level of the biological indicator is higher than the reference level for the biological indicator.

17. The method of claim 15, wherein the subject is determined to be one who would benefit from treatment with the FADS1 inhibitor compound where the subject's level of the biological indicator is lower than the reference level for the biological indicator.

18. The method of claim 1, wherein the FADS1-mediated disease or disorder is obesity, a metabolic disorder, a cardiovascular disorder, diabetes, dyslipidemia, non-alcoholic steatohepatitis (NASH), or a combination of any of the foregoing.

19.-21. (canceled)

22. The method claim 1, wherein the reference level of biological indicator is an average amount of biological indicator in a population of subjects having a Body Mass Index (BMI) of greater than or equal to 30.0.

23.-24. (canceled)

25. The method or compound of claim 1, wherein the FADS1 inhibitor compound is:
6-[1-(2,2,3,3,3-pentafluoropropyl)-1H-pyrazol-4-yl]-7-(trifluoromethyl)-5H-[1,3]thiazolo[3,2-a]pyrimidin-5-one;
2-methyl-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
1-methyl-6-[1-(2,2,3,3,3-pentafluoropropyl)-1H-pyrazol-4-yl]-7-(trifluoromethyl)-1H,2H,3H,5H-imidazo[1,2-a]pyrimidine-2,5-dione;
2-methyl-6-[1-(2,2,3,3,3-pentafluoropropyl)-1H-pyrazol-4-yl]-7-(trifluoromethyl)-5H-[1,3]thiazolo[3,2-a]pyrimidin-5-one;
3-methyl-6-[1-(2,2,3,3,3-pentafluoropropyl)-1H-pyrazol-4-yl]-7-(trifluoromethyl)-5H-[1,3]thiazolo[3,2-a]pyrimidin-5-one;
6-[1-(2,2,3,3,3-pentafluoropropyl)-1H-pyrazol-4-yl]-3,7-bis(trifluoromethyl)-5H-[1,3]thiazolo[3,2-a]pyrimidin-5-one;
2-fluoro-6-[1-(2,2,3,3,3-pentafluoropropyl)-1H-pyrazol-4-yl]-7-(trifluoromethyl)-5H-[1,3]thiazolo[3,2-a]pyrimidin-5-one;
7-(trifluoromethyl)-6-[1-(3,3,3-trifluoropropyl)-1H-pyrazol-4-yl]-5H-[1,3]thiazolo[3,2-a]pyrimidin-5-one;
2-chloro-6-[1-(2,2,3,3,3-pentafluoropropyl)-1H-pyrazol-4-yl]-7-(trifluoromethyl)-5H-[1,3]thiazolo[3,2-a]pyrimidin-5-one;
2-(methoxymethyl)-7-(trifluoromethyl)-6-[1-(3,3,3-trifluoropropyl)-1H-pyrazol-4-yl]-5H-[1,3,4]thiadiazolo[3,2-a]pyrimidin-5-one;
2-cyclopropyl-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
2-cyclopropyl-7-(trifluoromethyl)-6-[1-(3,3,3-trifluoropropyl)-1H-pyrazol-4-yl]-5H-[1,3,4]thiadiazolo[3,2-a]pyrimidin-5-one;
2,3-dimethyl-6-[1-(2,2,3,3,3-pentafluoropropyl)-1H-pyrazol-4-yl]-5-(trifluoromethyl)-3H,7H-[1,2,4]triazolo[1,5-a]pyrimidin-7-one;
2,3-dimethyl-5-(trifluoromethyl)-6-[1-(3,3,3-trifluoropropyl)-1H-pyrazol-4-yl]-3H,7H-[1,2,4]triazolo[1,5-a]pyrimidin-7-one;
7-ethyl-2-methyl-6-[1-(2,2,3,3,3-pentafluoropropyl)-1H-pyrazol-4-yl]-5H-[1,3,4]thiadiazolo[3,2-a]pyrimidin-5-one;
1,2-dimethyl-6-[1-(2,2,3,3,3-pentafluoropropyl)-1H-pyrazol-4-yl]-5-(trifluoromethyl)-1H,7H-pyrazolo[1,5-a]pyrimidin-7-one;
1-methyl-6-[1-(2,2,3,3,3-pentafluoropropyl)-1H-pyrazol-4-yl]-5-(trifluoromethyl)-1H,7H-pyrazolo[1,5-a]pyrimidin-7-one;
1,3-dimethyl-6-[1-(2,2,3,3,3-pentafluoropropyl)-1H-pyrazol-4-yl]-5-(trifluoromethyl)-1H,7H-pyrazolo[1,5-a]pyrimidin-7-one;
3-[1-(2,2,3,3,3-pentafluoropropyl)-1H-pyrazol-4-yl]-2-(trifluoromethyl)-4H,6H,7H,8H-pyrrolo[1,2-a]pyrimidin-4-one;
2-methyl-7-(trifluoromethyl)-6-[1-(3,3,3-trifluoropropyl)-1H-pyrazol-4-yl]-5H-[1,3,4]thiadiazolo[3,2-a]pyrimidin-5-one;
2-(trifluoromethyl)-3-[1-(3,3,3-trifluoropropyl)-1H-pyrazol-4-yl]-4H,6H,7H,8H-pyrrolo[1,2-a]pyrimidin-4-one;
6-[1-(2,2,3,3,3-pentafluoropropyl)-1H-pyrazol-4-yl]-7-(trifluoromethyl)-2H,3H,5H-[1,3]thiazolo[3,2-a]pyrimidin-5-one;
6-{1-[(2,2-difluorocyclopropyl)methyl]-1H-pyrazol-4-yl}-2-methyl-7-(trifluoromethyl)-5H-[1,3,4]thiadiazolo[3,2-a]pyrimidin-5-one;
2-methyl-6-[1-(2,2,3,3,3-pentafluoropropyl)-1H-pyrazol-4-yl]-7-(trifluoromethyl)-2H,3H,5H-[1,3]thiazolo[3,2-a]pyrimidin-5-one;
8-[1-(2,2,3,3,3-pentafluoropropyl)-1H-pyrazol-4-yl]-9-(trifluoromethyl)-6,10-diazatricyclo[4.4.0.0^{2,4}]deca-1(10),8-dien-7-one;
6-{1-[(3,3-difluorocyclobutyl)methyl]-1H-pyrazol-4-yl}-2-methyl-7-(trifluoromethyl)-5H-[1,3,4]thiadiazolo[3,2-a]pyrimidin-5-one;
1-methyl-6-[1-(2,2,3,3,3-pentafluoropropyl)-1H-pyrazol-4-yl]-7-(trifluoromethyl)-1H,5H-[1,2,4]triazolo[4,3-a]pyrimidin-5-one;
3-methyl-6-[1-(2,2,3,3,3-pentafluoropropyl)-1H-pyrazol-4-yl]-5-(trifluoromethyl)-3H,7H-[1,2,3,4]tetrazolo[1,5-a]pyrimidin-7-one;
2-methyl-6-{1-[(oxetan-3-yl)methyl]-1H-pyrazol-4-yl}-7-(trifluoromethyl)-5H-[1,3,4]thiadiazolo[3,2-a]pyrimidin-5-one;
2-methyl-6-[1-(2,2,3,3,3-pentafluoropropyl)-1H-pyrazol-3-yl]-7-(trifluoromethyl)-5H-[1,3,4]thiadiazolo[3,2-a]pyrimidin-5-one;
6-[4-(2,2,2-trifluoroethoxy)phenyl]-7-(trifluoromethyl)-5H-[1,3]thiazolo[3,2-a]pyrimidin-5-one;
2-(hydroxymethyl)-6-[1-(2,2,3,3,3-pentafluoropropyl)-1H-pyrazol-4-yl]-7-(trifluoromethyl)-5H-[1,3]thiazolo[3,2-a]pyrimidin-5-one;
2-(hydroxymethyl)-6-[4-(2,2,2-trifluoroethoxy)phenyl]-7-(trifluoromethyl)-5H-[1,3]thiazolo[3,2-a]pyrimidin-5-one;
2-chloro-6-[4-(2,2,2-trifluoroethoxy)phenyl]-7-(trifluoromethyl)-5H-[1,3]thiazolo[3,2-a]pyrimidin-5-one;
6-[1-(2,2,3,3,3-pentafluoropropyl)-1H-pyrazol-4-yl]-1-(propan-2-yl)-7-(trifluoromethyl)-1H,2H,3H,5H-imidazo[1,2-a]pyrimidine-2,5-dione;
1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
3-methyl-6-[1-(2,2,3,3,3-pentafluoropropyl)-1H-pyrazol-4-yl]-5-(trifluoromethyl)-3H,7H-[1,2,4]triazolo[1,5-a]pyrimidin-7-one;
2-methyl-6-[1-(2,2,3,3,3-pentafluoropropyl)-1H-pyrazol-4-yl]-7-(trifluoromethyl)-5H-[1,3]oxazolo[3,2-a]pyrimidin-5-one;
2-methyl-6-[4-(2,2,2-trifluoroethoxy)phenyl]-7-(trifluoromethyl)-5H-[1,3,4]thiadiazolo[3,2-a]pyrimidin-5-one;
7-ethoxy-2-methyl-6-[1-(2,2,3,3,3-pentafluoropropyl)-1H-pyrazol-4-yl]-5H-[1,3,4]thiadiazolo[3,2-a]pyrimidin-5-one;
2-(methoxymethyl)-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
2-methoxy-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
3-chloro-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-(hydroxymethyl)-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
2-(hydroxymethyl)-7-(trifluoromethyl)-6-[1-(3,3,3-trifluoropropyl)-1H-pyrazol-4-yl]-5H-[1,3,4]thiadiazolo[3,2-a]pyrimidin-5-one;
6-[1-(2,2,3,3,3-pentafluoropropyl)-1H-pyrazol-4-yl]-7-(trifluoromethyl)-1H,2H,3H,5H-imidazo[1,2-a]pyrimidine-2,5-dione;
2-chloro-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-chloro-6-[1-(2,2,3,3,3-pentafluoropropyl)-1H-pyrazol-4-yl]-7-(trifluoromethyl)-1H,5H-imidazo[1,2-a]pyrimidin-5-one;
2-cyclopropyl-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-chloro-1-methyl-7-(trifluoromethyl)-6-[1-(3,3,3-trifluoropropyl)-1H-pyrazol-4-yl]-1H,5H-imidazo[1,2-a]pyrimidin-5-one;
1,2-dimethyl-6-[1-(2,2,3,3,3-pentafluoropropyl)-1H-pyrazol-4-yl]-7-(trifluoromethyl)-1H,5H-imidazo[1,2-a]pyrimidin-5-one;
1,2-dimethyl-7-(trifluoromethyl)-6-[1-(3,3,3-trifluoropropyl)-1H-pyrazol-4-yl]-1H,5H-imidazo[1,2-a]pyrimidin-5-one;
1,2-dimethyl-6-[4-(2,2,2-trifluoroethoxy)phenyl]-7-(trifluoromethyl)-1H,5H-imidazo[1,2-a]pyrimidin-5-one;
2-(methoxymethyl)-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1-ethyl-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1-(2-methoxyethyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(propan-2-yl)-7-(trifluoromethyl)-1H,5H-imidazo[1,2-a]pyrimidin-5-one;
6-{1-[(2,2-difluorocyclopropyl)methyl]-1H-pyrazol-4-yl}-1,2-dimethyl-7-(trifluoromethyl)-1H,5H-imidazo[1,2-a]pyrimidin-5-one;
6-{1-[(3,3-difluorocyclobutyl)methyl]-1H-pyrazol-4-yl}-1,2-dimethyl-7-(trifluoromethyl)-1H,5H-imidazo[1,2-a]pyrimidin-5-one;
6-[1-(cyclopropylmethyl)-1H-pyrazol-4-yl]-1,2-dimethyl-7-(trifluoromethyl)-1H,5H-imidazo[1,2-a]pyrimidin-5-one;
1-(cyclopropylmethyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-(methoxymethyl)-1-methyl-7-(trifluoromethyl)-6-[1-(3,3,3-trifluoropropyl)-1H-pyrazol-4-yl]-1H,5H-imidazo[1,2-a]pyrimidin-5-one;
1-(2-hydroxypropyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1,2-dimethyl-6-{1-[(oxetan-3-yl)methyl]-1H-pyrazol-4-yl}-7-(trifluoromethyl)-1H,5H-imidazo[1,2-a]pyrimidin-5-one;
1-(cyclopropylmethyl)-2-methyl-7-(trifluoromethyl)-6-[1-(3,3,3-trifluoropropyl)-1H-pyrazol-4-yl]-1H,5H-imidazo[1,2-a]pyrimidin-5-one;
1-[2-(dimethylamino)ethyl]-2-methyl-7-(trifluoromethyl)-6-[1-(3,3,3-trifluoropropyl)-1H-pyrazol-4-yl]-1H,5H-imidazo[1,2-a]pyrimidin-5-one;
1-(cyclopropylmethyl)-2-methyl-6-[4-(2,2,2-trifluoroethoxy)phenyl]-7-(trifluoromethyl)-1H,5H-imidazo[1,2-a]pyrimidin-5-one;
1-[2-(dimethylamino)ethyl]-2-methyl-6-[4-(2,2,2-trifluoroethoxy)phenyl]-7-(trifluoromethyl)-1H,5H-imidazo[1,2-a]pyrimidin-5-one;
1,2-dimethyl-6-[1-(2,2,3,3,3-pentafluoropropyl)-1H-pyrazol-3-yl]-7-(trifluoromethyl)-1H,5H-imidazo[1,2-a]pyrimidin-5-one;
2-methoxy-1-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methoxy-1-methyl-7-(trifluoromethyl)-6-[1-(3,3,3-trifluoropropyl)-1H-pyrazol-4-yl]-1H,5H-imidazo[1,2-a]pyrimidin-5-one;
2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-7-(trifluoromethyl)-6-[1-(3,3,3-trifluoropropyl)-1H-pyrazol-4-yl]-1H,5H-imidazo[1,2-a]pyrimidin-5-one;
2-methyl-6-[4-(2,2,2-trifluoroethoxy)phenyl]-7-(trifluoromethyl)-1H,5H-imidazo[1,2-a]pyrimidin-5-one;
6-{1-[(2,2-difluorocyclopropyl)methyl]-1H-pyrazol-4-yl}-2-methyl-7-(trifluoromethyl)-1H,5H-imidazo[1,2-a]pyrimidin-5-one;
1-(²H₃)methyl-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1-(²H₃)methyl-2-methyl-7-(trifluoromethyl)-6-[1-(3,3,3-trifluoropropyl)-1H-pyrazol-4-yl]-1H,5H-imidazo[1,2-a]pyrimidin-5-one;
1-(2-hydroxyethyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
methyl 2-methyl-5-oxo-6-[1-(2,2,3,3,3-pentafluoropropyl)-1H-pyrazol-4-yl]-7-(trifluoromethyl)-1H,5H-imidazo[1,2-a]pyrimidine-1-carboxylate;
1-[(2,2-difluorocyclopropyl)methyl]-2-methyl-7-(trifluoromethyl)-6-[1-(3,3,3-trifluoropropyl)-1H-pyrazol-4-yl]-1H,5H-imidazo[1,2-a]pyrimidin-5-one;
1-[(3,3-difluorocyclobutyl)methyl]-2-methyl-7-(trifluoromethyl)-6-[1-(3,3,3-trifluoropropyl)-1H-pyrazol-4-yl]-1H,5H-imidazo[1,2-a]pyrimidin-5-one;
1-(2-hydroxyethyl)-2-methyl-7-(trifluoromethyl)-6-[1-(3,3,3-trifluoropropyl)-1H-pyrazol-4-yl]-1H,5H-imidazo[1,2-a]pyrimidin-5-one;
1-[2-(dimethylamino)ethyl]-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(prop-2-yn-1-yl)-7-(trifluoromethyl)-1H,5H-imidazo[1,2-a]pyrimidin-5-one;
2-{2-methyl-5-oxo-6-[1-(2,2,3,3,3-pentafluoropropyl)-1H-pyrazol-4-yl]-7-(trifluoromethyl)-1H,5H-imidazo[1,2-a]pyrimidin-1-yl}acetonitrile;
2-[2-methyl-5-oxo-7-(trifluoromethyl)-6-[1-(3,3,3-trifluoropropyl)-1H-pyrazol-4-yl]-1H,5H-imidazo[1,2-a]pyrimidin-1-yl]acetonitrile;
1-(2-hydroxy-2-methylpropyl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
1-[2-(1-hydroxycyclopropyl)ethyl]-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-1-[(oxetan-3-yl)methyl]-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-1-(oxetan-3-yl)-6-[1-(2,2,3,3,3-pentafluoropropyl)-1H-pyrazol-4-yl]-7-(trifluoromethyl)-1H,5H-imidazo[1,2-a]pyrimidin-5-one;
1,2-dimethyl-6-[1-(2,2,3,3,3-pentafluoropropyl)-1H-pyrazol-4-yl]-7-(trifluoromethyl)-1H,5H-imidazo[1,2-a]pyrimidine-5-thione;
2-methyl-6-[1-(2,2,3,3,3-pentafluoropropyl)-1H-pyrazol-4-yl]-1-(pyridin-2-yl)-7-(trifluoromethyl)-1H,5H-imidazo[1,2-a]pyrimidin-5-one;
2-methyl-1-(pyridin-2-yl)-7-(trifluoromethyl)-6-[1-(3,3,3-trifluoropropyl)-1H-pyrazol-4-yl]-1H,5H-imidazo[1,2-a]pyrimidin-5-one;
2-methyl-6-[1-(2,2,3,3,3-pentafluoropropyl)-1H-pyrazol-4-yl]-1-(pyrazin-2-yl)-7-(trifluoromethyl)-1H,5H-imidazo[1,2-a]pyrimidin-5-one;
2-methyl-1-(6-methylpyridin-2-yl)-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-1-(1-methyl-1H-pyrazol-4-yl)-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(pyridin-3-yl)-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-phenyl-7-(trifluoromethyl)-1H,5H-imidazo[1,2-a]pyrimidin-5-one;
1-(6-chloropyridin-2-yl)-2-methyl-6-[1-(2,2,3,3,3-pentafluoropropyl)-1H-pyrazol-4-yl]-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(pyridin-4-yl)-7-(trifluoromethyl)-1H,5H-imidazo[1,2-a]pyrimidin-5-one;
2-methyl-6-[1-(2,2,3,3,3-pentafluoropropyl)-1H-pyrazol-4-yl]-1-(1H-pyrazol-4-yl)-7-(trifluoromethyl)-1H,5H-imidazo[1,2-a]pyrimidin-5-one;
2-(fluoromethyl)-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
2-[(dimethylamino)methyl]-6-[1-(2,2,3,3,3-pentafluoropropyl)-1H-pyrazol-4-yl]-7-(trifluoromethyl)-5H-[1,3,4]thiadiazolo[3,2-a]pyrimidin-5-one;
6-(1-{[(1R)-2,2-difluorocyclopropyl]methyl}-1H-pyrazol-4-yl)-2-methyl-7-(trifluoromethyl)-1H,5H-imidazo[1,2-a]pyrimidin-5-one;
6-(1-{[(1S)-2,2-difluorocyclopropyl]methyl}-1H-pyrazol-4-yl)-2-methyl-7-(trifluoromethyl)-1H,5H-imidazo[1,2-a]pyrimidin-5-one;
6-(1-{[(1R)-2,2-difluorocyclopropyl]methyl}-1H-pyrazol-4-yl)-1,2-dimethyl-7-(trifluoromethyl)-1H,5H-imidazo[1,2-a]pyrimidin-5-one;
6-(1-{[(1S)-2,2-difluorocyclopropyl]methyl}-1H-pyrazol-4-yl)-1,2-dimethyl-7-(trifluoromethyl)-1H,5H-imidazo[1,2-a]pyrimidin-5-one;
6-(1-{[(1R)-2,2-difluorocyclopropyl]methyl}-1H-pyrazol-4-yl)-2-methyl-7-(trifluoromethyl)-5H-[1,3,4]thiadiazolo[3,2-a]pyrimidin-5-one;
6-(1-{[(1S)-2,2-difluorocyclopropyl]methyl}-1H-pyrazol-4-yl)-2-methyl-7-(trifluoromethyl)-5H-[1,3,4]thiadiazolo[3,2-a]pyrimidin-5-one;
(2R)-2-methyl-6-[1-(2,2,3,3,3-pentafluoropropyl)-1H-pyrazol-4-yl]-7-(trifluoromethyl)-2H,3H,5H-[1,3]thiazolo[3,2-a]pyrimidin-5-one;
(2S)-2-methyl-6-[1-(2,2,3,3,3-pentafluoropropyl)-1H-pyrazol-4-yl]-7-(trifluoromethyl)-2H,3H,5H,6H,7H-[1,3]thiazolo[3,2-a]pyrimidin-5-one;
1-{[(1R)-2,2-difluorocyclopropyl]methyl}-2-methyl-7-(trifluoromethyl)-6-[1-(3,3,3-trifluoropropyl)-1H-pyrazol-4-yl]-1H,5H-imidazo[1,2-a]pyrimidin-5-one;
1-{[(1S)-2,2-difluorocyclopropyl]methyl}-2-methyl-7-(trifluoromethyl)-6-[1-(3,3,3-trifluoropropyl)-1H-pyrazol-4-yl]-1H,5H-imidazo[1,2-a]pyrimidin-5-one;
(2R)-2-(4-(8-methoxy-4-oxo-2-(trifluoromethyl)-4H-pyrido[1,2-a]pyrimidin-3-yl)phenoxy)propanenitrile;
(2R)-2-(4-(8-methoxy-4-oxo-2-(trifluoromethyl)-4H-pyrimido[1,2-a]pyrimidin-3-yl)phenoxy)propanenitrile;
(2S)-2-(4-(8-methoxy-4-oxo-2-(trifluoromethyl)-4H-pyrido[1,2-a]pyrimidin-3-yl)phenoxy)propanenitrile;
(2S)-2-(4-(8-methoxy-4-oxo-2-(trifluoromethyl)-4H-pyrimido[1,2-a]pyrimidin-3-yl)phenoxy)propanenitrile;
(4-(8-methoxy-4-oxo-2-(trifluoromethyl)-4H-pyrido[1,2-a]pyrimidin-3-yl)-1H-pyrazol-1-yl)acetonitrile;
(4-(8-methoxy-4-oxo-2-(trifluoromethyl)-4H-pyrido[1,2-a]pyrimidin-3-yl)phenoxy)acetonitrile;
(4-(8-methoxy-4-oxo-2-(trifluoromethyl)-4H-pyrido[1,2-a]pyrimidin-3-yl)phenyl)acetonitrile;
(4-(8-methoxy-4-oxo-2-(trifluoromethyl)-4H-pyrimido[1,2-a]pyrimidin-3-yl)phenoxy)acetonitrile;
(4-oxo-3-(1-(2,2,3,3,3-pentafluoropropyl)-1H-pyrazol-4-yl)-2-(trifluoromethyl)-4H-pyrido[1,2-a]pyrimidin-8-yl)acetonitrile;
1-(chloromethyl)-7-[1-(2,2,3,3,3-pentafluoropropyl)-1H-pyrazol-4-yl]-8-(trifluoromethyl)-1H,2H,6H-pyrimido[1,2-a][1,3]diazine-2,6-dione;
1-(fluoromethyl)-7-[1-(2,2,3,3,3-pentafluoropropyl)-1H-pyrazol-4-yl]-8-(trifluoromethyl)-1H,2H,6H-[1,3]diazino[1,2-a]pyrimidine-2,6-dione;
1-(methyl-d₃)-7-(4-(2,2,2-trifluoroethoxy)phenyl)-8-(trifluoromethyl)-2H-pyrimido[1,2-a]pyrimidine-2,6(1H)-dione;
1-methyl-7-[1-(2,2,3,3,3-pentafluoropropyl)-1H-pyrazol-4-yl]-8-(trifluoromethyl)-1H,2H,6H-[1,3]diazino[1,2-a]pyrimidine-2,6-dione;
2-(4-(8-methoxy-4-oxo-2-(trifluoromethyl)-4H-pyrido[1,2-a]pyrimidin-3-yl)phenoxy)-2-methylpropanenitrile;
2-(4-(8-methoxy-4-oxo-2-(trifluoromethyl)-4H-pyrido[1,2-a]pyrimidin-3-yl)phenoxy)propanenitrile;
2-(4-(8-methoxy-4-oxo-2-(trifluoromethyl)-4H-pyrimido[1,2-a]pyrimidin-3-yl)phenoxy)propanenitrile;
2-(difluoromethyl)-3-(3-fluoro-4-(2,2,2-trifluoroethoxy)phenyl)-8-methoxy-4H-pyrido[1,2-a]pyrimidin-4-one;
2-(difluoromethyl)-4-oxo-3-(1-(2,2,3,3,3-pentafluoropropyl)-1H-pyrazol-4-yl)-4H-pyrido[1,2-a]pyrimidine-8-carbonitrile;
2-(difluoromethyl)-8-methoxy-3-(1-(3,3,3-trifluoropropyl)-1H-pyrazol-4-yl)-4H-pyrido[1,2-a]pyrimidin-4-one;
2-(difluoromethyl)-8-methoxy-3-(1-(4,4,4-trifluorobutyl)-1H-pyrazol-4-yl)-4H-pyrido[1,2-a]pyrimidin-4-one;
2-(difluoromethyl)-8-methoxy-3-(4-(2,2,2-trifluoroethoxy)phenyl)-4H-pyrido[1,2-a]pyrimidin-4-one;
2-(difluoromethyl)-8-methoxy-3-(6-(2,2,2-trifluoroethoxy)-3-pyridinyl)-4H-pyrido[1,2-a]pyrimidin-4-one;
2-(fluoromethyl)-4-oxo-3-(1-(2,2,3,3,3-pentafluoropropyl)-1H-pyrazol-4-yl)-4H-pyrido[1,2-a]pyrimidine-8-carbonitrile;
2-(fluoromethyl)-8-methoxy-3-(4-(2,2,2-trifluoroethoxy)phenyl)-4H-pyrido[1,2-a]pyrimidin-4-one;
2-(trifluoromethyl)-3-[1-(3,3,3-trifluoropropyl)-1H-pyrazol-4-yl]-4H,6H,7H,9H-pyrimido[2,1-c][1,4]oxazin-4-one;
2,8-dimethoxy-3-(4-(2,2,2-trifluoroethoxy)phenyl)-4H-pyrido[1,2-a]pyrimidin-4-one;
2-cyclopropyl-8-methoxy-3-(4-(2,2,2-trifluoroethoxy)phenyl)-4H-pyrido[1,2-a]pyrimidin-4-one;
2-ethoxy-8-methoxy-3-(4-(2,2,2-trifluoroethoxy)phenyl)-4H-pyrido[1,2-a]pyrimidin-4-one;
2-ethoxy-8-methoxy-3-[1-(2,2,3,3,3-pentafluoropropyl)-1H-pyrazol-4-yl]-4H-pyrido[1,2-a]pyrimidin-4-one;
2-ethyl-8-methoxy-3-(1-(2,2,3,3,3-pentafluoropropyl)-1H-pyrazol-4-yl)-4H-pyrido[1,2-a]pyrimidin-4-one;
2-ethyl-8-methoxy-3-(4-(2,2,2-trifluoroethoxy)phenyl)-4H-pyrido[1,2-a]pyrimidin-4-one;
2-ethyl-8-methoxy-3-(6-(2,2,2-trifluoroethoxy)-3-pyridinyl)-4H-pyrido[1,2-a]pyrimidin-4-one;
3-(1-(2,2,3,3,3-pentafluoropropyl)-1H-pyrazol-4-yl)-2-(trifluoromethyl)-4H-pyrido[1,2-a]pyrimidine-4,8(1H)-dione;
3-(1-(2,2-difluoropropyl)-1H-pyrazol-4-yl)-8-methoxy-2-(trifluoromethyl)-4H-pyrido[1,2-a]pyrimidin-4-one;
3-(1-(3-fluorophenyl)-1H-pyrazol-4-yl)-8-methoxy-2-(trifluoromethyl)-4H-pyrido[1,2-a]pyrimidin-4-one;
3-(1-(4-fluorophenyl)-1H-pyrazol-4-yl)-2-(trifluoromethyl)-4H-pyrido[1,2-a]pyrimidin-4-one;
3-(1-(4-fluorophenyl)-1H-pyrazol-4-yl)-8-methoxy-2-(trifluoromethyl)-4H-pyrido[1,2-a]pyrimidin-4-one;
3-(1-{[(1R)-2,2-difluorocyclopropyl]methyl}-1H-pyrazol-4-yl)-8-methoxy-2-(trifluoromethyl)-4H-pyrido[1,2-a]pyrimidin-4-one;
3-(1-{[(1S)-2,2-difluorocyclopropyl]methyl}-1H-pyrazol-4-yl)-8-methoxy-2-(trifluoromethyl)-4H-pyrido[1,2-a]pyrimidin-4-one;
3-(1-benzofuran-2-yl)-8-methoxy-2-(trifluoromethyl)-4H-pyrido[1,2-a]pyrimidin-4-one;
3-(1-cyclopropyl-1H-pyrazol-4-yl)-2-(trifluoromethyl)-4H-pyrido[1,2-a]pyrimidin-4-one;
3-(1-cyclopropyl-1H-pyrazol-4-yl)-8-methoxy-2-(trifluoromethyl)-4H-pyrido[1,2-a]pyrimidin-4-one;
3-(1-phenyl-1H-pyrazol-4-yl)-2-(trifluoromethyl)-4H-pyrido[1,2-a]pyrimidin-4-one;
3-(1-propyl-1H-pyrazol-4-yl)-2-(trifluoromethyl)-4H-pyrido[1,2-a]pyrimidin-4-one;
3-(2-chloro-4-(2,2,2-trifluoroethoxy)phenyl)-8-methoxy-2-(trifluoromethyl)-4H-pyrido[1,2-a]pyrimidin-4-one;
3-(2-fluoro-4-(2,2,2-trifluoroethoxy)phenyl)-2-(trifluoromethyl)-4H-pyrido[1,2-a]pyrimidin-4-one;
3-(2-fluoro-4-(2,2,2-trifluoroethoxy)phenyl)-8-methoxy-2-(trifluoromethyl)-4H-pyrido[1,2-a]pyrimidin-4-one;
3-(2-fluoro-4-(2,2,2-trifluoroethoxy)phenyl)-8-methoxy-2-(trifluoromethyl)-4H-pyrimido[1,2-a]pyrimidin-4-one;
3-(2-fluoro-4-(trifluoromethoxy)phenyl)-8-methoxy-2-(trifluoromethyl)-4H-pyrido[1,2-a]pyrimidin-4-one;
3-(2-fluoro-6-(2,2,2-trifluoroethoxy)-3-pyridinyl)-8-methoxy-2-(trifluoromethyl)-4H-pyrido[1,2-a]pyrimidin-4-one;
3-(3-chloro-4-(2,2,2-trifluoroethoxy)phenyl)-2-(trifluoromethyl)-4H-pyrido[1,2-a]pyrimidin-4-one;
3-(3-fluoro-4-(2,2,2-trifluoroethoxy)phenyl)-8-methoxy-2-(trifluoromethyl)-4H-pyrido[1,2-a]pyrimidin-4-one;
3-(3-fluoro-4-(2,2,2-trifluoroethoxy)phenyl)-8-methoxy-2-(trifluoromethyl)-4H-pyrimido[1,2-a]pyrimidin-4-one;
3-(4-(((1R)-2,2-difluorocyclopropyl)methoxy)phenyl)-8-methoxy-2-(trifluoromethyl)-4H-pyrido[1,2-a]pyrimidin-4-one;
3-(4-(((1S)-2,2-difluorocyclopropyl)methoxy)phenyl)-8-methoxy-2-(trifluoromethyl)-4H-pyrido[1,2-a]pyrimidin-4-one;
3-(4-((2,2-difluorocyclopropyl)methoxy)phenyl)-8-methoxy-2-(trifluoromethyl)-4H-pyrido[1,2-a]pyrimidin-4-one;
3-(4-(2,2,2-trifluoroethoxy)phenyl)-2-(trifluoromethyl)-4H-pyrido[1,2-a]pyrimidin-4-one;
3-(4-(2,2,2-trifluoroethoxy)phenyl)-2-(trifluoromethyl)-4H-pyrido[1,2-a]pyrimidine-4,8(1H)-dione;
3-(4-(2,2,2-trifluoroethoxy)phenyl)-2,8-bis(trifluoromethyl)-4H-pyrido[1,2-a]pyrimidin-4-one;
3-(4-(2,2-difluoroethoxy)-2-fluorophenyl)-8-methoxy-2-(trifluoromethyl)-4H-pyrido[1,2-a]pyrimidin-4-one;
3-(4-(2,2-difluoroethoxy)phenyl)-8-(methyloxy-d₃)-2-(trifluoromethyl)-4H-pyrido[1,2-a]pyrimidin-4-one;
3-(4-(2,2-difluoroethoxy)phenyl)-8-methoxy-2-(trifluoromethyl)-4H-pyrido[1,2-a]pyrimidin-4-one;
3-(4-(2,2-difluoropropoxy)phenyl)-8-methoxy-2-(trifluoromethyl)-4H-pyrido[1,2-a]pyrimidin-4-one;
3-(4-(2-fluoroethoxy)phenyl)-8-(methyloxy-d₃)-2-(trifluoromethyl)-4H-pyrido[1,2-a]pyrimidin-4-one;
3-(4-(2-fluoroethoxy)phenyl)-8-(methyloxy-d₃)-2-(trifluoromethyl)-4H-pyrimido[1,2-a]pyrimidin-4-one;
3-(4-(2-fluoroethoxy)phenyl)-8-methoxy-2-(trifluoromethyl)-4H-pyrido[1,2-a]pyrimidin-4-one;
3-(4-(2-fluoroethoxy)phenyl)-8-methoxy-2-(trifluoromethyl)-4H-pyrimido[1,2-a]pyrimidin-4-one;
3-(4-(8-methoxy-4-oxo-2-(trifluoromethyl)-4H-pyrido[1,2-a]pyrimidin-3-yl)-1H-pyrazol-1-yl)propanenitrile;
3-(4-(8-methoxy-4-oxo-2-(trifluoromethyl)-4H-pyrido[1,2-a]pyrimidin-3-yl)phenyl)propanenitrile;
3-(4-(cyclopropylmethoxy)-2-fluorophenyl)-8-methoxy-2-(trifluoromethyl)-4H-pyrido[1,2-a]pyrimidin-4-one;
3-(4-(cyclopropylmethoxy)phenyl)-8-(methyloxy-d₃)-2-(trifluoromethyl)-4H-pyrido[1,2-a]pyrimidin-4-one;
3-(4-(cyclopropylmethoxy)phenyl)-8-methoxy-2-(trifluoromethyl)-4H-pyrido[1,2-a]pyrimidin-4-one;
3-(4-(cyclopropylmethoxy)phenyl)-8-methoxy-2-(trifluoromethyl)-4H-pyrimido[1,2-a]pyrimidin-4-one;
3-(4-(difluoromethoxy)phenyl)-8-methoxy-2-(trifluoromethyl)-4H-pyrido[1,2-a]pyrimidin-4-one;
3-(5-(2,2,2-trifluoroethoxy)-2-pyridinyl)-2-(trifluoromethyl)-4H-pyrido[1,2-a]pyrimidin-4-one;
3-(5-fluoro-6-(2,2,2-trifluoroethoxy)-3-pyridinyl)-8-methoxy-2-(trifluoromethyl)-4H-pyrido[1,2-a]pyrimidin-4-one;
3-(6-(2,2,2-trifluoroethoxy)-3-pyridinyl)-2-(trifluoromethyl)-4H-pyrido[1,2-a]pyrimidin-4-one;
3-[1-(2,2,3,3,3-pentafluoropropyl)-1H-pyrazol-4-yl]-2-(trifluoromethyl)-4H,6H,7H,9H-pyrimido[2,1-c][1,4]oxazin-4-one;
3-[1-(2,2,3,3,3-pentafluoropropyl)-1H-pyrazol-4-yl]-2-(trifluoromethyl)-4H-pyrazino[1,2-a]pyrimidin-4-one;
3-[1-(2,2,3,3,3-pentafluoropropyl)-1H-pyrazol-4-yl]-2-(trifluoromethyl)-4H-pyrido[1,2-a]pyrimidin-4-one;
3-[1-(cyclopropylmethyl)-1H-pyrazol-4-yl]-8-methoxy-2-(trifluoromethyl)-4H-pyrido[1,2-a]pyrimidin-4-one;
3-[1-(cyclopropylmethyl)-1H-pyrazol-4-yl]-8-methyl-2-(trifluoromethyl)-4H-pyrimido[1,2-b]pyridazin-4-one;
3-[5-iodo-1-(2,2,3,3,3-pentafluoropropyl)-1H-1,2,3-triazol-4-yl]-8-methoxy-2-(trifluoromethyl)-4H-pyrido[1,2-a]pyrimidin-4-one;
3-{1-[(2,2-difluorocyclopropyl)methyl]-1H-pyrazol-4-yl}-7-fluoro-8-methoxy-2-(trifluoromethyl)-4H-pyrido[1,2-a]pyrimidin-4-one;
3-{1-[(2,2-difluorocyclopropyl)methyl]-1H-pyrazol-4-yl}-8-methoxy-2-(trifluoromethyl)-4H-pyrido[1,2-a]pyrimidin-4-one;
3-{1-[(2,2-difluorocyclopropyl)methyl]-1H-pyrazol-4-yl}-8-methyl-2-(trifluoromethyl)-4H-pyrimido[1,2-b]pyridazin-4-one;
3-{1-[(3,3-difluorocyclobutyl)methyl]-1H-pyrazol-4-yl}-8-methoxy-2-(trifluoromethyl)-4H-pyrido[1,2-a]pyrimidin-4-one;
3-{1-[(3,3-difluorocyclobutyl)methyl]-1H-pyrazol-4-yl}-8-methoxy-2-(trifluoromethyl)-4H-pyrimido[1,2-b]pyridazin-4-one;
3-{1-[(3,3-difluorocyclobutyl)methyl]-1H-pyrazol-4-yl}-8-methyl-2-(trifluoromethyl)-4H-pyrimido[1,2-b]pyridazin-4-one;
3-fluoro-1-methyl-7-[1-(2,2,3,3,3-pentafluoropropyl)-1H-pyrazol-4-yl]-8-(trifluoromethyl)-1H,2H,6H-[1,3]diazino[1,2-a]pyrimidine-2,6-dione;
4-oxo-3-(1-(2,2,3,3,3-pentafluoropropyl)-1H-pyrazol-4-yl)-2-(trifluoromethyl)-4H-pyrido[1,2-a]pyrimidine-8-carbonitrile;
4-oxo-3-(1-(2,2,3,3,3-pentafluoropropyl)-1H-pyrazol-4-yl)-2-(trifluoromethyl)-4H-pyrido[1,2-a]pyrimidine-8-carboxylic acid;
4-oxo-3-(4-(2,2,2-trifluoroethoxy)phenyl)-2-(trifluoromethyl)-4H-pyrido[1,2-a]pyrimidine-8-carbonitrile;
4-oxo-3-(4-(2,2,2-trifluoroethoxy)phenyl)-2-(trifluoromethyl)-4H-pyrido[1,2-a]pyrimidine-8-carboxamide;
4-oxo-3-(4-(2,2,2-trifluoroethoxy)phenyl)-2-(trifluoromethyl)-4H-pyrido[1,2-a]pyrimidine-8-carboxylic acid;
4-oxo-3-[1-(2,2,3,3,3-pentafluoropropyl)-1H-pyrazol-4-yl]-2-(trifluoromethyl)-4H-pyrido[1,2-a]pyrimidine-7-carbonitrile;
7-(1-(2,2,3,3,3-pentafluoropropyl)-1H-pyrazol-4-yl)-8-(trifluoromethyl)-2H-pyrimido[1,2-a]pyrimidine-2,6(1H)-dione;
7-(3-fluoro-4-(2,2,2-trifluoroethoxy)phenyl)-8-(trifluoromethyl)-2H-pyrimido[1,2-a]pyrimidine-2,6(1H)-dione;
7-(4-(2,2,2-trifluoroethoxy)phenyl)-8-(trifluoromethyl)-2H-pyrimido[1,2-a]pyrimidine-2,6(1H)-dione;
7-(4-(2-fluoroethoxy)phenyl)-8-(trifluoromethyl)-2H-pyrimido[1,2-a]pyrimidine-2,6(1H)-dione;
7-(methoxymethyl)-3-[1-(2,2,3,3,3-pentafluoropropyl)-1H-pyrazol-4-yl]-2-(trifluoromethyl)-4H-pyrido[1,2-a]pyrimidin-4-one;
7,8-dimethyl-2-(trifluoromethyl)-3-[1-(3,3,3-trifluoropropyl)-1H-pyrazol-4-yl]-4H-pyrimido[1,2-b]pyridazin-4-one;
7,8-dimethyl-3-[1-(2,2,3,3,3-pentafluoropropyl)-1H-pyrazol-4-yl]-2-(trifluoromethyl)-4H-pyrimido[1,2-b]pyridazin-4-one;
7,9-dimethyl-3-[1-(2,2,3,3,3-pentafluoropropyl)-1H-pyrazol-4-yl]-2-(trifluoromethyl)-4H-pyrazino[1,2-a]pyrimidin-4-one;
7-chloro-2-(trifluoromethyl)-3-[1-(3,3,3-trifluoropropyl)-1H-pyrazol-4-yl]-4H-pyrido[1,2-a]pyrimidin-4-one;
7-chloro-3-[1-(2,2,3,3,3-pentafluoropropyl)-1H-pyrazol-4-yl]-2-(trifluoromethyl)-4H-pyrazino[1,2-a]pyrimidin-4-one;
7-chloro-3-[1-(2,2,3,3,3-pentafluoropropyl)-1H-pyrazol-4-yl]-2-(trifluoromethyl)-4H-pyrido[1,2-a]pyrimidin-4-one;
7-chloro-8-methoxy-2-(trifluoromethyl)-3-[1-(3,3,3-trifluoropropyl)-1H-pyrazol-4-yl]-4H-pyrido[1,2-a]pyrimidin-4-one;
7-chloro-8-methoxy-3-[1-(2,2,3,3,3-pentafluoropropyl)-1H-pyrazol-4-yl]-2-(trifluoromethyl)-4H-[1,3]diazino[1,2-a]pyrimidin-4-one;
7-chloro-8-methoxy-3-[1-(2,2,3,3,3-pentafluoropropyl)-1H-pyrazol-4-yl]-2-(trifluoromethyl)-4H-pyrido[1,2-a]pyrimidin-4-one;
7-chloro-8-methyl-2-(trifluoromethyl)-3-[1-(3,3,3-trifluoropropyl)-1H-pyrazol-4-yl]-4H-pyrido[1,2-a]pyrimidin-4-one;
7-chloro-8-methyl-3-[1-(2,2,3,3,3-pentafluoropropyl)-1H-pyrazol-4-yl]-2-(trifluoromethyl)-4H-pyrido[1,2-a]pyrimidin-4-one;
7-chloro-8-methyl-3-[4-(2,2,2-trifluoroethoxy)phenyl]-2-(trifluoromethyl)-4H-pyrido[1,2-a]pyrimidin-4-one;
7-cyclopropyl-2-(trifluoromethyl)-3-[1-(3,3,3-trifluoropropyl)-1H-pyrazol-4-yl]-4H-pyrido[1,2-a]pyrimidin-4-one;
7-cyclopropyl-3-[1-(2,2,3,3,3-pentafluoropropyl)-1H-pyrazol-4-yl]-2-(trifluoromethyl)-4H-pyrido[1,2-a]pyrimidin-4-one;
7-fluoro-2-(trifluoromethyl)-3-[1-(3,3,3-trifluoropropyl)-1H-pyrazol-4-yl]-4H-pyrido[1,2-a]pyrimidin-4-one;
7-fluoro-3-[1-(2,2,3,3,3-pentafluoropropyl)-1H-pyrazol-4-yl]-2-(trifluoromethyl)-4H-pyrido[1,2-a]pyrimidin-4-one;
7-fluoro-8-hydroxy-3-[1-(2,2,3,3,3-pentafluoropropyl)-1H-pyrazol-4-yl]-2-(trifluoromethyl)-4H-pyrido[1,2-a]pyrimidin-4-one;
7-fluoro-8-methoxy-2-(trifluoromethyl)-3-[1-(3,3,3-trifluoropropyl)-1H-pyrazol-4-yl]-4H-pyrido[1,2-a]pyrimidin-4-one;
7-fluoro-8-methoxy-3-[1-(2,2,3,3,3-pentafluoropropyl)-1H-pyrazol-4-yl]-2-(trifluoromethyl)-4H-[1,3]diazino[1,2-a]pyrimidin-4-one;
7-fluoro-8-methoxy-3-[1-(2,2,3,3,3-pentafluoropropyl)-1H-pyrazol-4-yl]-2-(trifluoromethyl)-4H-pyrido[1,2-a]pyrimidin-4-one;
7-fluoro-8-methoxy-3-[4-(2,2,2-trifluoroethoxy)phenyl]-2-(trifluoromethyl)-4H-pyrido[1,2-a]pyrimidin-4-one;
7-fluoro-8-methyl-3-[1-(2,2,3,3,3-pentafluoropropyl)-1H-pyrazol-4-yl]-2-(trifluoromethyl)-4H-pyrido[1,2-a]pyrimidin-4-one;
7-methoxy-3-[1-(2,2,3,3,3-pentafluoropropyl)-1H-pyrazol-4-yl]-2-(trifluoromethyl)-4H-[1,3]diazino[1,2-a]pyrimidin-4-one;
7-methoxy-3-[1-(2,2,3,3,3-pentafluoropropyl)-1H-pyrazol-4-yl]-2-(trifluoromethyl)-4H-pyrazino[1,2-a]pyrimidin-4-one;
7-methoxy-3-[1-(2,2,3,3,3-pentafluoropropyl)-1H-pyrazol-4-yl]-2-(trifluoromethyl)-4H-pyrido[1,2-a]pyrimidin-4-one;
7-methyl-2-(trifluoromethyl)-3-[1-(3,3,3-trifluoropropyl)-1H-pyrazol-4-yl]-4H-pyrimido[1,2-b]pyridazin-4-one;
7-methyl-3-[1-(2,2,3,3,3-pentafluoropropyl)-1H-pyrazol-4-yl]-2-(trifluoromethyl)-4H-[1,3]diazino[1,2-a]pyrimidin-4-one;
7-methyl-3-[1-(2,2,3,3,3-pentafluoropropyl)-1H-pyrazol-4-yl]-2-(trifluoromethyl)-4H-pyrazino[1,2-a]pyrimidin-4-one;
7-methyl-3-[1-(2,2,3,3,3-pentafluoropropyl)-1H-pyrazol-4-yl]-2-(trifluoromethyl)-4H-pyrido[1,2-a]pyrimidin-4-one;
7-methyl-3-[1-(2,2,3,3,3-pentafluoropropyl)-1H-pyrazol-4-yl]-2-(trifluoromethyl)-4H-pyrimido[1,2-b]pyridazin-4-one;
8-((1R)-1-hydroxyethyl)-3-(1-(2,2,3,3,3-pentafluoropropyl)-1H-pyrazol-4-yl)-2-(trifluoromethyl)-4H-pyrido[1,2-a]pyrimidin-4-one;
8-((1R)-1-hydroxyethyl)-3-(4-(2,2,2-trifluoroethoxy)phenyl)-2-(trifluoromethyl)-4H-pyrido[1,2-a]pyrimidin-4-one;
8-((1S)-1-hydroxyethyl)-3-(1-(2,2,3,3,3-pentafluoropropyl)-1H-pyrazol-4-yl)-2-(trifluoromethyl)-4H-pyrido[1,2-a]pyrimidin-4-one;
8-((1S)-1-hydroxyethyl)-3-(4-(2,2,2-trifluoroethoxy)phenyl)-2-(trifluoromethyl)-4H-pyrido[1,2-a]pyrimidin-4-one;
8-((dimethylamino)methyl)-3-(1-(2,2,3,3,3-pentafluoropropyl)-1H-pyrazol-4-yl)-2-(trifluoromethyl)-4H-pyrido[1,2-a]pyrimidin-4-one;
8-(methyloxy-d₃)-2-(trifluoromethyl)-3-(1-(3,3,3-trifluoropropyl)-1H-pyrazol-4-yl)-4H-pyrido[1,2-a]pyrimidin-4-one;
8-((methylsulfanyl)methoxy)-3-(4-(2,2,2-trifluoroethoxy)phenyl)-2-(trifluoromethyl)-4H-pyrido[1,2-a]pyrimidin-4-one;
8-((R)-ethylsulfinyl)-3-(4-(2,2,2-trifluoroethoxy)phenyl)-2-(trifluoromethyl)-4H-pyrido[1,2-a]pyrimidin-4-one;
8-((R)-methylsulfinyl)-3-(1-(2,2,3,3,3-pentafluoropropyl)-1H-pyrazol-4-yl)-2-(trifluoromethyl)-4H-pyrido[1,2-a]pyrimidin-4-one;
8-((R)-methylsulfinyl)-3-(4-(2,2,2-trifluoroethoxy)phenyl)-2-(trifluoromethyl)-4H-pyrido[1,2-a]pyrimidin-4-one;
8-((S)-ethylsulfinyl)-3-(4-(2,2,2-trifluoroethoxy)phenyl)-2-(trifluoromethyl)-4H-pyrido[1,2-a]pyrimidin-4-one;
8-((S)-methylsulfinyl)-3-(1-(2,2,3,3,3-pentafluoropropyl)-1H-pyrazol-4-yl)-2-(trifluoromethyl)-4H-pyrido[1,2-a]pyrimidin-4-one;
8-((S)-methylsulfinyl)-3-(4-(2,2,2-trifluoroethoxy)phenyl)-2-(trifluoromethyl)-4H-pyrido[1,2-a]pyrimidin-4-one;
8-(1,3-oxazol-2-yl)-3-(4-(2,2,2-trifluoroethoxy)phenyl)-2-(trifluoromethyl)-4H-pyrido[1,2-a]pyrimidin-4-one;
8-(1-hydroxyethyl)-3-(1-(2,2,3,3,3-pentafluoropropyl)-1H-pyrazol-4-yl)-2-(trifluoromethyl)-4H-pyrido[1,2-a]pyrimidin-4-one;
8-(1-hydroxyethyl)-3-(4-(2,2,2-trifluoroethoxy)phenyl)-2-(trifluoromethyl)-4H-pyrido[1,2-a]pyrimidin-4-one;
8-(2-hydroxypropan-2-yl)-3-[1-(2,2,3,3,3-pentafluoropropyl)-1H-pyrazol-4-yl]-2-(trifluoromethyl)-4H-pyrido[1,2-a]pyrimidin-4-one;
8-(2-methyl-2-oxetanyl)-3-(4-(2,2,2-trifluoroethoxy)phenyl)-2-(trifluoromethyl)-4H-pyrido[1,2-a]pyrimidin-4-one;
8-(2-propanyl)-3-(4-(2,2,2-trifluoroethoxy)phenyl)-2-(trifluoromethyl)-4H-pyrido[1,2-a]pyrimidin-4-one;
8-(3-azetidinyl)-3-(4-(2,2,2-trifluoroethoxy)phenyl)-2-(trifluoromethyl)-4H-pyrido[1,2-a]pyrimidin-4-one;
8-(aminomethyl)-3-(1-(2,2,3,3,3-pentafluoropropyl)-1H-pyrazol-4-yl)-2-(trifluoromethyl)-4H-pyrido[1,2-a]pyrimidin-4-one;
8-(1-azetidinyl)-3-(1-(2,2,3,3,3-pentafluoropropyl)-1H-pyrazol-4-yl)-2-(trifluoromethyl)-4H-pyrido[1,2-a]pyrimidin-4-one;
8-(chloromethoxy)-3-[1-(2,2,3,3,3-pentafluoropropyl)-1H-pyrazol-4-yl]-2-(trifluoromethyl)-4H-pyrido[1,2-a]pyrimidin-4-one;
8-(chloromethoxy)-3-[1-(2,2,3,3,3-pentafluoropropyl)-1H-pyrazol-4-yl]-2-(trifluoromethyl)-4H-pyrimido[1,2-b]pyridazin-4-one;
8-(difluoromethoxy)-3-(4-(2,2,2-trifluoroethoxy)phenyl)-2-(trifluoromethyl)-4H-pyrido[1,2-a]pyrimidin-4-one;
8-(difluoromethyl)-3-(4-(2,2,2-trifluoroethoxy)phenyl)-2-(trifluoromethyl)-4H-pyrido[1,2-a]pyrimidin-4-one;
8-(dimethylamino)-3-(1-(2,2,3,3,3-pentafluoropropyl)-1H-pyrazol-4-yl)-2-(trifluoromethyl)-4H-pyrido[1,2-a]pyrimidin-4-one;
8-(dimethylamino)-3-(4-(2,2,2-trifluoroethoxy)phenyl)-2-(trifluoromethyl)-4H-pyrido[1,2-a]pyrimidin-4-one;
8-(ethylsulfinyl)-3-(4-(2,2,2-trifluoroethoxy)phenyl)-2-(trifluoromethyl)-4H-pyrido[1,2-a]pyrimidin-4-one;
8-(fluoromethoxy)-2-(trifluoromethyl)-3-(1-(3,3,3-trifluoropropyl)-1H-pyrazol-4-yl)-4H-pyrido[1,2-a]pyrimidin-4-one;
8-(fluoromethoxy)-3-(4-(2,2,2-trifluoroethoxy)phenyl)-2-(trifluoromethyl)-4H-pyrido[1,2-a]pyrimidin-4-one;
8-(fluoromethoxy)-3-[1-(2,2,3,3,3-pentafluoropropyl)-1H-pyrazol-4-yl]-2-(trifluoromethyl)-4H-pyrido[1,2-a]pyrimidin-4-one;
8-(fluoromethoxy)-3-[1-(2,2,3,3,3-pentafluoropropyl)-1H-pyrazol-4-yl]-2-(trifluoromethyl)-4H-pyrimido[1,2-b]pyridazin-4-one;
8-(fluoromethyl)-3-(1-(2,2,3,3,3-pentafluoropropyl)-1H-pyrazol-4-yl)-2-(trifluoromethyl)-4H-pyrido[1,2-a]pyrimidin-4-one;
8-(fluoromethyl)-3-(4-(2,2,2-trifluoroethoxy)phenyl)-2-(trifluoromethyl)-4H-pyrido[1,2-a]pyrimidin-4-one;
8-(hydroxymethyl)-3-(1-(2,2,3,3,3-pentafluoropropyl)-1H-pyrazol-4-yl)-2-(trifluoromethyl)-4H-pyrido[1,2-a]pyrimidin-4-one;
8-(hydroxymethyl)-3-(4-(2,2,2-trifluoroethoxy)phenyl)-2-(trifluoromethyl)-4H-pyrido[1,2-a]pyrimidin-4-one;
8-(methoxymethyl)-3-[1-(2,2,3,3,3-pentafluoropropyl)-1H-pyrazol-4-yl]-2-(trifluoromethyl)-4H-pyrido[1,2-a]pyrimidin-4-one;
8-(methylamino)-3-(1-(2,2,3,3,3-pentafluoropropyl)-1H-pyrazol-4-yl)-2-(trifluoromethyl)-4H-pyrido[1,2-a]pyrimidin-4-one;
8-(methylamino)-3-(4-(2,2,2-trifluoroethoxy)phenyl)-2-(trifluoromethyl)-4H-pyrido[1,2-a]pyrimidin-4-one;
8-(methylamino)-3-(4-(2,2,2-trifluoroethoxy)phenyl)-2-(trifluoromethyl)-4H-pyrimido[1,2-a]pyrimidin-4-one;
8-(methyl-d₃)-3-(4-(2,2,2-trifluoroethoxy)phenyl)-2-(trifluoromethyl)-4H-pyrido[1,2-a]pyrimidin-4-one;
8-(methyloxy-d₃)-3-(1-(2,2,3,3,3-pentafluoropropyl)-1H-pyrazol-4-yl)-2-(trifluoromethyl)-4H-pyrido[1,2-a]pyrimidin-4-one;
8-(methyloxy-d₃)-3-(1-(2,2,3,3,3-pentafluoropropyl)-1H-pyrazol-4-yl)-2-(trifluoromethyl)-4H-pyrimido[1,2-a]pyrimidin-4-one;
8-(methyloxy-d₃)-3-(1-(4,4,4-trifluorobutyl)-1H-pyrazol-4-yl)-2-(trifluoromethyl)-4H-pyrido[1,2-a]pyrimidin-4-one;
8-(methyloxy-d₃)-3-(4-(2,2,2-trifluoroethoxy)phenyl)-2-(trifluoromethyl)-4H-pyrido[1,2-a]pyrimidin-4-one;
8-(methyloxy-d₃)-3-(4-(2,2,2-trifluoroethoxy)phenyl)-2-(trifluoromethyl)-4H-pyrimido[1,2-a]pyrimidin-4-one;
8-(methyloxy-d₃)-3-(6-(2,2,2-trifluoroethoxy)-3-pyridinyl)-2-(trifluoromethyl)-4H-pyrido[1,2-a]pyrimidin-4-one;
8-(methylsulfanyl)-3-(1-(2,2,3,3,3-pentafluoropropyl)-1H-pyrazol-4-yl)-2-(trifluoromethyl)-4H-pyrido[1,2-a]pyrimidin-4-one;
8-(methylsulfanyl)-3-(4-(2,2,2-trifluoroethoxy)phenyl)-2-(trifluoromethyl)-4H-pyrido[1,2-a]pyrimidin-4-one;
8-(methylsulfinyl)-3-(1-(2,2,3,3,3-pentafluoropropyl)-1H-pyrazol-4-yl)-2-(trifluoromethyl)-4H-pyrido[1,2-a]pyrimidin-4-one;
8-(methylsulfinyl)-3-(4-(2,2,2-trifluoroethoxy)phenyl)-2-(trifluoromethyl)-4H-pyrido[1,2-a]pyrimidin-4-one;
8-(methylsulfonyl)-3-(4-(2,2,2-trifluoroethoxy)phenyl)-2-(trifluoromethyl)-4H-pyrido[1,2-a]pyrimidin-4-one;
8-acetyl-3-(4-(2,2,2-trifluoroethoxy)phenyl)-2-(trifluoromethyl)-4H-pyrido[1,2-a]pyrimidin-4-one;
8-amino-3-(1-(2,2,3,3,3-pentafluoropropyl)-1H-pyrazol-4-yl)-2-(trifluoromethyl)-4H-pyrido[1,2-a]pyrimidin-4-one;
8-amino-3-(4-(2,2,2-trifluoroethoxy)phenyl)-2-(trifluoromethyl)-4H-pyrido[1,2-a]pyrimidin-4-one;
8-amino-3-(4-(2,2,2-trifluoroethoxy)phenyl)-2-(trifluoromethyl)-4H-pyrimido[1,2-a]pyrimidin-4-one;
8-chloro-3-(1-(2,2,3,3,3-pentafluoropropyl)-1H-pyrazol-4-yl)-2-(trifluoromethyl)-4H-pyrido[1,2-a]pyrimidin-4-one;
8-chloro-3-(4-(2,2,2-trifluoroethoxy)phenyl)-2-(trifluoromethyl)-4H-pyrido[1,2-a]pyrimidin-4-one;
8-cyclopropyl-3-(4-(2,2,2-trifluoroethoxy)phenyl)-2-(trifluoromethyl)-4H-pyrido[1,2-a]pyrimidin-4-one;
8-cyclopropyl-3-(1-(2,2,3,3,3-pentafluoropropyl)-1H-pyrazol-4-yl)-2-(trifluoromethyl)-4H-pyrido[1,2-a]pyrimidin-4-one;
8-ethenyl-3-(4-(2,2,2-trifluoroethoxy)phenyl)-2-(trifluoromethyl)-4H-pyrido[1,2-a]pyrimidin-4-one;
8-ethoxy-3-(4-(2,2,2-trifluoroethoxy)phenyl)-2-(trifluoromethyl)-4H-pyrido[1,2-a]pyrimidin-4-one;
8-ethyl-3-(4-(2,2,2-trifluoroethoxy)phenyl)-2-(trifluoromethyl)-4H-pyrido[1,2-a]pyrimidin-4-one;
8-fluoro-3-(4-(2,2,2-trifluoroethoxy)phenyl)-2-(trifluoromethyl)-4H-pyrido[1,2-a]pyrimidin-4-one;
8-hydroxy-3-[1-(2,2,3,3,3-pentafluoropropyl)-1H-pyrazol-4-yl]-2-(trifluoromethyl)-4H-pyrimido[1,2-b]pyridazin-4-one;
8-methoxy-2-(trifluoromethyl)-3-(1-(3-(trifluoromethyl)phenyl)-1H-pyrazol-4-yl)-4H-pyrido[1,2-a]pyrimidin-4-one;
8-methoxy-2-(trifluoromethyl)-3-(1-(3,3,3-trifluoropropyl)-1H-imidazol-4-yl)-4H-pyrido[1,2-a]pyrimidin-4-one;
8-methoxy-2-(trifluoromethyl)-3-(1-(3,3,3-trifluoropropyl)-1H-pyrazol-4-yl)-4H-pyrido[1,2-a]pyrimidin-4-one;
8-methoxy-2-(trifluoromethyl)-3-(1-(3,3,3-trifluoropropyl)-1H-pyrazol-4-yl)-4H-pyrimido[1,2-a]pyrimidin-4-one;
8-methoxy-2-(trifluoromethyl)-3-(1-(4-(trifluoromethyl)phenyl)-1H-pyrazol-4-yl)-4H-pyrido[1,2-a]pyrimidin-4-one;
8-methoxy-2-(trifluoromethyl)-3-(4-(3,3,3-trifluoropropyl)phenyl)-4H-pyrido[1,2-a]pyrimidin-4-one;
8-methoxy-2-(trifluoromethyl)-3-[1-(3,3,3-trifluoropropyl)-1H-pyrazol-4-yl]-4H-pyrimido[1,2-b]pyridazin-4-one;
8-methoxy-2-(trifluoromethyl)-3-[3-(3,3,3-trifluoropropyl)-1,2-oxazol-5-yl]-4H-pyrido[1,2-a]pyrimidin-4-one;
8-methoxy-2-(trifluoromethyl)-3-[4-(3,3,3-trifluoropropyl)-1H-imidazol-1-yl]-4H-pyrido[1,2-a]pyrimidin-4-one;
8-methoxy-2-(trifluoromethyl)-3-[5-(3,3,3-trifluoropropyl)-1,3-thiazol-2-yl]-4H-pyrido[1,2-a]pyrimidin-4-one;
8-methoxy-2-methyl-3-(4-(2,2,2-trifluoroethoxy)phenyl)-4H-pyrido[1,2-a]pyrimidin-4-one;
8-methoxy-3-(1-(2,2,2-trifluoroethyl)-1H-pyrazol-4-yl)-2-(trifluoromethyl)-4H-pyrido[1,2-a]pyrimidin-4-one;
8-methoxy-3-(1-(2,2,3,3,3-pentafluoropropyl)-1H-pyrazol-4-yl)-2-(trifluoromethyl)-4H-pyrido[1,2-a]pyrimidin-4-one;
8-methoxy-3-(1-(2,2,3,3,3-pentafluoropropyl)-1H-pyrazol-4-yl)-2-(trifluoromethyl)-4H-pyrimido[1,2-a]pyrimidin-4-one;
8-methoxy-3-(1-(4,4,4-trifluorobutyl)-1H-pyrazol-4-yl)-2-(trifluoromethyl)-4H-pyrido[1,2-a]pyrimidin-4-one;
8-methoxy-3-(1-(4,4,4-trifluorobutyl)-1H-pyrazol-4-yl)-2-(trifluoromethyl)-4H-pyrimido[1,2-a]pyrimidin-4-one;
8-methoxy-3-(1-phenyl-1H-pyrazol-3-yl)-2-(trifluoromethyl)-4H-pyrido[1,2-a]pyrimidin-4-one;
8-methoxy-3-(1-phenyl-1H-pyrazol-4-yl)-2-(trifluoromethyl)-4H-pyrido[1,2-a]pyrimidin-4-one;
8-methoxy-3-(1-propyl-1H-pyrazol-4-yl)-2-(trifluoromethyl)-4H-pyrido[1,2-a]pyrimidin-4-one;
8-methoxy-3-(2-methyl-4-(2,2,2-trifluoroethoxy)phenyl)-2-(trifluoromethyl)-4H-pyrido[1,2-a]pyrimidin-4-one;
8-methoxy-3-(2-phenyl-1,3-oxazol-5-yl)-2-(trifluoromethyl)-4H-pyrido[1,2-a]pyrimidin-4-one;
8-methoxy-3-(3-phenyl-1,2-oxazol-5-yl)-2-(trifluoromethyl)-4H-pyrido[1,2-a]pyrimidin-4-one;
8-methoxy-3-(4-(2,2,2-trifluoroethoxy)-2-(trifluoromethyl)phenyl)-2-(trifluoromethyl)-4H-pyrido[1,2-a]pyrimidin-4-one;
8-methoxy-3-(4-(2,2,2-trifluoroethoxy)phenyl)-2-(trifluoromethyl)-4H-pyrido[1,2-a]pyrimidin-4-one;
8-methoxy-3-(4-(2,2,2-trifluoroethoxy)phenyl)-2-(trifluoromethyl)-4H-pyrimido[1,2-a]pyrimidin-4-one;
8-methoxy-3-(4-(2,2,3,3,3-pentafluoropropoxy)phenyl)-2-(trifluoromethyl)-4H-pyrido[1,2-a]pyrimidin-4-one;
8-methoxy-3-(4-(2,2,3,3-tetrafluoropropoxy)phenyl)-2-(trifluoromethyl)-4H-pyrido[1,2-a]pyrimidin-4-one;
8-methoxy-3-(4-(trifluoromethoxy)phenyl)-2-(trifluoromethyl)-4H-pyrido[1,2-a]pyrimidin-4-one;
8-methoxy-3-(4-(trifluoromethoxy)phenyl)-2-(trifluoromethyl)-4H-pyrimido[1,2-a]pyrimidin-4-one;
8-methoxy-3-(4-propylphenyl)-2-(trifluoromethyl)-4H-pyrido[1,2-a]pyrimidin-4-one;
8-methoxy-3-(5-propyl-1,2-oxazol-3-yl)-2-(trifluoromethyl)-4H-pyrido[1,2-a]pyrimidin-4-one;
8-methoxy-3-(6-(2,2,2-trifluoroethoxy)-3-pyridinyl)-2-(trifluoromethyl)-4H-pyrido[1,2-a]pyrimidin-4-one;
8-methoxy-3-(6-(2,2,2-trifluoroethoxy)-3-pyridinyl)-2-(trifluoromethyl)-4H-pyrimido[1,2-a]pyrimidin-4-one;
8-methoxy-3-(6-propyl-3-pyridinyl)-2-(trifluoromethyl)-4H-pyrido[1,2-a]pyrimidin-4-one;
8-methoxy-3-[1-(2,2,3,3,3-pentafluoropropyl)-1H-1,2,3-triazol-4-yl]-2-(trifluoromethyl)-4H-pyrido[1,2-a]pyrimidin-4-one;
8-methoxy-3-[1-(2,2,3,3,3-pentafluoropropyl)-1H-pyrazol-3-yl]-2-(trifluoromethyl)-4H-pyrido[1,2-a]pyrimidin-4-one;
8-methoxy-3-[1-(2,2,3,3,3-pentafluoropropyl)-1H-pyrazol-4-yl]-2-(trifluoromethyl)-4H-[1,3]diazino[1,6-a]pyrimidin-4-one;
8-methoxy-3-[1-(2,2,3,3,3-pentafluoropropyl)-1H-pyrazol-4-yl]-2-(trifluoromethyl)-4H-pyrimido[1,2-b]pyridazin-4-one;
8-methoxy-3-[2-(2,2,2-trifluoroethoxy)-1,3-thiazol-5-yl]-2-(trifluoromethyl)-4H-pyrido[1,2-a]pyrimidin-4-one;
8-methoxy-3-[2-(2,2,2-trifluoroethoxy)pyrimidin-5-yl]-2-(trifluoromethyl)-4H-[1,3]diazino[1,2-a]pyrimidin-4-one;
8-methoxy-3-[2-(2,2,2-trifluoroethoxy)pyrimidin-5-yl]-2-(trifluoromethyl)-4H-pyrido[1,2-a]pyrimidin-4-one;
8-methoxy-3-[2-(2,2,2-trifluoroethoxy)pyrimidin-5-yl]-2-(trifluoromethyl)-4H-pyrimido[1,2-b]pyridazin-4-one;
8-methoxy-3-[2-(2,2,3,3,3-pentafluoropropoxy)pyrimidin-5-yl]-2-(trifluoromethyl)-4H-pyrido[1,2-a]pyrimidin-4-one;
8-methoxy-3-[3-(2,2,3,3,3-pentafluoropropyl)-1,2-oxazol-5-yl]-2-(trifluoromethyl)-4H-pyrido[1,2-a]pyrimidin-4-one;
8-methoxy-3-[4-(2,2,2-trifluoroethoxy)-1,3-thiazol-2-yl]-2-(trifluoromethyl)-4H-pyrido[1,2-a]pyrimidin-4-one;
8-methoxy-3-[5-(2,2,2-trifluoroethoxy)-1,3-thiazol-2-yl]-2-(trifluoromethyl)-4H-pyrido[1,2-a]pyrimidin-4-one;
8-methoxy-3-1{1-[(oxetan-3-yl)methyl]-1H-pyrazol-4-yl}-2-(trifluoromethyl)-4H-pyrido[1,2-a]pyrimidin-4-one;
8-methoxy-6-methyl-3-[1-(2,2,3,3,3-pentafluoropropyl)-1H-pyrazol-4-yl]-2-(trifluoromethyl)-4H-pyrido[1,2-a]pyrimidin-4-one;
8-methyl-2-(trifluoromethyl)-3-[1-(3,3,3-trifluoropropyl)-1H-pyrazol-4-yl]-4H-pyrimido[1,2-b]pyridazin-4-one;
8-methyl-2-(trifluoromethyl)-3-[5-(3,3,3-trifluoropropyl)-1,2,4-oxadiazol-3-yl]-4H-pyrido[1,2-a]pyrimidin-4-one;
8-methyl-3-(4-(2,2,2-trifluoroethoxy)phenyl)-2-(trifluoromethyl)-4H-pyrido[1,2-a]pyrimidin-4-one;
8-methyl-3-[1-(2,2,3,3,3-pentafluoropropyl)-1H-pyrazol-4-yl]-2-(trifluoromethyl)-4H,6H,7H,8H,9H-pyrimido[1,2-a]pyrazin-4-one;
8-methyl-3-[1-(2,2,3,3,3-pentafluoropropyl)-1H-pyrazol-4-yl]-2-(trifluoromethyl)-4H-pyrimido[1,2-b]pyridazin-4-one;
8-methyl-3-[2-(2,2,2-trifluoroethoxy)pyrimidin-5-yl]-2-(trifluoromethyl)-4H-pyrimido[1,2-b]pyridazin-4-one;
8-methyl-3-[4-(2,2,2-trifluoroethoxy)phenyl]-2-(trifluoromethyl)-4H-pyrimido[1,2-b]pyridazin-4-one;
9-chloro-8-methoxy-3-[1-(2,2,3,3,3-pentafluoropropyl)-1H-pyrazol-4-yl]-2-(trifluoromethyl)-4H-pyrido[1,2-a]pyrimidin-4-one;
9-fluoro-8-methoxy-3-[1-(2,2,3,3,3-pentafluoropropyl)-1H-pyrazol-4-yl]-2-(trifluoromethyl)-4H-pyrido[1,2-a]pyrimidin-4-one;
9-methyl-3-[1-(2,2,3,3,3-pentafluoropropyl)-1H-pyrazol-4-yl]-2-(trifluoromethyl)-4H-pyrazino[1,2-a]pyrimidin-4-one;
methyl 4-oxo-3-(4-(2,2,2-trifluoroethoxy)phenyl)-2-(trifluoromethyl)-4H-pyrido[1,2-a]pyrimidine-8-carboxylate;
methyl(4-oxo-3-(1-(2,2,3,3,3-pentafluoropropyl)-1H-pyrazol-4-yl)-2-(trifluoromethyl)-4H-pyrido[1,2-a]pyrimidin-8-yl)carbamyl fluoride;
N-(4-oxo-3-(1-(2,2,3,3,3-pentafluoropropyl)-1H-pyrazol-4-yl)-2-(trifluoromethyl)-4H-pyrido[1,2-a]pyrimidin-8-yl)acetamide;
N-(4-oxo-3-(4-(2,2,2-trifluoroethoxy)phenyl)-2-(trifluoromethyl)-4H-pyrido[1,2-a]pyrimidin-8-yl)acetamide;
N,N-dimethyl-4-oxo-3-(4-(2,2,2-trifluoroethoxy)phenyl)-2-(trifluoromethyl)-4H-pyrido[1,2-a]pyrimidine-8-carboxamide;
N-ethyl-4-oxo-3-(4-(2,2,2-trifluoroethoxy)phenyl)-2-(trifluoromethyl)-4H-pyrido[1,2-a]pyrimidine-8-carboxamide;
N-methyl-4-oxo-3-(1-(2,2,3,3,3-pentafluoropropyl)-1H-pyrazol-4-yl)-2-(trifluoromethyl)-4H-pyrido[1,2-a]pyrimidine-8-carboxamide; or
N-methyl-4-oxo-3-(4-(2,2,2-trifluoroethoxy)phenyl)-2-(trifluoromethyl)-4H-pyrido[1,2-a]pyrimidine-8-carboxamide; or
a pharmaceutically acceptable salt of any of the foregoing.

26. (canceled)

27. A method of measuring a ratio of arachidonic acid (AA) and dihomo-gamma-linolenic acid (DGLA) in a subject;
wherein the AA is isotopically labeled and the DGLA is isotopically labeled;
wherein the ratio of AA to DGLA is measured by administering a dose of labeled DGLA to the subject and thereafter measuring a ratio of labeled AA to labeled DGLA.

28. The method of claim 27, wherein the labeled DGLA comprises DGLA that is isotopically enriched with ¹³C.

29. (canceled)

30. The method of claim 27, wherein the labeled DGLA includes labels at one or more or all of the carbons indicated with an “*” below:

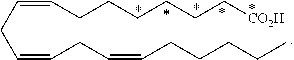 [SMILES: CCCCC/C=C\C/C=C\C/C=C\CCCCCCC(=O)O]

31. (canceled)

32. A compound represented by the following structure: 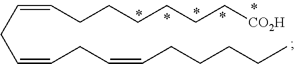 [SMILES: CCCCC/C=C\C/C=C\C/C=C\CCCCCCC(=O)O]
where
each “*” symbol indicates a position that may be isotopically enriched with ¹³C; and
at least one “*” position is isotopically enriched with ¹³C.

33. The compound of claim 32, wherein at least five “*” positions are isotopically enriched with ¹³C.

34. (canceled)

35. A method of manufacturing a compound represented by the following structure: 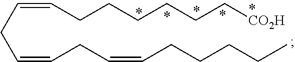 [SMILES: CCCCC/C=C\C/C=C\C/C=C\CCCCCCC(=O)O]
the method comprising reacting the following compound 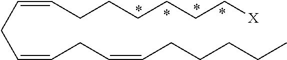
with KC*N;
where
each “*” symbol indicates a position that may be isotopically enriched with ¹³C and at least one “*” position is isotopically enriched with ¹³C; and
wherein X is a halogen.
